(19) United States
(12) Patent Application Publication
(10) Pub. No.: US 2026/0325713 A1
(43) Pub. Date: Oct. 22, 2026

(54) BIOFABRICATION AND BIOPROCESSING OF MICROBIAL CELLULOSE FOR BIOTEXTILES AND TISSUE ENGINEERING

(71) Applicant: THE TRUSTEES OF COLUMBIA UNIVERSITY IN THE CITY OF NEW YORK, New York, NY (US)

(72) Inventors: Helen H. LU, New York, NY (US); Romare M. ANTROBUS, New York, NY (US); Theanne N. SCHIROS, New York, NY (US)

(73) Assignee: THE TRUSTEES OF COLUMBIA UNIVERSITY IN THE CITY OF NEW YORK, New York, NY (US)

(21) Appl. No.: 19/693,013

(22) Filed: May 29, 2026

Related U.S. Application Data
(63) Continuation of application No. PCT/US2024/057970, filed on Nov. 29, 2024.
(60) Provisional application No. 63/604,801, filed on Nov. 30, 2023.

(51) Int. Cl. A61L 27/20; A61L 27/04

(52) U.S. Cl. CPC A61L 27/20; A61L 27/042; A61L 27/047

(57) ABSTRACT

A microbial cellulose material is provided comprising an acid, a metal ion, and microbial cellulose. The acid can be a polyphenol. The metal ion can be selected from a group consisting of iron ion (Fe²⁺), iron ion (Fe³⁺), copper ion (Cu²⁺), aluminum ion (Al³⁺), magnesium ion (Mg²⁺), zinc ion (Zn²⁺), nickel ion (Ni²⁺), germanium ion (Ge⁴⁺), titanium (Ti⁴⁺), molybdenum (Mo⁶⁺) and tungsten ion (W⁶⁺). The microbial cellulose can be a bioplasticized microbial cellulose.

This application claims the benefit of U.S. Provisional Application No. 63/604,801, filed Nov. 30, 2023, the content of which is hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### STATEMENT REGARDING FEDERALLY SPONSORED RESEARCH OR DEVELOPMENT

This invention was made with government support under AR073529 awarded by the National Institutes of Health, and 2011738 awarded by the National Science Foundation. The government has certain rights in the invention.

### BACKGROUND

Various green bioprocessing approaches for enhancing the mechanical performance of microbial cellulose (“mCellulose”) can be explored, for example, for generating textiles. Similar to most naturally derived biopolymers, as-fabricated mCellulose characteristically has enhanced hygroscopicity, resulting in instable mechanical properties. Traditional techniques aim to reduce water adsorption and improve mechanical stability of conventional textiles by utilizing synthetic plasticizers, which introduce human health and ecological toxicity, as well as compromise biodegradability.

Towards the design of biomaterial scaffolds for guiding cell-mediated tissue regeneration, matrices may meet key design criteria that not only implement biomimetic approaches without eliciting an inflammatory response by immune cells, but also minimize human health and environmental impacts. Though microbial cellulose is an extracellularly produced polysaccharide, the resulting biofilm encapsulates its inherent gram-negative bacterial manufacturers, preventing further use as a biomaterial for biomedical applications.

### BRIEF SUMMARY

A microbial cellulose material can be provided, comprising:
an acid;
a metal ion; and
microbial cellulose, wherein
   the acid is a polyphenol,
   the metal ion is selected from a group consisting of iron ion (Fe²⁺), iron ion (Fe³⁺), copper ion (Cu²⁺), aluminum ion (Al³⁺), magnesium ion (Mg²⁺), zinc ion (Zn²⁺), nickel ion (Ni²⁺), germanium ion (Ge⁴⁺), titanium (Ti⁴⁺), molybdenum (Mo⁶⁺) and tungsten ion (W⁶⁺), and the microbial cellulose is a bioplasticized microbial cellulose.

It may also be desirable to provide a process of preparing a microbial cellulose material, wherein the process comprises the steps of:
(a) preparing a bioplasticized microbial cellulose;
(b) bathing the bioplasticized microbial cellulose from step (a) in a polyphenol solution;
(c) bathing the bioplasticized microbial cellulose from step (b) in a metal ion solution; and
(d) optionally adding a base to the metal ion solution in step (c).

Further, a process of purifying a microbial cellulose film may be provided, comprising:
(a) bathing the microbial cellulose film in cellulase solution;
(b) bathing the microbial cellulose film from step (a) in an alcohol solution;
(c) bathing the microbial cellulose film from step (b) in a basic solution; and
(d) applying a high-pressure steam to the microbial cellulose film in step (c).

### BRIEF DESCRIPTION OF THE DRAWINGS

FIG. 1 Bioprocessing of Microbial Cellulose. Microbial cellulose (“mCellulose”) biofabricated by G. xylinus can be bioprocessed by utilizing green chemistry approaches that include sorbitol, glycerol, and tannic acid-iron complexation.

FIG. 2 Mass and Thickness Measurements of Bioprocessed Microbial Cellulose. Biofabricated microbial cellulose was bioprocessed with either (a) sorbitol (1, 2, and 5% w/v), (b) glycerol (1, 2, and 5% v/v), (c) glycerol (2% v/v) with tannic acid (1 and 2% w/v)(n=10, \hat{ }p<0.05, between groups), or (d) tannic acid-Fe(III) complexation (pH 1.99, 5.73, and 9.14)(n=6, \hat{ }p<0.05, between groups).

FIG. 3 Sorbitol-Plasticized Microbial Cellulose Characterization. Biofabricated microbial cellulose was bioprocessed with sorbitol (1, 2, and 5% w/v) via a facile immersion technique. (a) Nanofiber morphology was continually observed on microbial cellulose surfaces before and after sorbitol treatment(s) (SEM, n=3). (b) Evaluation of microbial cellulose tensile mechanical properties before and after sorbitol treatment show that ductility has a positive correlation with plasticizer concentration, while Young's Modulus significantly decreases. Toughness properties reveal anti-plasticizing behavior at 5% w/v (n=5, \hat{ }p<0.05, between groups). (c) The presence of sorbitol was confirmed by FTIR, with increased peak absorption corresponding to greater hydroxyl groups at higher concentrations at 3345 cm⁻¹ and 856 cm⁻¹. Lateral Order Index and Total Crystallinity Index decreased with increasing sorbitol concentration treatment (n=5, \hat{ }p<0.05, between groups).

FIG. 4 Glycerol-Plasticized Microbial Cellulose Characterization. Biofabricated microbial cellulose was bioprocessed with glycerol (1, 2, and 5% v/v) via a facile immersion technique. (a) Inherent nanofibrous morphology became less visible with increasing glycerol treatment from 1 to 5% v/v, as a result of plasticizer's homogenous coverage on mCellulose (SEM, n=3). (b) Evaluation of microbial cellulose tensile mechanical properties before and after glycerol treatment show that ductility has a positive correlation with plasticizer concentration, while Young's Modulus significantly decreases. Toughness properties reveal anti-plasticizing behavior at 5% v/v (n=5, \hat{ }≤p0.05, between groups). (c) The presence of glycerol was confirmed by FTIR, with increased peak absorption corresponding to greater hydroxyl groups at higher concentrations at 3345 cm⁻¹. Lateral Order Index and Total Crystallinity Index decreased after glycerol treatment (n=5, \hat{ }p<0.05, between groups).

FIG. 5 Evaluation of mCellulose Mechanical Properties After Glycerol-Tannic Acid Plasticization. Biofabricated microbial cellulose was consecutively bioprocessed with 2% v/v glycerol and tannic acid (1, 2% w/v) via a facile immersion technique. Evaluation of microbial cellulose tensile mechanical properties show that combined polyol-tannin treatments have similar Young's Modulus, tensile strength, toughness, and ductility to 2% v/v glycerol treated samples. (n=5, \hat{ }p<0.05, between groups). (n=5, \hat{ }p<0.05, between groups).

FIG. 6 Morphological and Tensile Mechanical Characterization of Tannic Acid-Iron Complexed mCellulose. (a) Biofabricated microbial cellulose was crosslinked with tannic acid (0.5% w/v) and FeCl₃ (0.5% w/v) at pH 1.99, pH 5.73, pH 9.14 (Scale bar: 5 mm). Inherent nanofibrous morphology became less visible with tannic acid treatment, as a result of tannin's coverage on mCellulose. Immersion of TA-treated mCellulose into FeCl₃ under pH conditions of 1.99 and 5.73 exhibited the formation of TA-Fe nanoparticles adhered to nanofibers. Further pH modification to 9.14 displayed randomly oriented nanofibers similar to as-fabricated MC 3 (SEM, n=3). (b) Evaluation of microbial cellulose tensile mechanical properties before and after tannic acid-iron complexation showed that coordination at pH 5.73 and 9.14 significantly increased toughness and ductility, while maintaining strength compared to as-fabricated groups. Further, complexation at pH 9.14 significantly reduced ultimate tensile stress, ductility, and toughness (n=6, \hat{ }p<0.05, between groups).

FIG. 7 Crystallinity of Tannic Acid-Iron Complexed mCellulose. Biofabricated microbial cellulose was crosslinked with tannic acid (0.5% w/v) and FeCl₃ (0.5% w/v) at pH 1.99, pH 5.73, pH 9.14. (a) Evaluation of mCellulose crystal structure before and after tannic acid-iron complexation at pH 5.73 confirmed the maintenance of cellulose I Bragg peaks at 2θ=14.6°, 16.9°, and 22.8°, corresponding to the (1-10), (110), and (200) planes. (b) The crystallinity index of pH 5.73 slightly increased compared to as-fabricated mCellulose (XRD, n=5, \hat{ }p<0.05, between groups).

FIG. 8 Viscoelastic Characterization of Tannic Acid-Iron Complexed mCellulose. Biofabricated microbial cellulose was crosslinked with tannic acid (0.5% w/v) and FeCl₃ (0.5% w/v) at pH 1.99, pH 5.73, pH 9.14. Complexation occurring at pH 1.99 significantly increased both elastic (storage) and shear modulus compared to as-fabricated mCellulose, while a further increase in pH to 5.73 and 9.14 continually reduced moduli (n=5, \hat{ }p<0.05, between groups).

FIG. 9 Chemical Structure and Elemental Characterization of Tannic Acid-Iron Complexed mCellulose. Biofabricated microbial cellulose was crosslinked with tannic acid (0.5% w/v) and FeCl₃ (0.5% w/v) at pH 1.99, pH 5.73, pH 9.14. (a) Tannin presence in tannic acid-treated microbial cellulose was confirmed by FTIR, with peaks corresponding to O—H groups, benzene ring C—C, and C—O/C—C at 3345 cm⁻¹, 1312 cm⁻¹, and 1205 cm⁻¹, respectively. (b) Complexation with iron at pH 1.99 further increased peak intensity around 3345 cm⁻¹, representing greater hydroxyl groups. However, an increase in pH to 5.73 and 9.14 decreased peak intensity of the hydroxyl broad band between 3600-3000 cm⁻¹, increased the relative absorbance of methyl stretching (1428 cm⁻¹), aromatic C—C groups (1312 cm⁻¹), and C—O/C—C groups (1205 cm⁻). (c) Lateral Order Index and Total Crystallinity Index increased after tannin-iron complexation treatment (n=5, \hat{ }p<0.05, between groups). (d-f) Iron presence in tannic acid-treated microbial cellulose was confirmed by EDXA and observed with spectral maps.

FIG. 10 Antibacterial Properties of Tannic Acid-Iron Complexed mCellulose. Biofabricated microbial cellulose was crosslinked with tannic acid (0.5% w/v) and FeCl₃ (0.5% w/v) at pH 1.99, pH 5.73, pH 9.14. A Kirby-Bauer disk diffusion assay revealed that as-fabricated samples exhibited no inhibition zones after 24 hours of culture, while dried mCellulose films only treated with 0.5% w/v tannic acid (MCTA) significantly increased inhibition zones, comparable to control disks containing 2% penicillin-streptomycin. Further, complexation with iron significantly decreased the inhibition zone, with no statistical difference of inhibition diameter between TA-Fe samples maintained below pH 3, and modified to pH 5-6 (n=5, \hat{ }p<0.05, between groups).

FIG. 11: Life Cycle Impact Assessment of Optimal Bioprocessing Strategies for mCellulose. Cradle-to-gate life cycle impact assessment for bioprocessing 150 cm² of microbial cellulose utilizing either 2% w/v sorbitol, 1% v/v glycerol, or tannic acid-Fe(III) complexation. (a) Comparison of human health and environmental impact categories for different bioprocessing strategies. Human health toxicity includes impacts accumulating from carcinogenics and hormonally active agents, while ecological damage is associated with acidification, ecotoxicity, eutrophication, global warming, and ozone depletion. (b) Breakdown of the total impact by category, in which the greatest impact is highlighted by percentage of the total impacts. Legend: Glycerol₁=Vegetable Oil, Glycerol₂=Epichlorohydrin with 10% NaOH, Glycerol₃=Epichlorohydrin Waste Oil, TA₁-Fe=Polyphenol Waste Biomass Extraction, TA₂-Fe=Hot Water Extraction, DOP=D1(2-ethylhexyl) phthalate.

FIG. 12 Mechanical Performance of Microbial Cellulose and Conventional Textiles. (a) Stress-strain profiles of microbial cellulose before (MC 1, MC 2, MC 3) and after optimal bioprocessing with either 2% w/v sorbitol, 1% v/v glycerol, or tannic acid-Fe(III) complexation. (b) Comparison of mCellulose tensile mechanical properties before and after optimal bioprocessing to conventional textiles (n=5, \hat{ }p<0.05, between groups).

FIG. 13 Biofabrication and Purification of Microbial Cellulose. (a) Under specific aerobic culturing conditions G. xylinus biofabricate unaligned cellulose nanofibrils (10-100 nm diameter) that coagulate into a three-dimensional layered biofilm (>98% water) at the air-culture media interface. (b) G. xylinus extracellularly-secrete microbial cellulose and become encapsulated by cellulose during biofabrication. (c) Microbial cellulose biofilms were purified by sequential immersion of biofilms into cellulase, 70% ethanol, and 0.1 M sodium hydroxide, followed by autoclaving. Scale bar=5 mm.

FIG. 14 Effect of Purification on G. xylinus Viability and Endotoxin Content. Microbial cellulose biofilms were purified by sequential immersion of biofilms into cellulase (1 hour), 70% ethanol (24 hours), and 0.1 M sodium hydroxide (24 hours), followed by autoclaving (250 °C, 30 minutes). (a) Quantitative comparison of overlapping fluorescence showed a significant increase (95%) in cell death once biofilms were immersed into 70% EtOH, in which low cell viability was maintained after NaOH and autoclaving treatment. (b) Significantly less DNA per mg of cellulose was measured in purified microbial cellulose compared to as-fabricated biofilms (n=5). (c) Endotoxin content was observed to significantly decrease after subsequent purification with EtOH and NaOH, with no significant differences with as-fabricated groups (n=5, \hat{ }p<0.05 between groups).

FIG. 15 Purified Microbial Cellulose Characterization. (a) Nanofiber morphology was continually observed on microbial cellulose surfaces before and after purification treatment(s) (SEM, n=3). Similar fiber diameters are observed between as-fabricated and purified groups (n=50 fibers/group). (b) The maintenance of representative microbial cellulose chemical groups (O—H, 3345 cm⁻¹; C—H, 2898 cm⁻, —OH, 1633 cm⁻¹; C—O—H/C—C/C—O—C, 1058 cm⁻¹) after purification was confirmed with FTIR-ATR (n=5). (c) Elemental composition of as-fabricated and purified microbial cellulose determined by energy dispersive x-ray analysis indicated significant decreases in relative sodium and phosphorus mass percentage after sterilization (EDXA, n=5). (d) Mechanical properties of mCellulose and pmCellulose indicated that elastic modulus, ultimate tensile strength (UTS), and yield strength decreased significantly after purification, while ductility was maintained (n=8, \hat{ }p<0.05 between groups).

FIG. 16 Evaluating mCellulose Biocompatibility Through Macrophage Culture. THP-1 macrophages cultured on purified microbial cellulose scaffolds maintained a naïve M0 phenotype, demonstrating that it does not induce a pro- or anti-inflammatory response from the cells. (a) Purified microbial cellulose supported cell viability throughout 4 days of culture (n=2/group). (b) Cell number was observed to be significantly lower on microbial cellulose scaffolds compared to unaligned PLGA:PCL microfibers by day 4 (seeding density: 2×10⁵ cells/cm², n=5). (c) Naïve M0 macrophages were observed to maintain a rounded morphology with small protrusions attaching to microbial cellulose matrices similar to unaligned PLGA:PCL cultures (n=2/group). (d) Comparable concentrations of TNF-α, IL-1β, IL-6, IL-10, and IL-1 RA per cell were found between purified microbial cellulose and PLGA:PCL matrices, indicating the biomaterial's biocompatibility (n=4, \hat{ }p<0.05 between groups).

FIG. 17 Evaluating mCellulose Biocompatibility Through Macrophage Culture. (a) As-fabricated and 70% EtOH treated microbial cellulose supported THP-1 macrophage cell viability throughout 2 days of culture (n=2/group). G. xylinus cells were observed to interact with macrophages on microbial cellulose surfaces. (b) Comparable total concentrations of TNF-α, IL-1β, IL-6, IL-10, and IL-IRA were found between as-fabricated, purified microbial cellulose, and PLGA:PCL matrices (n=4, \hat{ }p<0.05 between groups).

FIG. 18 Evaluating mCellulose Biocompatibility Through Fibroblast Culture. (a)-(b) Purified microbial cellulose scaffolds supported ACL fibroblast cell viability and proliferation throughout 14 days of culture. Fibroblasts were observed to maintain a rounded morphology at early timepoints when seeded on microbial cellulose matrices, and elongated by day 14. Cell number was observed to be significantly lower on microbial cellulose scaffolds compared to unaligned PLGA:PCL microfibers at every timepoint (seeding density: 3×10⁴ cells/cm², n=5, \hat{ }p<0.05 between groups, *p<0.05 between timepoints).

FIG. 19 Bioreactor Study Design. Schematic indicating study groups, differentiation and polarization schedule, as well as, mechanical loading (1% strain, 1 Hz, 90 minutes twice daily) schedule.

FIG. 20 Aligned vs. Unaligned: Electrospinning Synthetic PLGA:PCL Microfibers. The polymer blend 5:1 polylactide-co-glycolide (PLGA, 50:50): polycaprolactone (PCL) was electrospun with acetic acid into unaligned and aligned microfibers. (a) Scanning electron micrographs of unaligned and aligned PLGA:PCL microfibers. Similar fiber diameters are observed between orientations (n=50 fibers/group). (b) Microfiber matrices (5×4 cm) are cultured in a custom bioreactor apparatus with load applied via a stepper motor and linear actuator. The entire apparatus is housed within a cell culture incubator throughout the entire culturing period. (c) Mechanical properties of unaligned and aligned microfibers indicated that elastic modulus, ultimate tensile strength (UTS), and yield strength increased significantly with alignment, while ductility significantly decreased (n=8, \hat{ }p<0.05 between groups).

FIG. 21 Effects of PLGA:PCL Matrix Alignment on Macrophage Attachment. (a)-(b) Cell attachment was observed to be significantly lower on aligned PLGA:PCL microfibers compared to unaligned matrices, with only 20.3% of macrophages adhering after differentiation. (c) Significantly greater population doublings were measured when naïve macrophages were cultured on loaded aligned microfibers by day 2 (seeding density: 2×10⁵ cells/cm², n=5, \hat{ }p<0.05 between groups).

FIG. 22 Effects of Matrix Alignment, Chemical Induction, and Mechanical Stimulation on Macrophage Viability and Proliferation. Both aligned and unaligned PLGA:PCL microfibers supported cell viability and proliferation in unloaded and loaded environments. On unaligned matrices, cell density was observed to be comparable for naïve macrophages after two days of loading. When inducted into pro- or anti-inflammatory phenotypes, cell number was found to be significantly lower compared to unloaded naïve macrophages counterparts. Further, loading pro-inflammatory induced cells on unaligned meshes showed no effect on cell number. On aligned microfibers, naïve macrophages significantly increased in cell number (seeding density: 2×10⁵ cells/cm², n=5, \hat{ }p<0.05 between groups).

FIG. 23 Effects of Matrix Alignment, Chemical Induction, and Mechanical Stimulation on Macrophage Morphology. Distinct differences in macrophage morphology was observed based upon fiber alignment, chemical induction, and mechanical loading cues. Naïve M0 macrophages were observed to maintain a rounded morphology with small protrusions attaching to unaligned PLGA:PCL, in which loading had no effect on cell shape. Upon culturing on aligned matrices, M0 macrophages elongated in the direction of the fibers, however, mechanical stimulation returned cells to a rounded morphology. When induced to pro- and anti-inflammatory phenotypes cells became larger in size, and clustered with each other on unaligned and aligned scaffolds. While no differences were observed for M1 cells on unaligned matrices after mechanical loading, cells on loaded aligned scaffolds had distinctively different morphology with increased spreading and large presence of cellular debris potentially resembling matrix deposition (Magnification: 1.5 kx).

FIG. 24 Effects of Matrix Alignment, Chemical Induction, and Mechanical Stimulation on Macrophage Morphology. Distinct differences in macrophage morphology was observed based upon fiber alignment, chemical induction, and mechanical loading cues. Naïve M0 macrophages were observed to maintain a rounded morphology with small protrusions attaching to unaligned PLGA:PCL, in which loading had no effect on cell shape. Upon culturing on aligned matrices, M0 macrophages elongated in the direction of the fibers, however, mechanical stimulation returned cells to a rounded morphology. When induced to pro- and anti-inflammatory phenotypes cells became larger in size, and clustered with each other on unaligned and aligned scaffolds. While no differences were observed for M1 cells on unaligned matrices after mechanical loading, cells on loaded aligned scaffolds had distinctively different morphology with increased spreading and large presence of cellular debris potentially resembling matrix deposition (Magnification: 3 kx).

FIG. 25 Effects of Matrix Alignment, Chemical Induction, and Mechanical Stimulation on Collagen Matrix Deposition. Both aligned and unaligned PLGA:PCL microfibers supported macrophage collagen production in unloaded and loaded environments. On Day 4, a significant increase in collagen per cell was measured on aligned PLGA:PCL matrices after 2 days of mechanical loading macrophages that were induced to a pro-inflammatory phenotype (n=5, \hat{ }p<0.05 between groups).

FIG. 26 Effects of Matrix Alignment, Chemical Induction, and Mechanical Stimulation on Glycosaminoglycan Matrix Deposition. Both aligned and unaligned PLGA:PCL microfibers supported macrophage GAG production in unloaded and loaded environments. On Day 4, a significant increase in GAG per cell was measured on unaligned PLGA:PCL matrices after 2 days of culturing macrophages that were induced to a pro-inflammatory phenotype (n=5, \hat{ }p<0.05 between groups).

FIG. 27 Effects of PLGA:PCL Matrix Alignment on Macrophage Response: Secretion of Pro- and Anti-inflammatory Cytokines. Upon completion of macrophage differentiation on Day 2, significantly greater (a) Pro-inflammatory secretion of IL-6, and IL-1β per cell, and (b) Anti-inflammatory secretion of IL-10, IL-1 RA, and TARC per cell was measured from unaligned PLGA:PCL matrices compared to aligned microfibers (n=4, \hat{ }p<0.05 between groups).

FIG. 28 Effects of Matrix Alignment and Chemical Induction on Macrophage Response: Secretion of Pro- and Anti-inflammatory Cytokines. The effects of matrix alignment and polarization on macrophage response were determined by measuring cytokine secretion from naïve M0, and pro- (M1) and anti- (M2) inflammatory induced cells cultured upon aligned and unaligned PLGA:PCL microfibers for four days. (a) On unaligned fibers, concentrations of TNF-α, IL-1β, and IL-6 per cell were significantly higher in the M1 polarized group, while IL-10, IL-1RA, and TARC per cell were significantly increased in the M2 polarized group. (b) On aligned meshes significant increases in TNF-α, IL-1β, and IL-6 per cell were measured only in the pro-inflammatory M1 group. Induction to an anti- (M2) inflammatory state on aligned microfibers elevated IL-10 per cell concentrations. However, significantly more TARC per cell, an anti-inflammatory marker, was observed to be secreted from M1-induced cells on aligned PLGA:PCL (n=4, \hat{ }p<0.05 between groups).

FIG. 29 Effects of Matrix Alignment on Pro-Inflammatory Macrophage Response: Secretion of Pro- and Anti-inflammatory Cytokines. Upon culturing pro-inflammatory macrophages (M1) for 4 days on unaligned and aligned PLGA:PCL microfibers, (a) no differences were observed in pro-inflammatory secretion of TNF-α, IL-6, and IL-1β per cell. (b) On aligned matrices M1 Macrophages secreted significantly greater anti-inflammatory cytokines IL-10, IL-1RA, and TARC per cell compared to unaligned substrates (n=4, \hat{ }p<0.05 between groups).

FIG. 30 Effects of Matrix Alignment and Mechanical Stimulation on Macrophage Response: Secretion of Pro- and Anti-inflammatory Cytokines. The effects of matrix alignment and mechanical loading on macrophage response were determined by measuring cytokine secretion from naïve M0 cells cultured upon aligned and unaligned PLGA:PCL microfibers for two days. (a) On unaligned meshes, a significant decrease in concentration for TNF-α, IL-1β, IL-6, and TARC per cell was measured by day 4, with no observed differences with loading. (b) On aligned microfibers, a decrease in TNF-α and TARC per cell was found, with observed differences with loading. Levels of IL-1β per cell were significantly greater from loaded aligned meshes compared to unloaded groups, albeit comparable to day 2 concentrations (n=4, \hat{ }p<0.05 between groups, *p<0.05 between timepoints).

FIG. 31 Effects of Matrix Alignment, Chemical Induction, and Mechanical Stimulation on Macrophage Response: Secretion of Pro- and Anti-inflammatory Cytokines. The effects of matrix alignment, polarization, and mechanical loading on macrophage response were determined by measuring cytokine secretion from naïve M0, and pro- inflammatory (M1) induced cells cultured upon aligned and unaligned PLGA:PCL microfibers for four days. (a) On unaligned PLGA:PCL meshes, subsequent loading of M1 induced cells significantly reduced the secretion of M1 markers TNF-α, IL-1β, and IL-6 per cell, and significantly increased M2 secretion of IL-10, IL-IRA, and TARC per cell. (b) Conversely, on aligned microfibers loading of M1 induced cells through two days of culture significantly increased pro-inflammatory TNF-α per cell, while decreasing anti-inflammatory IL-10 and TARC per cell (n=4, \hat{ }p<0.05 between loading, *p<0.05 between induction).

FIG. 32 Biofabrication of Microbial Cellulose. All sugars supported the assembly of hydrated mCellulose biofilms, at the air-media interface, with cylindrical geometries and a light-brown color, as a result of the shape of the culturing vessel and the inherent HS medium, respectively. Purification and lyophilization of mCellulose resulted in dried, white biofilms.

FIG. 33 Culturing of G. xylinus for 100 hours with Glucose, Fructose, Sucrose, Mannitol, and Xylitol-based Medium. (a) All carbon sources supported microbial growth for over 100 hours. (b) Lag time of glucose, sucrose, mannitol, and xylitol groups were independently comparable between concentrations, while significantly greater lag times are observed in 8% fructose-based cultures compared to 2%. (c) Glucose-based cultures reduced microbial doubling time, irrespective to concentration. Microbial doubling time increased in fructose, sucrose, mannitol, and xylitol groups, while the effect was significantly mitigated in 8% sucrose and mannitol medium. Xylitol further measured greater doubling times at 2%, with a significant increase at 8%. (d) pH significantly decreased in glucose-based cultures over 96 hours compared to other carbon sources. (n=5, \hat{ }p<0.05 between groups, *p<0.05 between timepoints).

FIG. 34 Biofabrication of Microbial Cellulose: Glucose, fructose, sucrose, mannitol, and xylitol-based medium support cellulose production by G. xylinus over two weeks of culture. (a) Glucose: Significantly greater cellulose yield is measured in glucose groups over time by day 14, independent of concentration. (b) Fructose: Cellulose bioproduction significantly increased in the 8% group overtime, with significant differences between concentrations at day 14. (c) Sucrose: Significantly greater cellulose yield is observed in all groups by day 14, while significant differences are measured between concentrations at day 7 and 14. (d) Mannitol: Cellulose bioproduction significantly increased in 2% and 8% groups overtime, with significant differences between concentrations at day 14. (e) Xylitol: Cellulose yield significantly increased by day 14 in 8% groups, with no significant differences between concentrations. (n=4, \hat{ }p<0.05 between groups, *p<0.05 between timepoints). Scale bar: 1 cm.

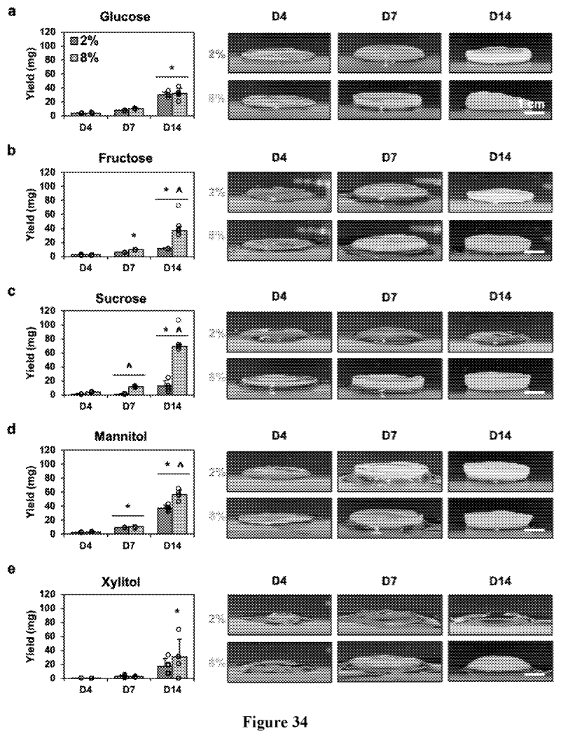

FIG. 35 Biofabrication of Microbial Cellulose Glucose, fructose, sucrose, mannitol, and xylitol-based medium support cellulose production by G. xylinus over two weeks of culture. Cellulose mass was greater in as-fabricated hydrated state compared to dried forms, and the swelling ratio was similar between all groups (>96% water), although minute differences were observed by day 14. (n=4, \hat{ }<0.05 between groups, *p<0.05 between timepoints).

FIG. 36 Nanofibril Morphology of Cellulose Produced From G. xylinus Cultures Utilizing Glucose, Fructose, Sucrose, Mannitol, and Xylitol-based Medium. Comparable (a) nanofibril morphology and (b) diameter (ø~44 nm) are observed as a function of carbon source and concentration (n=100 fibers/group). (c) Glucose and xylitol-based groups measure greater surface porosity compared to other carbon sources (n=6, \hat{ }<0.05, between groups).

FIG. 37 Tensile Mechanical Properties of Cellulose Produced from G. xylinus Cultures Utilizing Glucose, fructose, Sucrose, Mannitol, and Xylitol-based Medium. (a) and (b) mechanical properties demonstrate significantly increased ultimate tensile strength (UTS), yield strength, and elastic modulus in 8% groups, while ductility between all carbon sources and concentrations were comparable (n=8, \hat{ }<0.05 between groups).

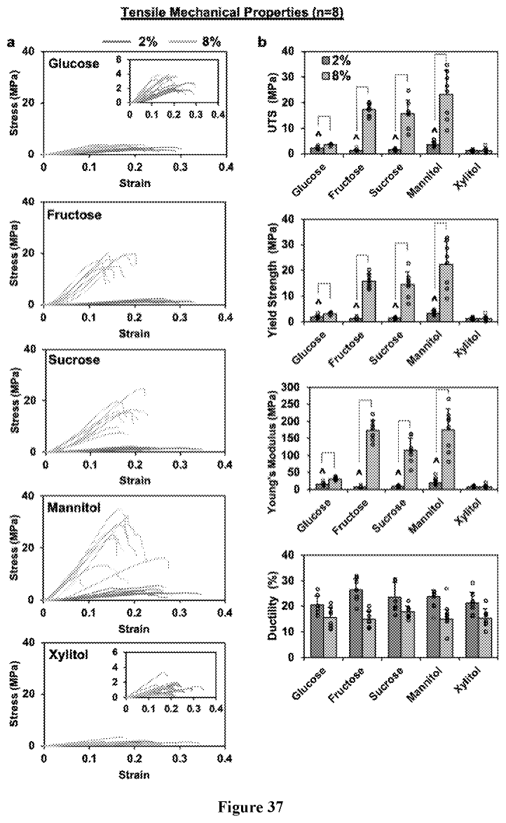

FIG. 38 Chemical Characterization of Microbial Cellulose (a) Chemical fingerprints of cellulose Iα and Iβ were confirmed at 750 cm-1 and 710 cm-1, respectively, revealed the ability for G. xylinus to synthesize both allomorphs independent to carbon source and concentration. (b) G. xylinus produced 34 to 44% Iα, however, differences in allomorph percentage were found between carbon sources. In 2% w/v groups, the fraction of Iα was significantly greater when synthesized with fructose, sucrose, and mannitol than glucose and xylitol cultures, though similar trends were not seen at 8% w/v (n=5, \hat{ }<0.05, between 2% groups).

FIG. 39 Crystal Structure of Cellulose Produced From G. xylinus Cultures Utilizing Glucose, Fructose, Sucrose, Mannitol, and Xylitol-based Medium. (a) Cellulose crystal structure is maintained in all carbon source groups, indicated by the presence of (1-10), (110), and (200) Bragg cellulose I peaks in x-ray diffraction (XRD). (b) Crystallinity of cellulose fabricated from 2% and 8% glucose, fructose, sucrose, and mannitol cultures were comparable (~85%), while crystallinity decreased significantly in xylitol groups (n=5, \hat{ }<0.05 between groups).

FIG. 40 Crystal Structure of Cellulose Produced From G. xylinus Cultures Utilizing Glucose, Fructose, Sucrose, Mannitol, and Xylitol-based Medium. (a) No significant increases of crystal sizes are observed between glucose, fructose, and sucrose groups at 2% and 8%. However, crystal size of cellulose significantly increased from 2% to 8% (5.20±0.25 vs 5.85±0.03 nm) in mannitol groups, while the opposite trend was observed in xylitol-based cultures (5.71±0.33 vs 5.23±0.24 nm; n=5, \hat{ }<0.05 between groups). (b) Cellulose biofabricated from glucose, fructose, and sucrose is rich in Iα, in which an increase of concentration from 2% to 8% reduced the Z score (n=5). While cellulose produced from 2% mannitol was predominantly rich with Iβ allomorphs, an increase in concentration to 8% produced Iα rich allomorphs. Alternately, 2% xylitol groups were rich with Iα allomorphs, however, 8% cultures produced cellulose rich in Iβ.

FIG. 41 Life Cycle Impact Assessment of Carbon Sources for Biofabricating Microbial Cellulose. Cradle-to-gate life cycle impact assessment for utilizing 0.9 g of sugar (glucose, fructose, sucrose, mannitol, and xylitol) for biofabricating microbial cellulose. (a) Comparison of human health and environmental impact categories for different sugars. Human health toxicity includes impacts accumulating from carcinogenics and hormonally active agents, while ecological damage is associated with acidification, ecotoxicity, eutrophication, global warming, and ozone depletion. (b) Breakdown of the total impact by category, in which the greatest impact is highlighted by percentage of the total impacts.

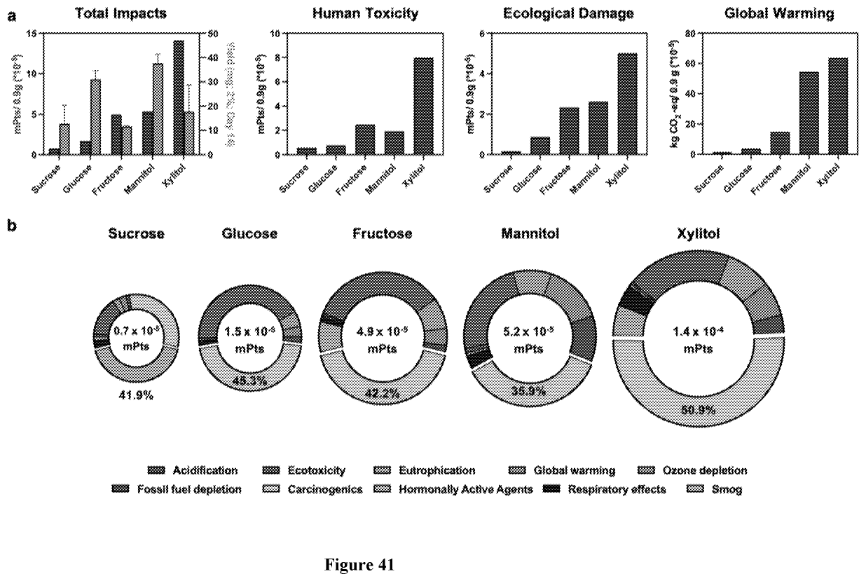

FIG. 42 Characterization of Microbial Cellulose Physical Properties: (A) Nanofiber morphology (SEM); (B) tensile properties; and (C) crystallinity (XRD) of microbial cellulose (MC), MC treated by lecithin (LT) and/or smoke tanning (LTS, S). Experimental groups (MC1 and MC2, S1 and S2) were produced from the same biosynthesis and processing conditions; the variation reflects the influence of MC hygroscopicity on mechanical properties which is overcome with lecithin tanning (LT).

FIG. 43 Physical Properties of As-fabricated and Treated Microbial Cellulose Compared to Cotton and Leather. Fiber diameter (n=100), degree of swelling (n=5), water contact angle (WCA, n=5), mechanical properties, including Young's Modulus, toughness, and ultimate tensile strength (UTS) (n=4), and crystallinity index and lattice (d-)spacing (n=5) obtained from X-ray diffraction for as-fabricated (MC) and processed (LT, S, LTS) mCellulose. (\hat{ }p<0.05, between groups).

FIG. 44 Elemental Characterization of As-fabricated and Treated mCellulose Elemental analysis with energy dispersive x-ray analysis (EDXA) indicated significantly increased amounts of phosphorus on LT surfaces compared to as-fabricated MC. (n=3, \hat{ }<0.05, between groups).

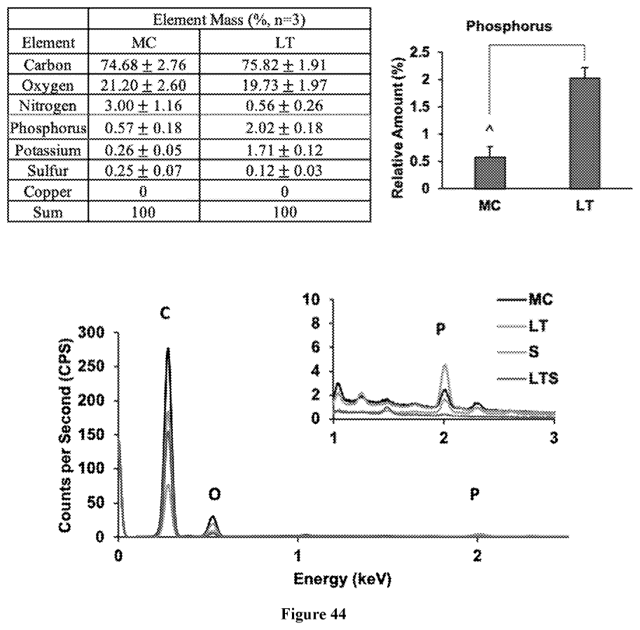

FIG. 45 Flame Retardance of mCellulose (A) Under a 2054 oC flame, lecithin-tanned (LT) microbial nanocellulose does not ignite and deflects the flame from the surface, while an aluminum brazing rod promptly melts under the flame. After repeated exposure until char formation is observed (charred), LT biotextiles remain intact under the surface ash (end). (B) The nanofiber morphology and layered microstructure of as-fabricated MC biosynthesized to sufficient thickness (0.9 mm (25 mm) in dehydrated (hydrated) state) also demonstrates flame retardance, evidence by resistance to material lost to combustion. (C) SEM micrograph showing surface morphology before (top) and in charred region after (bottom) flame testing; the torched region of the latter shows a morphology typical of a flame-retardant material. (D) Thermogravimetric analysis (TGA) curves (left) and data (right) of as-fabricated (MC) and treated (LT, S, LTS) biotextiles under nitrogen atmosphere shows the phospholipid treatment directs the combustion chemistry toward ash formation over the glucosan formation to promote flame retardance.

FIG. 46 Atomic and Molecular-level Chemical Analysis (A) X-ray photoelectron spectroscopy (XPS) and (B) Fourier Transform Infrared (FTIR) spectra of mCellulose before (MC) and after lecithin tanning (LT). Inset: schematic depiction of proposed crosslinking mechanism with phosphate, methylene and carboxyl group bridges (left to right) for LT.

FIG. 47 Lateral Order Index (LOI) and Total Crystallinity Index (TCI) obtained from FTIR. The data shows a slight increase in crystallinity, and order due to formation of chemical cross-links (phosphates, methylene and carboxyl groups) with lecithin tanning (LT compared to as-fabricated MC) throughout the bulk of the biomaterial.

FIG. 48 Environmental performance of glucose, fructose, sucrose, mannitol, and xylitol used as carbon sources for mCellulose biofabrication.

FIG. 49 Culturing of G. xylinus for 100 hours with glucose, fructose, sucrose, mannitol, and xylitol-based medium. (a) All carbon sources supported microbial growth for over 100 hours. (b) Lag time of glucose, sucrose, mannitol, and xylitol groups were independently comparable between concentrations, while significantly greater lag times are observed in 8% fructose-based cultures compared to 2%. (c) Glucose-based cultures reduced microbial doubling time, irrespective to concentration. Microbial doubling time increased in fructose, sucrose, mannitol, and xylitol groups, while the effect was significantly mitigated in 8% sucrose and mannitol medium. (d) Significant increase in cellulose yield was observed with glucose cultures by day 4. Higher carbon concentrations in sucrose and mannitol cultures significantly increased yield. (n=5, \hat{ }<0.05 between concentration, *p<0.05 between sugar types).

FIG. 50 Biofabrication of microbial cellulose: Glucose, fructose, sucrose, mannitol, and xylitolbased medium support cellulose production by G. xylinus for 14 days. (a) Significant increase in yield was observed with higher carbon concentration cultures, with the exception of glucose and xylitol-based cultures. (b) pH significantly decreased in glucose-based cultures over 96 hours compared to other carbon sources (n=5, \hat{ }<0.05 between groups, *p<0.05 between timepoints). (c) Respective images of microbial cellulose pellicles biofabricated from glucose, fructose, sucrose, mannitol, and xylitol-based medium.

FIG. 51 Nanofibril morphology of microbial cellulose. Comparable (a) nanofibril morphology and (b) diameter (φ~44 nm) are observed as a function of carbon source and concentration (n=100 fibers/group). (c) Glucose and xylitol-based groups measure greater surface porosity compared to other carbon sources (n=6, \hat{ }<0.05, between groups).

FIG. 52 Characterization of microbial cellulose physical properties. (a) Mechanical properties demonstrate significantly increased ultimate tensile strength (UTS), yield strength, and elastic modulus in 8% groups, while ductility between all carbon sources and concentrations were comparable (n=8, \hat{ }<0.05 between groups).

FIG. 53 Characterization of microbial cellulose structural properties. (a) The representative cellulose chemical groups (O—H, 3300 cm−1; C—H, 2900 cm−1; C═O, 1726 cm−1; C—O—C, 1183 cm−1; C—O, 1057 cm−1; C—O, 1035 cm−1) are observed, regardless of carbon source and concentration via Fourier transform infrared spectroscopy in attenuated total reflectance mode (FTIR-ATR, n=5). (b) Cellulose crystal structure is maintained in all carbon source groups, indicated by the presence of (1-10), (110), and (200) Bragg cellulose I peaks in x-ray diffraction (XRD). (c) Crystallinity of cellulose fabricated from 2% and 8% glucose, fructose, sucrose, and mannitol cultures were comparable (~85%), while crystallinity decreased significantly in xylitol groups (n=5, \hat{ }<0.05 between groups). (d) No significant increases of crystal sizes are observed between glucose, fructose, and sucrose groups at 2% and 8%. However, crystal size of cellulose significantly increased from 2% to 8% (5.20±0.25 vs 5.85±0.03 nm) in mnannitol groups, while the opposite trend was observed in xylitol-based cultures (5.71±0.33 vs 5.23±0.24 m; n=5, \hat{ }<0.05 between groups). (e) Cellulose biofabricated from glucose, fructose, and sucrose is rich in I_{α}, in which an increase of concentration from 2% to 8% reduced the Z score (n=5). While cellulose produced from 2% mannitol was predominantly rich with Iβ allomorphs, an increase in concentration to 8% produced Iα rich allomorphs. Alternately, 2% xylitol groups were rich with Iα allomorphs, however, 8% cultures produced cellulose rich in Iβ.

FIG. 54 Impact of carbon source of glucose, fructose, sucrose, mannitol and xylitol.

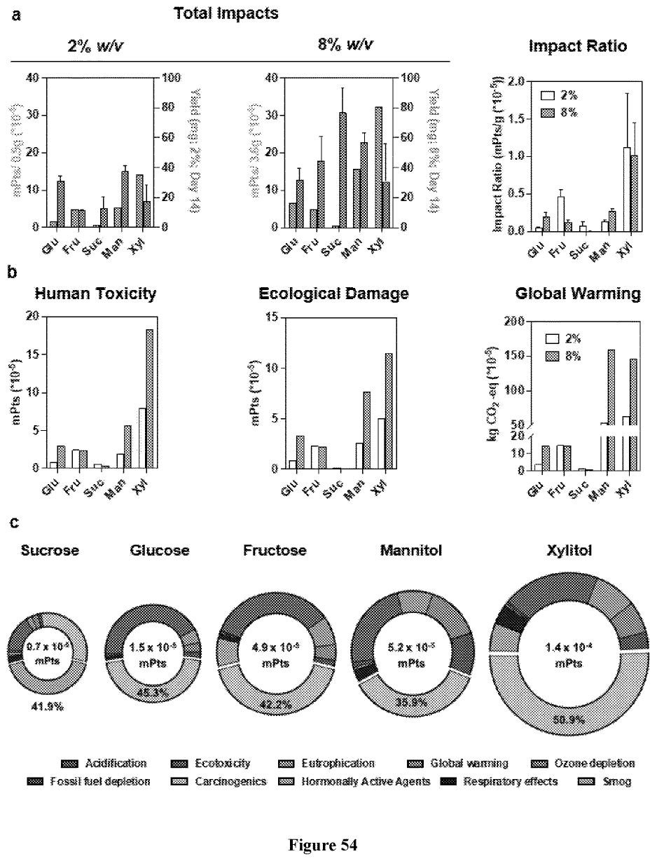

FIG. 55 Sorbitol-plasticized microbial cellulose characterization. Biofabricated microbial cellulose was bioprocessed with sorbitol (1, 2, and 5% w/v) via a facile immersion technique. (a) Nanofiber morphology was continually observed on microbial cellulose surfaces before and after sorbitol treatment(s) (SEM, n=3). (b) Evaluation of microbial cellulose tensile mechanical properties before and after sorbitol treatment show that ductility has a positive correlation with plasticizer concentration, while Young's Modulus significantly decreases. Toughness properties reveal anti-plasticizing behavior at 5% w/v (n=5, \hat{ }<0.05, between groups). (c) The presence of sorbitol was confirmed by FTIR, with increased peak absorption corresponding to greater hydroxyl groups at higher concentrations at 3345 cm−1 and 856 cm−1. Lateral Order Index and Total Crystallinity Index decreased with increasing sorbitol concentration treatment (n=5, \hat{ }<0.05, between groups).

FIG. 56 Glycerol-plasticized microbial cellulose characterization. Biofabricated microbial cellulose was bioprocessed with glycerol (1, 2, and 5% v/v) via a facile immersion technique. (a) Inherent nanofibrous morphology became less visible with increasing glycerol treatment from 1 to 5% v/v, as a result of plasticizer's homogenous coverage on microbial cellulose (SEM, n=3). (b) Evaluation of microbial cellulose tensile mechanical properties before and after glycerol treatment show that ductility has a positive correlation with plasticizer concentration, while Young's Modulus significantly decreases. Toughness properties reveal anti-plasticizing behavior at 5% v/v (n=5, \hat{ }<0.05, between groups). (c) The presence of glycerol was confirmed by FTIR, with increased peak absorption corresponding to greater hydroxyl groups at higher concentrations at 3345 cm-1. Lateral order index and total crystallinity index decreased after glycerol treatment (n=5, \hat{ }<0.05, between groups).

FIG. 57 Characterization of tannic acid-iron complexed microbial cellulose. Biofabricated microbial cellulose was crosslinked with tannic acid (0.5% w/v) and FeCl3 (0.5% w/v) at pH 1.99, pH 5.73, pH 9.14. (a) Inherent nanofibrous morphology became less visible with tannic acid treatment, as a result of tannin's coverage on cellulose. Immersion of TA-treated cellulose into FeCl₃ under pH conditions of 1.99 and 5.73 exhibited the formation of TA-Fe nanoparticles adhered to nanofibers. Further pH modification to 9.14 displayed randomly oriented nanofibers similar to as-fabricated MC (SEM, n=3). (b) Evaluation of microbial cellulose tensile mechanical properties before and after tannic acid-iron complexation showed that coordination at pH 5.73 and 9.14 significantly increased toughness and ductility, while maintaining strength compared to as-fabricated groups. Further, complexation at pH 9.14 significantly reduced ultimate tensile stress, ductility, and toughness (n=6, \hat{ }<0.05, between groups). (c) Complexation with iron at pH 1.99 further increased FTIR peak intensity around 3345 cm−1, representing greater hydroxyl groups. However, an increase in pH to 5.73 and 9.14 decreased peak intensity of the hydroxyl broad band between 3600-3000 cm−1, increased the relative absorbance of methyl stretching (1428 cm−1), aromatic C—C groups (1312 cm−1), and C—O/C—C groups (1205 cm−1). (d) Evaluation of cellulose crystal structure before and after tannic acid-iron complexation at pH 5.73 confirmed the maintenance of cellulose I Bragg peaks at 2θ=14.6o, 16.9o, and 22.8o, corresponding to the (1-10), (110), and (200) planes. The crystallinity index of pH 5.73 slightly increased compared to as-fabricated mCellulose (XRD, n=5, \hat{ }<0.05, between groups).

FIG. 58 Antibacterial properties of tannic acid-iron complexed microbial cellulose. A Kirby-Bauer disk diffusion assay revealed that as-fabricated samples exhibited no inhibition zones after 24 hours of culture, while dried mCellulose films only treated with 0.5% w/v tannic acid significantly increased inhibition zones, comparable to control disks containing 2% penicillin-streptomycin. Further, complexation with iron significantly decreased the inhibition zone, with no statistical difference of inhibition diameter between TA-Fe samples modified to pH 1.99 and 5.73 (n=5, \hat{ }<0.05, between groups).

FIG. 59 Life cycle impact assessment of carbon sources for biofabricating microbial cellulose. Cradle-to-gate life cycle impact assessment for utilizing 0.9 g of sugar (glucose, fructose, sucrose, mannitol, and xylitol) for biofabricating microbial cellulose. (a) Comparison of human health and environmental impact categories for different sugars. Human health toxicity includes impacts accumulating from carcinogenics and hormonally active agents, while ecological damage is associated with acidification, ecotoxicity, eutrophication, global warming, and ozone depletion. (b) Breakdown of the total impact by category, in which the greatest impact is highlighted by percentage of the total impacts.

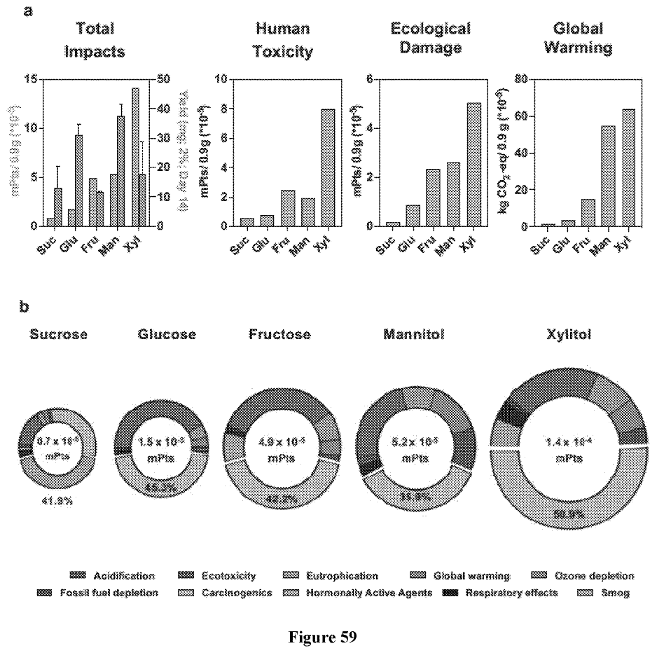

FIG. 60 Life cycle impact assessment of optimal bioprocessing strategies for microbial cellulose. Cradle-to-gate life cycle impact assessment for bioprocessing 150 cm2 of microbial cellulose utilizing either 2% w/v sorbitol, 1% v/v glycerol, or tannic acid-Fe(III) complexation. (a) Comparison of human health and environmental impact categories for different bioprocessing strategies. Human health toxicity includes impacts accumulating from carcinogenics and hormonally active agents, while ecological damage is associated with acidification, ecotoxicity, eutrophication, global warming, and ozone depletion. (b) Breakdown of the total impact by category, in which the greatest impact is highlighted by percentage of the total impacts. Legend: Glycerol1=Vegetable Oil, Glycerol2=Epichlorohydrin with 10% NaOH, Glycerol3=Epichlorohydrin Waste Oil, TA1-Fe=Polyphenol Waste Biomass Extraction, TA2-Fe=Hot Water Extraction, DOP=Di(2-ethylhexyl) phthalate.

FIG. 61 Biofabrication of microbial cellulose: Glucose, fructose, sucrose, mannitol, and xylitol-based medium support cellulose production by G. xylinus over two weeks of culture. (a) Glucose: Significantly greater cellulose yield is measured in glucose groups over time by day 14, independent of concentration. (b) Fructose: Cellulose bioproduction significantly increased in the 8% group overtime, with significant differences between concentrations at day 14. (c) Sucrose: Significantly greater cellulose yield is observed in all groups by day 14, while significant differences are measured between concentrations at day 7 and 14. (d) Mannitol: Cellulose bioproduction significantly increased in 2% and 8% groups overtime, with significant differences between concentrations at day 14. (e) Xylitol: Cellulose yield significantly increased by day 14 in 8% groups, with no significant differences between concentrations. (n=4, \hat{ }<0.05 between groups, *p<0.05 between timepoints). Scale bar: 1 cm.

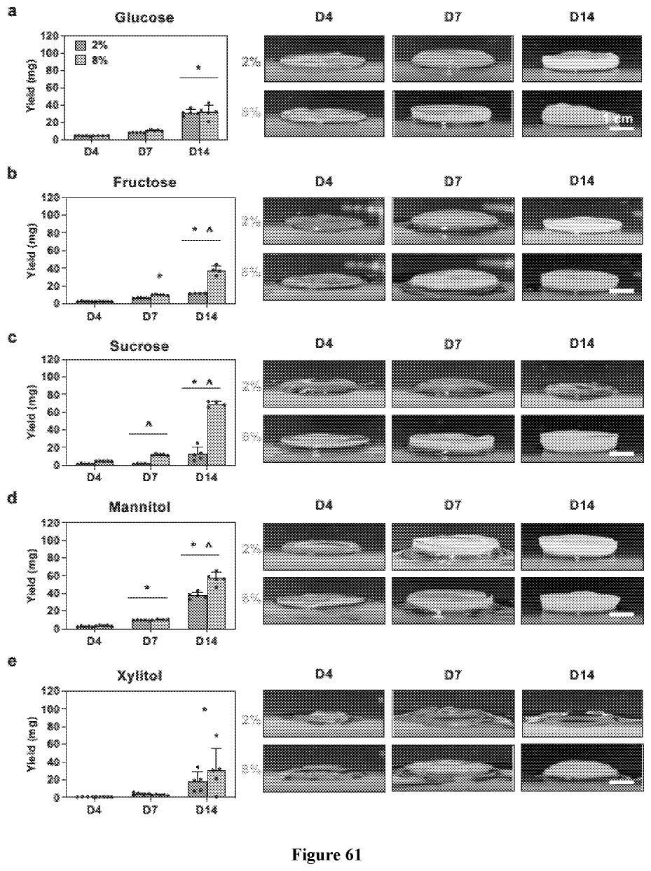

FIG. 62 Tensile Mechanical Properties of Cellulose Produced from G. xylinus Cultures Utilizing Glucose, fructose, Sucrose, Mannitol, and Xylitol-based Medium. (a) and (b) mechanical properties demonstrate significantly increased ultimate tensile strength (UTS), yield strength, and elastic modulus in 8% groups, while ductility between all carbon sources and concentrations were comparable (n=8, \hat{ }<0.05 between groups).

FIG. 63 Viscoelastic characterization of tannic acid-iron complexed microbial cellulose. Biofabricated microbial cellulose was crosslinked with tannic acid (0.5% w/v) and FeCl₃ (0.5% w/v) at pH 1.99, pH 5.73, pH 9.14. Complexation occurring at pH 1.99 significantly increased both elastic (storage) and shear modulus compared to as-fabricated mCellulose, while a further increase in pH to 5.73 and 9.14 continually reduced moduli (n=5, \hat{ }<0.05, between groups).

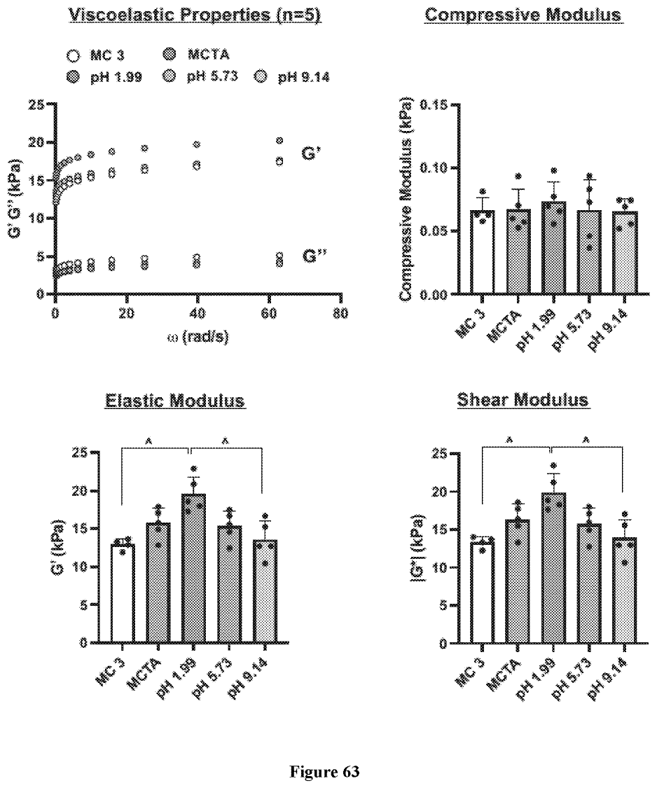

FIG. 64 Chemical structure and elemental characterization of tannic acid-iron complexed microbial cellulose. Biofabricated microbial cellulose was crosslinked with tannic acid (0.5% w/v) and FeCl3 (0.5% w/v) at pH 1.99, pH 5.73, pH 9.14. (a) Tannin presence in tannic acid-treated microbial cellulose was confirmed by FTIR, with peaks corresponding to O—H groups, benzene ring C—C, and C—O/CC at 3345 cm−1, 1312 cm−1, and 1205 cm−1, respectively. (b) Lateral Order Index and Total Crystallinity Index increased after tannin-iron complexation treatment (n=5, \hat{ }<0.05, between groups). (c-e) Iron presence in tannic acid-treated microbial cellulose was confirmed by EDXA and observed with spectral maps.

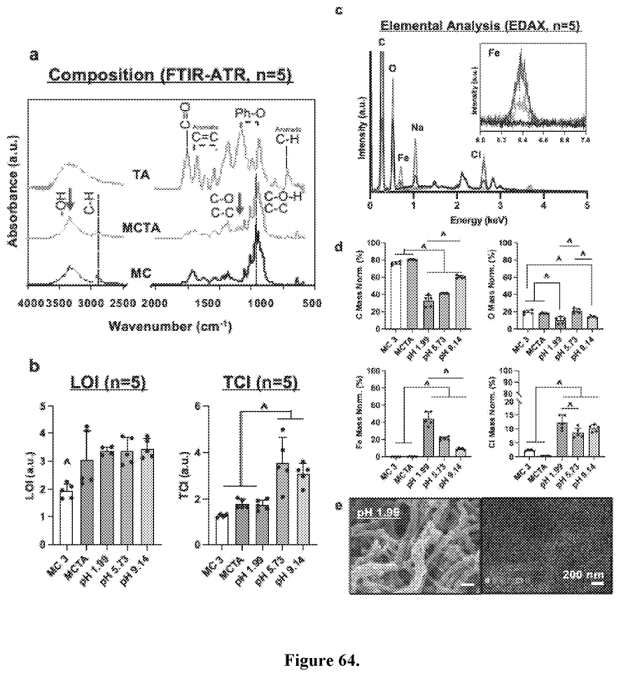

FIG. 65 Chemical Structure of Cellulose. Cellulose chains consist of physical crosslinks utilizing intra- and inter-molecular bonding. Biochemical pathway involved in the synthesis of microbial cellulose at the air-media interface. Carbon metabolism of glucose in aerobe microorganisms with the participation of four key enzymes: glucokinase, phosphogucomutase, glucose-1-phosphate uridyltransferase, and cellulose synthase. Resulting cellulose nanofibrils coagulate into a pellicle or 3D layered hydrogel at the air-media interface in static culture.

FIG. 66 Mannitol cultures produced fibers (~50 nm) with greater moduli and tensile strength. Dose-dependent increase in mechanical properties but not in ductility.

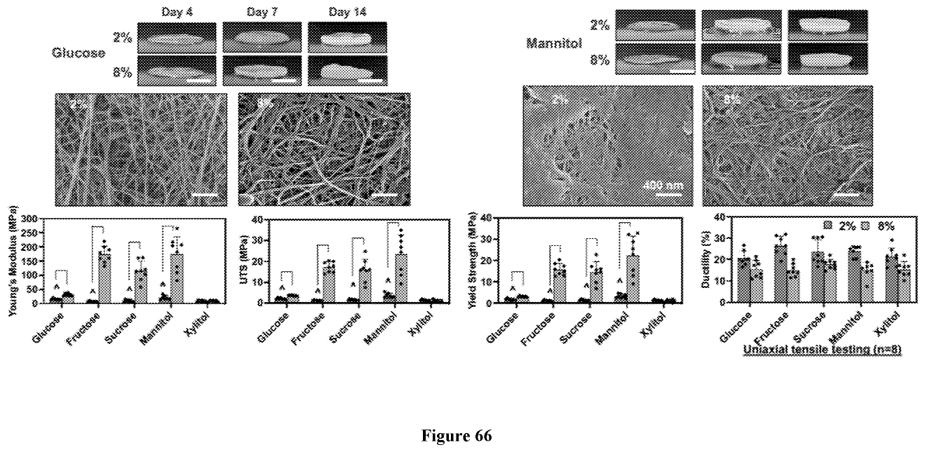

FIG. 67 Comparable fibers in terms of morphology, diameter, and chemistry Purification decreased sodium and phosphorus content. pmCellulose has lower modulus and tensile strength. Ductility maintained in hydrated state.

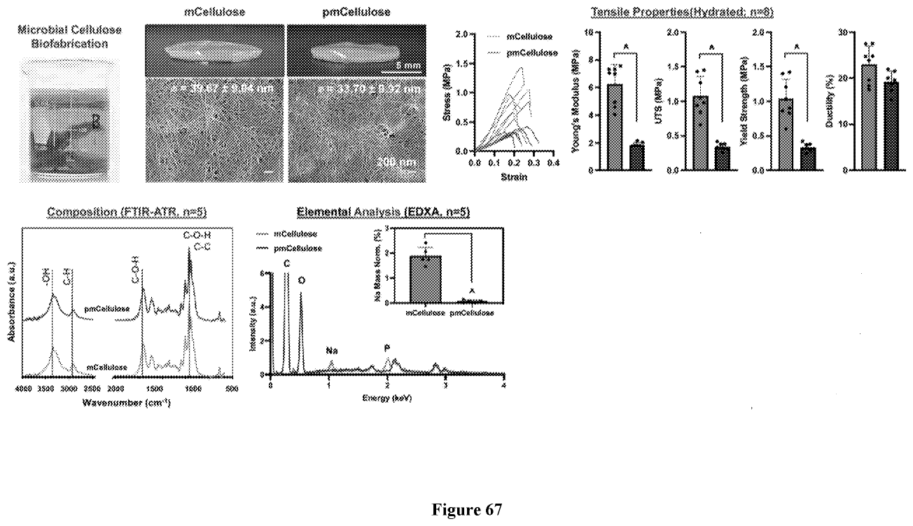

FIG. 68 Lower macrophage attachment compared to synthetic polymers Basal pro-inflammatory response. Biocompatible.

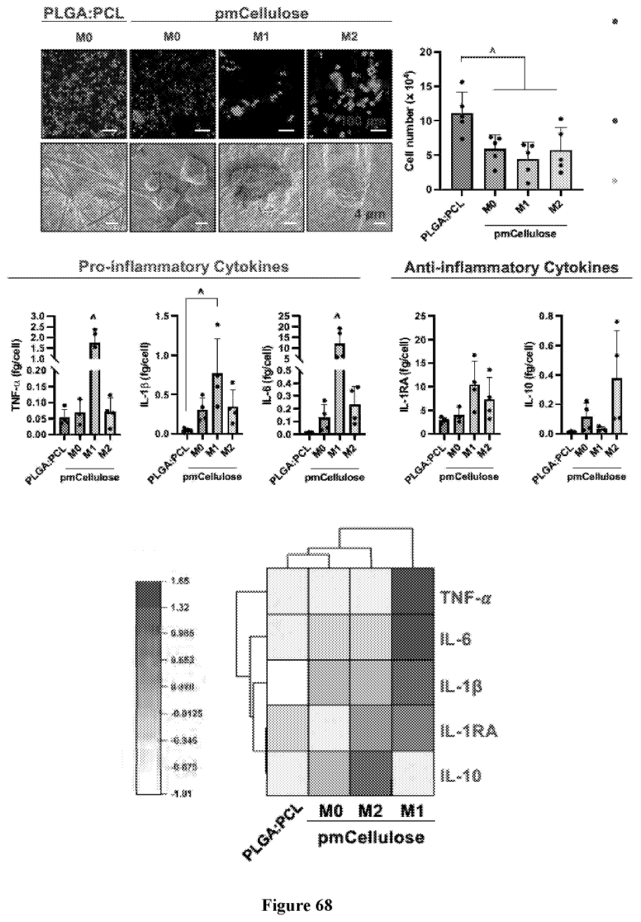

FIG. 69 LT biotextiles do not ignite when exposed to 2054 °C flame—Intact surface underneath char->exceptional flame retardance TGA exhibits cellulose characteristic three-step mass loss—LT lowers decomposition temperatures with greater char formation.

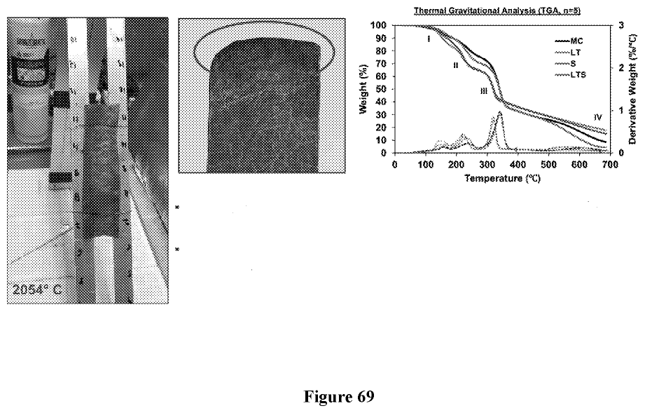

### DETAILED DESCRIPTION

Microbial cellulose (mCellulose) is a highly crystalline and chemically pure material, free of hemicellulose and lignin associated with plant cellulose, that is secreted by gram-negative bacteria, such as Acetobacter xylinum (A. xylinum). Under specific aerobic culturing conditions these bacteria biofabricate unaligned cellulose nanofibrils (10-100 nm diameter) that coagulate into a three-dimensional layered hydrogel (>98% water), known as a pellicle, at the air-culture media interface. Microbial cellulose has gained significant interest due to its inherent physiochemical properties, including high tensile strength, high degree of polymerization, crystallinity (80-90%), nanofiber morphology, swelling capability, moldability, biodegradability in composting environments, and biocompatibility. Owing to these unique properties, mCellulose is an advanced biomimetic material that is attractive for green biofabrication applications, such as sustainable material alternatives mitigating the large environmental impact of the textile industry, and as a tissue engineered scaffold to regenerate musculoskeletal tissues.

The textile industry is one of the most ecologically damaging industries, and biggest global contributors to climate change, accounting for 1.2 billion tons of CO₂ emissions per year. The industry's linear production model is dependent upon finite nonrenewable resources to manufacture synthetic fibers, including more than 50 million metric tons of polyester produced globally in 2016. Textile dyeing and finishing accounts for 20% of global water waste, making it the second largest industrial source of water pollution globally. Additionally, machine washing of synthetic textiles is the single largest source of microplastic pollution responsible for up to 35% of primary microplastics in marine environments. Microplastics are not only detected in sewage treatment plant effluents discharged into the sea, but also found in the soil of coastal shorelines. Textile finishing, such as leather tanning, requires multiple processing steps that utilize coal tar derivatives and formaldehyde to aesthetically improve the material, causing abundant environmental risks. Thus, ecological damage within each segment of the textile industry's linear production model contribute to the loss of biodiversity and climate instability.

Therefore, there is a pressing need for scalable, sustainable raw materials that support a circular economy, reducing toxicity (water and carbon footprints) throughout a product's life cycle. Microbial cellulose materials present a distinctive opportunity to achieve minimal-waste production through biosynthesis of cellulose that self-assemble into the shape of the growth vessel without impacting human health and ecotoxicity. As the culture medium comprises approximately 30-60% of the total microbial cellulose production cost, the ability to extract nutrients, including sugar, from agro-industrial waste as feedstock for the fermentation media further supports circularity of biofabrication at scale. However, microbial cellulose synthesis, post-production methods, and minimal-waste design must be further optimized to have tailored properties (physical, mechanical, chemical, and thermal) for textile production.

As microbial cellulose materials are highly attractive as sustainable alternative textiles, they have also emerged as biofabricated materials for medical applications such as tissue regeneration, skin and blood vessel substitutes, and drug delivery. Connective tissues, such as ligaments and tendons, are often injured due to low cell density and vascularization (oxygen and nutrient transport) causing poor regeneration capacity. For example, anterior cruciate ligament (ACL) rupture is the leading ligament injury in the United States, with over 250,000 reconstructions performed and more than $2.6 billion in healthcare costs annually. The ACL is the major intra-articular ligament in the knee that connects the femur and tibia to mediate load transfer and ensure joint stabilization. Unfortunately, the ACL has limited self-healing capacity and susceptibility to joint inflammation, hindering regeneration. Tissue engineering approaches utilize various biofabrication techniques to develop biodegradable scaffolds for guided cell-mediated regeneration, replacing tissue lost from disease or injury. In particular, electrospinning has become a dominant strategy for fabricating nanometer to micrometer diameter fibers from synthetic and natural polymers for cell culturing, producing scaffolds with an architectural network comparable to collagen fibrils within the extracellular matrix (ECM). However, traditional electrospinning procedures solubilize polymers with industrial solvents, such as dimethylformamide (DMF), dichloromethane (DCM), or trifluoroethanol (TFE), which all have associated ecological damage and health impacts, restricting them from use in pharmaceuticals by the U.S. Food and Drug Administration (FDA). Thus, green manufacturing with non-volatile biologically benign solvents (FDA Q3C Class 3), for tissue engineering is an emerging approach with potential to mimic the biological properties of the natural tissue, to promote regeneration, while mitigating negative environmental impacts. Of note, natural polymers (silk, collagen, hyaluronic acid, alginate, chitosan) are attractive materials for ACL regeneration because of their biocompatibility, similar structure to cellular extracellular matrices, and degradation ability. However, unlike synthetic counterparts, they exhibit poor mechanical properties, uncontrolled degradation, and low reproducibility during processing due to batch to batch variation, making them have limited performance for ligament and tendon regeneration. Therefore, there is a significant interest in developing biomimetic solutions for tissue regeneration that have mechanical properties similar to the native tissues, and are not limited by degradation rates, batch to batch variability, as well as negative environmental and health impacts.

Microbial cellulose scaffolds offer great biocompatibility, and physiochemical properties, with minimal impacts on human health and the environment. However, their inherent bacterial content, porosity, and degradation must be optimized for tissue engineering applications. To this end, textile bioengineering has emerged as an approach for both developing sustainable biomaterials and integrative ligament reconstruction. Due to microbial cellulose's unique properties, it is a biomimetic material utilized in food, cosmetic, textile, electrochemical and biomedical industries. For microbial cellulose to be suitable for these various applications, its material properties (mechanical, thermal, hydrophilicity, degradation, and surface architecture) must be tailored and optimized. To address these challenges, this invention investigated and determined design parameters for the development of microbial cellulose as non-medical and medical textiles. Inspired by the versatile nanoscale to macroscale properties of microbial cellulose materials, the working hypothesis of this invention is two-fold: For non-medical approaches, the development of green bioprocessing strategies as post-modification treatments for biofilms will alter material properties for suitable substitutes in the textile industry. For medical approaches, the development of a controlled purification methodology will support the utilization of microbial cellulose as biocompatible scaffold for tissue engineering applications, in comparison to conventional synthetic micro-fiber substrates. Thus, three specific aims and their respective hypotheses for this proposal are detailed below:

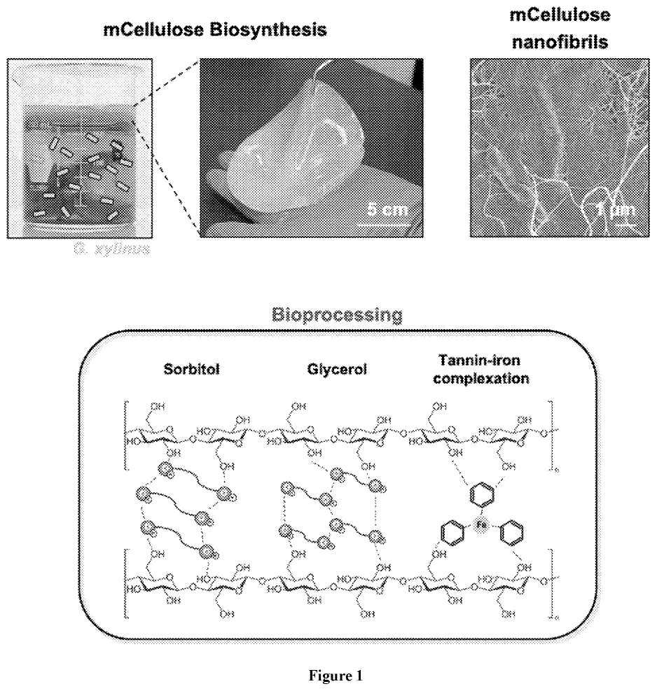

Cellulose is a linear β-1,4-linked glucose polymer produced by plants and various obligate aerobic microorganisms, making it the most abundant biomacromolecule on earth (FIG. 1.2). Plant-derived celluloses contain coexisting crystalline and amorphous domains due to crosslinking with other hetero-polysaccharides, such as hemicelluloses, lignin, and pectin conferring to the plant structure's strength and flexibility. Alternatively, microbial cellulose secreted mainly by gram-negative bacteria, such as Acetobacter xylinum, Aerobacter, and Agrobacterium, is a chemically pure network free of the prior polysaccharides. The synthesized cellulose forms an envelope that surrounds microbes known to not only protect against UV irradiation, high temperatures, desiccation, and harsh environmental conditions, but also promote bacterial adhesion, and nutrient transport for survival.

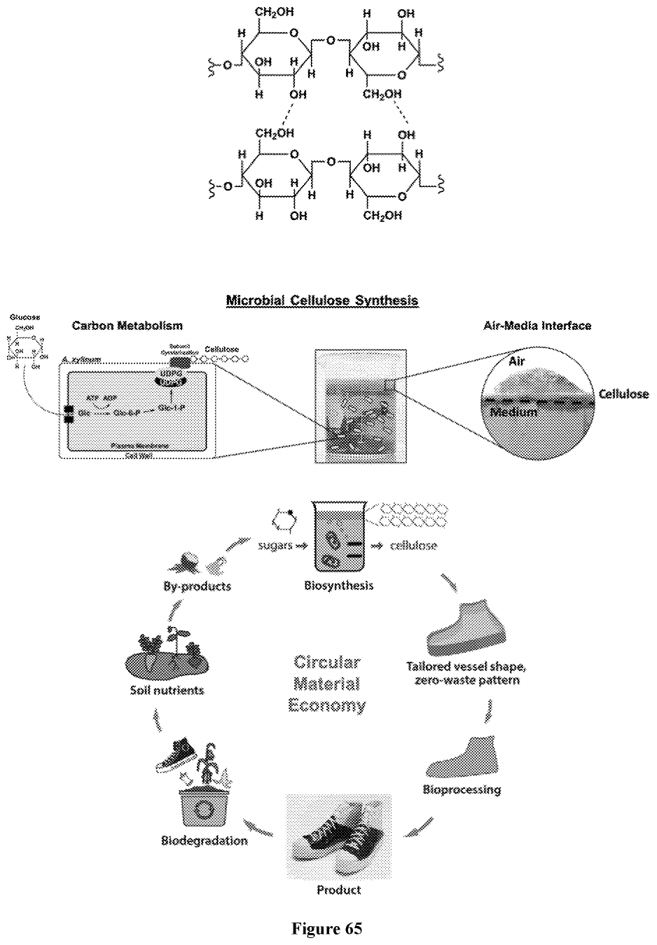

Microbial research has turned to the aerobic strain Acetobacter xylinum (A. xylinum) as the model microorganism for the production of microbial cellulose, as it is reported a single cell can polymerize ~1.5×10⁸ glucose units per hour. Under specific culturing conditions, these bacteria biosynthesize microbial cellulose by metabolizing a carbon source, such as glucose. This process requires four enzymes (glucokinase, phosphogucomutase, glucose-1-phosphate uridyltransferase, and cellulose synthase) with catalytic, regulatory, and accessory subunits to biosynthesize, translocate, and crystallize cellulose chains. Once absorbed by the bacteria, glucose is phosphorylated at carbon-6 site into glucose-6-phosphate (Glc-6-P) by glucokinase and isomerized into glucose-1-phosphate (Glc-1-P) by phosphogucomutase. Glucose-1-phosphate uridyltransferase catalyzes the synthesis of the precursor uridine diphosphate glucose (UDP-glucose), prior to the polymerization of glucose chains into linear 1,4-β-glucan chains by cellulose synthase (CS) within cytoplasmic biofilms (FIG. 65). Depending on the main carbon source in the environment or growth medium, different biochemical pathways and enzyme pathways are activated to synthesize the final polymer. Due to uncontrolled movement of bacteria, secretion of cellulose nanofibrils are randomly oriented (10-100 nm diameter) and coagulate into a 3D layered hydrogel (>90% water), known as apellicle, at the air-media interface in static culture (FIG. 65).

Microbial cellulose biofilms present unique properties including great tensile strength (as high as ~66 GPa elastic moduli), polymerization, crystallinity (80-90%), water holding capacity (holding up to hundreds of times its dry weight in water), hydrophilicity, biocompatibility, and moldability. Due to the absence of amorphous domains originally observed in plant-based cellulose, these excellent properties allow mCellulose to be an advanced biomimetic material currently replacing plant cellulose and synthetic polymers in textile, food, cosmetic, electrochemical, and biomedical industries. In contrast to conventional manufacturing processes, microbial nanocelluloses are biofabricated in a bottom-up approach via cellulose biosynthesis during oxidative fermentation in microbial culture, allowing for microbes to direct biomaterial formation. Biofabrication of the biofilm occurs at the air-media interface, where the stabilization of desired engineered morphologies, geometries, and capsules can be controlled. Further biofabrication techniques such as microfluidic technologies, or silicone templating have been applied to direct A. xylinum movement on a microscale to develop cellulose nanofibril-based three-dimensional materials. This customizability suggests its significant potential use across various applications focused upon material science. However, depending on the application, further modification of mCellulose biofilms is required to tailor properties with respect to mechanical stability, thermal stability, crystal structure, and surface functionalization. Design criteria for mCellulose as a next-generation biotextile require optimal properties that promote a closed loop product life cycle. Yet, for tissue engineering, these properties will differ, such that biocompatibility, degradation, hydrophilicity, and physiological mechanical strength are at the forefront. Thus, this invention will result in the development of strategically biofabricated and bioprocessed microbial cellulose as a biomaterial for textile and biomedical applications.

### Microbial Cellulose: An Emerging Biotextile

In the textile industry, microbial cellulose is a nonwoven, structured green material with a leather like appearance that can be biofabricated from symbiotic culture of bacteria and yeast. This natural alternative is setting the basis for a ‘green’ economy, minimizing environmental impacts with a closed loop circular life cycle (FIG. 65). In comparison to conventional textiles, this biofabrication-based life cycle consists of controllable microbial fermentation, post-synthesis modifications, biotextile applications, and biodegradation into natural glucose monomers that can be used as feedstock for new bacterial culture.

Microbial cellulose made with traditional cultivation media such as Hestrin & Schramm (HS) medium has been proven to be infeasible for large scale processes because it is expensive and requires additional resources (glucose, yeast extract, peptone) to support microbe metabolism. While it is reported that cellulose synthesizing organisms can metabolize carbon sources from dextran production or petrochemical waste, production from agricultural waste, including food surplus, fruit, wheat straw, and cotton-based residues, has been demonstrated. Briefly, Kongruang et al. demonstrated that fruit waste, produced by agricultural industries (coconut and pineapple juice) supplemented with yeast extract and ethanol, maintains cellulose yield, improves energy consumption and decreases production costs. Further analysis of cellulose indicated that composition of waste source did not alter morphology, structure, and crystallinity of traditionally grown substrates. Thus, agricultural waste composed of carbohydrates, and trace elements supports the biotextile circular life cycle at an industrial scale.

While cultivation of these microorganisms can lead to tailored garments within two weeks, further surface functionalization or polymer matrix biocomposites are required to improve tensile strength, elasticity, and surface hydrophilicity. Contact surface-blocking cultivation and panel-shaped cultivation implemented by Chan et al. served as viable techniques to maximize cellulose fabric utility for unfunctionalized garments, resulting in “zero” textile material waste. Surface treatment of cellulose biofilms with vinyl-triethoxy silane or 3-aminopropyl triethoxysilane, by acylation and acrylation, respectively, led to controlled hydrophobicity with contact angles ranging from 40°-80°. Alternatively, dose dependent application of natural enzymes, such as lecithin, on cellulose increased contact angle and hydrophobicity within similar ranges to synthetic treatments, but also decreased thermal stability. Biodegradation of pure cellulose can occur after 3 to 5 months depending on environmental conditions, in which material modifications can design materials for specific climates. Thus, microbial cellulose is presented as a sustainable biomaterial in the textile industry, considering that production is controlled by bacterial action, offering advantageous properties, such as growth from agricultural waste, tailor-shaped cultivation, and minimal-waste sustainability.

### Non-medical Textile Innovation and Anticipated Impact

Current approaches to reduce pollution and carbon emission generated by the textile industry are deemed to be inefficient to support sustainable development goals. Since pollution is observed during raw material production, garment manufacturing, consumer use, and product disposal, solutions must address all four phases to facilitate the improvement in environmental performance required to reach climate impact goals. This invention aims to investigate mCellulose biomaterials as a strategic biofabrication platform to transition textile production from an open to a closed circular loop life cycle. Innovation in this invention pertains to the use of microbes to direct biomaterial formation in a bottom-up approach as a novel biofabrication technique, which is unconventional in traditional manufacturing that relies on chemically intensive processing. Since mCellulose production and functionality is highly dependent upon its synthesis parameters and manufacturing processes, this platform will determine optimal cultivation techniques and natural biotextile modifications built on the canonical principles of green chemistry. The microbial strain A. xylinum will be fermented in various carbon sources to control the production of cellulose, while chemical and structural bio-based approaches will be considered to modulate pristine properties. Thus, providing insight in industrial-scaled biomaterial design with minimal waste approaches.

In addition to reducing pollution within each phase of the textile industry, the long-term impact of this invention lies within its ability to inform biofabrication-based design principles for other biomaterial applications, such as tissue engineering. The nanofibrous architecture of microbial cellulose closely mimics the native collagenous matrix observed in various human tissues, so there is great potential for its translation to medical settings. A natural polymer scaffold system based upon optimal cultivation techniques and natural biotextile modifications will provide design criteria for the repair of fibrous connective tissues (ligaments and tendons), and other complex tissue systems.

### Connective Tissue Engineering

Injuries to connective tissues, such as ligament and tendons, are highly prevalent, in which their limited self-healing capacity results in a fibrotic response known to form unaligned scar tissue that is mechanically inferior. For example, anterior cruciate ligament rupture is the leading ligament injury in the United States, with over 250,000 reconstructions performed and more than $2.6 billion in healthcare costs annually. Tissue engineering is a promising approach that aims to reestablish native tissue morphology and functionality with the use of specific structural scaffolding materials, cell lines, and growth factors. Various biofabrication techniques aim to develop biodegradable scaffolds for guided cell-mediated regeneration, replacing tissue lost from disease or injury. In particular, electrospinning is a dominant strategy for fabricating nanometer to micrometer diameter fibers from synthetic and natural polymers for cell culturing producing scaffolds with an architectural network comparable to collagen fibrils within the extracellular matrix. Synthetic-derived biomaterials (poly(glycolic acid) (PGA), poly(ε-caprolactone) (PCL), poly(L-lactic acid) (PLLA), poly(lactide-co-glycolide) (PLGA)), natural-derived biomaterials (silk, collagen, hyaluronic acid, alginate, chitosan), and composite systems (collagen-silk, PCL-PGA) have been used to support cell attachment, proliferation, and differentiation for connective tissue engineering. In particular, natural polymers are attractive materials for tissue engineering because of their biocompatibility, similar structure to cellular extracellular matrices, and degradation ability. However, unlike synthetic counterparts, they exhibit poor mechanical properties, uncontrolled degradation, and low reproducibility during processing due to batch to batch variation, making them have limited performance for ligament and tendon regeneration. In addition, traditional electrospinning procedures solubilize polymers with industrial toxic solvents, such as dimethylformamide, dichloromethane, or trifluoroethanol, which all have associated ecological damage and health impacts, restricting them from use in pharmaceuticals by the U.S. Food and Drug Administration. Thus, green manufacturing, with non-volatile biologically benign solvents (FDA Q3C Class 3), for tissue engineering has become an emerging approach to mimic the biological properties of the natural tissue, and promote regeneration, while mitigating negative environmental impacts. Therefore, there is a significant interest in biofabricating biomimetic materials with low environmental impacts for connective tissue regeneration that addresses the limitations of natural-derived biomaterials, while supporting cell bioactivity.

### Microbial Cellulose Biomaterials as a Scaffold for Tissue Engineering

Microbial cellulose is a natural polysaccharide that has shown immense potential as a biomimetic scaffold for tissue engineered skin, cartilage, blood vessels, menisci, bone, and neuronal constructs. Once synthesized by A. xylinum, the biofabricated nanocellulose has unique properties, such as tailored mechanical strength, hydrophilic surfaces, nanofibrous morphology resembling tissues' native extracellular matrix, and moldability, required for regenerative scaffolds to support cell attachment, proliferation, and biomolecule secretion. While chondrocyte-seeded unmodified mCellulose scaffolds supported cell growth, physiological morphology, and enhanced expression of collagen type II genes, Vielreicher et al. also demonstrated that unmodified microbial cellulose, synthesized by a different strain of microbe, facilitated MSC attachment within scaffolds creating niches that enhanced the formation of an organized type I collagen network. Fibroblast-seeded scaffolds modified with micro-channels subject to biomimetic compressive loads resulted in guided tissue growth and collagen alignment similarly observed in native menisci. These findings propose that mCellulose scaffolds provide a favorable environment for cells to remain viable, and further functionalization will enhance long-term in-vivo responses.

However, microbial cellulose is inherently limited by its production by gram-negative bacteria that contain endotoxins that can elicit inflammatory responses, non-biodegradability in the human body, and its dense fibril network that prevents cell infiltration, hindering further application for medical applications. Due to the absence of enzymatic and chemical mechanisms in the human body that can hydrolyze cellulose's linear β-1,4-glycosidic linkages, the polymer is not biodegradable in-vivo, which impedes the ability of newly formed tissue to replace it over time. Current approaches to achieve in-vitro and in-vivo biodegradation include oxidizing the polymer into 2,3-dialdehyde cellulose, in which chemical modification of the biomaterial supported fibroblast attachment, viability, and deposition of collagen type III and fibronectin. Additionally, as a natural approach, absorption of enzymatic cellulases onto microbial cellulose scaffolds, bio-inspired from bacteria and fungi, was developed as a technique for dose-dependent hydrolyzation. Modulation of cellulase content supported tensile strengths between 1 to 18 MPa for up to 24 days, and additionally allowed for greater cell infiltration for 4 weeks into implanted scaffolds when compared to nondegradable constructs. Alternatively, Zamborowska et al. demonstrated that addition of 300-500 μm porogens during the fermentation process enhanced biomaterial porosity (~100 μm) with increased osteoprogenitor cell infiltration and increased mineral deposition within pores throughout 14 days of culture. Microbial cellulose is an attractive biomaterial for a vast range of tissue engineering applications due to its distinctive nanofibril structure, variable tensile strength, hydrophilicity, cellular affinity, and moldability. Thus, these results suggest that this biomimetic platform has great potential for tissue engineering strategies.

### Medical Textile Innovation and Anticipated Impact

Microbial cellulose is an emerging biomaterial in tissue engineering applications due to the natural polymer's appealing properties that include simple production, high crystallinity, tunable tensile strengths, hydrophilicity, and moldability. Most importantly, mCellulose's three-dimensional nanofibril structure closely resembles tissue's native collagenous matrix, which makes the material have high potential to facilitate cell attachment, proliferation, migration, and ECM deposition. However, the presence of bacteria and corresponding endotoxins, lack of in-vivo degradability, and poor cell infiltration due to dense fibril organization must be addressed prior to making cellulose an ideal material for tissue engineering scaffolds. This invention aims to explore mCellulose as a tissue engineering biomaterial platform for the regeneration of fibrous connective tissues, such as anterior cruciate ligaments. Microbial cellulose scaffolds will be exposed to a purifying procedure for reducing endotoxin content, in which biocompatibility will be determined with human THP-1 macrophage culture, and bovine ACL fibroblasts. Innovation of this invention pertains to use of microbes to direct biomaterial formation in a bottom-up approach as a novel biofabrication technique, which is unconventional in traditional biofabrication for tissue engineering. Therefore, the long-term impacts of this invention will provide design criteria for the repair of other complex tissue systems (cartilage, meniscus, tendon, and bone).

A microbial cellulose material can be provided comprising:
(a) an acid;
(b) a metal ion; and
(c) microbial cellulose,
wherein the
   (i) acid is a polyphenol;
   (ii) metal ion is selected from a group consisting of iron ion (Fe²⁺), iron ion (Fe³⁺) cooper ion (Cu²⁺), aluminum ion (Al³⁺), magnesium ion (Mg²⁺), zinc ion (Zn²⁺), nickel ion (Ni²⁺), germanium ion (Ge⁴⁺), titanium (Ti⁴⁺), molybdenum (Mo⁶⁺), and tungsten ion (W⁶⁺); and
   (iii) microbial cellulose is a bioplasticized microbial cellulose.

In some embodiments, polyphenol is phenolic acid.

In some embodiments, metal ion is Fe²⁺, Fe³⁺, Al³⁺, Cu²⁺, Mg²⁺, or Zn²⁺.

In some embodiments, microbial cellulose is bioplasticized by polyol, monosaccharide, oligosaccharide, lipid, or hyperbranched polyester.

In some embodiments, phenolic acid is selected from a group consisting of hydrolysable tannin, condensed tannin phlorotannin, tannic acid, caffeic acid, ferulic acid, protocatechuic acid, p-hydroxybenzoic acid, vanillic acid, p-coumaric acid, gallic acid, syringic acid, and sinapinic acid.

In some embodiments, metal ion is Fe²⁺, Fe³⁺, Cu²⁺, Mg²⁺, or Zn²⁺.

In some embodiments, microbial cellulose is bioplasticized by polyol.

In some embodiments, phenolic acid is selected from a group consisting of hydrolysable tannin, condensed tannin phlorotannin, and tannic acid.

In some embodiments, metal ion is Fe²⁺, Fe³⁺, Mg²⁺, or Zn²⁺.

In some embodiments, polyol is selected from a group consisting of glycerol, sorbitol, erythritol, polyester polyols, xylitol, isomalt, lactitol, maltitol, polycaprolactone polyol, mannitol, and polypropylene glycol.

In some embodiments, phenolic acid is tannic acid.

In some embodiments, metal ion is Fe³⁺.

In some embodiments, polyol is glycerol or sorbitol.

In some embodiments, Young's modulus of the microbial cellulose material is from 20 MPa to 300 MPa.

In some embodiments, ultimate tensile strength of the microbial cellulose material is from 0.1 Mpa to 10 Mpa.

In some embodiments, toughness of the microbial cellulose material is from 0.1*10⁴ joule per cubic metre (J·m⁻³) to 180*10⁴ J·Tm⁻³.

In some embodiments, ductility of the microbial cellulose material is from 1% to 40%.

In some embodiments, Young's modulus of the microbial cellulose material is from 20 MPa to 60 MPa.

In some embodiments, ultimate tensile strength of the microbial cellulose material is from 2 Mpa to 5 Mpa.

In some embodiments, toughness of the microbial cellulose material is from 10*10⁴ J·m⁻³ to 50*10⁴ J·m⁻³.

In some embodiments, ductility of the microbial cellulose material is from 15% to 25%.

In some embodiments, wherein when the pH of the complex is from 4-6.5, Young's modulus of the microbial cellulose material is from 30 MPa to 60 MPa.

In some embodiments, wherein when the pH of the complex is from 4-6.5, ultimate tensile strength of the microbial cellulose material is from 2 Mpa to 7 Mpa.

In some embodiments, wherein when the pH of the complex is from 4-6.5, toughness of the microbial cellulose material is from 50*10⁴ J·m⁻³ to 180*10⁴ J·m⁻³.

In some embodiments, wherein when the pH of the complex is from 4-6.5, ductility of the microbial cellulose material is from 25% to 35%.

In some embodiments, when the pH of the complex is from 8-10, Young's modulus of the microbial cellulose material is from 60 MPa to 250 MPa.

In some embodiments, when the pH of the complex is from 8-10, ultimate tensile strength of the microbial cellulose material is from 0.1 Mpa to 3.5 Mpa.

In some embodiments, when the pH of the complex is from 8-10, toughness of the microbial cellulose material is from 0.1*10⁴ J·m⁻³ to 5*10⁴ J·m⁻³.

In some embodiments, when the pH of the complex is from 8-10, ductility of the microbial cellulose material is from 2% to 5%.

In some embodiments, microbial cellulose material has an anti-bacteria activity.

In some embodiments, microbial cellulose material has a 4-8 mm zone of inhibition.

In some embodiments, microbial cellulose material has a 6-10 mm zone of inhibition.

In addition, a process of preparing the microbial cellulose material may be provided, wherein the process comprises:
(a) preparing a bioplasticized microbial cellulose;
(b) bathing the bioplasticized microbial cellulose from step (a) in a polyphenol solution;
(c) bathing the bioplasticized microbial cellulose from step (b) in a metal ion solution; and
(d) optionally adding a base to the metal ion solution in step (c).

In some embodiments, the bioplasticized microbial cellulose from step (a) is first bathed in water and then dried before step (b).

In some embodiments, in step (a), the bioplasticized microbial cellulose is bathed in the polyphenol solution for a period of 12-72 hours.

In some embodiments, the bioplasticized microbial cellulose is bathed in the polyphenol solution for a period of 12-36 hours.

In some embodiments, the bioplasticized microbial cellulose is bathed in the polyphenol solution for a period of 12-24 hours.

In some embodiments, the bioplasticized microbial cellulose is bathed in the polyphenol solution for a period of 24 hours.

In some embodiments, the bioplasticized microbial cellulose from step (b) is first bathed in water and then dried before step (c).

In some embodiments, in step (b), the bioplasticized microbial cellulose is bathed in the metal ion solution for a period of 0.5-12 hours.

In some embodiments, in step (b), the bioplasticized microbial cellulose is bathed in the metal ion solution for a period of 0.5-6 hours.

In some embodiments, in step (b), the bioplasticized microbial cellulose is bathed in the metal ion solution for a period of 1-3 hours.

In some embodiments, in step (b), the bioplasticized microbial cellulose is bathed in the metal ion solution for a period of 2 hours.

In some embodiments, bioplasticized microbial cellulose is prepared by bathing hydrated microbial cellulose in a polyol solution.

In some embodiments, the hydrated microbial cellulose is bathed in the polyol solution for a period of 12-72 hours.

In some embodiments, the hydrated microbial cellulose is bathed in the polyol solution for a period of 12-36 hours.

In some embodiments, the hydrated microbial cellulose is bathed in the polyol solution for a period of 12-24 hours.

In some embodiments, the hydrated microbial cellulose is bathed in the polyol solution for a period of 24 hours.

In some embodiments, polyol is selected from a group consisting of glycerol, sorbitol, erythritol, polyester polyols, xylitol, isomalt, lactitol, maltitol, polycaprolactone polyol, mannitol, and polypropylene glycol.

In some embodiments, polyol is glycerol or sorbitol.

In some embodiments, concentration of glycerol or sorbitol is 0.1%-20% weight per volume (w/v).

In some embodiments, concentration of glycerol or sorbitol is 0.5%-10% weight per volume (w/v).

In some embodiments, concentration of glycerol or sorbitol is 1%-5% weightper volume (w/v).

In some embodiments, polyphenol is a phenolic acid.

In some embodiments, phenolic acid is selected from a group consisting of hydrolysable tannin, condensed tannin phlorotannin, tannic acid, caffeic acid, ferulic acid, protocatechuic acid, p-hydroxybenzoic acid, vanillic acid, p-coumaric acid, gallic acid, syringic acid, and sinapinic acid.

In some embodiments, phenolic acid is selected from a group consisting of hydrolysable tannin, condensed tannin phlorotannin, and tannic acid.

In some embodiments, phenolic acid is tannic acid.

In some embodiments, tannic acid has a concentration of 0.1-10% w/v; preferably, 0.1-5% w/v, more preferably, 0.1-2 w/v.

In some embodiments, metal ion is Fe²⁺, Fe³⁺, Cu²⁺, Al³⁺, Mg²⁺, Zn²⁺, Ni²⁺, Ge⁴⁺, Ti⁴⁺, Mo⁶⁺, or W⁶⁺.

In some embodiments, metal ion is Fe²⁺, Fe³⁺, Al³⁺, Cu²⁺, Mg²⁺, or Zn²⁺.

In some embodiments, metal ion is Fe²⁺, Fe³⁺, Mg²⁺, or Zn²⁺.

In some embodiments, metal ion is Fe³⁺.

In some embodiments, metal ion solution is iron chloride (FeCl₃), iron oxide (Fe(OH)₃), iron fluoride (FeF₃), iron thiocyanates (Fe(SCN)₃), or iron salicylate (C₂₁H₁₅FeO₉).

In some embodiments, metal ion solution is FeCl₃, Fe(OH)₃, or FeF₃.

In some embodiments, metal ion solution is FeCl₃, or FeF₃.

In some embodiments, metal ion solution is FeCl₃.

In some embodiments, the base in step (d) is an inorganic base.

In some embodiments, inorganic base is sodium hydroxide (NaOH), potassium hydroxide (KOH), magnesium hydroxide (Mg(OH)₂), calcium hydroxide (Ca(OH)₂), or ammonia (NH₃).

In some embodiments, inorganic base is NaOH, or KOH.

In some embodiments, inorganic base is NaOH.

In some embodiments, the process further comprises rinsing and drying the bioplasticized microbial cellulose from step (c).

A process of purifying a microbial cellulose film may be provided, comprising:
(e) bathing the microbial cellulose film in cellulase solution;
(f) bathing the microbial cellulose film from step (a) in an alcohol solution;
(g) bathing the microbial cellulose film from step (b) in a basic solution; and
(h) applying a high-pressure steam to the microbial cellulose film in step (c).

In some embodiments, the microbial cellulose film in step (a) is hydrated.

In some embodiments, the microbial cellulose film in step (a) is washed with water before step (b).

In some embodiments, the microbial cellulose film in step (a) is bathed in the cellulase solution for a period of 0.5-10 hours.

In some embodiments, the microbial cellulose film in step (a) is bathed in the cellulase solution for a period of 0.5-5 hours.

In some embodiments, the microbial cellulose film in step (a) is bathed in the cellulase solution for a period of 0.5-2 hours.

In some embodiments, the microbial cellulose film in step (a) is bathed in the cellulase solution for 1 hour.

In some embodiments, the microbial cellulose film in step (c) is washed with water before step (d).

In some embodiments, the microbial cellulose film in step (c) is washed with water for a period of 12-72 hours.

In some embodiments, the microbial cellulose film in step (c) is washed with water for a period of 12-36 hours.

In some embodiments, the microbial cellulose film in step (c) is washed with water for a period of 12-24 hour.

In some embodiments, the microbial cellulose film in step (c) is washed with water for 24 hours.

In some embodiments, the microbial cellulose film is bathed in the basic solution for a period of 12-72 hours,

In some embodiments, the microbial cellulose film is bathed in the basic solution for a period of 12-36 hours.

In some embodiments, the microbial cellulose film is bathed in the basic solution for a period of 12-24 hours.

In some embodiments, the microbial cellulose film is bathed in the basic solution for a period for 24 hours.

In some embodiments, high-pressure steam is applied to the microbial cellulose in step (c) for a period of 5-120 minutes.

In some embodiments, high-pressure steam is applied to the microbial cellulose in step (c) for a period of 10-60 minutes.

In some embodiments, high-pressure steam is applied to the microbial cellulose in step (c) for a period of 20-40 minutes.

In some embodiments, high-pressure steam is applied to the microbial cellulose in step (c) for a period of 30 minutes.

In some embodiments, the concentration of cellulase solution is 0.1-10% volume/volume (v/v).

In some embodiments, the concentration of cellulase solution is 0.1-5% v/v.

In some embodiments, the concentration of cellulase solution is 0.5-2% v/v.

In some embodiments, the concentration of cellulase solution is 1-2% v/v.

In some embodiments, the concentration of cellulase solution is 1% v/v.

In some embodiments, the alcohol solution in step (b) is ethanol, methanol, isopropanol, ethylene glycol, or glycerol.

In some embodiments, the alcohol solution in step (b) is ethanol, methanol, or isopropanol.

In some embodiments, the alcohol solution in step (b) is ethanol.

In some embodiments, the concentration of ethanol is 50-99%.

In some embodiments, the concentration of ethanol is 60-90%.

In some embodiments, the concentration of ethanol is 60-80%.

In some embodiments, the concentration of ethanol is 80%.

In some embodiments, the basic solution is NaOH, KOH, Mg(OH)₂, Ca(OH)₂, or NH₃.

In some embodiments, the basic solution is NaOH, or KOH.

In some embodiments, the basic solution is NaOH.

In some embodiments, concentration of NaOH is 0.01-5M.

In some embodiments, concentration of NaOH is 0.01-2M.

In some embodiments, concentration of NaOH is 0.05M-1M.

In some embodiments, concentration of NaOH is 0.1 M.

In some embodiments, in step (d), the high-pressure steam is applied at a temperature of 100-400 °C.

In some embodiments, in step (d), the high-pressure steam is applied at a temperature of 150-350 °C.

In some embodiments, in step (d), the high-pressure steam is applied at a temperature of 200-300 °C.

In some embodiments, in step (d), the high-pressure steam is applied at a temperature of 200 °C.

In some embodiments, in step (d), the high-pressure steam has a pressure over 15 psi, 50 psi, 100 psi, 200 psi, 250 psi, 500 psi, or 800 psi.

In some embodiments, Young's modulus of the purified microbial cellulose film is from 1 MPa to 10 MPa.

In some embodiments, Young's modulus of the purified microbial cellulose film is from 1.2 MPa to 5 MPa.

In some embodiments, Young's modulus of the purified microbial cellulose film is from 1.2 MPa to 3 MPa.

In some embodiments, Young's modulus of the purified microbial cellulose film is from 1.5 MPa to 2 MPa.

In some embodiments, ultimate tensile strength of the purified microbial cellulose film is from 0.1 Mpa to 5 Mpa.

In some embodiments, ultimate tensile strength of the purified microbial cellulose film is from 0.2 MPa to 3 MPa.

In some embodiments, ultimate tensile strength of the purified microbial cellulose film is from 0.2 MPa to 2 MPa.

In some embodiments, ultimate tensile strength of the purified microbial cellulose film is from 0.2 MPa to 1 MPa.

In some embodiments, ultimate tensile strength of the purified microbial cellulose film is from 0.2 MPa to 0.5 MPa.

In some embodiments, ultimate tensile strength of the purified microbial cellulose film is from 0.2 MPa to 0.4 MPa.

In some embodiments, yield strength of the purified microbial cellulose film is from 0.1 MPa to 5 MPa.

In some embodiments, yield strength of the purified microbial cellulose film is from 0.1 MPa to 3 MPa.

In some embodiments, yield strength of the purified microbial cellulose film is from 0.1 MPa to 2 MPa.

In some embodiments, yield strength of the purified microbial cellulose film is from 0.2 MPa to 1 MPa.

In some embodiments, yield strength of the purified microbial cellulose film is from 0.2 MPa to 0.5 MPa.

In some embodiments, yield strength of the purified microbial cellulose film is from 0.2 MPa to 0.4 MPa.

In some embodiments, ductility of the purified microbial cellulose film is about 10%-50% of the ductility of the microbial cellulose film before purification.

In some embodiments, the purified microbial cellulose film supports fibroblast viability.

In some embodiments, the purified microbial cellulose film does not stimulate a pro-inflammatory response from macrophages; preferably, the macrophage is naïve THP-1 macrophages.

A process of producing a microfiber mesh may be provided, comprising blending polylactide-co-glycolide (PLGA) with polycaprolactone (PCL).

In some embodiments, the weight ratio between PLGA and PCL is 100:1.

In some embodiments, the weight ratio between PLGA and PCL is 50:1.

In some embodiments, the weight ratio between PLGA and PCL is 25:1.

In some embodiments, the weight ratio between PLGA and PCL is 10:1.

In some embodiments, the weight ratio between PLGA and PCL is 5:1.

In some embodiments, PLGA and PCL is blended in an acid.

In some embodiments, the acid is acetic acid.

In some embodiments, the acetic acid is glacial acetic acid.

In some embodiments, the process further comprises ejecting blended PLGA and PCL onto a stationary surface and electrospinning the blended PLGA and PCL to produce the microfiber mesh.

In some embodiments, the microfiber mesh is unaligned.

In some embodiments, the process further comprises ejecting blended PLGA and PCL onto a moving surface and electrospinning the blended PLGA and PCL to produce the microfiber mesh.

In some embodiments, the microfiber mesh is aligned.

In some embodiments, the microfiber mesh produced in step (a) has a fiber diameter of 0.5-2.5 μm; preferably 0.8-2 μm.

In some embodiments, the microfiber mesh produced in step (a) has a Young's modulus of 200-500 MPa; preferably 250-450 MPa.

In some embodiments, the microfiber mesh produced in step (a) has a tensile strength of 5-15 MPa; preferably 7-10 MPa.

In some embodiments, microfiber mesh produced in step (a) has a yield strength of 5-15 MPa; preferably 6-12 MPa.

In some embodiments, microfiber mesh produced in step (a), when loaded with macrophages, reduces the secretion of pro-inflammatory cytokines, and enhances anti-inflammatory protein production; preferably, the macrophages are M1-induced macrophages.

In some embodiments, microfiber mesh produced in step (b) has a fiber diameter or 0.5-2.5 μm; preferably 0.8-2 μm.

In some embodiments, microfiber mesh produced in step (b) has a Young's modulus of 10-30 MPa; preferably 15-25 MPa.

In some embodiments, microfiber mesh produced in step (b) has a tensile strength of 0.2-1.5 MPa; preferably 0.6-0.9 MPa.

In some embodiments, microfiber mesh produced in step (b) has a yield strength of 0.2-0.7 MPa; preferably 0.3-0.6 MPa.

In some embodiments, microfiber mesh produced in step (b), when loaded with macrophages, elicits a pro-inflammatory response, and reduces anti-inflammatory cytokine secretion, preferably, the macrophages are M1-induced macrophages.

In some embodiments, the microfiber mesh promotes macrophage activation.

This invention investigated and determined design parameters for the development of microbial cellulose as non-medical and medical textiles. Inspired by the versatile nanoscale to macroscale properties of microbial cellulose materials, the working hypothesis of this invention was two-fold: For non-medical approaches, the development of green bioprocessing strategies as post-modification treatments for biofilms will alter material properties for suitable substitutes in the textile industry. For medical approaches, the development of a controlled purification methodology will support the utilization of microbial cellulose as biocompatible scaffold for tissue engineering applications, in comparison to conventional synthetic micro-fiber substrates. To evaluate these hypotheses, this thesis explored three specific aims.

Aim 1 focused on investigating fermentation parameters to control the growth of the obligate aerobic microbe G. xylinus, and its effect on the subsequent synthesis and bottom-up assembly of microbial cellulose. To this end, the effect of monosaccharides (glucose, fructose), disaccharides (sucrose), and sugar alcohols (mannitol, xylitol) as carbon sources at varying concentrations were evaluated to modulate the production of microbial cellulose. In this aim it was observed that while glucose, fructose, sucrose, mannitol, and xylitol supported microbial growth, differences observed in subsequent cellulose production, surface porosity, crystal structure, and mechanical behavior confirmed the modification of material properties during biosynthesis. Collectively, this invention provided insights into the use of microbial biosynthesis for the production of both non-medical or medical biotextiles.

Following the biofabrication of nanofibril cellulose, Aim 2 focused on developing microbial cellulose as a natural alternative for conventional textiles by assessing the effect of bio-inspired treatments with reduced human health and environmental impacts to modify cellulose performance properties. Initially, inspired by traditional brain and smoke tanning known to crosslink collagen fibrils into leather, this invention determined the effect of green bioprocessing with soybean-based lecithin phosphatidylcholine to stabilize microbial cellulose's mechanical properties without altering cellulose chemical composition and nanofibrous morphology. Next, the effect of green-based plasticizers (sorbitol and glycerol), and metal-based crosslinking post-processing treatments were evaluated to enhance microbial cellulose performance properties and functionality. Collectively, this invention harnessed green bioprocessing techniques coupled with microbial biofabrication to develop microbial cellulose with enhanced and stabilized mechanical behavior, without the significant human health and environmental impacts observed with conventional textiles.

Aim 3 focused on developing microbial cellulose as a tissue engineering scaffold, as well as, elucidating the effect of fibrous matrix alignment, chemical induction, and mechanical stimulation on macrophage response. A purification procedure was assessed to prepare microbial cellulose as a biocompatible, biomimetic nanofibrous scaffold that is suitable for in-vitro cell culture. Following, the use of fibrous scaffolds for immunomodulation was determined by evaluating the coupled effects of fibrous matrix alignment, chemical induction, and mechanical stimulation on macrophage phenotype. Building on these understandings, Aim 3 evaluated microbial cellulose as a tissue engineering scaffold with future potential to be used for immunomodulatory applications.

### Aim 1: Biofabrication of Microbial Cellulose

Aim 1 focused on determining the role of carbon source and concentration on modulating microbial cellulose production by G. xylinus. Here, glucose, fructose, sucrose, mannitol, and xylitol at 2% and 8% w/v were utilized within Hestrin-Schramm medium to biofabricate microbial cellulose, in which bacterial cell response and biomaterial properties were assessed. It was determined that while all sugars supported G. xylinus growth with different doubling rates, the resulting biosynthesis of mCellulose was greatly affected. Conventionally utilized glucose was observed to not only support lower lag and population doubling times, but also reduced pH in culture media, indicative of gluconic acid formation, which limited cellulose yield. On the other hand, throughout 14 days of culture, mannitol and sucrose enhanced cellulose production at 2% and 8% w/v, respectively, suggesting an adaptation mechanism that enhanced target metabolic pathways. All mCellulose biofabricated from different sugars exhibited similar nanofibrous morphology and chemical compositions, however, surface porosity, crystal structure, and mechanical testing confirmed the modification of material properties during biosynthesis. Though differences in yield between carbon sources was minimized at higher sugar concentrations, the overall increase in biosynthesis led to a significant increase in Young's Modulus and ultimate tensile strength. Moreover, modification of carbon source and concentration in culture medium can be used to synthesize specific dominance of cellulose allomorphs dependent upon crystal size, albeit not reflected in tensile mechanical properties. In addition, a cradle-to-gate life cycle impact assessment was implemented, in which sucrose offered significant improvement in reducing human health and environmental toxicity compared to all other sugars for 2% w/v cultures. Overall, the ability to not only tune yield, but also performance properties by biofabricating with carbon sources that have varying human health and environmental impacts, incentivizes the transition to a circular materials economy, offering a versatile platform for non-medical and medical textile applications.

### Aim 2: Development of Non-medical Microbial Cellulose Textiles

Aim 2 investigated the development of microbial cellulose as a regenerative, high performance alternative for conventional textiles. Several bio-inspired treatments with reduced human health and environmental impacts were selected to modify cellulose performance properties. Initially, the effect of green bioprocessing with soybean-based lecithin phosphatidylcholine to stabilize microbial cellulose's mechanical properties was assessed. It was demonstrated that lecithin treatment modified microbial cellulose via bio-phosphorylation yielding enhanced tensile strength and ductility, as a result of crosslinking through hydroxyl, phosphate and methylene groups. Additionally, phosphorylation redirected the combustion pathway from levoglucosan production towards the formation of char as an insulating oxygen barrier, promoting flame retardance.

The ability to couple biofabrication with green bioprocessing techniques was also evaluated using bioplasticizing and crosslinking strategies. Two post-processing approaches utilizing glycerol and sorbitol as bioplasticizers enhanced microbial cellulose toughness in a concentration dependent manner, eliminating the use of conventional endocrine-disrupting phthalates for improving performance properties. An alternative crosslinking procedure incorporating tannic acid and iron ion complexation modified microbial cellulose with apparent antimicrobial properties, as well as, enhanced toughness and strength, which was strongly dependent upon pH conditions. Life cycle assessment revealed that green bioprocessing techniques significantly reduced environmental impacts compared to the conventional plasticizer di-2ethylhexyl phthalate, known to be applied during the production of polyvinyl chloride plastic. Ultimately, this invention demonstrated the ability of green chemistry to mechanically stabilize and functionalize mCellulose, further elucidating the potential of this biofabrication platform to support a circular materials economy.

### Aim 3: Development of Tissue Engineering Microbial Cellulose Scaffolds

Aim 3 focused on assessing microbial cellulose as a biocompatible tissue engineering scaffold, as well as, elucidating the effect of microenvironment stimulatory cues on macrophage response. A purification procedure was assessed to prepare microbial cellulose as a biocompatible, biomimetic nanofibrous scaffold that is suitable for in-vitro cell culture. A sterilization procedure, based upon conventional chemical and high-pressure steam approaches, was investigated for purifying microbial cellulose biofilms from bacterial and pro-inflammatory-inducing endotoxin content. It was demonstrated that sequential immersion of biofilms into cellulase, 70% ethanol, and 0.1 M sodium hydroxide, followed by autoclaving, reduced both bacterial cell viability and endotoxin concentration, albeit this strategy was not able to reduce toxin concentrations lower than FDA's requirement for medical devices. To this end, purified microbial cellulose did not stimulate a pro-inflammatory response from naïve THP-1 macrophages, and supported fibroblast viability and proliferation for two weeks. Overall, this invention demonstrated the potential of microbial cellulose as a biocompatible scaffold for tissue engineering applications.

Following, the use of fibrous scaffolds for immunomodulation was determined by evaluating the role of PLGA:PCL fibrous matrix alignment, chemical induction, and mechanical stimulation on macrophage phenotype. It was shown that guidance of macrophage phenotype with mechanical stimulation was only possible for pro-inflammatory induced cells, in which matrix alignment dictated modulation towards greater pro- or anti-inflammatory response. On loaded unaligned matrices, pro-inflammatory macrophages secreted greater anti-inflammatory cytokines per cell, while on loaded aligned matrices these cells conversely produced more pro-inflammatory proteins. Although it was observed that microfiber matrix and chemical induction can separately promote macrophage activation, it was the combined stimulation between polarization and mechanical loading that played an important role in immunomodulation of macrophage response, in which further tuning of fiber alignment can alter cell phenotype.

### Use of Industrial Waste Streams for Microbial Cellulose Biofabrication

Although microbial cellulose production can be strictly controlled by environmental parameters, such as nutrient availability, temperature, agitation, and oxygenation, these same factors can also limit this biofabrication platform by genetically modifying the bacterial producers and prevent subsequent polymer synthesis. As a result, static cultivation of microbial cellulose synthesis requires controlled production processes that minimize batch-to-batch biomaterial variability. In this thesis, several carbon sources, such as glucose, fructose, sucrose, mannitol, and xylitol, at varying concentrations were investigated to direct microbial cellulose synthesis, as well as modulate material properties. Due to the complexity of acetic acid bacteria's carbon metabolism, this biofabrication platform allows for utilizing a vast catalogue of carbon sources with different human toxicity and environmental impacts to synthesize the same biopolymer, albeit at varying efficiencies. One potential approach to further mitigate human toxicity and environmental impacts is to source nutrients for biosynthesis media from agro-industrial waste streams. Thus, this invention determined the viability of agro-industrial waste streams as low impact waste-to-resource strategies for biofabricating high performance microbial cellulose biotextiles within a circular economy.

### Use of Bioplasticizers for Microbial Cellulose Biofabrication

This thesis has focused on demonstrating the potential of combining microbial cellulose biofabrication with green post-processing via bioplasticizers for developing high performance biotextiles. Despite utilizing green strategies throughout the development of microbial cellulose biotextiles, both biofabrication and bioprocessing procedures contribute human toxicity and environmental impacts. To mitigate large impacts at scale, one potential approach is to utilize bioplasticizers as carbon sources in biosynthesis media, rather than as post-processing techniques. In this invention it was shown that immersing microbial cellulose into sorbitol and glycerol improved ductility by increasing the space between cellulose molecules, supporting greater mobility of polymeric chains. Thus, this invention determined the effect of bioplasticizers, such as sorbitol and glycerol, to simultaneously be metabolized into cellulose by G. xylinus, while modulating material properties by increasing mobility between biopolymer chains.

### Further Evaluation of Tannic Acid—Metal Complexation

Tannic acid—metal complexation was investigated in this thesis as an alternative post-processing strategy to modulate microbial cellulose mechanical properties. In this approach, tannic acid and iron chloride were utilized to determine the effects of pH-tunable coordinate interactions with cellulose. While mechanical properties were assessed after altering pH into acidic, neutral, and basic environments, it is also possible to further tune these covalent interactions by adjusting the concentrations of tannic acid and FeCl₃, resulting in optimal control of cellulose ductility, strength, and antimicrobial activity. Additionally, other metals, such as zinc, aluminum, and magnesium, can be complexed with tannic acid and cellulose, offering an immense platform to functionalize microbial cellulose biotextiles for non-medical and medical applications. Notably, metals, such as zinc, play a critical role in physiologic cellular environments, in which they can be found in many tissues and immune system. Thus, invention further explored the ability for tannic acid—metal functionalized microbial cellulose scaffolds to elicit cellular responses for connective tissue injury and repair.

### Design of Biodegradable Microbial Cellulose Scaffolds

Tissue engineering biofabrication approaches aim to develop scaffolds that are both biocompatible and biodegradable for guided cell-mediated regeneration, replacing tissue lost from disease or injury. Although microbial cellulose in-vitro biocompatibility has been established in this thesis through macrophage and fibroblast culture, its lack of in-vivo biodegradability in eukaryotic systems has been suggested as a limitation for the design of an optimal fiber-based matrix for tissue regeneration applications. Briefly, the absence of enzymatic and chemical mechanisms in mammalian cells or within the extracellular matrix that can hydrolyze cellulose's linear β-1,4-glycosidic linkages hinders the ability of newly formed tissue to replace it over time. Future work can evaluate low human toxicity and environmental impact strategies to modify cellulose chemical structure for tailored degradation in in-vivo environments, without altering inherent nanofibrous morphology.

### Control of Microbial Cellulose Geometry and Fiber Alignment

During static cultivation, aerobic cellulose producing bacteria are free to move in any direction without restriction at the air-to-medium interface, resulting in the random distribution of cellulose nanofibrils that coagulate into an unaligned matrix. Since the formation of microbial cellulose is subjected to the air-to-medium interface throughout static cultivation, this biofabrication platform is limited by its dependence upon area intensive processing that produces uncontrolled biomaterial geometry and fiber alignment. Additionally, statically biofabricated microbial cellulose biofilms are highly dependent on the geometry of the culture vessel, further limiting material size.

In this invention it was demonstrated that aligned matrices not only have enhanced tensile mechanical properties, but also promote different cell responses from macrophages compared to unaligned orientations. Thus, invention explores strategies, such as electrical stimulation, to control microbial cellulose fiber alignment during G. xylinus culture, providing insight on further tailoring of textile properties for both non-medical and tissue engineering approaches. Furthermore, to address the limitations of flat microbial cellulose biomaterials, future studies may explore the use of air-permeable silicone-based vessels for bacterial culture and subsequent synthesis of cellulose nanofibrils.

### DEFINITIONS

### List of Abbreviations

2ME: 2-Mercaptoethanol
3D: 3-dimensional
ACL: Anterior Cruciate Ligament
aFGF: Acidic Fibroblast Growth Factor
Amp-B: Amphotericin B
ANOVA: Analysis of variance
ASTM: American Society for Testing and Materials
ATR: Attenuated Total Reflectance
CFC: Chlorofluorocarbon
CI: Crystallinity index
CTUe: Comparative Toxic Unit
DCM: Dichloromethane
DEHP: di(2-ethylhexyl)phthalate
DINP: di(isononyl)phthalate
DMEM: Dulbecco's Modified Eagle Medium
DMF: Dimethylformamide
DMMB: Dimethylmethylene blue
DNA: Deoxyribose Nuclear Acid
DOP: Di-2ethylhexyl Phthalate
ECH: Epichlorohydrin
ECM: Extracellular Matrix
EDXA: Energy-Dispersive X-ray Spectroscopy
EPA: Environmental Protection Agency
EtOH: Ethanol
EU: Endotoxin Units
FBS: Fetal Bovine Serum
FDA: Food and Drug Administration
FTIR: Fourier Transform Infrared Spectroscopy
FTIR-ATR: Fourier Transform Infrared Spectroscopy Attenuated Total Reflectance
GAG: Glycosaminoglycan
G/S: Gentamicin Sulfate
HMDS: Hexamethyldisilazane
HS: Hestrin-Schramm
hPBMCs: Human Peripheral Blood Mononuclear Cells
IFN-γ: Interferon-γ
IL: Interleukin
ISO: International Organization for Standardization
LFF: Long fine focus
LB: Luria Broth Base
LCA: Life Cycle Assessment
LCIA: Life Cycle Impact Assessment
LDPE: Low Density Polyethylene
LPS: Lipopolysaccharide
M0: naïve Macrophage
M1: Pro-inflammatory Macrophage
M2: Anti-inflammatory Macrophage
mCellulose: Microbial Cellulose
mPts: milliPoints
MMP: Matrix metalloproteinase
MSC: Mesenchymal Stem Cell
NaOH: Sodium Hydroxide
NBF: Neutral Buffered Formalin
NEAA: Non-Essential Amino Acids
PBS: Phosphate Buffered Saline
PCL: poly(ε-caprolactone)
PGA: poly(glycolic acid)
PLGA: poly(lactide-co-glycolide)
PLLA: poly(L-lactic acid)
PMA: Phorbol 12-myristate 13-acetate
P/S: Penicillin-Streptomycin
PVC: Polyvinyl Chloride
RPMI: Roswell Park Memorial Institute
SCOBY: Symbiotic Colony of Bacteria and Yeast
SEM: Scanning Electron Microscopy
TA: Tannic Acid
TAFe: Tannic Acid—Iron
TGA: Thermogravimetric Analyzer
TLR-4: Toll-like Receptor 4
TNF: Tumor Necrosis Factor
TRACI: Tool for the Reduction and Assessment of Chemical and other Environmental Impacts
UTS: Ultimate Tensile Strength
UV: Ultraviolet
VEGF: Vascular Endothelial Growth Factor
XRD: X-ray Powder Diffraction

As used herein, “zone of inhibition” (ZOI), also known as a zone of clearing or a halo assay, refers to the clear zone surrounding an antimicrobial agent. These ZOIs result from a complete absence of bacteria on, or within a confluent bacterial lawn.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, would control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. In embodiments, about means within a standard deviation using measurements generally acceptable in the art. In embodiments, about means a range extending to +/−10% of the specified value. In embodiments, about includes the specified value. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It would be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter may at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

As used herein, “zone of inhibition” (ZOI), also known as a zone of clearing or a halo assay, refers to the clear zone surrounding an antimicrobial agent. These ZOIs result from a complete absence of bacteria on, or within a confluent bacterial lawn.

### General

For the foregoing embodiments, each embodiment disclosed herein is contemplated as being applicable to each of the other disclosed embodiments.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention would become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXAMPLES

### Example 1

Example 1 evaluate the effect of carbon sources at varying concentrations on the production of microbial cellulose. It is hypothesized that glucose would enhance cellulose yield relative to disaccharides and sugar alcohols, and material properties would be carbon source dependent. Further, it is anticipated that higher carbon source concentrations utilized during culture would result in greater cellulose yield.

### Materials and Methods

### Microbial Cellulose Biosynthesis

The Gluconacetobacter xylinus strain ATCC 31174 was acquired for microbial cellulose biofabrication. Hestrin-Schramm (HS) culture medium (0.5% w/v yeast extract [Fisher Scientific], 0.5% w/v peptone [Fisher Scientific], 0.27% w/v sodium phosphate dibasic anhydrous [Fisher Scientific]), was used for bacterial culture. The medium pH was adjusted to 4.5 using citric acid (Fisher Scientific). All culture medium was sterilized via autoclaving at 250 °C for 20 minutes.

Prior to assessing the effect of carbon sources and concentration on cellulose production, G. xylinus was pre-cultured in sterile HS medium containing 2% w/v mannitol for 72 hours at 30 °C under static conditions. Microbial cellulose was biofabricated by inoculating (5% v/v) the preculture into sterile HS medium containing varying sugars (glucose [Fisher Scientific], fructose [ADM], sucrose [Sigma-Aldrich], mannitol [Fisher Scientific], and xylitol [Fisher Scientific]). Both low (2% w/v) and high (8% w/v) concentrations of each carbon source was investigated. All cultures were held under static conditions at 30 °C for 14 days, in which 2-D layer by layer production of cellulose biofilms (Ø=30 mm) were biofabricated overtime.

### Bacterial Growth Characterization

G. xylinus growth curves were determined by assessing turbidity, by inoculating (5% w/v) the preculture into sterile HS medium, containing either glucose, fructose, sucrose, mannitol, or xylitol, within a 96-well plate and immediately measuring the absorbance for 100 hours via a spectrophotometer (BioTek Synergy 5, 30 °C, OD: 600 nm, n=5). Bacterial cell lag time (n=5) was calculated by evaluating the amount of time utilized to get to an absorbance of 0.1, prior to the exponential phase. Additionally, cell doubling time (n=5) was measured from the growth curve's exponential phase with the following equation:

$$Doubling Time = [ \frac{( Ln ( 2 ) )}{b} ] ,$$

where b represents the exponential component (Y=Ae^{bx}).

### Microbial Cellulose Purification and Yield Quantification

At selected timepoints (up to 14 days), mCellulose samples were removed from the air-media interface and subjected to a purification procedure, while the culture medium was collected for pH analysis. The pH of culture medium was assessed with a pH meter (Thermo Fisher Scientific) at each timepoint (n=5). mCellulose biofilms were washed three times with deionized water to remove residual sugars and medium components. To lyse G. xylinus cells on the surface and within the cellulose network, pellicles were heated at 90 °C. in diH₂O for 1 hour, followed by a 0.1 M NaOH (Sigma-Aldrich) bath for 2 hours. All pellicles were continuously washed with deionized water to further remove medium components from the cellulose until the pH returned to neutral conditions. Hydrated microbial cellulose samples were placed in a −80 °C. freezer for 24 hours and then lyophilized in a freeze dryer system (Labconco FreeZone) for 48 hours at −84 °C and 2.0×10⁻² mbar. Lyophilized microbial cellulose samples biofabricated from varying carbon sources and concentrations were weighed on an analytical balance to determine cellulose yield (n=5).

### Nanofiber Characterization

mCellulose samples biosynthesized for 14 days were washed with deionized water three times and characterized for surface morphology, tensile properties, chemical composition, and crystal structure.

Microbial cellulose morphology, fiber diameter, and porosity was assessed by performing scanning electron microscopy (SEM, 3 kV, Zeiss Sigma VP) on the surface of samples (Ø=10 mm) that were sputter coated with 10 nm of gold-palladium to reduce charging effects (Cressington 108 Auto, Cressington Scientific). Fiber diameter was quantified by analyzing randomly selected fiber segments in SEM micrographs (10,000X, n=100 fibers/group) using ImageJ software (National Institutes of Health). Additionally, surface porosity was measured by assessing spacing between fibers (35,000X, n 6 micrographs/group) via ImageJ.

Uniaxial tensile testing was conducted based upon the ASTM International standard tensile test method for plastics (D638-14). Microbial cellulose samples (5×1 cm, n=8) were secured with clamps and mounted in a uniaxial tensile testing machine (Instron) equipped with a 100 N load cell. Biofilms were maintained to have an average gauge length of 30 mm, and were tested to failure at a strain rate of 5 mm/minute. Microbial cellulose elastic modulus, toughness, ultimate tensile strength, and ductility were determined from the resulting stress-strain curve.

Microbial cellulose chemical composition was determined by using Fourier transform infrared spectroscopy in attenuated total reflectance mode (FTIR-ATR, n=5, LUMOS). All spectra were collected from 4000-600 cm⁻¹ (spectral resolution 2 cm⁻¹, 200 scans). Baseline subtraction using vibrational spectroscopy OPUS software (Bruker) was implemented, and all spectra were assessed for characteristic cellulose peaks. Further FTIR analysis was performed to determine the fraction of cellulose crystal structure as I_{α} (triclinic) in relation to the Is (monoclinic) allomorph using the ratio of band areas between A₇₅₀ and A₇₁₀:

$${f}_{I α} = \frac{{A}_{750}}{{A}_{750} + {A}_{710}} * 100$$

The curve area of I_{α} (A₇₅₀: 750 cm⁻¹) and I_{β} (A₇₁₀: 710 cm⁻¹) was calculated using Gaussian functions via a custom MatLab code.

Microbial cellulose crystal structure was assessed by X-ray diffraction (XRD, n=5, Panalytical XPert3 Powder XRD, Malvern Panalytical). Samples were positioned onto a zero-diffraction plate (silicon, p-type, β-doped, MTI Corporation, Richmond, CA) and scanned using a diffractometer with a 3-kV generator, X-ray tube, fully ceramic Cu Long fine focus (LFF), vertical goniometer (theta-theta) and a PIXcel id detector. Diffractograms were measured through a 1 cm copper-based beam mask from 5-40° (2θ) with a step size of 0.013°, scanning rate of 1.68°/min, and a beam path utilizing a 1/2° divergence slit. Background subtraction was applied using Highscore XRD Software. A XRD peak deconvolution method was utilized as a curve-fitting process in Igor-Pro software to calculate the crystallinity of microbial cellulose samples. Crystalline and amorphous peaks were identified and separated in XRD spectra. Gaussian functions were used for the deconvolution of spectra into three crystalline peaks (100, 010, and 110), and one amorphous peak centered at a 2θ value of ~18.5 degrees. A Vaugt function was used to fit the crystalline peak identified as 200. Crystallinity index (CI) was calculated from the ratio of the area of crystalline regions (A_{cry}) to the total peak area (Aₜₒₜₐₗ):

$$C ( \% ) = \frac{{A}_{cry}}{{A}_{total}} * 100$$

Crystallite size was determined by calculating the Scherrer width via the Scherrer equation:

$$C S = \frac{k λ}{FWHM * cos ( θ )} ,$$

where dₕₖₗ is the size of the crystallite (nm), k is the Scherrer constant of 0.94, λ is the x-ray wavelength set at 0.154 nm, and θ is the diffraction angle.
Additionally, the interplanar lattice spacing (d-spacing) was determined by Bragg's equation:

$${d}_{hkl} = \frac{λ}{2 sin ( θ )} ,$$

in which dₕₖₗ is the lattice spacing of the crystallographic planes, λ is the x-ray wavelength (0.154 nm), and θ is the corresponding Bragg angle.
Discriminant analysis was performed to categorize cellulose crystalline structure as either dominantly I_{α} (triclinic) or I_{β} (monoclinic) allomorphs. The Z-discriminant function which classifies between I_{α} or I_{β}, using two equatorial d-spacings, without error is given by:

Z=1693d₁(nm)−902d₂(nm)−549

, where d₁ and d₂ are the d-spacing of peaks 100, and 010, respectively. Accordingly, Z>0 indicates that cellulose is I_{α} dominant, while Z<0 indicates an I_{β} rich allomorph.

### Carbon Source Impact Analysis

Life cycle impact assessment (LCA) analysis is a quantitative technique utilized to determine the environmental and human health impacts within a product's lifespan, including raw material extraction, manufacturing, distribution, consumer use, and disposal. The International Organization for Standardization (ISO) for Environmental Management of Life Cycle Assessment—Principles and Frame work (ISO 14040, Geneva, Switzerland) was used to perform cradle-to-gate life cycle impact assessment. For each carbon source of interest, manufacturing impacts were determined using databases within Sustainable Minds© Life Cycle Assessment (Cambridge, MA) and EcoInvent (Zurich, Switzerland) software. Following, relevant impact categories classified based upon ecological and human health damage were selected using the U.S. Environmental Protection Agency's (EPA) Tool for the Reduction and Assessment of Chemical and other Environmental Impacts (TRACI), providing characterization factors for life cycle impact assessment (LCIA) methodology. Ecological damage encompassed impact categories such as acidification (kg SO₂ eq.), ecotoxicity (CTUe), eutrophication (kg N eq.), global warming (kg CO₂ eq.), ozone depletion (kg CFC⁻¹¹ eq.), while human health damage included carcinogenics and non-carcinogenics (hormonally active agents (HAA), CTUh), respiratory effects (kg PM2.5 eq., fine particulates), smog formation (kg (ground level) 03 eq.) and fossil fuel depletion (MJ surplus).

The cradle-to-gate LCA was implemented to compare the human health and environmental impacts of manufacturing the various carbon sources, including glucose, fructose, sucrose, mannitol, and xylitol, used to biofabricate mCellulose biofilms. Based upon prior experimental procedures for biofabricating mCellulose in 2% w/v cultures, the functional unit implemented into LCA was the yielded amount of cellulose after 14 days when 0.9 g of each sugar was used during fermentation (glucose: 30.98 mg, fructose: 10.88 mg, sucrose: 12.78 mg, mannitol: 41.75 mg, xylitol: 17.68 mg). All impact categories were converted into respective impacts expressed in milliPoints per functional unit (mPts/cm²) by using a combination of normalization and weighting factors. Briefly, one point (1000 mPts) represents the average annual environmental impact, including material production and consumption activities, of a consumer in the United States. Normalization was implemented to express the prior impacts relative to mPts as a reference by calculating normalization factors from characterization factors within the TRACI 2.1 LCIA model. Additionally, the relative weighting of each impact category was calculated to scale LCA results of measured materials with the environmental performance of competing products. Here, the life cycle assessment was partially performed to only encompass raw material extraction and manufacturing impacts, while further distribution, consumer use, and disposal impacts were not incorporated.

Lastly, each carbon source's total impact was segmented into 10 categories encompassing both ecological damage and human health damage. As defined by the software, ecological damage is comprised of acidification (kg SO₂ eq), ecotoxicity (CTUe), eutrophication (kg N eq), global warming (kg CO₂ eq), and ozone depletion (kg CFC-11 kg). Human health damage includes carcinogenics and non-carcinogenics (both in units of CTUh), respiratory effects (kg PM2.5 eq, fine particulates), and smog (kg (ground level O₃ eq), while resource depletion exclusively refers to extraction of fossil fuels (MJ surplus). Impacts are visually displayed within a donut chart to provide insights of the largest hotspot, and further organized in hierarchical order of total impacts, however, respective plot sizes are not scaled.

### Reagents and Materials

All reagents and materials were supplied from Fisher Scientific, unless otherwise stated. Statistical Analysis

Results are reported as mean±standard deviation, with ‘n’ equal to the number of replicates per study group. One-way analysis of variance (ANOVA) was utilized to determine the effect of carbon source on nanofiber diameter, and tensile mechanical properties. Multi-way ANOVA was used to determine the effect of carbon source on cellulose production, and medium pH between groups and temporally. The Tukey-Kramer post-hoc test was used for all pair-wise comparisons with significance was attained at p<0.05. All statistical analyses were performed by utilizing JMP-IN (4.0.4, SAS Institute, Inc.).

### Results

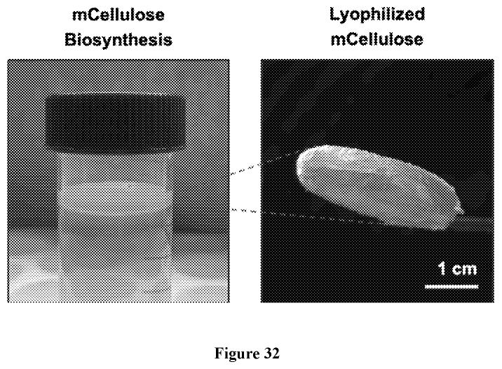

Effect of Carbon Source and Concentration on G. xylinus Growth
To investigate the effects of carbon source and concentration on microbial growth overtime, the turbidity of culture medium incorporating either glucose, fructose, sucrose, mannitol, or xylitol at 2% and 8% w/v was measured (FIG. 32). While viability was maintained with all carbon sources for over 100 hours, significantly lower lag times were measured at 6.00±0.70 and 7.20±2.16 hours in 2% and 8% w/v glucose-based cultures, respectively, when compared to other sugars (n=5, FIG. 32). Surprisingly, differences in lag time between concentrations were only observed in fructose-based cultures (n=5, \hat{ }p<0.05). Additionally, microbial population doubling reduced to 6 hours during the exponential phase when supplemented with glucose, indicative of greater proliferation (n=5, FIG. 32). However, no significant differences were observed between low and high glucose concentrations. Utilization of fructose, sucrose, and mannitol increased population doubling times, in which higher concentrations for each significantly mitigated this effect. Notably, xylitol-based cultures had the largest population doublings ranging from 29 to 44 hours in 2% and 8% w/v, respectively.

Acidity measurements within medium revealed that pH decreased over time in glucose-based cultures, with significantly greater acidic environment formed at higher concentrations by day 7 (n=5, FIG. 32). Albeit at lower magnitudes, pH of 8% w/v sucrose-based medium similarly decreased by day 4, likely due to the metabolism of the disaccharide into glucose monomers, however, differences were not observed at 2% w/v (n=5, \hat{ }p<0.05). The pH did not differ significantly between all other groups (fructose, mannitol, and xylitol) and was maintained at the initial measurement of 4.5.

### Microbial Cellulose Bioproduction via Varying Carbon Sources

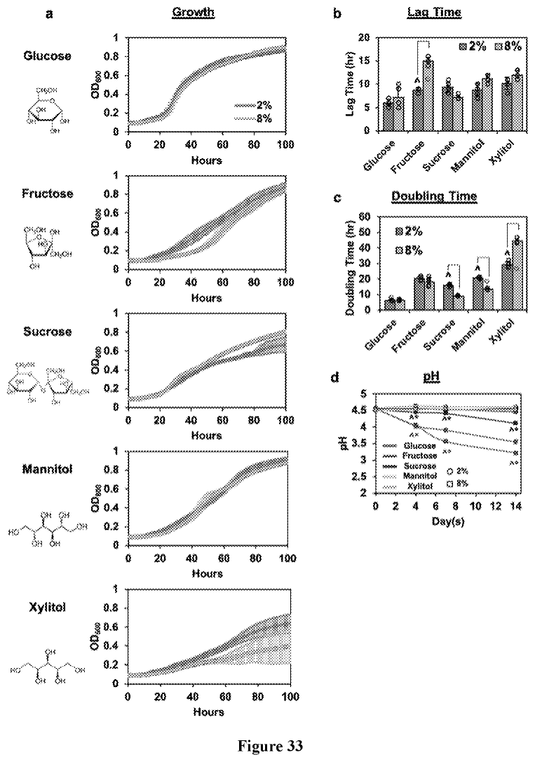

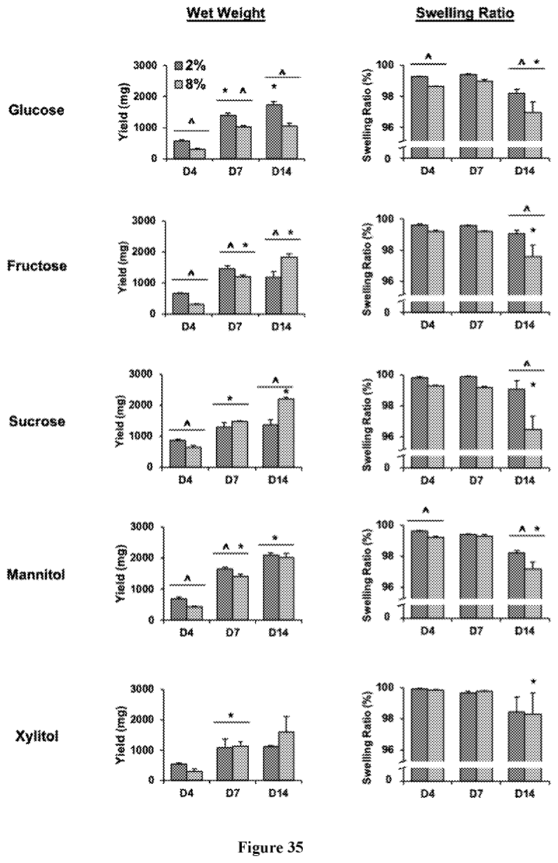

To determine the effects of carbon source and concentration on cellulose production over time, the yield of mCellulose biofabricated from glucose, fructose, sucrose, mannitol, and xylitol at 2% and 8% w/v was measured. Throughout 14 days, all sugars supported the biofabrication of hydrated mCellulose biofilms, at the air-media interface, with cylindrical geometries and a light-brown color, as a result of the shape of the culturing vessel and a Maillard reaction during culture, respectively (FIG. 33). Purification and lyophilization of mCellulose resulted in dried, white biofilms. While synthesis was observed with all carbon sources, cellulose production significantly enhanced at a higher concentration (8% w/v) by day 14 when utilizing fructose, sucrose, and mannitol (n=5), with no differences seen in glucose and xylitol groups. In 2% w/v groups, mCellulose yield by day 14 was 30.98±3.61, 11.61±0.33, 12.78±7.62, 37.58±3.75, and 17.68±11.01 mg for glucose, fructose, sucrose, mannitol, and xylitol, respectively, resulting in significantly enhanced production with mannitol (n=5). Moreover, cellulose production via 2% w/v mannitol-based media significantly increased overtime (n=5). An increase in concentration to 8% w/v yielded 32.00±7.80, 37.47±5.17, 69.40±3.14, 56.82±6.64, and 30.84±25.23 mg in glucose, fructose, sucrose, mannitol, and xylitol groups, respectively, resulting in sucrose supporting greater production between groups (n=5), and over time by day 7 (n=5). Of note, cellulose mass was greater in as-fabricated hydrated state compared to dried forms, in which the swelling ratio was similar between all groups (>96% water), although minute differences were observed by day 14 (FIG. 35).

### Microbial Cellulose Nanofiber Characterization

The structural and chemical properties of mCellulose biofabricated from glucose, fructose, sucrose, mannitol, and xylitol-based medium for 14 days of culture were assessed to determine the effects of carbon source and concentration on nanofibril formation.

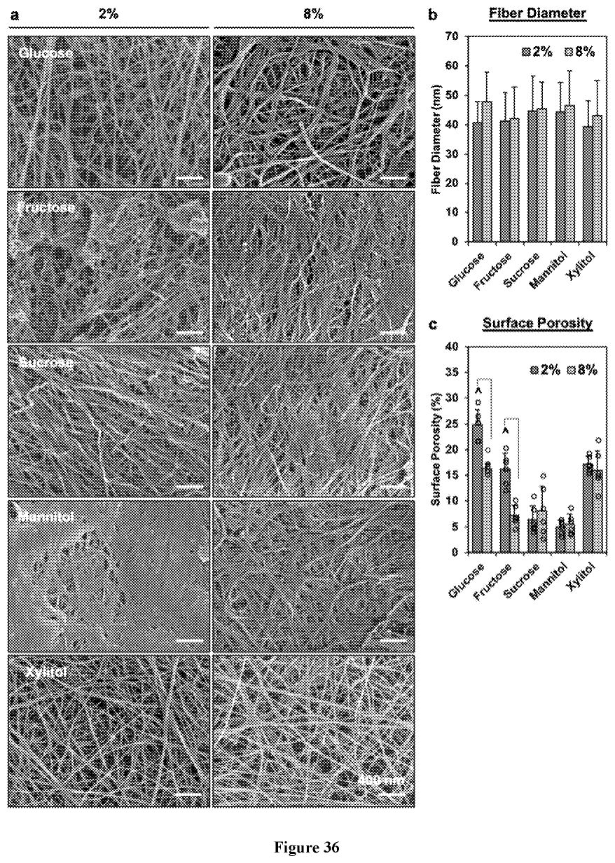

All carbon sources biofabricated biofilms with unaligned nanofibrous morphologies evident via scanning electron microscopy (SEM, FIG. 36). The conventional 2% w/v glucose-based HS media was used to yield nanofibers with a diameter measuring 40.82±7.17 nm, with no significant difference in either fiber morphology or diameter when alternative monosaccharides, disaccharides, and poly-alcohols at 2% or 8% w/v were utilized (FIG. 36). Of note, the surface porosity between individual nanofibers was significantly reduced in fructose (8% w/v), sucrose (2% and 8% w/v), and mannitol (2% and 8% w/v) groups, indicating mCellulose structure and fiber production was affected by carbon source (n=6, FIG. 36).

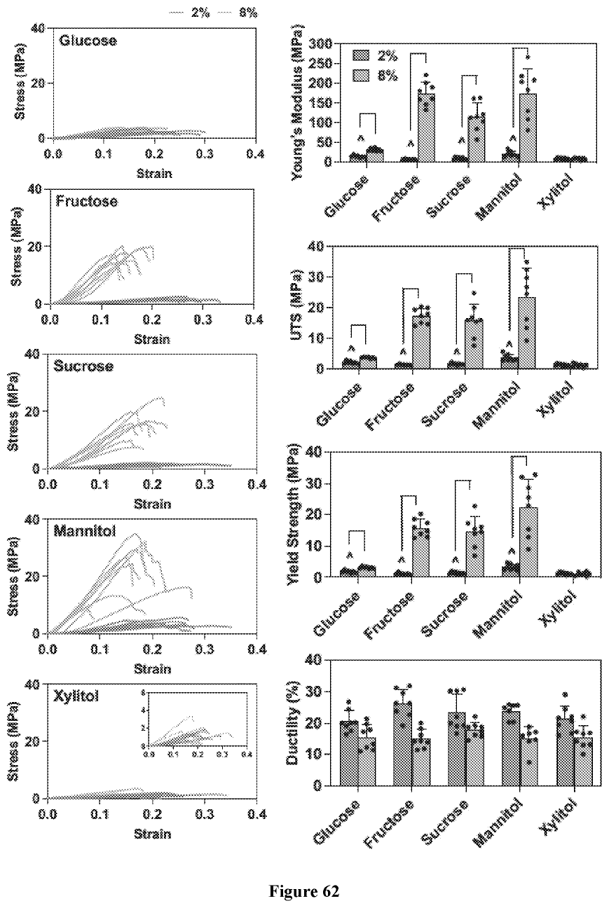

After morphological characterization, the mechanical properties were evaluated for dried mCellulose biofilms biofabricated from various carbon sources and concentrations (FIG. 62). While the ductility remained similar between carbon source and concentration (2% and 8% w/v), the ultimate tensile strength (Glucose: 2.08±0.40 vs. 3.61±0.26 MPa; Fructose: 1.33±0.21 vs. 17.26±2.49 MPa; Sucrose: 1.52±0.36 vs. 15.73±5.37 MPa; Mannitol: 3.63±1.07 vs. 23.34±9.40 MPa), Young's Modulus (Glucose: 14.52±2.75 vs. 31.21±4.53 MPa; Fructose: 7.10±1.04 vs. 173.69±29.53 MPa; Sucrose: 8.95±3.27 vs. 114.60±35.47 MPa; Mannitol: 20.32±7.66 vs. 174.67±62.68 MPa), and yield strength (Glucose: 1.76±0.39 vs. 3.10±0.32 MPa; Fructose: 1.15±0.25 vs. 15.79±2.77 MPa; Sucrose: 1.35±0.36 vs. 14.57±4.79 MPa; Mannitol: 3.28±0.89 vs. 22.39±9.01 MPa) significantly increased at 8% w/v (n=8), with the exception of xylitol.

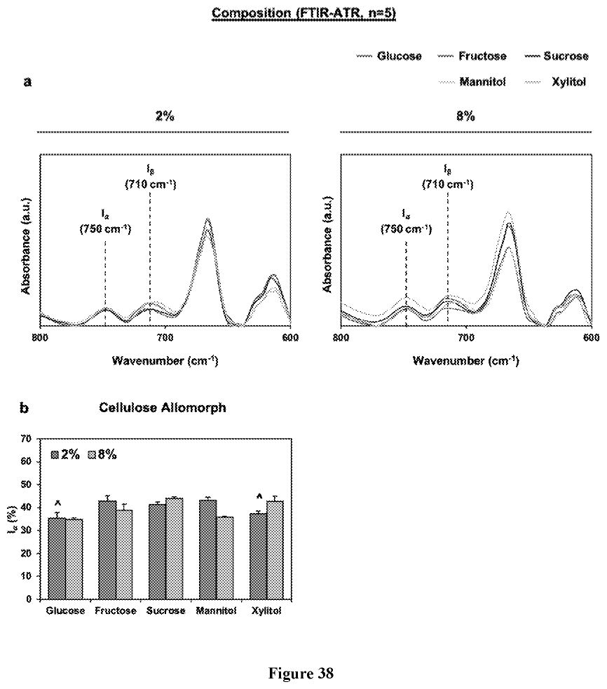

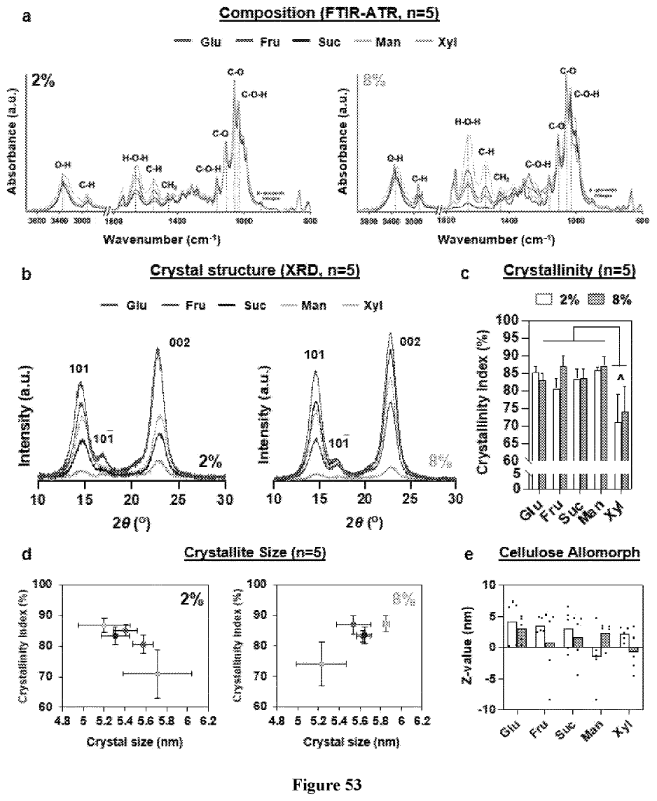

The chemical composition of mCellulose biofabricated from various carbon sources was assessed using FTIR-ATR (FIG. 53). Although native cellulose I is a composite of two crystalline configurations comprised of cellulose I_{α} (triclinic) and I_{β} (monoclinic), all FTIR spectra exhibited similar vibrational bands, with larger differences in peak intensities observed at higher concentrations. Chemical fingerprints of cellulose I were confirmed via ATR (n=5), in which characteristic peaks for hydroxyl O—H stretching (3300 cm⁻¹), methyl C—H stretching (2900 cm⁻¹, 1434 cm⁻¹), antisymmetric bridge C—O—H stretching (1161 cm⁻¹), C—O bending (1106 cm⁻¹, 1057 cm⁻¹), as well as β-glucosidic linkages between monomer units (895 cm⁻¹) were observed. Further, the presence of peaks for cellulose I_{α} and I_{β} at 750 cm⁻¹ and 710 cm⁻¹, respectively, revealed the ability for G. xylinus to synthesize both allomorphs independent to carbon source and concentration (FIG. 38 a). It was observed that ATCC 31174 produced 34 to 44% I_{α}, with comparable fractions reported for cellulose biofabricated from alternate A. xylinus strains, however, differences in allomorph percentage were found between carbon sources (FIG. 38b). In 2% w/v groups, the fraction of I_{α} was significantly greater when synthesized with fructose, sucrose, and mannitol than glucose and xylitol cultures, though similar trends were not seen at 8% w/v (n=5).

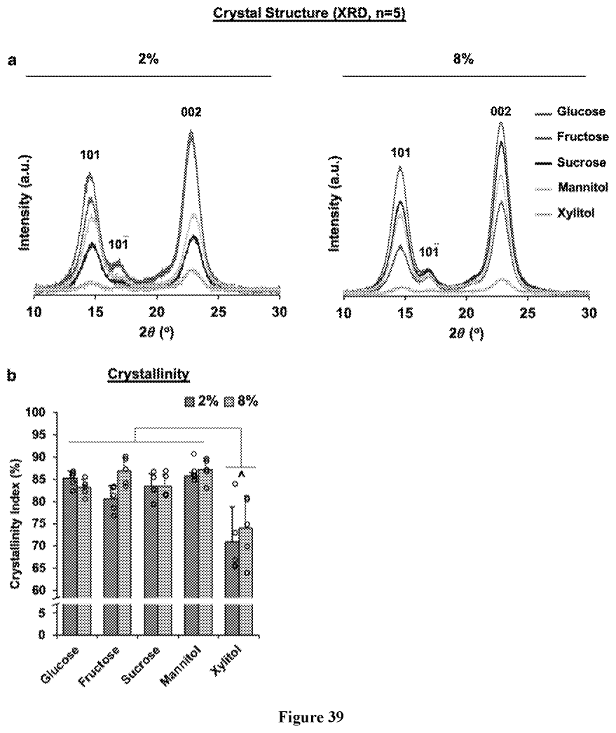

Surprisingly, differences in cellulose crystal structure were observed with X-ray diffraction analysis (XRD, FIG. 39). While all fibers synthesized with varying carbon sources and concentrations exhibited cellulose I Bragg peaks at 2θ=14.6°, 16.9°, and 22.8°, corresponding to the (1-10), (110), and (200) planes (FIG. 39 a), only the inferior mechanical properties of xylitol-based groups are reflected in bulk microstructure crystallinity measurements. A peak deconvolution method demonstrated comparable crystallinity indexes between glucose, fructose, sucrose, and mannitol groups (85%), along with a significant decrease to 70.97±7.87 and 74.01±7.19% in 2% and 8% w/v xylitol, respectively (n=5, FIG. 39 b).

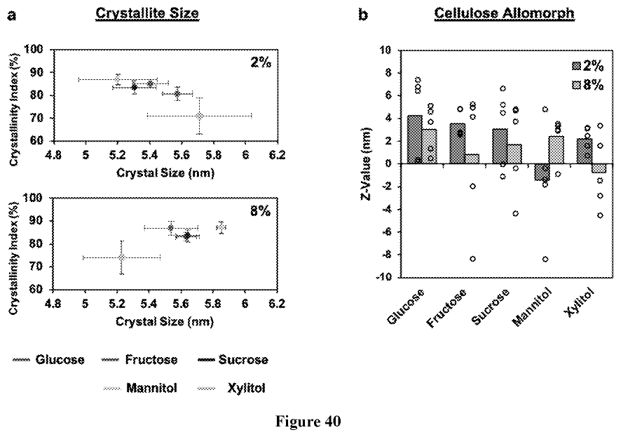

Further, crystallinity index values do not exhibit a correlation with corresponding crystallite sizes in glucose, fructose, and sucrose-based groups at either concentration (FIG. 40 a). Although greater crystal sizes were observed in mCellulose synthesized from glucose and sucrose with an increase to 8% w/v, no significant differences were found. On the other hand, crystallite size of cellulose produced from mannitol was found to significantly increase with increasing concentration (5.20±0.25 vs. 5.85±0.03 nm, n=5), while xylitol was found to significantly decrease (5.71±0.33 vs. 5.23±0.24 nm, n=5).

Discriminant analysis of two d-spacings (d₁ and d₂), also known as Z-value, was measured to determine differences in mCellulose allomorph structure based upon biofabrication with various carbon sources (FIG. 40 b). Cellulose synthesized from glucose, fructose, and sucrose were observed to be mainly rich in I_{α} (Z>0; bacterial-algal), thus supporting the findings of comparable crystallite sizes. It was found that the mass fraction of I_{α} decreased with increasing concentration of the prior sugars within the culture medium, corresponding to promoted crystallization of the I_{β} allomorph. Interestingly, the 2% w/v mannitol and 8% w/v xylitol groups, with the lowest crystal sizes, showed Z-values less than zero which indicate that the mCellulose produced belonged to the I_{β}-dominant type (cotton-ramie). However, a respective increase and decrease in concentration of the prior groups resulted in I_{α}-rich structures with greater crystal sizes.

### Carbon Source Impact Assessment

A cradle-to-gate life cycle impact assessment (LCA) was implemented to compare the human health and environmental manufacturing impacts between various carbon sources utilized for mCellulose biofabrication, such as glucose, fructose, sucrose, mannitol, and xylitol. Both normalization and weighting factors were applied to convert the manufacturing impacts of 10 categories (Human Health: fossil fuel depletion, carcinogenics, hormonally active agents, respiratory effects, and smog; Environmental: acidification, ecotoxicity, eutrophication, global warming, ozone depletion) into milliPoints per yielded amount of cellulose after 14 days.

In terms of the human health and environmental impacts between carbon sources, poly-alcohols, xylitol and mannitol, have the largest total and global warming impacts when modelled against glucose, fructose, and sucrose for 2% w/v cultures (a). Specifically, xylitol, a natural sugar alcohol that is often used as a sugar substitute, was observed to have the highest total impact (14.1×10⁻⁵ mPts) in comparison to other groups. Of note, manufacturing impacts of xylitol sourced by biomass hydrolysis significantly contributed to human toxicity (7.98×10⁻⁵ mPts) as a result of carcinogenic production (50.9%) relative to all other categories. Further, mannitol drastically reduces total impacts by more than 50% to 5.34×10⁻⁵ mPts, however, carcinogenic production remains the leading category, accounting for 35.9% of total impact. In terms of the human health and environmental impacts generated by monosaccharide and disaccharides, sucrose was observed to significantly reduce total impacts, as well as global warming impacts. Here, sucrose was demonstrated to be extracted from sugar cane at a sugar refinery, which resulted in a more than ten times reduction in total impacts compared to xylitol (0.77×10⁻⁵ vs 14.1×10⁻¹ mPts). Interestingly, hormonally active agents were found to be the leading category for sucrose, accounting for 41.9% of total impact.

### Example 2

TABLE 1
Portfolio of Mechanical Properties Tunable Based on Sugar Source During Synthesis.

| | | Young's | Ultimate Tensile | Yield Strength | |
|---|---|---|---|---|---|
| Sugar Source | Concentration | Modulus (MPa) | Strength (MPa) | (MPa) | Ductility (%) |
| Glucose | 2% | 15.78 ± 4.39 | 2.25 ± 0.58 | 1.95 ± 0.64 | 20.60 ± 3.41 |
| | 8% | 31.21 ± 4.54\hat{ } | 3.47 ± 0.49\hat{ } | 2.98 ± 0.46\hat{ } | 15.52 ± 4.02 |
| Fructose | 2% | 7.78 ± 2.12 | 1.47 ± 0.42 | 1.29 ± 0.43 | 26.39 ± 4.41 |
| | 8% | 173.69 ± 29.54\hat{ } | 17.26 ± 2.49\hat{ } | 15.79 ± 2.77\hat{ } | 14.99 ± 3.00 |
| Sucrose | 2% | 8.96 ± 3.27 | 1.53 ± 0.36 | 1.36 ± 0.36 | 23.50 ± 5.88 |
| | 8% | 114.61 ± 35.48\hat{ } | 15.74 ± 5.37\hat{ } | 14.57 ± 4.80\hat{ } | 17.82 ± 2.36 |
| Mannitol | 2% | 23.48 ± 11.40 | 3.63 ± 1.07 | 3.28 ± 0.89 | 22.72 ± 3.80 |
| | 8% | 174.67 ± 62.69\hat{ } | 23.34 ± 9.40\hat{ } | 22.40 ± 9.02\hat{ } | 16.48 ± 5.38 |
| Xylitol | 2% | 8.15 ± 2.93 | 1.26 ± 0.39 | 1.21 ± 0.39 | 21.26 ± 4.19 |
| | 8% | 9.74 ± 5.31 | 1.40 ± 0.99 | 1.36 ± 0.99 | 15.42 ± 3.60 |

### Materials and Methods

### Microbial Cellulose Biosynthesis

Microbial cellulose biofilms were prepared in culture media containing 5.8% w/v sucrose, 2% w/v green tea as a nitrogen source, and Symbiotic Colony Bacteria and Yeast (Fermentaholics©). Culture media was inoculated with 10% w/v starter culture consisting of a mix between the bacteria and yeast. A static culture was maintained at room temperature until a pellicle at least 2 cm thick was formed at the air-cultivation media interface. As-fabricated mCellulose pellicles were rinsed with deionized water three times to remove residual sugars and dried for 72 hours on a polypropylene tray at room temperature.

Post-Production Treatment of mCellulose

Hydrated as-fabricated mCellulose pellicles were washed with deionized water three times and immersed in a lecithin emulsion for 24-48 hours (LT). Briefly, the lecithin emulsion was developed by blending 5% w/v lecithin (soy or sunflower seed, 97% phosphatidylcholine) with 20% v/v sunflower seed oil in water at a high speed for 60 seconds. Following, treated mCellulose were rinsed with deionized water three times to remove non-attached lecithin emulsion, and dried for 72 hours on a polypropylene tray at room temperature.

Additional smoke treatment was performed on dried mCellulose samples with (S) and without (LTS) lecithin tanning to determine the effect of aldehydes to seal the emulsion and complete the tanning process. mCellulose samples were placed into an electric smoker (Smoke Hollow 26142E) with smoking wood chips and exposed to hydrocarbon-rich smoke for 1 hour with the temperature between 160-210 °F.

Characterization of Lecithin-Tanned mCellulose

As-fabricated (MC) and treated (LT, S, LTS) mCellulose biotextiles were characterized for surface morphology, tensile properties, crystal structures, as well as its thermal, chemical, and elemental compositions.

Microbial cellulose morphology was assessed by using a scanning electron microscope (Zeiss Sigma VP, Oberkochen, Germany; 3 kV; n=5). Briefly, hydrated microbial cellulose samples were initially placed in a −20 °C. freezer for 24 hours and lyophilized in a freeze dryer system (Labconco FreeZone, Kansas City, MO, USA) for 24 hours at −84 °C and 2.0×10⁻² mbar. Prior to imaging, samples were sputter coated (Cressington 108, Watford, UK) with 30 nm of gold. The fiber diameter of each sample was measured by analyzing randomly selected fiber segments in SEM images using NIH ImageJ software (Bethesda, MD, USA; n=100).

Microbial cellulose hydrophilicity before and after treatment(s) was measured via swelling degree and water contact angle (WCA; n=5). The swelling degree of mCellulose samples was analyzed by placing dried, pre-weighed microbial cellulose discs (diameter: 4.5 mm, n=5) in 5 mL of deionized water at room temperature for 24 hours. The degree of swelling of microbial cellulose was measured after 24 h. The degree of swelling was calculated as the following:

$$Degree of Swelling = [ \frac{( Wet Weight - Dry Weight )}{Dry Weight} ] * 100$$

Water contact angle was conducted by depositing deionized water (10 μL) onto mCellulose surfaces, in which representative angles were measured using a goniometer (Ramé-hart, Inc., Mountain Lakes, NJ, USA) with a fiber optic illuminator (Dolan-Jenner Industries, Inc., Woburn, MA, USA). Contact angles less than 90° are characteristic of hydrophilic surfaces, while greater than 90° are indicative of hydrophobic properties.

Uniaxial tensile testing was conducted based upon the ASTM International standard tensile test method for plastics (D638-14). Microbial cellulose samples (n=4) were secured with custom clamps and mounted in a uniaxial tensile testing machine (Instron, Model 1321, Norwood, MA, USA) equipped with a 25 kN load cell. Biotextiles were maintained to have an average gauge length of 2 inches and were tested to failure. Microbial cellulose elastic modulus, toughness, and ultimate tensile strength were determined from the resulting stress-strain curve.

Microbial cellulose crystal structure was assessed by X-ray diffraction (XRD, n=5, Panalytical XPert3 Powder XRD, Malvern Panalytical). As-fabricated and processed (LT, LTS, S) samples (5×2 cm) were positioned onto a zero-diffraction plate (silicon, p-type, β-doped, MTI Corporation, Richmond, CA) and scanned using a diffractometer with a 3-kV generator, X-ray tube, fully ceramic Cu Long fine focus (LFF), vertical goniometer (theta-theta) and a PIXcel id detector. Diffractograms were measured through a 1 cm copper-based beam mask from 5-100° (2θ) with a step size of 0.026°, scanning rate of 10.7°/min, and a beam path utilizing a 1/2° divergence slit. Background and baseline subtraction were applied using Highscore XRD Software. A XRD peak deconvolution method was utilized as a curve-fitting process in Igor-Pro software to calculate the crystallinity index of microbial cellulose samples. Crystalline and amorphous peaks were identified and separated in XRD spectra. Gaussian functions were used for the deconvolution of spectra into four crystalline peaks (101, 10_{1̅}, 021, and 200), and one amorphous peak centered at a 20 value of ~18.5 degrees. Crystallinity index (CI) was calculated from the ratio of the area of crystalline regions (A_{cry}) to the total peak area (Aₜₒₜₐₗ):

$$C ( \% ) = \frac{{A}_{cry}}{{A}_{total}} * 100$$

Crystallite size was determined by calculating the Scherrer width via the Scherrer equation:

$${d}_{hkl} = \frac{k λ}{FWHM * cos ( θ )} ,$$

where dₕₖₗ is the size of the crystallite (nm), k is the Scherrer constant of 0.94, λ is the x-ray wavelength set at 0.154 nm, and θ is the diffraction angle.
Additionally, the interplanar lattice spacing (d-spacing) was determined by Bragg's equation:

$${d}_{hkl} = \frac{λ}{2 sin ( θ )} ,$$

in which dₕₖₗ is the lattice spacing of the crystallographic planes, λ is the x-ray wavelength (0.154 nm), and θ is the corresponding Bragg angle.

Following, microbial cellulose thermal properties were evaluated based upon the ASTM International Standard Test Method for Flammability of Apparel Textiles (D1230-94) to determine the ease of material ignition, and the duration of flame spreading. Briefly, as-fabricated mCellulose (1×6″) was secured with custom clamps and mounted at 45° in a custom apparatus. A 3,730 °F/2054.4 °C flame, produced by liquid propane (Map-Pro Cylinder; Bernzomatic) as a fuel source, was applied at 45° to one end of the mCellulose samples. The resulting morphology of charred mCellulose was assessed by using a scanning electron microscopy (3 kV).

Additionally, thermal properties and associated char formation were assessed by thermogravimetric analysis (TGA 550, TA Instruments; New Castle, DE). Initially, mCellulose samples were first cored with a biopsy punch (Sklar Surgical Instruments, West Chester, PA) to obtain 4.5 mm diameter discs. TGA analysis was performed at a heating rate of 10 °C/min over the temperature range of 25 °C to 700 °C. under flowing nitrogen (40 mL/min), in which final weights indicated the formation of char residue.

Molecular and elemental level insights on chemical structure and its relation to mechanical, hydrophilic, and thermal properties were determined by X-ray photoelectron (XPS), Fourier transform infrared (FTIR), and Energy-dispersive X-ray (EDXA) spectroscopy.

Molecular characterization detailing the surface bonding of lecithin tanned and as-fabricated MC was determined by XPS (PHI 5500, Chanhassen, MN, USA) after samples were vacuum annealed at 75 °C for 24 hours. The spectra were recorded using a monochromatic Mg—Ka radiation X-ray source (1253.6 eV) and the analyzer pass energy was set to 25 eV. The sample chamber was set to 50 W operating at 15 kV voltage and a base pressure of 2×10⁸ torr. The XPS spectra were collected in the range from 0 to 1200 eV, with a resolution of 0.1-1.0 eV. A Shirley background subtracted was performed to remove the inelastic background of the carbon (C 1s), oxygen (O 1s), and phosphorous (P 2p) electron core spectra and data was analyzed using commercial curve fitting software Igor64 (WaveMetrics, Portland, OR, USA). The binding energy scale was calibrated using the Au 4f 4/7 line of 10 nm thick gold, electron beam evaporated into 5 mm wide strips onto two parallel edges of the LT and MC samples and confirmed against the C is binding energy for adventitious carbon on Au (285 eV) as well as published XPS data for microbial and plant cellulose. Based on these values, MC and LT XPS data were shifted −1.98 and −2.88 eV to higher binding energy, respectively. A smoothing factor of 10 was applied to the P 2p XPS data for ease of comparison (no smoothing was applied to C is or O 1s data).

Microbial cellulose chemical composition was determined by using Fourier transform infrared spectroscopy in attenuated total reflectance mode (FTIR-ATR, n=5, LUMOS). All spectra were collected from 4000-600 cm⁻¹ (spectral resolution 4 cm⁻¹, 200 scans). Baseline subtraction using vibrational spectroscopy OPUS software (Bruker) was implemented, and all spectra were assessed for characteristic cellulose peaks. Further FTIR analysis was performed to determine the relative amount of crystallinity in mCellulose. While the peak around 1420-1430 cm⁻¹ has been found to be associated with the presence of crystalline structures, the absorbance at 898 cm⁻¹ is associated with cellulose's amorphous structures. The ratio between 1429 cm⁻¹ to 897 cm⁻¹ is defined as the lateral order index (LOI) required to assess cellulose's overall degree of order:

$$L O I = \frac{{A}_{1420}}{{A}_{893}}$$

Additionally, the ratio between absorbances at 1372 cm⁻¹ to 2900 cm⁻¹ is defined as total crystallinity index (TCI) and is related to the degree of crystallinity in cellulose.

$$T C I = \frac{{A}_{1375}}{{A}_{2900}}$$

Finally, elemental composition on mCellulose surfaces was determined by energy dispersive x-ray analysis (Bruker XFlash® 6×30 Detector, Esprit 2.1 software, Billerica, MA, USA). Biotextiles were sputter coated (Cressington 108, Watford, UK) with copper, and analyzed for the presence of phosphorus (n=5, 10 kV, 5 minutes).

### Reagents and Materials

All reagents and materials were supplied from Fisher Scientific, unless otherwise stated

### Statistical Analysis

All results are reported as mean±standard deviation, with ‘n’ equal to the number of replicates per study group. One-way analysis of variance (ANOVA) was utilized to determine significance between groups. The Tukey-Kramer post-hoc test was performed for all pair-wise comparisons with significance attained at p<0.05. All statistical analyses were performed by utilizing JMP-IN (4.0.4, SAS Institute, Inc.).

### Result

### Microbial Cellulose Characterization

The effect of lecithin treatment on microbial cellulose in comparison to aldehyde smoke treatment and a combination of both processes is determined. As-fabricated, lecithin “tanned”, aldehyde tanned (smoked), and sequentially lecithin and aldehyde tanned mCellulose are denoted MC, LT, S, and LTS, respectively.

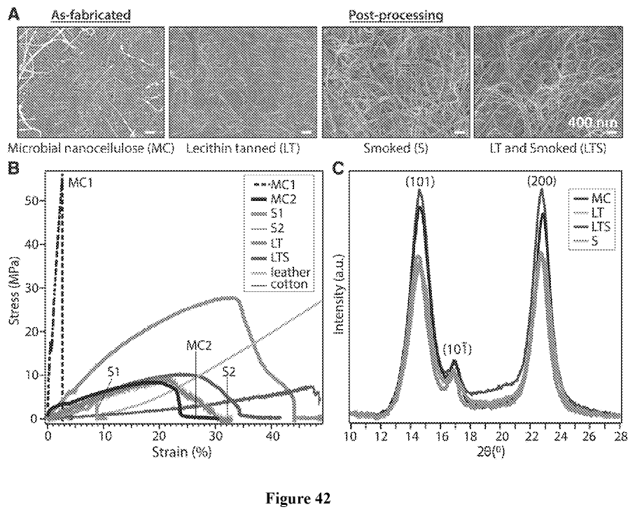

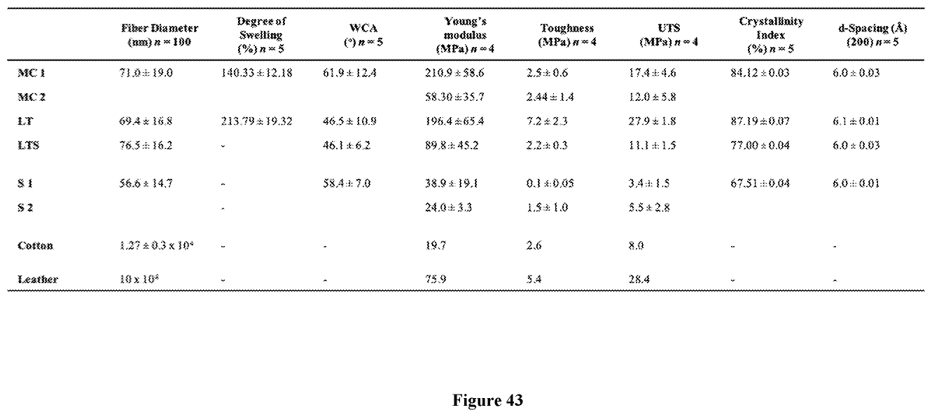

A dense, unaligned nanofibrous morphology was observed with scanning electron microscopy on as-fabricated MC surfaces, with an average diameter of 71.00±19.00 nm (n=100; FIG. 42 a). Separate and sequential green bio-processing treatment with lecithin and smoke aldehyde tanning produced no significant differences in fiber size compared to MC (FIG. 43). Both as-fabricated and LT exhibited similar well-organized three-dimensional unaligned fibrous networks, indicating that the lecithin emulsion did not create a surface coating and was rinsed off after treatment. However, elemental analysis with energy dispersive x-ray analysis (EDXA) indicated significantly increased amounts of phosphorus on LT surfaces compared to as-fabricated MC (2.02% vs. 0.57%; n=3, \hat{ }p<0.05), supporting the bio-phosphorylation interaction between lecithin and mCellulose. Thus, subsequent investigation in swelling, tensile, thermal, and chemical properties may assist to understand material modifications.

Next, mCellulose hydrophilicity was assessed by evaluating the degree of swelling and water contact angle after lecithin and aldehyde tanning. Dried as-fabricated microbial cellulose was allowed to swell in 5 mL of deionized water at room temperature for 24 hours, in which the biopolymer's hydrophilic network containing large amount of hydroxyl groups was indicated by a degree of swelling of 140.33±12.18%. Further lecithin treatment significantly increased mCellulose swelling (213.79±19.32%), suggesting an enhanced hydrophilic network (n=5, \hat{ }p<0.05, FIG. 43). Not surprisingly, this improved hydration state was reflected in surface contact angle, with smaller angles reported in LT (MC: 61.9±12.4% vs. LT: 46.5±10.9%), however, significant differences were not observed between all groups (FIG. 43). Moreover, significant changes observed in LT bulk swelling rather than surface contact angle supports morphological analysis, indicating lecithin emulsion was rinsed off after treatment.

Common to naturally occurring biopolymers, as-fabricated MC exhibited varied stress-strain behaviors under uniaxial tensile testing as a result of its hygroscopicity (FIG. 42b). Although as-fabricated MC was biosynthesized with consistent culturing parameters, elastic moduli ranged between 58.30±35.71 MPa (MC 2) and 210.91±58.61 MPa (MC 1). Aldehyde tanning similarly exhibited variable mechanical properties, albeit with significantly decreased elastic modulus, toughness, and maximum stress (\hat{ }p<0.05 between groups). On the other hand, treatment with lecithin created a stable mechanical state that significantly increased toughness, and maximum stress to 7.2±2.3 MPa and 27.9±1.8 MPa, respectively (n=4, \hat{ }p<0.05). Compared to conventional textiles, LT was observed to have higher tensile strength than leather and cotton up to strain values of 33% and 45%, respectively (FIG. 43). LT biotextiles had toughness values, a reflection of both strength and ductility, comparable to leather. Although LTS maintained stable mechanical properties, its elastic modulus, toughness, and maximum stress significantly decreased compared to LT (\hat{ }p<0.05 between groups).

Not surprisingly, differences in crystal structure are subtly reflected in the tensile mechanical properties of treated mCellulose measured by X-ray diffraction. While all groups exhibited cellulose I Bragg peaks at 2θ=14.6°, 16.9°, and 22.8°, corresponding to the (1-10), (110), and (200) planes, as well as similar d-spacings, a peak deconvolution method indicated only a minor increase in crystallinity index in LT (MC: 84.12±0.03% vs. LT: 87.19±0.07%) was accompanied with enhanced mechanical behavior (n=5, FIG. 42c). In contrast, aldehyde tanning significantly reduced crystallinity index to 67.51±0.04%, supporting its inferior and brittle mechanical properties (n=5, \hat{ }p<0.05, FIG. 43). Albeit at lower magnitudes, smoke tanning with lecithin treatment (LTS) similarly decreased in crystallinity index to 77.00±0.04% in comparison to MC and LT. Collectively, the tensile properties and crystallinity index of LT and S suggests that these tanning treatments alter mCellulose structure differently, without modifying surface morphology.

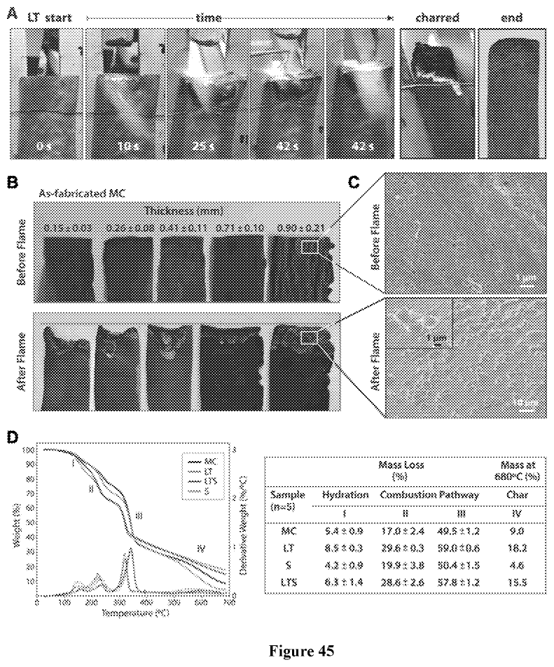

Since phosphorous compounds have been reported to reduce polymer flammability, thermal properties of microbial cellulose, as a result of phosphorylation with LT, were determined. Surprisingly, when exposed to a direct 2054 oC flame for 55 seconds, LT did not ignite. In comparison, a control aluminum rod melted under the flame after 40 seconds, while phosphorylated-based cellulose continued to deflect the flame, without propagation. Removal of char on LT samples indicated an intact bulk surface comprised of a three-dimensional layered structure, suggesting superior flame-retardant capacity. Thus, the influence of as-fabricated mCellulose's inherent layered microstructure on flame retardance was also determined under similar flame testing conditions. Notably, flame testing of as-fabricated MC samples of varying thicknesses revealed that the mass loss due to combustion decreases with increasing mCellulose thickness until 0.9 mm, at which point mass loss was minimal, although greater than for LT biotextiles of the same thickness (FIG. 45b). Further morphological analysis with scanning electron microscopy revealed the formation of intumescent bubbles on the nanofibrous surfaces of charred as-fabricated MC of sufficient thicknesses (>25 mm and 0.9 mm in hydrated and dried forms, respectively), and phosphorylated LT (FIG. 45c).

Thermogravimetric analysis (TGA) was performed under a nitrogen atmosphere from 25-680 °C. to further assess the thermal decomposition behavior and thermal stability of microbial cellulose before and after lecithin treatment (FIG. 45d). TGA analysis of as-fabricated MC shows a three-step mass loss: (I) the initial evaporation of free and bound water between 25-200 oC; (II-III) polymer decomposition (210-240 °C); and (IV) the production of either pyrolysis-based levoglucosan or flame-resistant char (300-360 OC). Lecithin treated samples maintained a higher hydration state due to significantly greater amounts of moisture-based weight (8.5±0.3%) lost by 200 °C. Additionally, higher mass loss was observed for LT relative to MC in regions II (29.55±0.25% vs. 16.96±2.37%) at 232 °C and III (58.99±0.55% vs. 49.51±1.21%) at 340 °C. Finally, region IV (680 °C) indicated that aliphatic compounds were decomposed into char with a greater residual mass for LT than MC (18.2% vs. 9.0%). This suggests that the inherent nano- and microscale layered assembly of mCellulose, as well as, chemical modification with lecithin promoted non-toxic flame retardancy and stability.

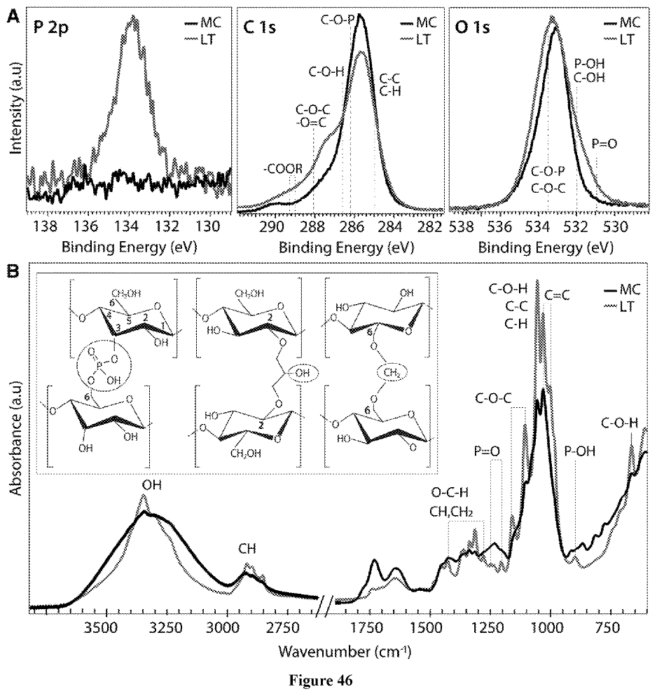

Molecular level insights on the chemical origin of the improved mechanical and thermal properties for lecithin-based mCellulose were revealed by X-ray photoelectron (XPS) and Fourier transform infrared (FTIR) spectroscopy. Reviewing the XPS spectra, a new peak positioned at 134 eV indicated that lecithin tanning introduced phosphorus (P 2p) in oxidized states (FIG. 46a). The carbon is photoelectron spectra revealed an increase in carboxyls, esters and anhydrides, centered at binding energies of ~286.6 (C—O—H), 288.1 (C—O—C), and 289.1 eV (—COOR), respectively. Additionally, oxygen is spectra revealed mCellulose phosphorus modification, with P═O (530.9 eV) and C—O—P bonds (533.5 eV).

Chemical fingerprints of mCellulose observed with FTIR provided mechanistic insights on cellulose crosslinking with lecithin tanning (FIG. 46b). The appearance of new peaks that represent the deposition of phosphatidylcholine molecules were identified including P═O resonances at 1207.4, 1230 and 1252 cm⁻¹, and a —P—OH stretch vibration at 898 cm⁻¹. Further, the sharpening of —OH peaks characteristic of the cellulose skeleton at 3450-3000 cm⁻¹ and 664 cm⁻¹, and methyl and methylene resonances, including C—H vibrational bands at 1437-1245 cm⁻¹ and 3000-2853 cm⁻¹ and the C—H deformation (1428, 1370, and 1316 cm⁻¹), CH₂ (1184-1104 cm⁻¹) and C—O—H groups (690 cm⁻¹) were observed for LT in comparison to MC.

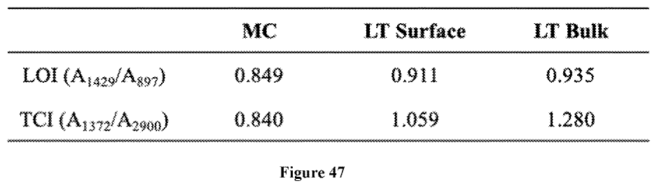

Cellulose is a polymer consisting of chains with both crystalline (ordered) and amorphous (disordered) regions, in which further analysis of FTIR spectroscopy was used to determine the relative amount of crystallinity in MC and LT. Treatment of microbial cellulose with lecithin increased LOI and TCI parameters, thus, indicating a higher degree of overall order with greater crystallinity for LT compared to MC on the surface and throughout the bulk of the biomaterial (FIG. 47).

Together, these results demonstrate that lecithin enzyme tanning changes the bonding environment of microbial cellulose, such that phosphatidylcholine tanning modified cellulose crosslinking, enhancing mechanical, hydrophilic, and thermal properties.

### Example 3

The effect of green-based plasticizer and crosslinking post-processing treatments were assessed to enhance the mechanical performance of mCellulose. To attain improved ductility, hydrated microbial cellulose pellicles were exposed to various concentrations of sorbitol and glycerol. It is hypothesized that bioplasticizer treatment would increase biomaterial percent elongation at failure while decreasing mechanical strength in a concentration dependent manner, in which optimal parameters would not compromise nanofibrous structure or chemistry. To achieve simultaneous stability in strength and ductility, tannic acid was incorporated into microbial cellulose and further coordinated with iron ions as crosslinking agent. Here, optimal pH conditions are determined for controlling the complexation between cellulose, tannic acid, and iron that supports mechanical performance. Furthermore, it is anticipated that optimal pH parameters would promote chelation between cellulose, tannin and iron, forming stable cross-linkages between the biopolymer's nanofibrous layers. Following, the morphology, tensile mechanical behavior, viscoelastic properties, chemical and crystal structure, and anti-microbial properties of microbial cellulose biofilms coordinated with tannic acid and iron ions were characterized. A cradle-to-gate life cycle assessment was utilized to compare the environmental and human health impacts of sorbitol, glycerol, and tannic acid as green chemistry strategies for mCellulose production. Ultimately, this application would support the development of mCellulose biomaterials with tailored mechanics via bio-based plasticizers and tannin-metal complexation, demonstrating the potential of green bioprocessing to achieve sustainable development goals.

### Materials and Methods

### Microbial Cellulose Biosynthesis and Purification

The Gluconacetobacter xylinus strain ATCC 31174 was acquired for microbial cellulose biofabrication. Microbial cellulose pellicles were prepared in Hestrin-Schramm (HS) culture medium containing 2% w/v mannitol, 0.5% w/v yeast extract, 0.5% w/v peptone, and 0.27% w/v sodium phosphate dibasic anhydrous. The medium pH was adjusted to 4.5 using citric acid. Following, all medium was sterilized via autoclaving (Buxton) at 250 °C for 20 minutes.

Prior to mCellulose biofabrication, G. xylinus was pre-cultured in sterile HS medium containing 2% w/v mannitol for 72 hours at 30 °C. under static conditions. Following, the preculture was inoculated (5% w/v) into sterile HS medium (2% w/v mannitol) within sterilized Pyrex dishes (34×23×5 cm). Static fermentation at 30 °C. was maintained for 14 days, in which 2-D layer by layer production of mCellulose occurred at the air-liquid medium interface, resulting in pellicles with a thickness of 0.7 cm.

After 14 days, mCellulose was harvested from the air-to-medium interface and subjected to a purification protocol. Briefly, formed biofilms were washed three times with deionized water (diH₂O) to remove residual sugars and medium components. To lyse G. xylinus cells on the surface and within the cellulose network, pellicles were immersed into 70% ethanol for 24 hours, followed by a 0.1 M NaOH (Sigma-Aldrich) bath for 24 hours. Next, all pellicles were submerged into diH₂O for 48 hours to return to pH=7 and further remove medium components from the cellulose.

Preparation of Bioplasticized mCellulose

As a post-production bioplasticizer treatment, mCellulose were rinsed with deionized water before and after purification, and directly immersed into either sorbitol or glycerol baths at varying concentrations for 24 hours. Two independent batches of as-fabricated mCellulose were prepared, utilizing identical culturing conditions described above, for each bioplasticizer treatment. Purified as-fabricated mCellulose from each treatment served as controls (MC 1: Sorbitol; MC 2: Glycerol) and were dried at room temperature (~23 °C). The effect of sorbitol as a plasticizer was assessed by immersing hydrated mCellulose (11×11×0.7 cm) into sorbitol baths (300 mL) with concentrations set at 1%, 2%, and 5% w/v. After removal from the immersion, treated pellicles were soaked in deionized water (300 mL) for 10 minutes to remove excess plasticizer, and air dried at room temperature under the same conditions as as-fabricated mCellulose. A similar procedure was performed to determine the effect of glycerol as a plasticizer, in which mCellulose was submerged into various glycerol solutions (1%, 2%, and 5% v/v; 300 mL).

Microbial cellulose pellicles were also processed with tannic acid following glycerol treatment to facilitate crosslinking between the biopolymer and plasticizer through covalent bonding. Hydrated glycerol-plasticized mCellulose samples were exposed to further tannic acid treatment, in which pellicles were immersed into TA baths (300 mL) for 24 hours, washed with diH₂O for 10 minutes, and air dried at room temperature. Exposure of mCellulose to various TA concentrations of 1% and 2% w/v was investigated to seal in glycerol for improved mechanical performance.

Preparation of Tannin-Iron-based mCellulose Complexes

A third method for modifying mCellulose nanofibrous network was evaluated by tannic acid complexation with iron metal ions (TA-Fe) as a bridging crosslinking agent. An independent batch of mCellulose was biofabricated for this treatment, in which purified as-fabricated pellicles served as a third control (MC 3) to examine batch-to-batch variability.

Briefly, hydrated purified mCellulose (11×11×0.7 cm) was immersed into 300 mL of 0.5% w/v tannic acid for 24 hours (MCTA). Subsequently, the pellicle was washed with diH₂O for 10 minutes, immersed into an iron chloride (FeCl₃) solution with a concentration of 0.5% w/v, and continuously stirred at 300 rpm. A sodium hydroxide solution (2 M) was added to the FeCl₃ bath to raise the pH to either 5 or 9. After 2 hours, the reaction was halted by removing the treated mCellulose from the solution, rinsed with diH₂O for 10 minutes to remove unreacted species, and air dried at room temperature. Microbial cellulose controls were prepared without adding TA, FeCl₃, or NaOH solutions, and subjected to similar drying conditions.

### Microbial Cellulose Fiber Characterization

Microbial cellulose was characterized using discs (Ø: 10 mm) cut from dried as-fabricated, bioplasticized, and crosslinked samples with a biopsy punch (Sklar Surgical Instruments, West Chester, PA). mCellulose fiber morphology was assessed by performing scanning electron microscopy (SEM, 3 keV, Zeiss Sigma VP, Carl Zeiss A G, Oberkochen, Germany) on the surface of samples that were sputter coated (10 nm, 20 s) with gold-palladium to improve sample conductivity and reduce charging effects (Cressington 108 Auto, Cressington Scientific, Watford, UK). Energy dispersive x-ray analysis (EDXA, XFlash® 6×30 Detector, Espirit 2.1 software, Bruker, Billerica, MA) was performed at 10 keV for 300 s to acquire quantitative spectra, elemental mass normalization, and colorized micrographs for the elements carbon (yellow), oxygen (green), sodium (lime-green), chlorine (orange), and iron (purple).

Uniaxial tensile testing was conducted on microbial cellulose biofilms based upon the ASTM International standard tensile test method for plastics (D638-14) (Moffat et al., 2009; Lu et al., 2005). Briefly, dried as-fabricated and post-fabricated mCellulose samples (5×1 cm, n=5) were weighed, and thickness was measured using digital calipers. The samples were secured with clamps and mounted onto a microtester device (Instron, Model 5848) equipped with a 100 N load cell. Samples were maintained to have an average gauge length of 30 mm, and were tested to failure at a strain rate of 5 mm/minute. The resulting microbial cellulose stress-strain curves were utilized to calculate elastic modulus from the slope of the linear region, ultimate tensile strength from the maximum stress achieved prior to sample failure, toughness from the area under the curve, and ductility as the percent elongation at failure. Microbial cellulose tensile properties before and after treatment(s) were compared to the mechanical performance of conventional textiles, including cotton, silk, polyester, and low-density polyethylene (LDPE). Briefly, conventional textiles (10×2 cm, n=5) were mounted with custom clamps onto a uniaxial tensile testing machine (Instron, Model 1321) with a gauge length of 5 cm, and were tested to failure. Textile elastic modulus, ultimate tensile strength, toughness, and ductility was measured from stress-strain curves, as previously described.

Viscoelastic measurements of microbial cellulose samples (n=5), before and after crosslinking with tannic acid and iron chloride, was determined with a controlled shear-strain rheometer (ARES-LS1, TA Instruments, New Castle, DE) fitted with parallel plate geometries (Ø: 12.7 mm). Briefly, hydrated as-fabricated and TA-Fe mCellulose samples were cut into 10 mm discs via a biopsy punch (Sklar Surgical Instruments), and loaded onto a stage between two flat platens fitted with abrasive paper to prevent sample slippage. The upper platen was lowered to contact the sample with a normal force of 1 N and PBS was immediately added to prevent dehydration. The equilibrium compressive modulus (E_{eq}) was calculated at 15% strain (ε):

$${E}_{eq} = \frac{{σ}_{z}}{ε} ; {σ}_{z} = \frac{Δ F}{π {d}^{2} / 4} ,$$

in which σ_{z} and ΔF are the normal stress and equilibrium normal force change during axial compression, respectively. A dynamic frequency test was performed (0.01 to 10 Hz), in which the elastic modulus (G*) and shear modulus (|G*|) was measured at 1 Hz.

Dried microbial cellulose chemical composition was determined by using attenuated total reflectance mode during Fourier transform infrared spectroscopy (FTIR-ATR, n=5, LUMOS). All spectra were collected from 600-4000 cm⁻¹ (200 scans, spectral resolution 2 cm⁻¹) and assessed for characteristic cellulose peaks (Schiros et al., 2022; Atykyan et al., 2020), as well as, characteristic sorbitol (Wang et al., 2012; Moshin et al., 2011; Somashekarappa et al., 2013; Sun et al., 2018), glycerol (Cielecka et al., 2019; Sun, Meng, et al., 2018; Sun, Liang et al., 2018; Chen et al., 2008; AlOmar et al., 2016), and tannic acid (Zhang, Sun, et al., 2020; Ninan et al., 2016; Lee et al., 2018; Muhoza et al., 2019; Iglesias, Jaén, 2001) peaks. Further FTIR analysis was performed to determine the relative amount of crystallinity in mCellulose (Poletto et al., 2014; Fan et al., 2012). While the peak around 1420-1430 cm⁻¹ has been found to be associated with the presence of crystalline structures, the absorbance at 898 cm⁻¹ is associated with cellulose's amorphous structures (Oh et al., 2005; O'Connor et al., 1958; Carrillo et al., 2004). The ratio between 1429 cm⁻¹ to 897 cm⁻¹ is defined as the lateral order index (LOI) required to assess cellulose's overall degree of order:

$$L O I = \frac{{A}_{1420}}{{A}_{893}}$$

Additionally, the ratio between absorbances at 1372 cm⁻¹ to 2900 cm⁻¹ is defined as total crystallinity index (TCI) and is related to the degree of crystallinity in cellulose.

$$T C I = \frac{{A}_{1375}}{{A}_{2900}}$$

Microbial cellulose crystal structure was assessed by X-ray diffraction (XRD, n=5, Panalytical XPert3 Powder XRD, Malvern Panalytical). Samples were positioned onto a zero-diffraction plate (silicon, p-type, β-doped, MTI Corporation, Richmond, CA) and scanned using a diffractometer with a 3-kV generator, X-ray tube, fully ceramic Cu Long fine focus (LFF), vertical goniometer (theta-theta) and a PIXcel Id detector. Diffractograms were measured through a 1 cm copper-based beam mask from 5-80° (2θ) with a step size of 0.026°, scanning rate of 1.68°/min, and a beam path utilizing a 1/2° divergence slit. Background subtraction was applied using Highscore XRD Software. A XRD peak deconvolution method (Park et al., 2010) was utilized as a curve-fitting process in Igor-Pro software to calculate the crystallinity of microbial cellulose samples. Briefly, characteristic crystalline and amorphous peaks were identified and separated in XRD spectra. Gaussian functions were used for the deconvolution of spectra into three crystalline peaks (100, 010, and 110), and one amorphous peak centered at a 2θ value of ~18.5 degrees. A Vaugt function was used to fit the crystalline peak identified as 200. Crystallinity index (CI) was calculated from the ratio of the area of crystalline regions (A_{cry}) to the total peak area (Aₜₒₜₐₗ):

$$C ( \% ) = \frac{{A}_{cry}}{{A}_{total}} * 100$$

Crystallite size was determined by calculating the Scherrer width via the Scherrer equation:

$$C S = \frac{k λ}{FWHM * cos ( θ )} ,$$

where dₕₖₗ is the size of the crystallite (nm), k is the Scherrer constant of 0.94, λ is the x-ray wavelength set at 0.154 nm, and θ is the diffraction angle.

Additionally, the interplanar lattice spacing (d-spacing) was determined by Bragg's equation:

$${d}_{hkl} = \frac{λ}{2 sin ( θ )} ,$$

in which dₕₖₗ is the lattice spacing of the crystallographic planes, X is the x-ray wavelength (0.154 nm), and θ is the corresponding Bragg angle.

### Antibacterial Response

Antimicrobial activity of microbial cellulose, before and after crosslinking with tannic acid and iron chloride, against Escherichia coli (NiCo21 [DE3] Competent E. coli, New England BioLabs) was determined using a Kirby-Bauer disk diffusion assay (n=5). Microbial cellulose was biofabricated, purified, and treated with tannic acid and iron chloride as previously described. Sodium hydroxide (2 M) was used to tune the pH to acidic (pH<3) and basic (5<pH<6) conditions. Dried as-fabricated, TA-only (0.5% TA, MCTA), and TA-Fe mCellulose samples were cut into 5 mm discs via a biopsy punch (Sklar Surgical Instruments) and sterilized with ultraviolet light for 15 minutes on each side. As a control, filter paper disks (Whatman #1, Ø: 5 mm) absorbed and dried with 20 μL of 2% Penicillin-Streptomycin (P/S) were prepared and subjected to similar sterilization procedure as previously described. E. coli was pre-cultured in Luria broth base (LB) for 17 hours at 37 °C under shaking conditions, until an OD 600 of 1.3 was achieved, equivalent to 2.6×10⁸ CFU/mL. A 1000 μL bacterial suspension, was uniformly spread on prepared LB agar plates (Ø: 100 mm). All sterilized samples were carefully positioned onto the agar plates and incubated at 37 °C for 24 hours. After 24 hours, the inhibition zone diameters were measured using ImageJ.

### Plasticizer Impact Analysis

Life cycle impact assessment (LCA) analysis is a quantitative technique utilized to determine the environmental and human health impacts within a product's lifespan, including raw material extraction, manufacturing, distribution, consumer use, and disposal. The International Organization for Standardization (ISO) for Environmental Management of Life Cycle Assessment—Principles and Frame work (ISO 14040, Geneva, Switzerland) was used to perform cradle-to-gate life cycle impact assessment (Krishna et al., 2017). For each bioprocessing compound of interest, manufacturing impacts were determined using databases within Sustainable Minds© Life Cycle Assessment (Cambridge, MA) and EcoInvent (Zurich, Switzerland) software (Wernet et al., 2016). Following, relevant impact categories classified based upon ecological and human health damage were selected using the U.S. Environmental Protection Agency's (EPA) Tool for the Reduction and Assessment of Chemical and other Environmental Impacts (TRACI), providing characterization factors for life cycle impact assessment (LCIA) methodology. Ecological damage encompassed impact categories such as acidification (kg SO₂ eq.), ecotoxicity (CTUe), eutrophication (kg N eq.), global warming (kg CO₂ eq.), ozone depletion (kg CFC⁻¹¹ eq.), while human health damage included carcinogenics and non-carcinogenics (hormonally active agents (HAA), CTUh), respiratory effects (kg PM2.5 eq., fine particulates), smog formation (kg (ground level) O₃ eq.) and fossil fuel depletion (MJ surplus).

The cradle-to-gate LCA was implemented to compare the human health and environmental impacts of various bioprocessing compounds, such as glycerol, sorbitol, and tannic acid-Fe(III), used to modulate microbial cellulose mechanical performance. Based upon prior experimental post-production bioplasticizing procedures, the functional unit implemented into LCA was 150 cm² of mCellulose material. In addition, di-2ethylhexyl phthalate (DOP), a common plasticizer applied during the production of polyvinyl chloride (PVC) plastic, was utilized as a control to highlight the human health and environmental impacts of post-processing treatments within the textile industry. To establish a direct comparison with this application's bioprocessing treatments, the functional unit implemented into LCA for di-2ethylhexyl phthalate was for the production of 150 cm² of PVC plastic, in which a 1.7 to 1 ratio of attached DOP to PVC was determined from literature (Yuan, Cheng, 2017). All impact categories were converted into respective impacts expressed in milliPoints per functional unit (mPts/cm²) by using a combination of normalization and weighting factors (Ryberg et al., 2014). Briefly, one point (1000 mPts) represents the average annual environmental impact, including material production and consumption activities, of a consumer in the United States. Normalization was implemented to express the prior impacts relative to mPts as a reference by calculating normalization factors from characterization factors within the TRACI 2.1 LCIA model. Additionally, the relative weighting of each impact category was calculated to scale LCA results of measured materials with the environmental performance of competing products. Here, the life cycle assessment was partially performed to only encompass raw material extraction and manufacturing impacts, while further distribution, consumer use, and disposal impacts were not incorporated.

The environmental and human health impacts of post-processing with optimal glycerol, sorbitol, and tannic acid-Fe(III) treatments were determined for 150 cm² of biofabricated microbial cellulose. Initially, EcoInvent database software was used to obtain a system of required materials (Wernet et al., 2016). The material inputs for sorbitol plasticization of mCellulose included 6 g sorbitol (2% w/v), produced from enzymatic hydrolysis, within 300 mL of water. The material inputs for glycerol plasticization included 3.78 g glycerol (1% v/v) within 300 mL of water, in which glycerol was either sourced from vegetable oil produced at an esterification plant, epichlorohydrin (ECH) with 10% NaOH, or epichlorohydrin waste oil. The material inputs for tannic acid-Fe(III) crosslinking included 1.5 g tannic acid (0.5% w/v), sourced from either polyphenols found in waste biomass (TA₁-Fe) orhotwater extraction (TA₂-Fe), and 0.127 oz. FeCl₃ (0.5% w/v) within 300 mL of water. Lastly, the environmental and human health impacts of the plasticizer di-2ethylhexyl phthalate was determined for 150 cm² of PVC plastic, in which the material inputs were based upon a 1.7 to 1 ratio of attached DOP to PVC, measured as 351.9 g of DOP for 207 g of PVC.

Lastly, each post-processing treatment's total impact was segmented into 10 categories encompassing both ecological damage and human health damage, and visually displayed within a donut chart to provide insights of the largest impact hotspot. Of note, all donut charts are organized in hierarchical order of total impacts, however, respective plot sizes are not scaled.

### Reagents and Materials

All reagents and materials were supplied from Fisher Scientific, unless otherwise stated.

### Statistical Analysis

All results are reported as mean±standard deviation, with ‘n’ equal to the number of replicates per study group. One-way analysis of variance (ANOVA) was performed to determine the effect of plasticizer and crosslinking type on mCellulose mass, thickness, tensile mechanical properties, viscoelastic properties, crystallinity, lateral order index, total crystallinity index, elemental composition, and zone of inhibition. The Tukey-Kramer post-hoc test was performed for all pair-wise comparisons with significance attained at p<0.05. All statistical analyses were performed by utilizing JMP-IN (4.0.4, SAS Institute, Inc.) and Prism Software (GraphPad).

### Results

### Evaluating Sorbitol as a Bioplasticizer

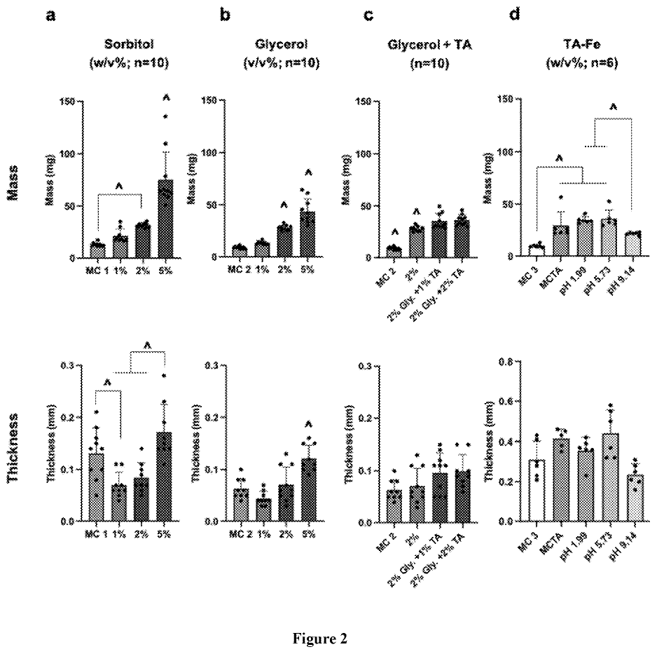

Microbial cellulose was successfully biofabricated after 14 days of G. xylinus culture in 2% w/v mannitol at 30 °C. Following biofabrication, mCellulose was treated with 1, 2, and 5% w/v sorbitol, and resulting biofilms were characterized to evaluate the plasticizer's capability to modulate the biopolymer's mechanical behavior. Initially, mCellulose mass and thickness was measured before and after sorbitol treatment(s) (FIG. 2a). It was observed that as-fabricated mCellulose (MC 1) mass (12.84±1.75 mg) significantly increased at 2% w/v sorbitol to 33.08±5.29 mg, however, thickness slightly decreased. With sorbitol concentration increasing to 5% w/v, mass and thickness significantly increased to 75.31±26.55 mg and 0.19±0.08 mm, respectively, in comparison to both 1% and 2% w/v treated samples.

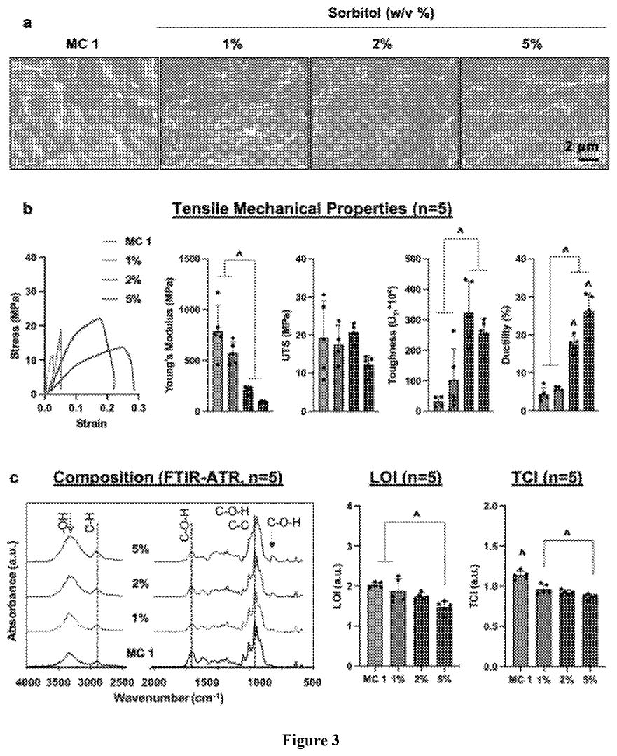

Next, mCellulose surface morphology was evaluated by SEM, in which as-fabricated groups had a densely packed fibrous surface with low porosity comparable to the G. xylinus cultures prepared with mannitol in (FIG. 3a). Fibrous structure was continually observed without any presence of plasticizer coverage as sorbitol incorporation into the mCellulose network increased.

Dried mechanical properties of MC 1 and sorbitol-treated mCellulose were evaluated under uniaxial tension (FIG. 3b). It was observed that as-fabricated microbial cellulose had a high young's moduli measuring at 787.98±253.55 MPa, with an accompanying low ductility of 4.31±1.76%. Comparatively, sorbitol-treated samples exhibited lower young's moduli, however, elongation at break was accordingly enhanced. As sorbitol incorporation increased from 1 to 2% w/v, Young's Modulus significantly decreased from 573.48±111.67 to 248.12±85.20 MPa, while ductility significantly enhanced from 6.77±2.20% to 17.48±2.18%. mCellulose plasticized with 5% w/v sorbitol significantly increased ductility, measuring at 26.26±4.75%, compared to all other groups, though its Young's Modulus was statistically significantly lower than MC 1 and 1% w/v samples. Correspondingly, the toughness was also calculated for MC 1, 1, 2, and 5% w/v sorbitol, measuring 31.11±17.17, 101.96±103.25, 322.71±103.76, and 256.29±48.87 (* 10⁴) U_{T}, respectively, in which significant increases were measured at 2 and 5% w/v incorporation. The ultimate tensile stress was observed to be comparable between all groups, regardless to the incorporation of sorbitol.

Lastly, the chemical properties of mCellulose before and after sorbitol treatment was assessed by FTIR-ATR (FIG. 3c). Characteristic peaks of mCellulose were identified at 3345 cm⁻¹ (O—H hydroxyl stretching), 2898 cm⁻¹ (C—H methyl stretching), 1633 cm⁻¹ (—OH symmetrical stretching), and 1058 cm⁻¹ C—O—H/C—C stretching) (Schiros et al., 2022; Atykyan et al., 2020). For the spectra of sorbitol-plasticized mCellulose, the absorptions between 3600-3000 cm⁻¹ and 902-845 cm⁻¹, attributed to O—H hydroxyl stretching, increased in peak intensity as plasticizer concentration increased, likely due to the large amounts of —OH groups observed in sorbitol (Wang et al., 2012; Moshin et al., 2011; Somashekarappa et al., 2013; Sun, Liang et al., 2018). Additionally, a small increase in peak intensity and shift in peak location from 2898 cm⁻¹ to 2916 cm⁻¹ was observed, correlating with the C—H vibrational stretching in sorbitol molecules. Further FTIR analysis utilizing peak ratios to measure the crystallinity index confirmed vibrational changes throughout the bulk of mCellulose due to increasing sorbitol incorporation. It was observed that both lateral order index and total crystallinity index decreased with increasing sorbitol concentration treatment, with significant differences initially found by 5% w/v for LOI and 1% w/v for TCI compared to MC 1.

### Evaluating Glycerol as a Bioplasticizer

A secondary batch of microbial cellulose (MC 2) was biofabricated for 14 days in 2% w/v mannitol at 30 °C, and further preparation with glycerol was evaluated to determine the bioplasticizing effect on the resulting biopolymer structure, chemistry, and mechanical performance. The incorporation of glycerol into the mCellulose network after immersion into 1, 2, and 5% v/v baths was evaluated by recording sample mass and thickness (FIG. 2b). It was observed that mCellulose mass was positively correlated to increasing glycerol concentration, with significant differences at 2% (27.66±2.53 mg) and 5% w/v (43.65±11.96 mg) when compared to MC 2 (8.88±1.19 mg). However, significant increases in mCellulose thickness (MC 2: 0.06±0.02 mm) only occurred within 5% v/v groups to 0.12±0.03 mm.

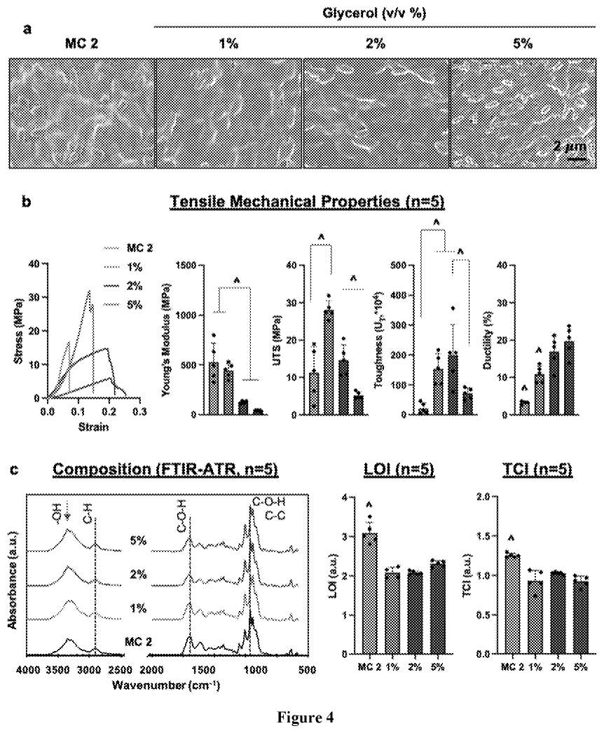

Surface architecture and fiber morphology of as-fabricated and glycerol-treated microbial cellulose were imaged by scanning electron microscopy (FIG. 4a). MC 2 exhibited a dense randomly organized fibrous structure morphologically identical to MC 1 and G. xylinus cultures prepared with mannitol. In contrast, the inherent fibrous structure became less visible with increasing glycerol treatment from 1 to 5% v/v, as a result of plasticizer's homogenous coverage on mCellulose. Interestingly, while fiber morphology was still present at 1% v/v, 2 and 5% v/v groups displayed a smooth layer, subjected to the formation of micro-cracks and pores when exposed to the accelerating voltage of SEM.

Dry mechanical properties were measured for both as-fabricated and glycerol-treated (1, 2, and 5% v/v) mCellulose (FIG. 4b). As-fabricated groups showed a comparable tradeoff between young's moduli (527.76±190.26 MPa) and ductility (3.23±0.43%) to MC 1. With increasing glycerol concentration from 0 to 1% v/v, young's moduli remained comparable around 485 MPa, however, ductility was significantly enhanced to 10.89±2.20%. Greater glycerol treatments of 2 and 5% v/v significantly decreased young's moduli to 125.42±13.86 MPa and 39.83±6.06 MPa, respectively, in which subsequent ductility exhibited a positive correlation to concentration up to 19.74±3.99%. As a result, mCellulose toughness was observed to significantly increase up to 2% v/v (198.58±103.30*10⁴ U_{T}) in comparison to MC 2 (20.88±18.67*10⁴ U_{T}), but lower moduli reduced toughness values at 5% v/v. Similarly, ultimate tensile strength significantly increased at 1% v/v, measuring 28.10±2.46 MPa, followed by statistically lower values, measured in higher glycerol concentrations.

The chemical composition of mCellulose treated with and without glycerol was assessed using FTIR-ATR (FIG. 4c). Similar to MC 1, characteristic mCellulose peaks were identified, including 3345 cm−¹ (O—H hydroxyl stretching), 2898 cm⁻¹ (C—H methyl stretching), 1633 cm⁻¹ (—OH symmetrical stretching), and 1058 cm⁻¹ C—O—H/C—C stretching), supporting that no chemical modifications to as-fabricated samples from varying biofabricated cultures occurred due to purification procedures (Schiros et al., 2022; Atykyan et al., 2020). Incorporation of glycerol to mCellulose increased peak intensity of the absorptions between 3600-3000 cm⁻¹, indicating an increase in the presence of hydroxyl groups (Cielecka et al., 2019; Sun, Meng et al., 2018; Sun, Liang et al., 2018; Chen et al., 2008; AlOmar et al., 2016). Furthermore, LOI and TCI measurements significantly decreased after bioplasticizing mCellulose (MC 2) with glycerol (1, 2, and 5% v/v), revealing the formation of less ordered structures with lower degrees of crystallinity.

### Determining Tannic Acid as a Crosslinker

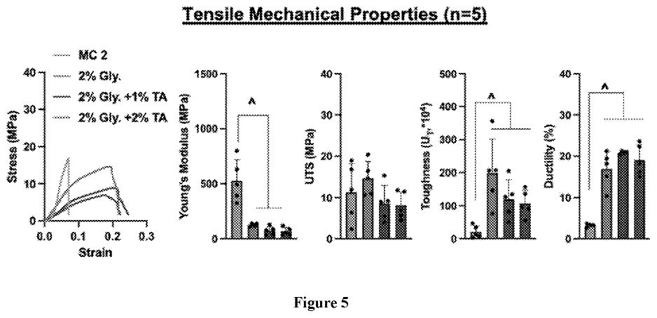

After reporting glycerol-treated mCellulose's characteristic reduced strength with improved ductility, a consecutive immersion procedure with tannic acid was chosen to maintain strength without loss in elongation at break (FIG. 5). Here, mCellulose prepared using both glycerol (2% v/v) and tannic acid (1 or 2% w/v) were characterized to investigate mechanical performance. It was observed that mCellulose (MC 2) significantly increased in mass with 2% v/v glycerol treatment, however, successive tannic acid immersion in 1 or 2% w/v showed comparable masses around ~27.66±2.53 mg (FIG. 2c). Similarly, no significant modification in sample thickness (~0.07 mm) occurred after both plasticizer and tannin immersion.

Dry mechanical properties of as-fabricated and treated mCellulose evaluated under uniaxial tension revealed that TA-glycerol-based samples had comparable Young's Moduli, ultimate tensile stress, toughness, and ductility to the 2% v/v glycerol- only treated samples, measuring around 125.42±13.86 MPa, 14.62±4.07 MPa, 198.58±103.30*10⁴ U_{T}, and 16.89±4.26%, respectively (FIG. 5b).

Evaluating mCellulose Crosslinking via Tannic Acid—Iron Complexes

A third batch of microbial cellulose (MC 3) was biofabricated for 14 days in 2% w/v mannitol at 30 °C, and was successfully crosslinked at various pH (1.99, 5.73, and 9.14) after consecutive immersions in 0.5% w/v tannic acid and 0.5% w/v iron chloride baths for 1 day and 2 hours, respectively. Following crosslinking, TA-Fe-mCellulose was characterized to evaluate the mechanical performance of tannin-metal-cellulose complexation as a green bioprocessing technique, in which all results were compared to control groups (as-fabricated [MC 3] and MCTA (0.5% w/v TA).

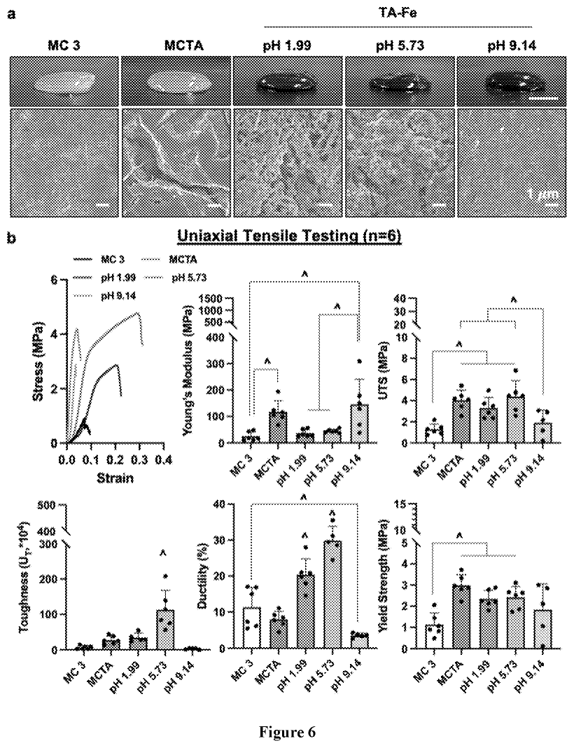

Initially, mCellulose bulk macroscale (wet) and fibrous nanofiber (dry) morphology was assessed by representative photographs and SEM, respectively (FIG. 6a). As-fabricated mCellulose purified after biofabrication revealed pellicles with semi-transparent white color. Upon immersion in 0.5% w/v tannic acid, hydrated mCellulose became light brown, and additional crosslinking with iron (III) formed black pellicles at each pH. MC 3 exhibited a dense randomly organized nanofibrous structure morphologically identical to MC 1, MC 2, and G. xylinus cultures prepared with mannitol. In contrast, the nanofibers observed in MCTA groups were non-homogeneously covered with aggregates of the tannin. Immersion of TA-treated mCellulose into FeCl₃ under pH conditions of 1.99 and 5.73 exhibited the formation of TA-Fe nanoparticles adhered to nanofibers. Further pH modification to 9.14 displayed randomly oriented nanofibers similar to as-fabricated MC 3, revealing the loss of TA coverage and nanoparticle features.

Upon drying, mCellulose mass and thickness was recorded to determine bulk incorporation of tannin-metal complexes (FIG. 2d). The mass of dried microbial cellulose biofilms (9.9±1.89 mg) significantly increased to 29.13±13.58 mg when immersed into 0.5% w/v TA, however, complexation with iron at pH 1.99 and 5.73 did not statistically increase the bulk weight. Further changes to pH 9.14 significantly reduced mass to 21.5±1.44 mg, comparable to as-fabricated biofilms. Moreover, thickness was maintained around ~0.31±0.10 mm for each treatment.

Following, dried mechanical properties of mCellulose were evaluated utilizing uniaxial tensile testing (FIG. 6b). It was observed that as-fabricated MC 3 had decreased young's moduli, tensile stress, and yield strength compared to MC 2 and MC 1, albeit culturing conditions were maintained in 2% w/v mannitol at 30 °C for 14 days. mCellulose significantly increased in Young's Modulus (117.24±42.11 MPa), ultimate tensile stress (4.03±0.96 MPa), and yield strength (2.99±0.51 MPa) with an initial 0.5% w/v TA treatment, though ductility was held at approximately 7.92±2.26%. Further complexation with iron at pH 1.99 and 5.73 decreased young's moduli to values comparable to as-fabricated groups, while maintaining tensile strength and yield strength. Ductility and toughness significantly increased in a pH dependent manner from pH 1.99 to 5.73, measuring a final 29.85±4.03% and 112.76±55.10 U_{T} (*10⁴), respectively. Tannin-iron complexation with cellulose at pH 9.14 not only significantly reduced ultimate tensile stress, ductility, and toughness, but also enhanced Young's Moduli to 145.40±95.28 MPa comparable to MCTA groups.

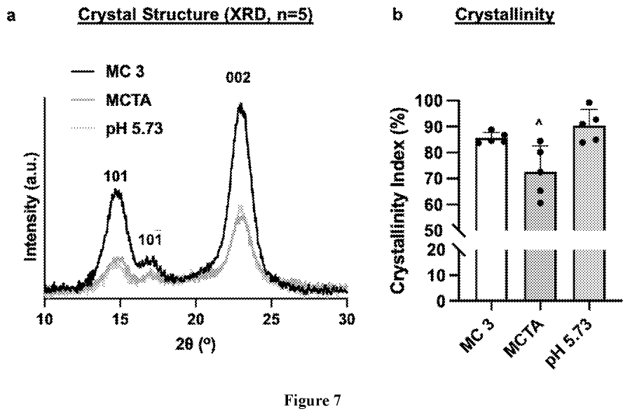

Microbial cellulose crystal structure was assessed by X-ray diffraction (FIG. 7a). While all groups exhibited cellulose I Bragg peaks at 2θ=14.6°, 16.9°, and 22.8°, corresponding to the (1-10), (110), and (200) planes (Samuel, Adefusika, 2019), a peak deconvolution method indicated a minor increase in crystallinity index in mCellulose complexed with tannic acid and iron at pH 5.73 (MC 3: 85.61±2.15% vs. pH 5.73: 90.34±6.25%; FIG. 7b). Collectively, the tensile properties and crystallinity index of MC 3 and pH 5.73 suggests that complexation with iron alters mCellulose bulk structure and mechanical behavior, as a result of chemical crosslinking.

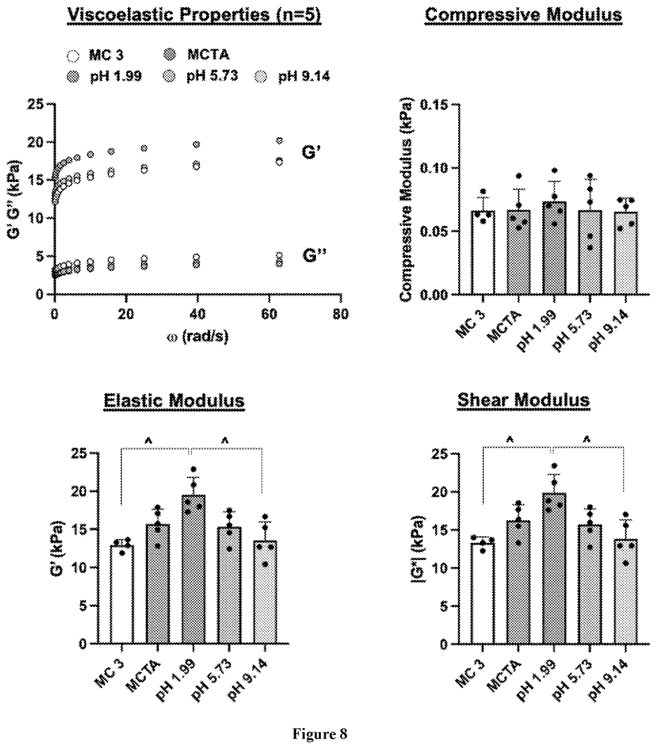

To further determine the effect of complexation between microbial cellulose, tannic acid, and iron on mechanical behavior, the viscoelastic properties of hydrated as-fabricated and treated groups were evaluated via shear-strain controlled rheometry (FIG. 8). Complexation occurring at pH 1.99 significantly increased both elastic (storage) and shear modulus to 19.53±2.31 kPa and 19.87±2.41 kPa, respectively, while a further increase in pH to 5.73 and 9.14 continually reduced moduli. Increased moduli in response to consecutive 0.5% w/v TA and 0.5% w/v FeCl₃ treatment indicated increased crosslinking density with bulk mCellulose, in which complexation was pH dependent, thus correlating with dry tensile properties.

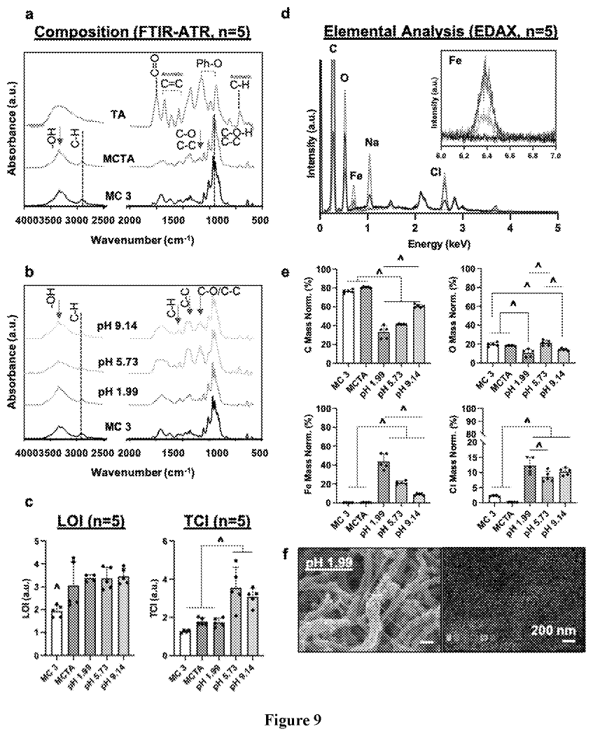

The chemical composition of mCellulose treated with and without tannic acid was assessed using FTIR-ATR (FIG. 9a). Characteristic as-fabricated mCellulose peaks were identified, including 3345 cm⁻¹ (O—H hydroxyl stretching), 2898 cm⁻¹ (C—H methyl stretching of CH₂ and CH₃), 1633 cm⁻¹ (—OH symmetrical stretching), and 1058 cm⁻¹ C—O—H/C—C/C—O—C pyranose ring stretching), supporting that no chemical modifications to MC 3 from varying batches of biofabricated cultures occurred (Schiros et al., 2022; Atykyan et al., 2020). The chemical structure of pure tannic acid was also confirmed by ATR, where characteristic peaks for phenolic hydroxyl (—OH) groups, carboxylic esters (C═O), aromatic C═C groups, aromatic C—C group, and benzene ring vibrations were observed at broad bands between 3600-3000 cm⁻¹ , 1692 cm⁻¹, 1605/1529/1444 cm⁻¹, 1312 cm⁻¹, and 1174/1033/1012 cm⁻¹ respectively (Zhang, Sun, et al., 2020; Ninan et al., 2016; Lee et al., 2018; Muhoza et al., 2019; Iglesias, Jaén, 2001). Microbial cellulose treated with 0.5% w/v TA maintained the biopolymer's characteristic spectra, while introducing small increases in peak intensities at 3345 cm⁻¹ (O—H groups), 1312 cm⁻¹ (benzene ring C—C), and 1205 cm⁻¹ (C—O, C—C), thus, indicating incorporation of the tannin. Complexation with iron at pH 1.99 further increased peak intensity around 3345 cm⁻¹, representing greater hydroxyl groups (FIG. 9b). However, an increase in pH to 5.73 and 9.14 irreversibly decreased peak intensity of the hydroxyl broad band between 3600-3000 cm⁻¹, increased the relative absorbance of methyl stretching (1428 cm⁻¹), aromatic C—C groups (1312 cm⁻¹), and C—O/C—C groups (1205 cm⁻¹), while maintaining characteristic peaks assigned for mCellulose. Additionally, FTIR peak ratios between 1429 cm⁻¹ and 897 cm⁻¹, expressed as the Lateral Order Index, indicated a significantly higher degree of overall order in cellulose structures after treatment with tannic acid and iron compared to MC 3 (FIG. 9c). Total crystallinity index measurements, dictated by peak ratios between 1372 cm⁻¹ and 2900 cm⁻¹, revealed an increased degree of crystallinity when tannin-iron complexation occurred at pH 5.73, thus supporting differences in crystal structure observed with X-ray diffraction.

Furthermore, elemental composition of mCellulose was evaluated by EDXA to determine the incorporation of iron in conjunction with tannic acid (FIG. 9d). Characteristic peaks for carbon (C) and oxygen (O) for as-fabricated microbial cellulose was observed at k alpha values 0.27 keV and 0.52 keV, respectively. Complexation with iron introduced a peak within spectra at 6.40 keV, however, iron incorporation was found be pH dependent, in which an increase in pH from 1.99 to 9.14 decreased peak intensity and significantly decreased metal mass fraction (FIG. 9e). Spectral maps further indicated homogenous incorporation of iron on the bulk mCellulose surface when crosslinked with iron at pH 1.99 (FIG. 9f).

### Antibacterial Activity

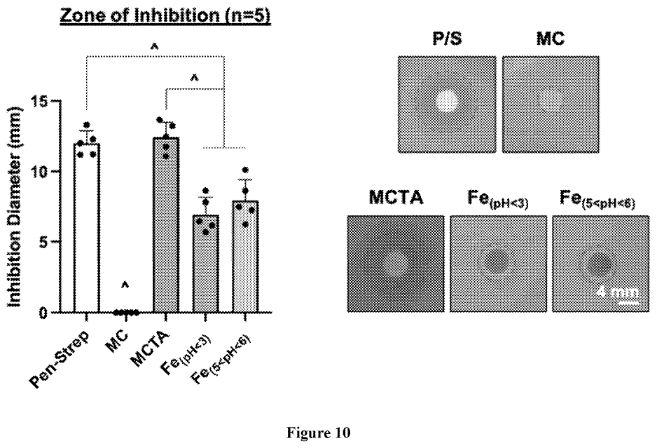

A Kirby-Bauer disk diffusion assay was performed to determine TA-Fe-mCellulose's antimicrobial effect on gram-negative E. coli (FIG. 10). Although as-fabricated samples exhibited no inhibition zones after 24 hours of culture, dried mCellulose films only treated with 0.5% w/v tannic acid significantly increased inhibition zones to 12.44±1.05 mm, which was comparable to control disks containing 2% penicillin-streptomycin. As complexation between tannic acid and iron was formed within mCellulose, the inhibition zone significantly decreased to 6.95±1.23 mm. However, there was no statistical difference of inhibition diameter between TA-Fe samples maintained below pH 3 and modified to pH 5-6. Green Bioprocessing: Plasticizer Impact Assessment

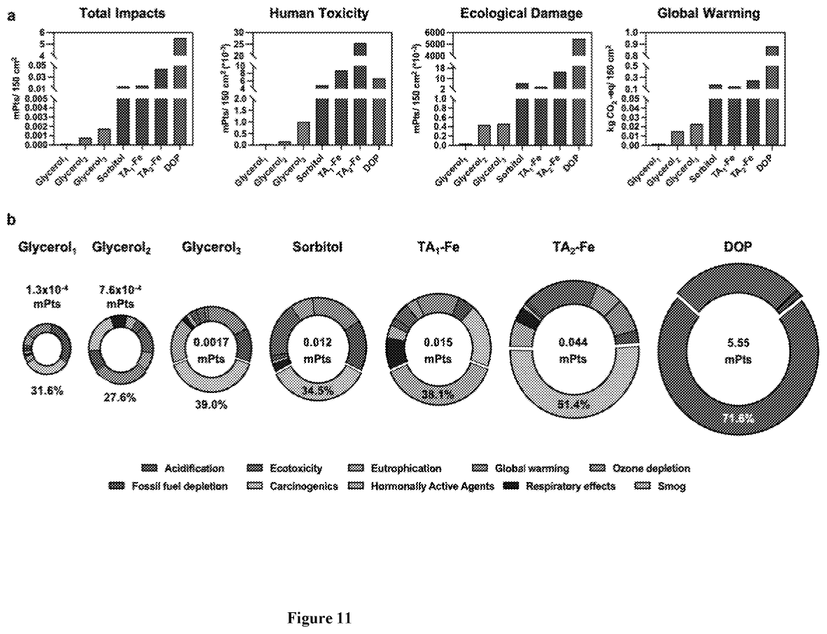

A cradle-to-gate life cycle impact assessment (LCA) was implemented to compare the human health and environmental impacts between various optimal post-production treatments for microbial cellulose, such as glycerol, sorbitol, and tannic acid-Fe(III), to the traditional plasticizer di-2ethylhexyl phthalate (FIG. 11). Both normalization (Ryberg et al., 2014) and weighting factors (Gloria et al., 2007) were applied to convert the manufacturing impacts of 10 categories (Human Health: fossil fuel depletion, carcinogenics, hormonally active agents, respiratory effects, and smog; Environmental: acidification, ecotoxicity, eutrophication, global warming, ozone depletion) into milliPoints per 150 cm² of mCellulose. Di-2ethylhexyl phthalate (DOP), a plasticizer used in the production of most polyvinyl chloride plastic, was observed to have the largest total impact (5.55 mPts) in comparison to glycerol, sorbitol, and tannic acid-Fe(III) (FIG. 11a). Of note, the dramatic impact measured from di-2ethylhexyl phthalate is significantly contributed by ecological damage (5.47 mPts) caused by acidification (71.6%) relative to all other impact categories.

In terms the impacts between green bioprocessing strategies, tannic acid—iron (III)—based methods were observed to have the largest total impacts when modelled against sorbitol (2% w/v) and glycerol (1% v/v). However, depending on the source of tannic acid, resulting human toxicity, ecological damage, and global warming impacts were varied. Here, tannic acid was demonstrated to be extracted from either polyphenol waste biomass (TA₁-Fe) or hot water (TA₂-Fe), in which sourcing from the latter resulted in three times greater total impacts (0.044>0.015 mPts). Interestingly, it was found that tannic acid contributes significantly more to these impacts than its counterpart iron chloride, however, differences were observed in terms of each sources major influence to human toxicity. While carcinogenics accounted for 51.4% of tannic acid sourced from hot water, it was revealed that hormonally active agents accounted for 38.1% of tannic acid extracted from polyphenol waste biomass. Further LCA analysis indicated that TA-Fe's processing has a comparable carbon footprint (0.144-0.249 kg CO₂-eq per 150 cm²) to sorbitol treatment, in which both were one hundred times greater than glycerol procedures.

Sorbitol manufactured by enzymatic hydrolysis slightly reduced total impacts to 0.012 mPts, while ecological damage was measured to be the process' largest impact category (0.0064 mPts). The use of glycerol as a plasticizer post-production treatment offers a significant improvement in human health and ecological performance, as well as global warming impacts (0.0017 kg CO₂-eq per 150 cm²) compared to sorbitol and tannic acid-iron ion procedures. Notably, the source of glycerol also altered the resulting impacts, albeit values remained between ten to one-hundred times lower than sorbitol and TA-Fe. While the production of glycerol manufactured by epichlorohydrin obtained in waste oil (glycerol₃) resulted in the highest total impacts, measuring at 0.0017 mPts, glycerol from vegetable oil at an esterification plant (glycerol1) decreased total impacts to 1.3×10⁻⁴ mPts. This pronounced reduction in impacts with glycerol can be accounted for the elimination of human toxicity comprised by carcinogenics, which were observed to be 34.5% and 51.4% of the bulk total impacts within sorbitol and TA₂-Fe, respectively. However, carcinogenics was continuously measured to be the largest impact for both glycerol produced from vegetable oil (glycerol, 31.6%), and epichlorohydrin waste oil (glycerol₃, 39.0%).

TABLE 2
Portfolio of Mechanical Properties Tunable Based on
Green Processing with Tanin-Metal Complexation.

| | | Young's | Ultimate Tensile | Toughness | |
|---|---|---|---|---|---|
| Group | pH | Modulus (MPa) | Strength (MPa) | (U_{T}, *10⁴) | Ductility (%) |
| As Fabricated | — | 24.85 ± 16.95 | 1.11 ± 0.29 | 7.25 ± 4.88 | 11.28 ± 5.57 |
| 0.5% Tannic Acid | — | 117.24 ± 42.12\hat{ } | 4.03 ± 0.96\hat{ } | 27.93 ± 11.30 | 7.92 ± 2.26 |
| Only | | | | | |
| 0.5% Tannic | 1.99 | 36.55 ± 14.05 | 3.30 ± 1.00\hat{ } | 34.48 ± 11.92 | 20.34 ± 4.44\hat{ } |
| Acid + | 5.73 | 44.82 ± 6.99 | 4.38 ± 1.50\hat{ } | 112.76 ± 55.10\hat{ } | 29.85 ± 4.03\hat{ } |
| 0.5% FeCl₃ | 9.14 | 145.40 ± 95.28\hat{ } | 1.91 ± 1.21 | 2.40 ± 2.01 | 3.53 ± 0.59\hat{ } |

TABLE 3
Post-fabrication processing with tannic acid-iron complexation
tuned microbial cellulose mechanical properties based
on altering pH condition methodologies.

| Group | pH | Zone of Inhibition (mm) |
|---|---|---|
| Pen-Strep (2%) | — | 12.00 ± 0.88\hat{ } |
| As Fabricated | — | 0.00 ± 0.00 |
| 0.5% Tannic Acid Only | — | 12.44 ± 1.05\hat{ } |
| 0.5% Tannic Acid + | 1.8 | 6.95 ± 1.23\hat{ } |
| 0.5% FeCl₃ | 6. | 7.95 ± 1.49\hat{ } |

TABLE 4
Comparison of Treated Microbial Cellulose Mechanical Properties to Conventional Textiles.

| | | Young's Modulus | Ultimate Tensile | Toughness | |
|---|---|---|---|---|---|
| Textile | Concentration | (MPa) | Strength (MPa) | (U_{T}, *10⁴) | Ductility (%) |
| Microbial | 0% | 787.98 ± 253.55 | 19.33 ± 9.67 | 31.11 ± 17.17 | 4.31 ± 1.76 |
| Cellulose | 1% | 573.48 ± 111.67 | 17.56 ± 5.01 | 101.96 ± 103.25 | 5.80 ± 0.56 |
| (Sorbitol- | 2% | 212.49 ± 34.92 | 20.76 ± 2.49 | 322.71 ± 103.76 | 17.48 ± 2.18 |
| treated) | 5% | 91.20 ± 6.79 | 12.24 ± 2.37 | 256.29 ± 48.87 | 26.26 ± 4.75 |
| Microbial | 0% | 527.76 ± 190.26 | 11.27 ± 6.95 | 20.89 ± 18.67 | 3.23 ± 0.43 |
| Cellulose | 1% | 443.77 ± 73.29 | 28.10 ± 2.46 | 153.43 ± 50.87 | 10.89 ± 2.20 |
| (Glycerol- | 2% | 125.42 ± 13.86 | 14.62 ± 4.07 | 198.58 ± 103.30 | 16.89 ± 4.26 |
| treated) | 5% | 39.83 ± 6.06 | 5.20 ± 0.95 | 71.01 ± 18.53 | 19.74 ± 3.99 |
| Microbial | 0% | 24.85 ± 16.95 | 1.11 ± 0.29 | 7.25 ± 4.88 | 11.28 ± 5.57 |
| Cellulose | 0.5% TA Only | 117.24 ± 42.12 | 4.03 ± 0.96 | 27.93 ± 11.30 | 7.92 ± 2.26 |
| (Tannin Metal - | 0.5% TA + 0.5% | 36.55 ± 14.05 | 3.30 ± 1.00 | 34.48 ± 11.92 | 20.34 ± 4.44 |
| treated) | FeCl₃ (pH 1.99) | | | | |
| | 0.5% TA + 0.5% | 44.82 ± 6.99 | 4.38 ± 1.50 | 112.76 ± 55.10 | 29.85 ± 4.03 |
| | FeCl₃ (pH 5.73) | | | | |
| | 0.5% TA + 0.5% | 145.40 ± 95.28 | 1.91 ± 1.21 | 2.40 ± 2.01 | 3.53 ± 0.59 |
| | FeCl₃ (pH 9.14) | | | | |
| Plastic (LDPE) | — | 158.87 ± 70.40 | 8.85 ± 2.43 | 429.32 ± 232.97 | 62.42 ± 15.09 |
| Polyester | — | 126.55 ± 13.80 | 51.31 ± 4.59 | 1244.55 ± 90.14 | 46.85 ± 2.68 |
| Cotton | — | 396.46 ± 130.07 | 24.93 ± 6.13 | 111.53 ± 15.48 | 9.15 ± 1.45 |

TABLE 5
Comparison of Treated Microbial Cellulose Mechanical Properties to Conventional Textiles.

| | | Young's Modulus | Ultimate Tensile | Toughness | |
|---|---|---|---|---|---|
| Textile | Concentration | (MPa) | Strength (MPa) | (U_{T}, *10⁴) | Ductility (%) |
| Microbial Cellulose | 0% | 787.98 ± 253.55 | 19.33 ± 9.67 | 31.11 ± 17.17 | 4.31 ± 1.76 |
| (Sorbitol-treated) | 1% | 573.48 ± 111.67 | 17.56 ± 5.01 | 101.96 ± 103.25 | 5.80 ± 0.56 |
| | 2% | 212.49 ± 34.92 | 20.76 ± 2.49 | 322.71 ± 103.76 | 17.48 ± 2.18 |
| | 5% | 91.20 ± 6.79 | 12.24 ± 2.37 | 256.29 ± 48.87 | 26.26 ± 4.75 |
| Microbial Cellulose | 0% | 527.76 ± 190.26 | 11.27 ± 5.95 | 20.89 ± 18.67 | 3.23 ± 0.43 |
| (Glycerol-treated) | 1% | 443.77 ± 73.29 | 28.10 ± 2.46 | 153.43 ± 50.87 | 10.89 ± 2.20 |
| | 2% | 125.42 ± 13.86 | 14.62 ± 4.07 | 196.58 ± 103.30 | 16. [symbol] ± 4.26 |
| | 5% | 39.83 ± 6.06 | 5.20 ± 0.95 | 71.01 ± 18.53 | 19.74 ± 3.99 |
| Microbial Cellulose | 0% | 24.85 ± 16.95 | 1.11 ± 0.29 | 7.25 ± 4.88 | 11.28 ± 5.57 |
| (Tannin Metal - | 0.5% TA Only | 117.24 ± 42.12 | 4.03 ± 0.96 | 27.93 ± 11.30 | 7.92 ± 2.26 |
| treated) | 0.5% TA + 0.5% FeCl₃ | 36.55 ± 14.05 | 3.30 ± 1.00 | 34.48 ± 11.92 | 20.34 ± 4.44 |
| | (pH 1.99) | | | | |
| | 0.5% TA + 0.5% FeCl₃ | 44.82 ± 6.99 | 4.38 ± 1.50 | 112.76 ± 55.10 | 29.85 ± 4. [symbol] |
| | (pH 5.73) | | | | |
| | 0.5% TA + 0.5% FeCl₃ | 145.40 ± 95.28 | 1.91 ± 1.21 | 2.40 ± 2.01 | 3.53 ± 0.59 |
| | (pH 9.14) | | | | |
| Plastic (LDPE) | — | [symbol] | [symbol] | [symbol] | [symbol] |
| Polyester | — | [symbol] | [symbol] | [symbol] | [symbol] |
| Cotton | — | [symbol] | [symbol] | [symbol] | [symbol] |

[symbol]

indicates data missing or illegible when filed

A purification procedure was developed to support the preparation of microbial cellulose into a biocompatible, biomimetic nanofibrous scaffold that is suitable for in-vitro cell culture. To this end, the impact of purifying mCellulose was assessed by G. xylinus viability, as well as, biocompatibility was evaluated by the inflammatory response of naïve THP-1 macrophages, and through anterior cruciate ligament fibroblast culture.

The effects of combined chemical induction, microfiber matrix alignment, and physiological mechanical stimulation on THP-1 macrophage response and polarization towards pro- and anti-inflammatory phenotypes was also evaluated. While fibrous matrix microenvironment cues, such as fiber orientation, mechanical properties, and surface chemistry and energy, are typically used to influence macrophage behavior, mechanical loading as an alternative or supplementary strategy can offer enhanced modulation for guiding polarization. It was further determined if fiber alignment or mechanical stimulation dominantly determines cell response.

To mitigate the shortcomings of bacterial-based cellulose for biomedical applications, the development of a post-processing purification procedure was evaluated, which would facilitate improved biomaterial biocompatibility. Several chemical and high-pressure steam sterilization techniques were used to prepare microbial cellulose as a scaffold that supports cell-based biofabrication by reducing bacterial presence and endotoxin content, without compromising nanofibrous structure or chemistry. Biocompatibility was assessed by culturing naïve THP-1 macrophages on as-fabricated and purified microbial cellulose scaffolds, in which resulting pro- or anti-inflammatory responses would be evaluated. It is hypothesized that purified mCellulose would not illicit an inflammatory response from naïve macrophages, demonstrating the production of a biocompatible scaffold. Additionally, the ability for purified microbial cellulose to support ACL fibroblast viability and growth was evaluated to further support the biocompatibility of the nanofibrous system. Microbial cellulose has immense potential as a biomaterial that meets the design criteria for repairing complex tissue systems.

### Materials and Methods

### Microbial Cellulose Biosynthesis

The Gluconacetobacter xylinus strain ATCC 31174 was acquired for microbial cellulose biofabrication. Hestrin-Schramm culture medium (0.5% w/v yeast extract [Fisher Scientific], 0.5% w/v peptone [Fisher Scientific], 0.27% w/v sodium phosphate dibasic anhydrous [Fisher Scientific]), was used for bacterial culture. The medium pH was adjusted to 4.5 using citric acid (Fisher Scientific). All culture medium was sterilized via autoclaving at 250 °C for 20 minutes.

Prior to mCellulose biofabrication, G. xylinus was pre-cultured in sterile HS medium containing 2% w/v mannitol for 72 hours at 30 °C. under static conditions. Following, the preculture was inoculated (5% w/v) into sterile HS medium (2% w/v mannitol) within sterilized Pyrex dishes (34×23×5 cm). Static fermentation at 30 °C. was maintained for 14 days, in which 2-D layer by layer production of mCellulose occurred at the air-liquid medium interface, resulting in pellicles with a thickness of 0.7 cm.

mCellulose Purification Method Development

Microbial cellulose biofilms were purified by performing a series of chemical and high-pressure steam sterilization techniques. After fabrication, hydrated biofilms were initially rinsed in deionized water to remove surface residual sugars from the culture medium. Following, mCellulose was submerged into a 1% v/v cellulase (Trichoderma reesei, 6200-7580 IU/g, U.S. Biological, Salem, MA) bath for 1 hour to enzymatically weaken the dense nanofibril cellulose matrix, potentially allowing for more penetration during chemical sterilization. Next, biofilms were repeatedly washed with deionized water to remove enzymatic remnants, and immediately immersed into 70% ethanol for 24 hours, and then into 0.1M sodium hydroxide (NaOH) for a subsequent 24 hours to facilitate bacterial cell lysis. All biofilms were continuously submerged into fresh deionized water every 2 hours for 24 hours to further remove medium and basic components from the cellulose until the pH returned to neutral conditions. Last, mCellulose were subjected to high-pressure steam sterilization via autoclaving on liquid cycle (250 °C) for 30 minutes. Resulting purified mCellulose was stored in autoclaved deionized water at 4 °C to prevent bacterial growth prior to use for eukaryotic cell culture.

Purified mCellulose Characterization

Bacterial cell viability (n=10) within microbial cellulose was visualized using Live/Dead staining (Molecular Probes, Eugene, OR) according to the manufacturer's protocol. Briefly, throughout the purification procedure microbial cellulose samples were stained with Live/Dead reagent for 30 minutes at 25 °C and protected from light. Stained samples were rinsed with deionized water and immediately imaged by confocal microscopy (Fluoview FV1000, Olympus) at 488 nm (excitation) and 594 nm (emission) wavelengths to assess cell viability and death, respectively. Following, overlapping pixel percentage between z-stacked images (n=10) from live and dead channels was calculated using MATLAB software.

To determine the bacterial cell content (n=5) within mCellulose throughout the purification process, a Quanti-iT™ PicoGreen® dsDNA assay kit (Molecular Probes) was utilized. Hydrated microbial cellulose biofilms (as-fabricated, EtOH-treated, and purified) were prepared into 1 cm² sections, rinsed once with PBS and stored in 500 μL 0.1% v/v Triton X (Sigma-Aldrich) at −20 °C. Before analysis, samples were thawed to room temperature, homogenized by vortexing, further ultrasonicated with a sonicating probe (Microson Ultrasonic Cell Disruptor XL-2000, Farmingdale, NY) at 5 watts for 15 seconds to lyse cells. All samples were desiccated and dehydrated for 12 hours in a CentriVap Concentrator until completely dry and digested for 18 hours at 65 °C with papain (8.3 activity units/mL, Sigma-Aldrich) in 0.1 M sodium acetate (Sigma-Aldrich), 10 mM cysteine-HCl (Sigma-Aldrich), and 50 mM ethylenediaminetetraacetate (Sigma-Aldrich). After digestion, total bacterial content for 1 cm² sections, dictated by fluorescence intensity, was measured using a microplate reader (SpectraFluor Plus, Tecan, Research Triangle Park, NC, USA) with an excitation wavelength of 485 nm and an emission wavelength of 535 nm.

To assess mCellulose endotoxin content (n=5), a Pierce Limulus Amebocyte Lysate (LAL) chromogenic endotoxin quantification assay kit (Thermo Scientific) was used following the manufacturer's suggest protocol. Hydrated microbial cellulose biofilms (as-fabricated, EtOH-treated, and purified) were prepared into 1 cm² sections, frozen at −80 °C for 24 hours, and lyophilized in a freeze dryer system (Labconco FreeZone) for 24 hours at −84 °C and 2.0×10² mbar (Pigaleva et al., 2019). Freeze-dried mCellulose samples were re-hydrated in endotoxin-free water (1 mL) and vortexed (Vortex Genie 2, Scientific Industries) for 48 hours. Finally, a thousand-fold dilution of the prior extraction medium with endotoxin-free water was prepared for the LAL assay. Absorbance was measured at 405 nm with a microplate reader (BioTek Synergy 5), in which endotoxin content for all mCellulose samples were determined by correlating measured optical density to E. coli endotoxin standard.

Microbial cellulose was characterized using discs (Ø: 10 mm) cut from dried as-fabricated and purified samples with a biopsy punch (Sklar Surgical Instruments, West Chester, PA). mCellulose fiber morphology was assessed by performing scanning electron microscopy (SEM, n=3, 3 keV, Zeiss Sigma VP, Carl Zeiss AG, Oberkochen, Germany) on the surface of samples that were sputter coated (10 nm, 20 s) with gold-palladium to improve sample conductivity and reduce charging effects (Cressington 108 Auto, Cressington Scientific, Watford, UK). The fiber diameter of as-fabricated and purified samples was measured by analyzing randomly selected fiber segments in SEM images using NIH ImageJ software (Bethesda, MD, USA; n=50). Elemental composition of as-fabricated and purified mCellulose surfaces was determined by energy dispersive x-ray analysis (EDXA, XFlash® 6×30 Detector, Espirit 2.1 software, Bruker, Billerica, MA) was performed at 10 keV for 300 s to acquire quantitative spectra, and elemental mass normalization for sodium (n=5).

Microbial cellulose chemical composition was determined by using attenuated total reflectance mode during Fourier transform infrared spectroscopy (FTIR-ATR, n=5, LUMOS). All spectra were collected from 600-4000 cm⁻¹ (200 scans, spectral resolution 2 cm⁻¹) and assessed for characteristic cellulose peaks (Schiros et al., 2022; Atykyan et al., 2020).

Uniaxial tensile testing was conducted on microbial cellulose biofilms based upon the ASTM International standard tensile test method for plastics (D638-14) (Moffat et al., 2009; Lu et al., 2005). Briefly, hydrated as-fabricated and purified mCellulose samples (5×1 cm, n=8) were weighed, and thickness was measured using digital calipers. The samples were secured with clamps and mounted onto a microtester device (Instron, Model 5848) equipped with a 100 N load cell. Samples were maintained to have an average gauge length of 30 mm and were tested to failure at a strain rate of 5 mm/minute. The resulting microbial cellulose stress-strain curves were utilized to calculate elastic modulus from the slope of the linear region, ultimate tensile strength from the maximum stress achieved prior to sample failure, toughness from the area under the curve, and ductility as the percent elongation at failure.

### Synthetic Fiber Fabrication

Blends of polylactide-co-glycolide (PLGA, 50:50, M_{w}≈38-54 kDa, Sigma-Aldrich) and polycaprolactone (PCL, Mₙ≈70-90 kDa, Sigma-Aldrich) (Mosher et al., 2021) were fabricated into meshes via a standardized electrospinning protocol (Moffat et al., 2009; Reneker, Chun, 1996). Briefly, PLGA and PCL were solubilized at a weight ratio of 5:1 in glacial acetic acid (Sigma-Aldrich), resulting in an 32% w/v polymer solution. The polymer melts were vortexed (Vortex Genie 2, Scientific Industries) for 24 hours, and loaded into a 5 mL syringe with a 18G stainless steel blunt-tip needle. To fabricate unaligned meshes, blended solutions were ejected from a syringe pump at 0.75 mL/hour towards a grounded stationary plate positioned 12 cm away, and then electrospun at a voltage of 10 kV. Unaligned fibers were electrospun until 0.1-0.14 mm thick meshes were obtained, in which resulting microfibers had a diameter of 1.36±0.56 μm (n=50).

### Cells and Cell Culture

Human THP-1 monocytes were acquired commercially (ATCC, TIB-202) for in-vitro culture. Cells were maintained in suspension within non-tissue culture treated flasks (25 cm², Nunc™) and cultured with Roswell Park Memorial Institute-1640 (RPMI-1640) medium fully supplemented with 10% fetal bovine serum (FBS; Atlanta Biologicals, Norcross, GA, USA), 1% penicillin/streptomycin (P/S; 10,000 U/mL penicillin, 10 mg/mL streptomycin; Cellgro-Mediatech), and 0.05 mM 2-mercaptoethanol (2-ME; Sigma-Aldrich) at 37 °C and 5% CO₂. To prevent cell density from getting to high (1×10⁶ cells/mL), which has been reported to promote cell death and loss of differentiation potential, the culture medium was changed every three days by removing the cell suspension from flasks, and centrifuging at 500 rpm for 5 minutes. The supernatant was carefully aspirated without disturbing the cell pellet, in which the monocytes were resuspended in F/S RPMI-1640 at a density of 3×10⁵ cells/mL. Passage 5 THP-1 monocytes were used for all subsequent studies.

Prior to cell culture, synthetic (PLGA:PCL) and microbial cellulose scaffolds (as-fabricated, EtOH-treated, and purified) were prepared into 1 cm² sections and sterilized by ultraviolet radiation (10 minutes/side). All scaffolds were incubated in RPMI supplemented with 10% FBS, and 1% P/S for 24 hours at 37 °C and 5% CO₂ to encourage cell attachment. THP-1 monocytes were seeded onto scaffold surfaces and differentiated into naïve macrophages utilizing the following protocol. Briefly, THP-1 monocytes grown to confluency in suspension culture were centrifuged at 500 rpm for 5 minutes to obtain a cell pellet. While the supernatant was discarded, cells were resuspended in F/S RPMI-1640. Monocytes were seeded onto scaffolds at a density of 200,000 cells/cm², allowed to attach for 20 minutes at 37 °C and 5% CO₂, and differentiated for 24 hours at 37 °C and 5% CO₂using F/S RPMI-1640 supplemented with 25 nM phorbol 12-myristate 13-acetate (PMA) (Daigneault et al., 2010; Park et al., 2007). After differentiation, all samples were rinsed twice with sterile PBS to remove non-adherent cells. Following, cells seeded on purified mCellulose were either allowed to rest (M0) with F/S RPMI-1640 or were subjected to pro- (M1) or anti-(M2) inflammatory polarization media for 24 hours (Chanput et al., 2013; Changput et al., 2014). While pro-inflammatory polarization medium contained F/S RPMI supplemented with 20 ng/mL Interferon-γ (IFN-γ) and 100 ng/mL lipopolysaccharide (LPS), anti-inflammatory medium consisted of F/S RPMI+20 ng/mL Interleukin-4 (IL-4). After resting or polarization, media was aspirated, cells were washed with PBS once (in polarized groups only), and replenished with fresh F/S RPMI. Subsequently, all cell-seeded scaffolds were incubated with F/S RPMI and cultured at 37 °C and 5% CO₂ for 2 days.

Following, neonatal (1-7 days old) bovine metacarpophalangeal knee joints were obtained from a local abattoir (Green Village Packing Company, Fort Lee, NJ, USA) to obtain primary anterior cruciate ligament fibroblasts for further evaluation of Cellulose biocompatibility. Prior to harvest, knee joints were inspected to ensure that the joint capsule was not compromised, and immediately sterilized by soaking in detergent and deionized water for 40 minutes, followed by 70% ethanol for 20 minutes. In an aseptic laboratory hood, the joint was opened with a #22 scalpel blade (Feather, Thermo Fisher Scientific) and kept moist with gauze soaked in sterilized deionized water to prevent tissue from drying out. Using aseptic techniques, the synovial capsule was entered, and anterior cruciate ligaments were isolated. During tissue isolation transections were made away from ACL insertion sites located at the femur and tibia. The ligament was gently scraped with a #22 scalpel blade to remove the sheath and diced into smaller pieces (~1 cm²). Bulk tissue was positioned onto tissue culture treated petri dishes (Ø: 100 mm) and attached for 20 minutes at 37 °C and 5% CO₂. Following, all tissue was suspended in Dulbecco's Modified Eagle Medium (DMEM) medium fully supplemented with 10% fetal bovine serum (FBS, Atlanta Biologicals, Minneapolis, MN, USA), 2% penicillin/streptomycin (P/S, 10,000 U/mL penicillin, 10 mg/mL streptomycin), 0.2% gentamicin sulfate, and 0.2% antifungal (250 μg/mL amphotericin B) and cells were allowed to migrate out of bulk tissue onto tissue culture dishes for 14 days at 37 °C and 5% CO₂, achieving 70% confluency. To obtain a homogenous fibroblast cell population, bulk tissue was re-plated onto new tissue culture dishes to initiate a second migration, and subsequently expanded up to passage 2 for seeding all subsequent studies.

Prior to cell culture, synthetic (PLGA:PCL) and purified microbial cellulose scaffolds were prepared into 7 mm diameter sections and sterilized by ultraviolet radiation (10 minutes/side). All scaffolds were incubated in F/S DMEM for 24 hours at 37 °C to encourage cell attachment. ACL fibroblasts were seeded onto scaffolds at a density of 30,000 cells/cm² and allowed to attach for 20 minutes at 37 °C and 5% CO₂. Subsequently, all cell-seeded scaffolds were incubated with F/S DMEM and cultured at 37 °C and 5% CO₂ for 14 days, with media replenished three times weekly.

Evaluating mCellulose Biocompatibility with Cell Response

Macrophage and fibroblast cell viability (n=2) was determined by a Live/Dead Viability Cytotoxicity Assay Kit (Molecular Probes, Eugene, OR, USA), following the manufacturer's protocol. At each timepoint, scaffolds were rinsed once with PBS and stained with Live/Dead reagent for 11 minutes at 37 °C and 5% CO₂ in the dark. Samples were further rinsed in PBS and immediately imaged using confocal microscopy (Olympus Fluoview FV1000) at 488 nm (excitation) and 515 nm (emission) wavelengths to assess cell viability and death, respectively.

Cell proliferation (n=5) was assessed by measuring the total DNA content of lysed, ultrasonicated, and papain-digested cellular scaffolds using Quanti-iT™ PicoGreen® dsDNA assay kit (Molecular Probes). Briefly, at each timepoint, scaffolds were rinsed once with PBS and stored in 500 μL 0.1% v/v Triton X (Sigma-Aldrich) at −20 °C. Before analysis, samples were thawed to room temperature, homogenized by vortexing, further ultrasonicated with a sonicating probe (Microson Ultrasonic Cell Disruptor XL-2000, Farmingdale, NY) at 5 watts for 15 seconds to lyse cells. All samples were desiccated and dehydrated for 12 hours in a CentriVap Concentrator until completely dry, and digested for 18 hours at 65 °C with papain (8.3 activity units/mL, Sigma-Aldrich) in 0.1 M sodium acetate (Sigma-Aldrich), 10 mM cysteine-HCl (Sigma-Aldrich), and 50 mM ethylenediaminetetraacetate (Sigma-Aldrich). After digestion, total DNA content was measured using a florescence microplate reader (SpectraFluor Plus, Tecan, Research Triangle Park, NC, USA) with an excitation wavelength of 485 nm and an emission wavelength of 535 nm. Measured florescence intensity of each sample was correlated to a DNA standard curve, followed by using a conversion factor of 7.7 pg DNA/cell (Kim et al., 1988) to determine the cell number. Non-cellular purified mCellulose scaffolds, subjected to one freeze/thaw cycle, lysis, ultrasonication, and papain-digestion were used as controls to aid in background subtraction from cellular scaffolds.

### Cell Morphology Analysis

Cell morphology on the surface of unaligned and aligned meshes was evaluated by SEM to support the determination of macrophage polarization state. After two days of cell culture, meshes (n=6) were rinsed once with PBS, fixed in 10% neutral buffered formalin supplemented with 1% CPC for 30 minutes, and washed once with deionized water. Following fixation, samples were dehydrated in series of ethanol washes at room temperature. Briefly, samples were immersed twice into 50% EtOH, followed by incubation in 60%, 70%, 90%, and 100% (twice) EtOH for 10 minutes each. After ethanol-based dehydration, samples were immersed twice into hexamethyldisilazane (HMDS) for ten minutes. Finally, HMDS was removed and samples were air dried for 24 hours in a chemical fume hood prior to imaging. All samples were sputter coated (Cressington 108 Auto, Cressington Scientific) with 10 nm of gold-palladium to increase surface conductivity and reduce charging effects, and imaged by scanning electron microscopy (SEM, 2 kV, Zeiss Sigma VP).

### LEGENDplex Analysis

The LEGENDplex Human Macrophage/Microglia 13-Plex Panel (Biolegend, San Diego, CA) was used to determine macrophage response (n=4) to synthetic (PLGA:PCL) and microbial cellulose scaffolds (as-fabricated, EtOH-treated, and purified). Following macrophage cell culture, culture media was immediately collected and frozen at −80 °C for evaluation of cytokine synthesis. Before analysis, samples were thawed to room temperature, and homogenized by vortexing, in which following plate arrangement, and reagent preparation were conducted using the manufacturer's protocol. Subsequent media analytes were measured for pro-inflammatory factors, including IL-12p70, TNF-α, IL-6, IL-1β, IL-12p40, IL-23, IFN-γ, and IP-10, as well as, anti-inflammatory markers, including IL-4, IL-10, IL-6, Arginase, TARC, and IL-IRA, using an Apollo ZE5 flow cytometer (Bio-Rad, Hercules, CA).

Sample analysis was performed using LEGENDplex Data Analysis Software Suite, in which flow cytometry data analysis was conducted following the manufacturer's protocol. All analytes that provided measurements above the limit of detection were quantitatively adjusted by normalizing per cell, from DNA content, for each scaffold group. Of note, analytes below the assay's limit of detection were not assessed. Resulting data was additionally z-score normalized to qualitatively present each analyte within cluster heat maps using Prism Software (GraphPad). Briefly, z-scores were measured by subtracting individual analyte values from the average result across all groups for that respective analyte, and then dividing by the grouped standard deviation. OriginPro 2022b (OriginLab, MA) was utilized in conjunction with the add-in feature “Heat Map with Dendrograms” to create a heat map including clustering analysis based upon using a Ward clustering method and Euclidean distance calculation. Finalized cluster heat maps were modified to include z-score normalized analytes within a low (white) to high (red) intensity scale.

### Reagents and Materials

All reagents and materials were supplied from Fisher Scientific, unless otherwise stated.

### Statistical Analysis

All results are reported as mean±standard deviation, with ‘n’ equal to the number of replicates per study group. Welch one-way analysis of variance (ANOVA) was performed to determine the effect of purification on bacterial and endotoxin content. The Brown-Forsythe post-hoc test was utilized for all pair-wise comparisons with significance attained at p<0.05. One-way ANOVA was performed to determine the effect of purification on bacterial cell viability (pixel overlap percentage), fiber diameter, tensile mechanical properties, sodium mass normalization (EDXA), macrophage cell number, and macrophage cytokine activity. Two-way ANOVA was utilized to determine the temporal effects of scaffold type on fibroblast cell number. The Tukey-Kramer post-hoc test was performed for all pair-wise comparisons with significance attained at p<0.05. All statistical analyses were performed by utilizing JMP-IN software (4.0.4, SAS Institute, Inc.) and Prism Software (GraphPad).

### Results

### Evaluating Microbial Cellulose Purification

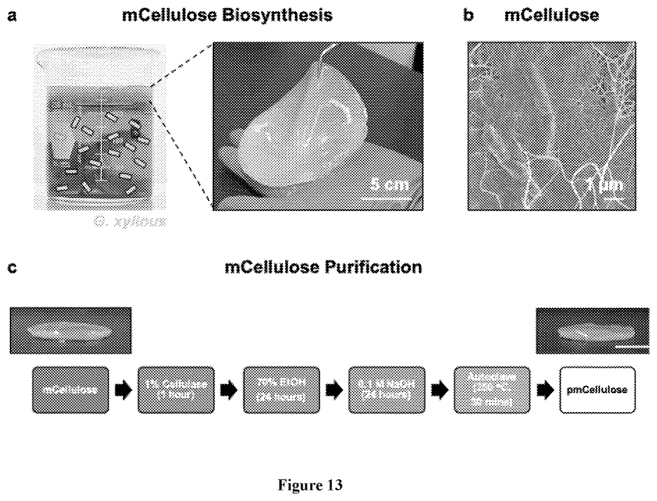

Microbial cellulose biofilms were subjected to a post-processing purification procedure, detailing sequential immersion into 1% v/v cellulase for 1 hour, 70% v/v ethanol for 24 hours, 0.1M NaOH for 24 hours, deionized water for 24 hours, and autoclaving (250 °C) for 20 minutes (FIG. 13).

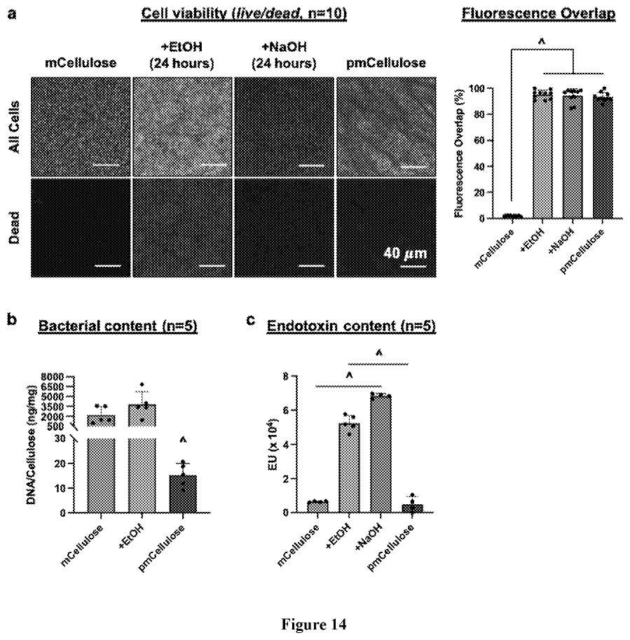

Bacterial cell viability in response to purification was analyzed by determining the percentage of overlapping pixels between all cells (intact and damaged membranes) and lysed cells (damaged membranes) within z stacked Live/Dead images (FIG. 14a). After the 14-day static culture period, G. xylinus cells were found to remain viable within the bulk of hydrated as-fabricated mCellulose. Quantitative comparison of overlapping fluorescence showed a significant increase in cell death once biofilms were immersed into ethanol (70% v/v) for 24 hours, measuring at +EtOH: 95.11±3.23% vs. mCellulose: 1.91±0.29%. Low cell viability was maintained after NaOH (0.1M) and autoclaving treatment. In addition, bacterial content in response to purification was assessed by determining the amount of DNA within as-fabricated and purified microbial cellulose biofilms. After three days of sterilization, pmCellulose was observed to significantly decrease in DNA per mg of biofilm (pmCellulose: 15.17±4.75 ng/mg vs. mCellulose: 2210.91±1256.99 ng/mg) (FIG. 14b).

Endotoxins were observed in all microbial cellulose biofilms throughout the purification procedure (FIG. 14c). While endotoxin content was initially low in as-fabricated mCellulose, immersion into ethanol (70% v/v), and further treatment with NaOH continually increased pyrogenic lipopolysaccharide release, with significant differences in each group (mCellulose: 0.64±0.03 (*10⁴) EU vs.+EtOH: 5.23±0.46 (*10⁴) EU vs.+NaOH: 6.83±0.14 (*10⁴)) EU. Completion of the purification procedure showed that endotoxin content was significantly decreased to 0.49±0.46 (*10⁴) EU after autoclaving treatment, with no differences with as-fabricated groups.

Purified mCellulose Characterization

Following purification, biofilms were characterized to determine if morphological, chemical, elemental, and tensile mechanical properties were maintained compared to as-fabricated mCellulose.

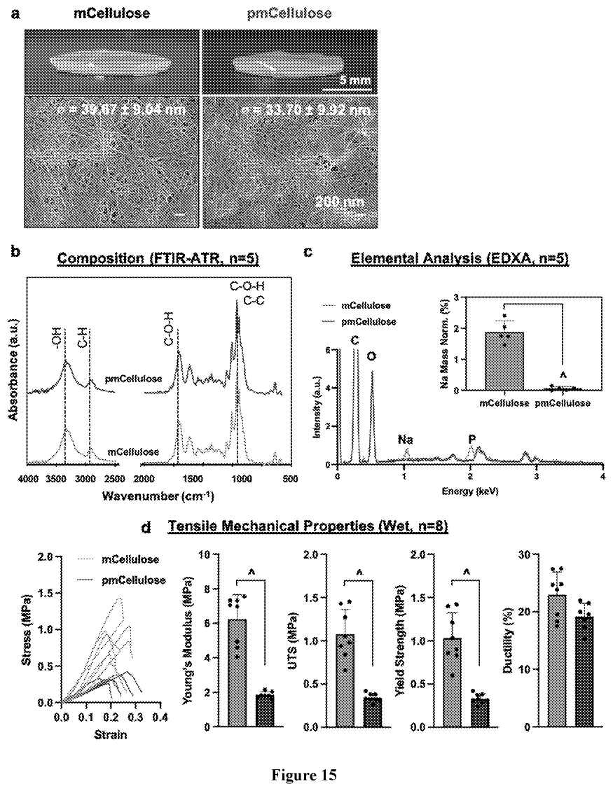

Initially, mCellulose macroscale structure (wet) and fibrous nanofiber (dry) morphology was assessed by representative photographs and SEM, respectively (FIG. 15a). As-fabricated microbial cellulose exhibited a characteristic brown color as a result of the Maillard reaction during culture (Kaminski et al., 2020), while purification revealed biofilms with a semi-transparent white color. Evaluation of mCellulose fiber morphology confirmed that as-fabricated groups had a densely packed fibrous surface with low porosity, which was representative to G. xylinus cultures prepared with mannitol. Further purification with chemical and steam sterilization yielded comparable fibrous structure with no significant differences in fiber diameter relative to as-fabricated (mCellulose: 39.67±9.04 nm; pmCellulose: 33.70±9.92 nm).

The maintenance of microbial cellulose chemical properties after purification was confirmed with FTIR-ATR (FIG. 15b). Characteristic as-fabricated mCellulose peaks were identified, including 3345 cm⁻¹ (O—H hydroxyl stretching), 2898 cm⁻¹ (C—H methyl stretching of CH₂ and CH₃), 1633 cm⁻¹ (—OH symmetrical stretching), and 1058 cm⁻¹ C—O—H/C—C/C—O—C pyranose ring stretching) (Schiros et al., 2022; Atykyan et al., 2020).

Additionally, elemental composition of mCellulose was evaluated by EDXA to determine the presence of sodium as a result of sodium hydroxide treatment (FIG. 15c). Characteristic peaks for carbon (C) and oxygen (O) were observed for as-fabricated and purified samples at k alpha values 0.27 keV and 0.52 keV, respectively. EDXA intensity profiles confirmed a minute sodium (1.88±0.36%) and phosphorus (3.44±0.602%) presence on mCellulose surfaces, however purification significantly decreased the relative mass percentage to negligible amounts at 0.064±0.060%, and 0.00±0.00%, respectively.

Lastly, wet mechanical properties of mCellulose were evaluated utilizing uniaxial tensile testing (FIG. 15d). It was observed that purified microbial cellulose had significantly lower Young's Modulus, ultimate tensile stress, and yield strength than as-fabricated groups, measuring 1.85±0.19 MPa, 0.34±0.05 MPa, and 0.33±0.06 MPa, respectively. In contrast, ductility was observed to be maintained around 20% before and after treatment.

Evaluating mCellulose Biocompatibility: THP-1 Macrophages

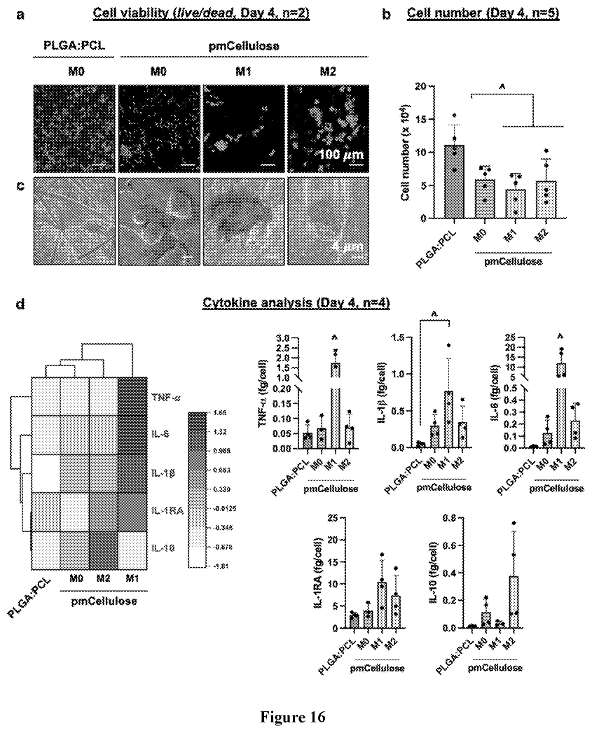

Human THP-1 monocytes were seeded, and differentiated into naïve macrophages on PLGA:PCL, as-fabricated (mCellulose), 70% v/v ethanol treated mCellulose (+EtOH), and purified microbial cellulose (pmCellulose) scaffolds to evaluate biocompatibility (FIG. 16). Here, synthetic PLGA:PCL scaffolds served as biocompatible controls. It was observed that cells remained viable on all microbial cellulose scaffolds, regardless of the presence of G. xylinus (FIG. 16a). On PLGA:PCL microfibers, a significantly greater number of cells were measured after 4 days of culture (FIG. 16b). While cell density was lower on purified mCellulose, significant differences were not observed between differentiated M0, and polarized M1, M2 groups.

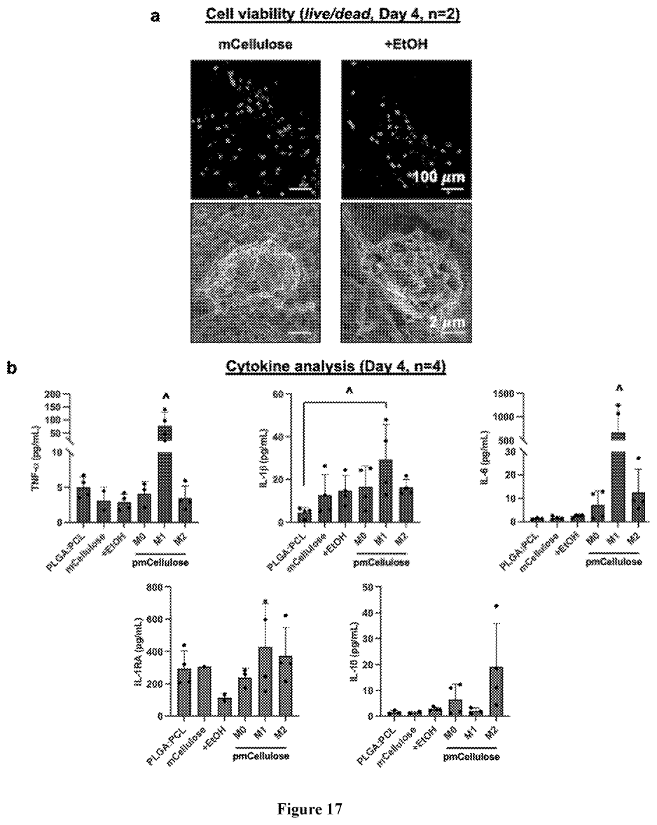

Macrophage cell morphology on synthetic PLGA:PCL microfibers and naturally-biofabricated mCellulose nanofibers was investigated by scanning electron microscopy (FIG. 16c). Cells were observed to maintain a round morphology with small protrusions attaching to PLGA:PCL, mCellulose, 70% v/v ethanol treated mCellulose, and pmCellulose fibrous scaffolds. Of note, macrophages cultured on as-fabricated microbial cellulose were covered by G. xylinus cells that produced the non-purified biofilm matrix (FIG. 17a). When induced to M1 or M2 phenotypes on pmCellulose scaffolds, cells became larger in size, and clustered with each other. However, M1 cells became elongated, while M2 morphologies remained round.

Pro- and anti-inflammatory cytokines secreted by macrophages into culture medium were assessed by the LEGENDplex Human Macrophage/Microglia 13-plex panel (FIG. 16d; FIG. 17b). Although the panel has the affinity to measure concentrations of pro-inflammatory proteins (IL-12p70, TNF-α, IL-6, IL-1β, IL-12p40, IL-23, IFN-γ, and IP-10), as well as, anti-inflammatory proteins (IL-4, IL-10, IL-6, arginase, TARC, and IL-IRA), only TNF-α, IL-1β, IL-6, IL-10, and IL-IRA were detected after two days of culture. In terms of total cytokine production, comparable concentrations (pg/mL) of TNF-α, IL-1β, IL-6, as well as, IL-10, and IL-IRA, were found from PLGA:PCL, mCellulose, 70% v/v ethanol treated mCellulose, and pmCellulose (FIG. 17b). Normalization to macrophage cell number only showed a significant increase in TNF-α, and IL-6 in pmCellulose groups polarized to M1 (FIG. 16d). However, polarization of THP-1 cells to M2 did not significantly affect the secretion of anti-inflammatory proteins (IL-10, and IL-IRA).

Evaluating mCellulose Biocompatibility: ACL Fibroblast Culture

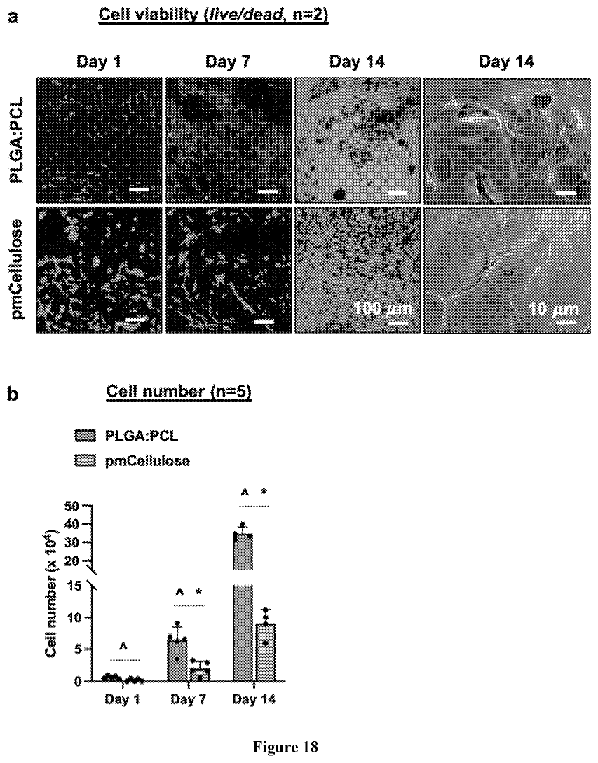

Bovine anterior cruciate ligament fibroblasts were isolated and cultured onto PLGA and purified microbial cellulose scaffolds for 14 days (FIG. 18). It was observed that cells remained viable and proliferated on both synthetic and natural platforms, in which significantly more cells were found on PLGA:PCL scaffolds at each time point. Examination of fibroblast cell morphology on PLGA:PCL microfibers and microbial cellulose nanofibers was investigated by scanning electron microscopy. Upon cell attachment, a random orientation of cells was observed when cultured on both unaligned groups, however, cells cultured upon pmCellulose became larger, and exhibited a different rounded morphology without protrusions. Throughout 14 days of culture, fibroblasts seeded on pmCellulose were observed to have spindled morphologies similar to cells on PLGA:PCL substrate.

### Example 5

The effects of chemical induction, microfiber matrix alignment and mechanical stimulation on macrophage response (adhesion and morphology), and polarization towards pro- and anti-inflammatory phenotypes were investigated. It is hypothesized that fiber alignment and tensile stimulation will have a combined effect on directing THP-1 macrophage response and inflammatory activation, with respect to initial cell adhesion, and subsequent release of pro- and anti-inflammatory cytokines. Specifically, the attachment, proliferation, morphology, and cytokine secretion of THP-1 macrophages were assessed on unaligned and aligned microfibers, with and without dynamic tensile load stimulation, over a two-day culturing period. The outcomes elucidated substrate and culturing parameters to modulate macrophage phenotype, and systematically determined if matrix alignment or mechanical stimulation dominantly determines cell response.

### Materials and Methods

### Mesh Fabrication

Blends of polylactide-co-glycolide (PLGA, 50:50, M_{w}≈38-54 kDa, Sigma-Aldrich) and polycaprolactone (PCL, Mₙ≈70-90 kDa, Sigma-Aldrich) (Mosher et al., 2021) were fabricated into meshes via a standardized electrospinning protocol (Moffat et al., 2009; Reneker, Chun, 1996). Briefly, PLGA and PCL were solubilized at a weight ratio of 5:1 in glacial acetic acid (Sigma-Aldrich), resulting in an 32% w/v polymer solution. The polymer melts were vortexed (Vortex Genie 2, Scientific Industries) for 24 hours, and loaded into a 5 mL syringe with a 18G stainless steel blunt-tip needle. To fabricate unaligned meshes, blended solutions were ejected from a syringe pump at 0.75 mL/hour towards a grounded stationary plate positioned 12 cm away, and then electrospun at 10 kV. Aligned fibers were formed by ejecting the polymer blend at 1 mL/hour towards a grounded rotating (2200 RPM) mandrel located 13 cm away from a needle subjected to 15 kV. Unaligned and aligned fibers were electrospun until 0.1-0.14 mm thick meshes were obtained.

### Mesh Characterization

Microfiber mesh morphology, and fiber diameter was assessed by performing scanning electron microscopy (SEM, 2 kV, Zeiss Sigma VP) on the surface of samples (Ø=10 mm) that were sputter coated with 10 nm of gold-palladium to reduce charging effects (Cressington 108 Auto, Cressington Scientific). Fiber diameter was quantified by analyzing randomly selected fiber segments in SEM micrographs (8,000X, n=50 fibers/group) using ImageJ software (National Institutes of Health).

Uniaxial tensile testing was conducted based upon the ASTM International standard tensile test method for plastics (D638-14). Scaffolds (5×1 cm, n=8) were secured with clamps and mounted in a uniaxial tensile testing machine (Instron) equipped with a 100 N load cell. All samples were maintained to have an average gauge length of 30 mm, and were tested to failure at a strain rate of 5 mm/minute. Scaffold elastic modulus, ultimate tensile strength, yield strength, and ductility were determined from the resulting stress-strain curve.

### Cells and Cell Culture

Human THP-1 monocytes were acquired commercially (ATCC, TIB-202) for in-vitro culture. Cells were maintained in suspension within non-tissue culture treated flasks (25 cm², Nunc™) and cultured with Roswell Park Memorial Institute-1640 (RPMI-1640) medium fully supplemented with 10% fetal bovine serum (FBS; Atlanta Biologicals, Norcross, GA, USA), 1% penicillin/streptomycin (P/S; 10,000 U/mL penicillin, 10 mg/mL streptomycin; Cellgro-Mediatech), and 0.05 mM 2-mercaptoethanol (2-ME; Sigma-Aldrich) at 37 °C and 5% CO₂. To prevent cell density from getting to high (1×10⁶ cells/mL), which has been reported to promote cell death and loss of differentiation potential, the culture medium was changed every three days by removing the cell suspension from flasks, and centrifuging at 500 rpm for 5 minutes. The supernatant was carefully aspirated without disturbing the cell pellet, in which the monocytes were resuspended in F/S RPMI-1640 at a density of 3×10⁵ cells/mL. Passage 5 THP-1 monocytes were used for all subsequent studies.

Prior to cell culture, the bioreactor apparatus was sterilized by autoclaving at 250 °C. (20 minutes) and subsequent ultraviolet radiation (10 minutes/side). Following, unaligned and aligned PLGA:PCL scaffolds (6×5 cm) were sterilized by ultraviolet radiation (10 minutes/side), and then assembled into the custom bioreactor cartridges. Scaffolds were incubated in RPMI supplemented with 10% FBS, and 1% P/S for 24 hours at 37 °C and 5% CO₂ to encourage cell attachment. THP-1 monocytes were seeded onto scaffold surfaces and differentiated into naïve macrophages utilizing the following protocol. Briefly, THP-1 monocytes grown to confluency in suspension culture were centrifuged at 500 rpm for 5 minutes to obtain a cell pellet. While the supernatant was discarded, cells were resuspended in F/S RPMI-1640. Monocytes were seeded onto scaffolds at a density of 200,000 cells/cm², allowed to attach for 20 minutes at 37 °C and 5% CO₂, and differentiated for 24 hours at 37 °C using F/S RPMI-1640 supplemented with 25 nM phorbol 12-myristate 13-acetate (PMA). After differentiation, all samples were rinsed twice with PBS to remove non-adherent cells. Following, cells were either allowed to rest (M0) with F/S RPMI-1640 or were subjected to pro- (M1) or anti- (M2) inflammatory polarization media for 24 hours. While pro-inflammatory polarization medium contained F/S RPMI supplemented with 20 ng/mL Interferon-γ (IFN-γ) and 100 ng/mL lipopolysaccharide (LPS), anti-inflammatory medium consisted of F/S RPMI+20 ng/mL Interleukin-4 (IL-4). After resting or polarization, media was aspirated, cells were washed with PBS once (in polarized groups only), and replenished with fresh F/S RPMI.

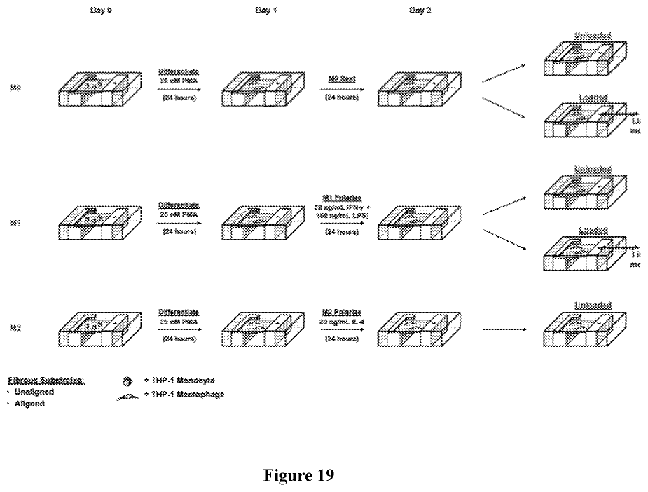

After the pre-culture period, meshes designated for mechanical stimulation were transferred into the bioreactor apparatus, in which a physiologically relevant loading regimen (De et al., 2000) consisting of 1% applied strain at 1 Hz was applied for 90 minutes twice daily (10.5 hours rest between cycles) (FIG. 19) (Subramony et al., 2014; Subramony et al., 2013). This loading regimen was implemented based upon previous work that reported optimal in-vitro tissue formation (Nirmalanandhan et al., 2008) occurred when physiologic strains were applied at a frequency of 1 Hz, as well as cell mechanosensitivity was maintained when a rest period was implemented between strain cycles (Riboh et al., 2008; Hanson et al., 2009). Meshes cultured in bioreactor cartridges without loading served as controls. All cell cultures on scaffolds were incubated at 37 °C and 5% CO₂ for the duration of the study. Cell viability, attachment, proliferation, morphology, and cytokine secretion were evaluated at day 0 and 2 of culture with and without mechanical stimulation. At each timepoint, samples were harvested by cutting the scaffold into 20 uniform segments (1×1 cm²/segment), and distributed for subsequent analyses.

### Evaluating Cell Response

Cell viability (n=2) on unaligned and aligned meshes was determined by a Live/Dead Viability Cytotoxicity Assay Kit (Molecular Probes, Eugene, OR, USA), following the manufacturer's protocol. At each timepoint, scaffolds were rinsed once with PBS and stained with Live/Dead reagent for 11 minutes at 37 °C and 5% CO₂ in the dark. Samples were further rinsed in PBS and immediately imaged using confocal microscopy (Olympus Fluoview FV1000) at 488 nm (excitation) and 515 nm (emission) wavelengths to assess cell viability and death, respectively.

Cell proliferation (n=5) was assessed by measuring the total DNA content of lysed, ultrasonicated, and papain-digested cellular scaffolds using Quanti-iT™ PicoGreen® dsDNA assay kit (Molecular Probes). Briefly, at each timepoint, scaffolds were rinsed once with PBS and stored in 500 μL 0.1% v/v Triton X (Sigma-Aldrich) at −20 °C. Before analysis, samples were thawed to room temperature, homogenized by vortexing, further ultrasonicated with a sonicating probe (Microson Ultrasonic Cell Disruptor XL-2000, Farmingdale, NY) at 5 watts for 15 seconds to lyse cells. All samples were desiccated and dehydrated for 12 hours in a CentriVap Concentrator until completely dry, and digested for 18 hours at 65 °C with papain (8.3 activity units/mL, Sigma-Aldrich) in 0.1 M sodium acetate (Sigma-Aldrich), 10 mM cysteine-HCl (Sigma-Aldrich), and 50 mM ethylenediaminetetraacetate (Sigma-Aldrich). After digestion, total DNA content was measured using a florescence microplate reader (SpectraFluor Plus, Tecan, Research Triangle Park, NC, USA) with an excitation wavelength of 485 nm and an emission wavelength of 535 nm. Measured florescence intensity of each sample was correlated to a DNA standard curve, followed by using a conversion factor of 7.7 pg DNA/cell (Kim et al., 1988) to determine the cell number.

Total collagen and proteoglycan matrix deposition (n=5) were assessed after sample lysates were papain-digested for 18 hours using a hydroxyproline assay and a modified 1,9-dimethylmethylene blue dye-binding assay, respectively.

Collagen production (n=5) was assessed by a hydroxyproline assay (Reddy, Enwemeka, 1996). Aliquots from each sample's digest (125 μL) were further dehydrated in Centrivap concentrator (Labconco) at 25 °C for 4 hours. Collagen was hydrolyzed with 25 μL of 2N sodium hydroxide (Sigma-Aldrich) and autoclaved at 250 °C for 25 minutes on a liquid cycle. The hydrolysate was oxidized by suspending in Chloramine-T reagent (225 μL; Sigma-Aldrich) for 25 minutes at room temperature. Ehrlich's reagent (250 μL), containing 15 w/v % p-dimethylaminobenzaldehyde in 2:1 v/v isopropanol/percholoric acid (Sigma-Aldric), was added to each sample and allowed to react for 20 minutes at 65 °C Lastly, samples were lightly vortexed and absorbance was measured at 555 nm on a microplate reader (Tecan SpectraFluor Plus). Absorbance measurements for each sample was correlated to a standard curve prepared from bovine collagen I solution (Biocolor, Carrickfergus, UK) to determine collagen content.

Glycosaminoglycan (GAG) production (n=5) was assessed by a modified 1,9-dimethylmethylene blue (DMMB) dye-binding assay (Enobakhare et al., 1996; Farndale et al., 1982). After digestion, lysates were allowed to return to room temperature prior to measuring proteoglycan content. Within 5 minutes, each sample was mixed with DMMB dye and absorbance was measured using a microplate reader (μQuant, Bio-Tek, Winooski, VT, USA) at a dual wavelength of 540 nm and 595 nm. The difference between the two absorbances was used to improve signal sensitivity. Absorbance measurements were correlated to a standard curve prepared from chondroitin-6-sulfate (Sigma-Aldrich) to determine proteoglycan content.

### Cell Morphology Analysis

Cell morphology on the surface of unaligned and aligned meshes was evaluated by SEM to support the determination of macrophage polarization state. After two days of cell culture, meshes (n=6) were rinsed once with PBS, fixed in 10% neutral buffered formalin supplemented with 1% CPC for 30 minutes, and washed once with deionized water. Following fixation, samples were dehydrated in series of ethanol washes at room temperature. Briefly, samples were immersed twice into 50% EtOH, followed by incubation in 60%, 70%, 90%, and 100% (twice) EtOH for 10 minutes each. After ethanol-based dehydration, samples were immersed twice into hexamethyldisilazane (HMDS) for ten minutes. Finally, HMDS was removed and samples were air dried for 24 hours in a chemical fume hood prior to imaging. All samples were sputter coated (Cressington 108 Auto, Cressington Scientific) with 10 nm of gold-palladium to increase surface conductivity and reduce charging effects, and imaged by scanning electron microscopy (SEM, 2 kV, Zeiss Sigma VP).

### LEGENDplex Analysis

The LEGENDplex Human Macrophage/Microglia 13-Plex Panel (Biolegend, San Diego, CA) was used to determine macrophage response (n=4) to mechanical loading, fiber alignment, and chemical stimulation. Following macrophage cell culture, culture media was immediately collected and frozen at −80 °C for evaluation of cytokine synthesis. Before analysis, samples were thawed to room temperature, and homogenized by vortexing, in which following plate arrangement, and reagent preparation were conducted using the manufacturer's protocol. Subsequent media analytes were measured for pro-inflammatory factors, including IL-12p70, TNF-α, IL-6, IL-1β, IL-12p40, IL-23, IFN-γ, and IP-10, as well as, anti-inflammatory markers, including IL-4, IL-10, IL-6, Arginase, TARC, and IL-IRA, using an Apollo ZE5 flow cytometer (Bio-Rad, Hercules, CA).

Sample analysis was performed using LEGENDplex Data Analysis Software Suite, in which flow cytometry data analysis was conducted following the manufacturer's protocol. All analytes that provided measurements above the limit of detection were quantitatively presented as concentrations (pg/mL or ng/mL). Of note, analytes below the assay's limit of detection were not assessed. Resulting data was additionally z-score normalized to qualitatively present each analyte within cluster heat maps using Prism Software (GraphPad). Briefly, z-scores were measured by subtracting individual analyte values from the average result across all groups for that respective analyte, and then dividing by the grouped standard deviation. OriginPro 2022b (OriginLab, MA) was utilized in conjunction with the add-in feature “Heat Map with Dendrograms” to create a heat map including clustering analysis based upon using a Ward clustering method and Euclidean distance calculation. Finalized cluster heat maps were modified to include z-score normalized analytes within a low (white) to high (red) intensity scale.

### Reagents

All reagents and materials were supplied from Fisher Scientific, unless otherwise stated.

### Statistical Analysis

All results are reported as mean±standard deviation, with ‘n’ equal to the number of replicates per study group. One-way analysis of variance (ANOVA) was performed to determine the effect of fiber alignment on fiber diameter and tensile mechanical properties, as well as, the effect of fiber alignment, mechanical loading, or chemical induction on cell number, population doubling, GAG and collagen deposition, and cytokine secretion. Two-way ANOVA was utilized to determine the temporal effects of fiber alignment, mechanical loading, or chemical stimulation on cell number, and cytokine secretion. The Tukey-Kramer post-hoc test was performed for all pair-wise comparisons with significance attained at p<0.05. All statistical analyses were performed by utilizing JMP-IN software (4.0.4, SAS Institute, Inc.) and Prism Software (GraphPad).

### Results

### PLGA:PCL Characterization

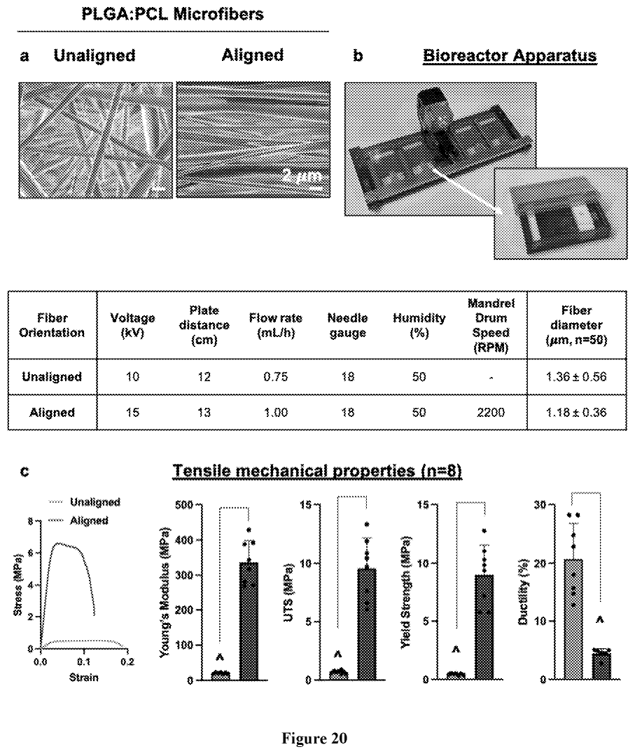

Synthetic PLGA:PCL microfibers were fabricated with aligned and unaligned orientations with green electrospinning methods that utilized acetic acid as a solvent. While electrospinning towards a stationary plate yielded unaligned fibers with a diameter of 1.36±0.56 μm, aligned fibers collected on a spinning mandrel resulted in a fiber diameter of 1.18±0.36 μm, in which no significant differences were observed in morphology or diameter (FIG. 20a). Tensile mechanical properties of electrospun meshes revealed that Young's Moduli (336.49±61.74 vs. 20.03±2.03 MPa), ultimate tensile strength (9.57±2.59 vs. 0.69±0.14 MPa), and yield strength (9.00±2.54 vs. 0.46±0.07 MPa) significantly increased with aligned orientation, while ductility was observed to significantly decrease (4.48±0.79 vs. 20.68±6.13 MPa) (FIG. 20c).

### Cell Attachment and Viability

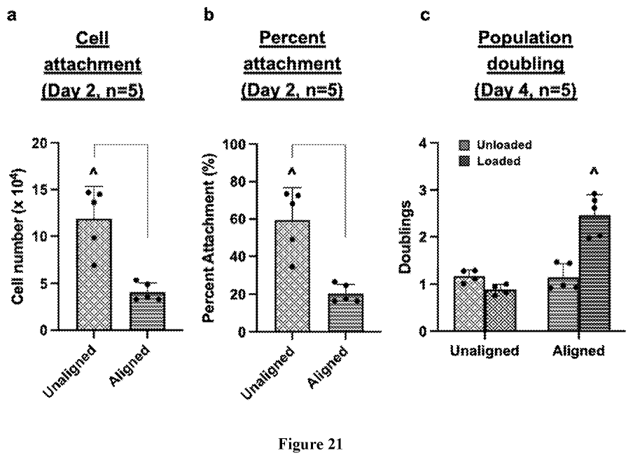

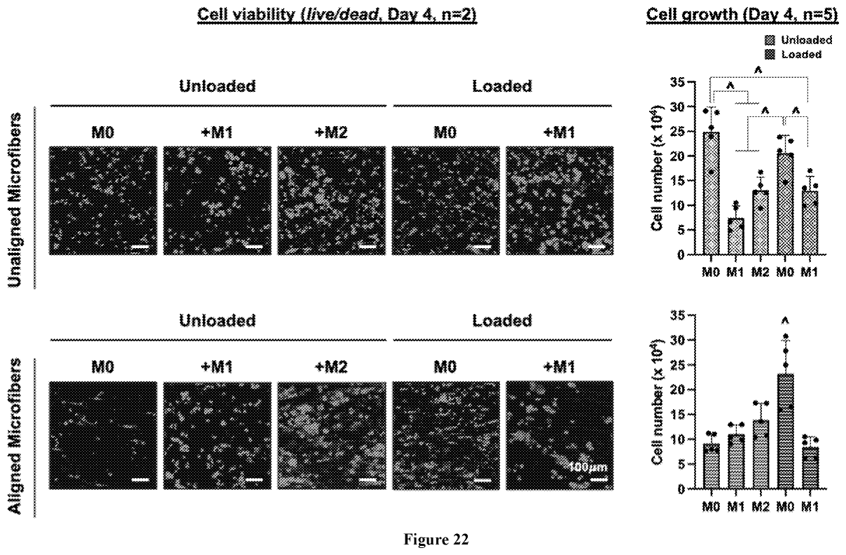

Human THP-1 monocytes were seeded and differentiated into naïve macrophages on aligned and unaligned PLGA:PCL microfibers in a custom designed loading bioreactor. Cell attachment was significantly lower on aligned fibers compared to unaligned substrates (FIG. 21a). Consequently, attachment efficiency was measured to be significantly lower on aligned meshes, measuring at a total of 20% cells adhering (FIG. 21b). Through four days of culture, it was observed that cells remained viable and proliferated on both unaligned and aligned microfibers (FIG. 22). On unaligned scaffolds, cell density was observed to be comparable for naïve macrophages after two days of loading. When inducted into pro- or anti-inflammatory phenotypes, cell number was found to be significantly lower compared to unloaded naïve macrophages counterparts. Further examination of loading pro-inflammatory induced cells on unaligned meshes showed no effect on cell number. In contrast, naïve macrophages significantly increased in cell number with more population doublings on loaded aligned microfibers (FIG. 21c; FIG. 22).

### Cell Morphology

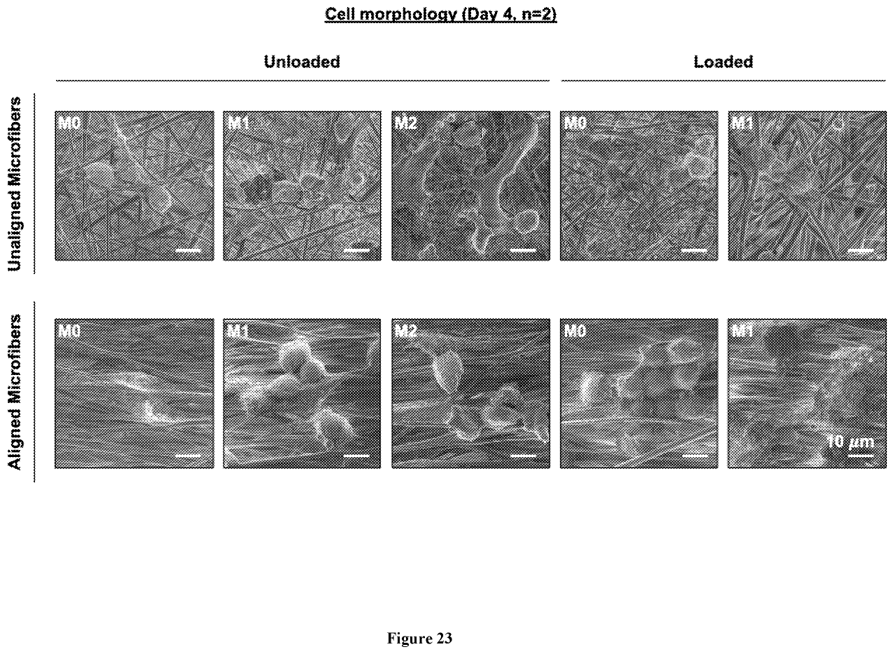

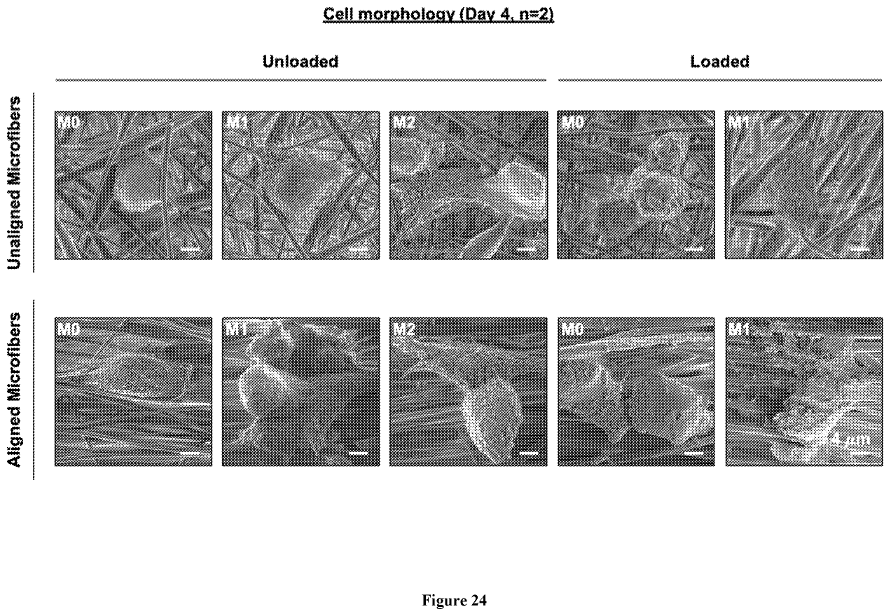

Macrophage cell morphology on unaligned and aligned PLGA:PCL microfibers was investigated by scanning electron microscopy (FIG. 23; FIG. 24). Naïve M0 macrophages were observed to maintain a rounded morphology with small protrusions attaching to unaligned PLGA:PCL, in which loading had no effect on cell shape. Upon culturing on aligned matrices, M0 macrophages elongated in the direction of the fibers, however, mechanical stimulation returned cells to a rounded morphology. In contrast, when induced to an M1 phenotype, cells became larger in size, and clustered with each other on unaligned and aligned scaffolds. While no differences were observed for M1 cells on unaligned matrices after mechanical loading, cells on loaded aligned scaffolds had distinctively different morphology with increased spreading and large presence of cellular debris potentially resembling matrix deposition. Further, when induced to an M2 phenotype, cells similarly became larger in size, and clustered with each other on both unloaded unaligned and aligned scaffolds. Of note, M2 cells were observed to either have round or spindle-like morphologies, in which cells elongated in the direction of the fibers on aligned scaffolds.

### Matrix Deposition

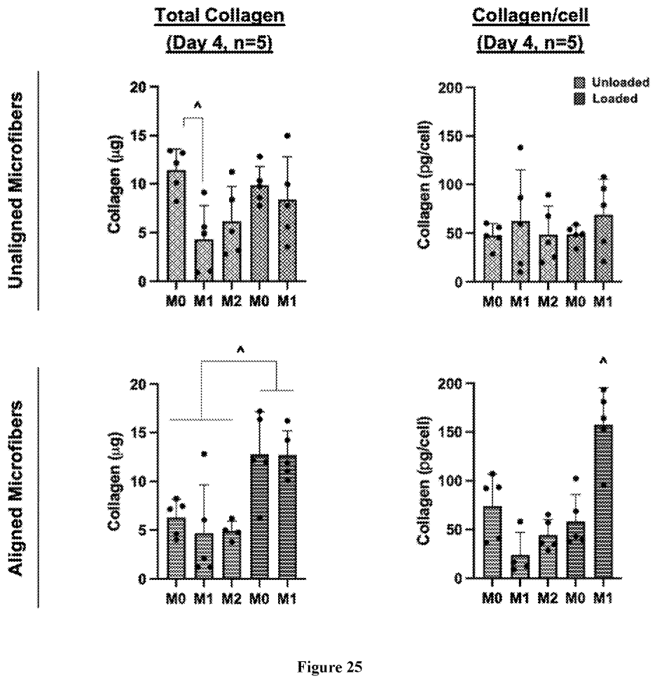

Collagen production from naïve and polarized cells was observed on aligned and unaligned meshes in both unloaded and loaded groups (FIG. 25). In terms of total collagen production, synthesis on unaligned unloaded scaffolds was significantly greater for naïve macrophages compared to pro-inflammatory induced cells, in which differences were not observed with loading. In contrast, a significant increase in collagen synthesis was measured after two days of loading M0 and M1-induced cells on aligned microfibers. Further normalization to cell number showed no differences between unloaded and loaded groups, as well as, between naïve and polarized cell groups on unaligned meshes, while loading M1-induced cells on aligned microfibers significantly produced the greatest amount of collagen.

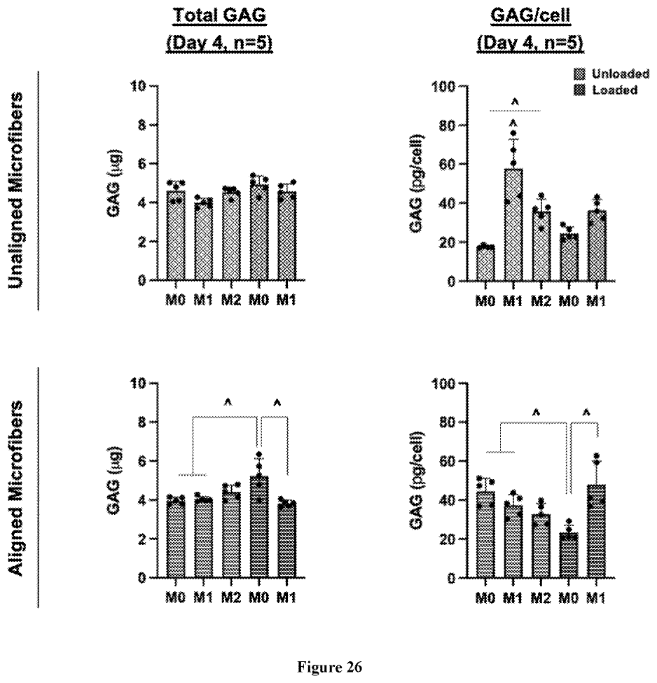

Proteoglycan production from näive and polarized cells was observed on aligned and unaligned meshes in both unloaded and loaded groups (FIG. 26). Total GAG production was similar for all groups on unaligned microfibers, however, on aligned meshes significantly greater production was measured after loading M0 cells compared to unloaded M0 and loaded M1 groups. Normalization to cell density showed that pro-inflammatory induced cells on unloaded unaligned meshes significantly produced the highest amount of GAG per cell. Moreover, mechanical loading of naïve cells on aligned microfibers had significantly lower GAG production per cell when compared to unloaded M0 and loaded M1 groups.

### Cytokine Secretion

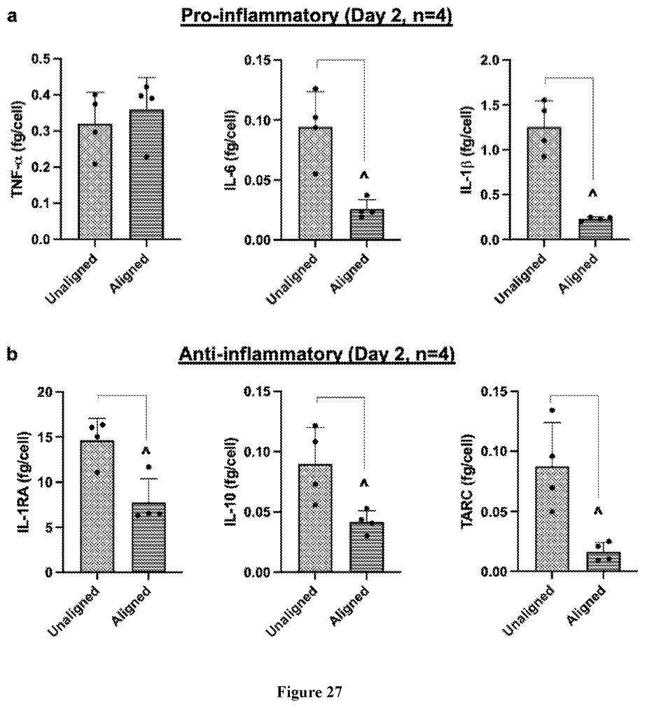

Pro- and anti-inflammatory cytokines secreted by macrophages into culture medium were assessed by the LEGENDplex Human Macrophage/Microglia 13-plex panel (FIG. 27-FIG. 31). Although the panel has the affinity to measure concentrations of pro-inflammatory proteins (IL-12p70, TNF-α, IL-6, IL-1β, IL-12p40, IL-23, IFN-γ, and IP-10), as well as, anti-inflammatory proteins (IL-4, IL-10, IL-6, arginase, TARC, and IL-1RA), only TNF-α, IL-1β, IL-6, IL-10, IL-1RA, and TARC were detected throughout four days of culture.

The effects of matrix alignment on macrophage response were investigated by measuring cytokine secretion from naïve cells seeded upon aligned and unaligned PLGA:PCL microfibers after completion of differentiation and polarization on Day 2 (FIG. 27). For unaligned PLGA:PCL groups, there was significantly more IL-1β, IL-6, IL-10, IL-1RA, and TARC per cell compared to aligned fibers. Of note, there were no observed differences in TNF-α per cell between unaligned and aligned meshes.

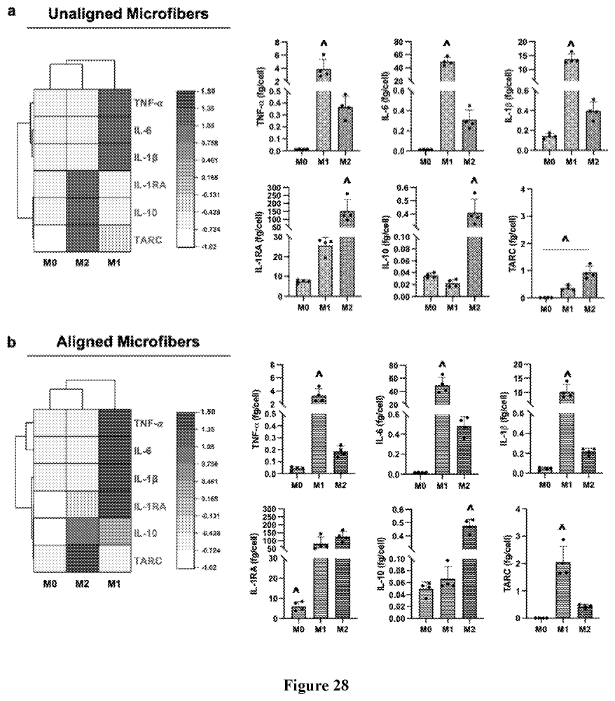

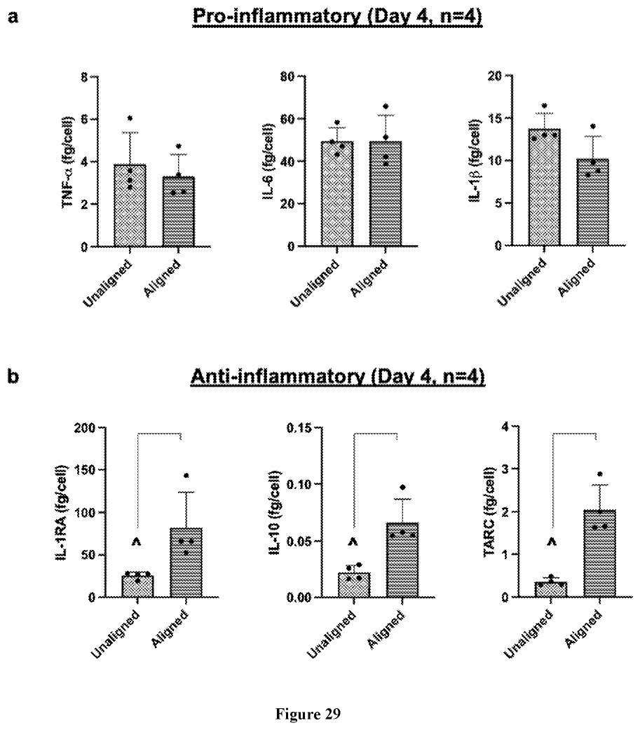

Further, the effects of polarization and matrix alignment on macrophage response were determined by measuring cytokine secretion from näive M0, and pro- (M1) and anti- (M2) inflammatory induced cells cultured upon aligned and unaligned PLGA:PCL microfibers for four days (FIG. 28). For macrophages on unaligned fibers, concentrations of TNF-α, IL-1β, and IL-6 per cell were significantly higher in the M1 polarized group, while IL-10 and IL-1RA per cell were significantly increased in the M2 polarized group. Levels of TARC per cell were observed to be significantly different between naïve M0, M1, and M2 groups, with elevated concentrations found in the latter. Similarly, on aligned meshes significant increases in TNF-α, IL-1β, and IL-6 per cell were measured only in the pro-inflammatory M1 group. As expected, induction to an anti- (M2) inflammatory state on aligned microfibers elevated IL-10 per cell concentrations. However, significantly more TARC per cell, an anti-inflammatory marker, was observed to be secreted from M1-induced cells on aligned PLGA:PCL. Interestingly, M1-induced macrophages seeded on aligned scaffolds significantly increased IL-10, IL-1RA, and TARC per-cell secretion of anti-inflammatory cytokines compared to unaligned groups (FIG. 29).

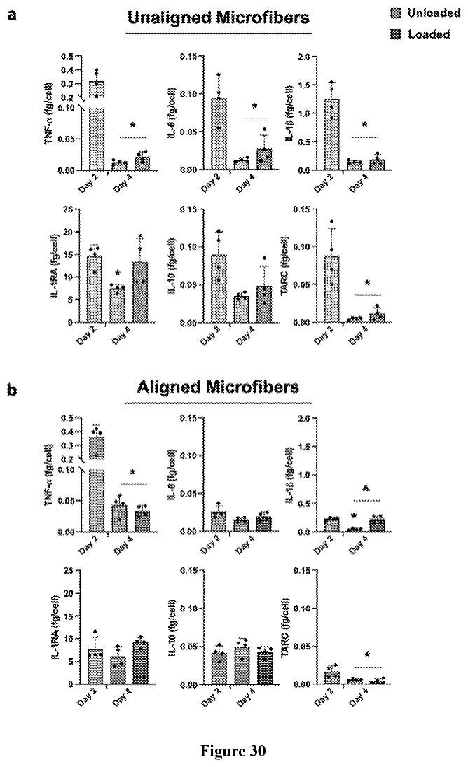

The effects of mechanical loading and matrix alignment on macrophage response were determined by measuring cytokine secretion from naïve M0 cells cultured upon aligned and unaligned PLGA:PCL microfibers in a custom designed loading bioreactor (FIG. 30). On unaligned meshes, a significant decrease in concentration for TNF-α, IL-1β, IL-6, and TARC per cell was measured by day 4, with no observed differences with loading. Similarly, a decrease in TNF-α and TARC per cell was found on aligned PLGA:PCL. Interestingly, levels of IL-10 per cell were significantly greater from loaded aligned meshes compared to unloaded groups, albeit comparable to day 2 concentrations.

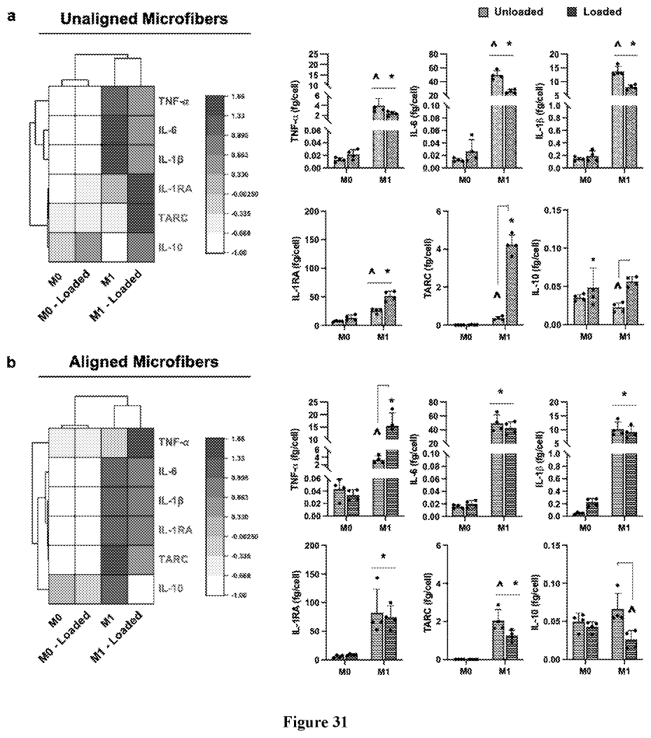

Finally, the combined effect of polarization, mechanical loading, and matrix alignment on macrophage response was determined by measuring cytokine secretion from naïve M0, and pro- (M1) inflammatory induced cells cultured upon aligned and unaligned PLGA:PCL microfibers in a custom designed loading bioreactor (FIG. 31). On unaligned PLGA:PCL meshes, significantly more TNF-α, IL-10, and IL-6 per cell was observed in M1 polarized groups compared to näive M0 cultures. There were no measured differences in the secretion of pro- (M1) and anti- (M2) inflammatory proteins between groups mechanically loading and unloading naïve M0 cells. In contrast, subsequent loading of M1 induced cells, not only significantly reduced the secretion of M1 markers TNF-α, IL-1β, and IL-6 per cell, but also significantly increased M2 secretion of IL-10, IL-1RA, and TARC per cell. For aligned microfibers, there were no observed differences in the secretion of M1 and M2 proteins between groups mechanically loading and unloading naïve M0 cells. Conversely, loading of M1 induced cells through two days of culture significantly increased pro-inflammatory TNF-α per cell, while decreasing anti-inflammatory IL-10 and TARC per cell.

### Discussion

The textile industry's linear model of production and reliance upon nonrenewable resources to manufacture synthetic fibers, dyes, and finishing agents make it one of the most polluting industries. Similarly, medical textiles fabricated in the tissue engineering field also utilize harsh solvents during development, not only limiting their biocompatibility and scalability, but also creating environmental concern in large, industrial volumes. Consequently, new fabrication strategies may assist to design functional biomaterials that support a sustainable and circular material economy.

Inspired by the complexity of nature and its robust regenerative potential, biofabrication has emerged as a strategy to produce biologically functional products with structural organization from living cells (eukaryotic or prokaryotic), hybrid tissue constructs, and/or biomaterials either through top-down (bioprinting) or bottom-up (bio-assembly) approaches. Motivated by a ‘green’ bio-economy, various cellular and biofabrication technologies (3D printing, and green electrospinning) provide significant opportunities for developing regenerative, non-toxic biomaterials with a closed loop life cycle. To address the challenge of developing sustainable biomaterials and tissue engineering textiles, it may be desirable to focus on harnessing microbial biosynthesis of nanocellulose for the development of regenerative, high performance biotextiles. Specifically, the design of microbial cellulose performance biotextiles and a tissue engineering scaffold will initially be achieved by static culture of Acetobacter xylinum bacteria that secrete cellulose nanofibrmis, coagulating into a three-dimensional layered hydrogel. Furthermore, it was desirable to investigate and optimize design parameters to tailor performance properties of mCellulose for: 1) regenerative, multi-functional biotextiles; and 2) a biocompatible scaffold for supporting in-vitro eukaryotic cell viability and basal-inflammatory response. These approaches are innovative in that they take advantage of utilizing globally found microbes to biofabricate multifunctional materials with tailorable nano-, micro- and macro-scale properties. Thus, the impact extends beyond biotextile development and connective tissue repair, as it would establish biomaterial design criteria for electrical, optical, and magnetic nanomaterials, as well as for other soft tissue-to-bone interfaces, cartilaginous tissues, and complex tissue systems.

It was desirable to determine the influence of carbon source for biofabricating mCellulose that meet target material properties, with broad applicability for the production of both non-medical and medical biotextiles. Gluconacetobacter xylinus can fabricate nanofibrous cellulose biofilms under a wide range of culturing conditions, thus making a versatile platform for tailoring material and structural properties during biosynthesis. Further, the effects of monosaccharides, disaccharides, and sugar alcohols as carbon sources at varying concentrations on cellulose production, were determined. Resulting mCellulose biofilms were characterized based upon their yield, and nanofibrous morphology, as well as their structural and chemical composition.

Also, it was desirable to focus on developing and optimizing both bioplasticizing, and crosslinking methods via immersion procedures that utilize green chemicals such as glycerol and sorbitol, and tannic acid (TA), respectively. Resulting mCellulose was characterized based upon their mechanical properties, surface morphology, and chemical and elemental compositions. Bioplasticizing and crosslinking of mCellulose offers modulation and stabilized mechanical behavior, without chemically modifying the bulk material with toxic and negative climate impacts. Life cycle impact assessment for the manufacturing of each green processing method was investigated to compare human health, environmental, and global warming impacts.

Microbial cellulose is a highly crystalline biopolymer produced extracellularly by obligate aerobic bacteria, such as Gluconacetobacter xylinus. Under static conditions, these bacteria biosynthesize cellulose nanofibrils that self-assemble into a three-dimensional layered pellicle with high water content (>98%) at the air-media interface, not only allowing for protection against UV irradiation, high temperatures, and desiccation, but also promoting bacterial adhesion, and nutrient transport for survival. Although identical in chemical structure to plant-based cellulose, mCellulose is distinctly characterized by high degrees of polymerization and crystallinity that support excellent mechanical behavior, and the possibility to further tailor structural and chemical properties during biosynthesis. Specifically, the unaligned nanofibrous biofilm is a direct result of the microbe's metabolism of sugars, in which depending on the carbon source in the environment different biochemical and enzymatic pathways are promoted to synthesize the final polymer. Briefly, when glucose is within the microbe's environment it is metabolized into glucose-6-phosphate, glucose-1-phosphate, UDP-glucose, and polymerized into 1,4-β-glucan chains due to a series of enzymatic reactions. However, differences in carbon source bioavailability, molecular weights, and chemical structure altered cellulose production rates and structural characteristics. Thus, it is desirableto biofabricate mCellulose from various carbon sources and concentrations to evaluate both bacterial growth and tailored biomaterial biophysical properties. To do this, G. xylinus would be used as a model organism for mCellulose biosynthesis and the feasibility of utilizing monosaccharides, disaccharides, and sugar alcohols at varying concentrations for production would be assessed. Completion of this study would elucidate facile culturing methods, as a means of carbon source, for readily engineering mCellulose to meet target material properties, with broad applicability for the production of both medical or non-medical biotextiles.

The role of carbon source and concentration on modulating microbial cellulose production by G. xylinus was determined. Conventional Hestrin-Schramm medium utilizing 2% w/v glucose for microbial based cellulose synthesis resulted in limited modulation of bacterial carbon metabolism, thus impeding the mechanism of polymer yield, as well as structural, and chemical material properties. In particular, various carbon sources and concentrations were evaluated for promoting cellulose biofabrication, as both sugar metabolism and cellulose biosynthesis are associated through similar biochemical enzymatic pathways. Here, glucose, fructose, sucrose, mannitol, and xylitol at 2% and 8% w/v were utilized within HS medium to biofabricate microbial cellulose throughout 14 days of culture, in which bacterial cell response and biomaterial properties were assessed.

Initial bacterial cell response studies show that both 2% and 8% w/v concentrations of glucose not only have lower microbial lag and doubling time compared to other sugars, but also significantly decrease medium pH throughout the first 100 hours of culture. Glucose offers significant advantages to promote early cell proliferation, possibly as a result of the direct metabolic pathway into glycolysis and increased acidic environment from gluconic acid production. However, although G. xylinus is an acidophilic bacterium that can survive in pH ranges between 3.5 to 6.3, it has been shown that a low pH environment (<pH 4) has a significant effect on metabolic enzyme activity, reducing cell viability and growth during culture. Additionally, differences in doubling time between glucose and mannitol were observed to be minimized, without affecting pH, by increasing the latter's concentrations to promote cell growth, indicating that microbial cell response can be modulated by carbon source and concentration. G. xylinus cell division has been shown to directly control cellulose fibril formation. As cells proliferate their subsequent cellulose ribbons are continually produced, branching into new fibrils without breaking, thus, optimal culture conditions that enhance cell division, produce shorter fibril lengths between branching points, supporting denser biofilms. Collectively, although this data exhibits that various carbon sources support the growth of G. xylinus, significant differences measured at lower population doublings with glucose suggests that this monosaccharide may be beneficial to optimize cellulose synthesis.

Here, in terms of mCellulose yield, it was observed that mannitol at low concentrations significantly increased cellulose production throughout 14 days of culture. Cellulose production by Komagataeibacter has been shown to be enhanced in mannitol as a result of an up-regulation of the fructose-mannose metabolic pathway, a non-conventional chemical production pathway, caused by continuous cultivation in mannitol over 200 days. However, in previous work by Wang et al. investigating bacterial cellulose synthesis by Komagataeibacter from varying carbon sources, 2% fructose and mannitol were observed to support the highest yields in 14 days, rather than glucose and sucrose, albeit pre-cultures (24 hours) utilized glucose. Thus, given that mannitol was utilized in pre-culture, these results similarly suggest that G. xylinus adaptation mechanism optimizes specific mannitol-utilization pathways to maximize cellulose yield, but not bacterial growth. Additionally, when higher concentrations at 8% w/v were utilized, cellulose production only significantly increased in fructose, sucrose, and mannitol cultures. Thus, it is possible, that the effect of carbon source and concentration on cellulose synthesis is acetic acid bacterial species dependent, in which G. xylinus has a specific affinity to upregulate guanosine 5′-triphosphate 3′-diphosphate (pppGpp) genes, also known as a stringent response, responsible for cellular reprogramming to optimize resource allocation, when subjected to environmental shifts. Notably, it is likely that with additional environmental stresses such as a significant decrease in pH (<pH 4) in glucose-based media, mCellulose yield was hindered at high concentrations due to a lack of adaptation mechanisms to tune metabolic pathways. Collectively, these data demonstrate that various carbon sources simultaneously support G. xylinus growth and cellulose production at different rates, indicating it is desirable to further characterize the nanofibril mCellulose matrix.

Not surprisingly, differences observed in cellulose production are reflected in the tensile mechanical properties and surface porosity of mCellulose. Microbial cellulose has been reported to have outstanding young's moduli of 66 GPa elastic moduli, however, the relationship between associated mechanical properties to nanofibril production and arrangement, as a result of carbon source and concentration, has not been explored. Collectively, significantly greater tensile mechanical properties in 2% w/v groups with mannitol was supported by its greater cellulose production by day 14, as well as, its low surface porosity (5%), indicating that mannitol synthesized denser biofilms. However, differences in material properties and surface porosity between fructose, sucrose, and mannitol groups were minimized at higher sugar concentrations, although sucrose resulted in an 85% and 22% increase in cellulose production compared to fructose and mannitol, respectively. Additionally, differences in material properties were not found in xylitol groups between concentrations, supporting that reduced yields with high porosity can be associated with inferior mechanical behavior. This is similar to the results observed by Ishihara et al., who found that bacterial cellulose production efficiency was significantly reduced in seventeen strains of acetic acid bacteria when cultured with D-xylose.

Native cellulose I is a composite of two crystalline configurations comprised of cellulose I_{α} (triclinic) and Is (monoclinic), however, allomorph ratio within the nanofibril has been shown to be species-dependent, and essential for structural stability. Here, molecular level insights on chemical structure and its relation to varied mechanical properties as a result of carbon source and concentration were determined by FTIR and X-ray diffraction. Although different carbon sources and concentrations yielded significant differences in cellulose production and material properties, all FTIR spectra exhibited similar vibrational bands, with larger differences in peak intensities observed at higher concentrations.

Discriminant analysis of two d-spacings (d₁ and d₂), also known as Z-value, may be credited to differences in mCellulose structure. Cellulose synthesized from glucose, fructose, and sucrose were observed to be mainly rich in I_{α} (Z>0; bacterial-algal), thus supporting the findings of comparable crystallite sizes. It was found that the mass fraction of I_{α} decreased with increasing concentration of the prior sugars within the culture medium, corresponding to promoted crystallization of the I_{β} allomorph. This unique allomorph response to concentration may be attributed to greater amounts of excreted water-soluble polysaccharides adhering to fibril subunits that are extruded into medium during biosynthesis, which has been shown to interfere with the aggregation of adjacent microfibrils into cellulose ribbons. Specifically, the production of acetan and levan has been reported to be dependent upon the carbon substrate, resulting in the interruption of cellulose crystallization that not only reduce hydrogen bonding between microfibrils, but also favor smaller crystal and enhanced Is formation. Interestingly, the 2% w/v mannitol and 8% w/v xylitol groups, with the lowest crystal sizes, showed Z-values less than zero which indicate that the mCellulose produced belonged to the I_{β}-dominant type (cotton-ramie). However, a respective increase and decrease in concentration of the prior groups resulted in I_{α}-rich structures with greater crystal sizes. Thus, it is clearly found that the production of particular cellulose allomorphs is correlated with crystal size, such that cellulose I_{β} is dominantly synthesized as smaller crystallite sized nanofibrils, while I_{α} are larger crystallite sized fibers. To this end, the weaker interchain hydrogen bonding network between adjacent cellulose sheets characterized in 2% w/v mannitol, containing the predominate I_{β} allomorph, did not support its superior mechanical behavior.

Taken together, these results show that although glucose, fructose, sucrose, mannitol, and xylitol support G. xylinus growth with different doubling rates, the resulting biosynthesis of mCellulose is greatly affected. The production of nanofibril cellulose by microbes is a part of its carbon metabolism, in which direct pathways consist of the phosphorylation of monosaccharides to the central hexose phosphate metabolite, glucose-6-phosphate. Consequently, glucose and fructose are conventionally utilized as carbon sources, while only the prior sugar was observed to not only support lower lag and population doubling times, but also reduce pH in culture media, limiting cellulose yield at higher concentrations. Alternative carbon sources, such as disaccharides and polyalcohols, were further metabolized within the pentose cycle and gluconeogenic pathway prior to cellulose biosynthesis, however, mannitol and sucrose enhanced 14-day production at 2% and 8% w/v, respectively. Thus, optimal yields achieved with non-conventional carbon sources potentially suggests an adaptation mechanism, evolving target metabolic pathways to enhance biopolymer production. A significant challenge in mCellulose biofabrication is the evaluation of nanofibril properties based upon G. xylinus' metabolic network. All mCellulose biofabricated from different sugars exhibited similar nanofibrous morphology and chemistry, however, surface porosity, crystal structure, and mechanical testing confirmed the modification of material properties during biosynthesis. Though differences in yield between carbon sources was minimized at higher sugar concentrations, the overall increase in biosynthesis led to a significant increase in Young's Modulus and ultimate tensile strength. Moreover, modification of carbon source and concentration in culture medium can be used to synthesize specific dominance of cellulose allomorphs dependent upon crystal size, albeit not reflected in tensile mechanical properties.

A life cycle impact assessment was performed using databases within EcoInvent to evaluate the human toxicity and ecological damage impacts of manufacturing carbon sources (glucose, fructose, sucrose, mannitol, and xylitol). Notably, impacts from each sugar have low magnitudes due to benchtop biofabrication, however, these data provide insights for green biofabrication at scale. Upon normalizing to the respective amount of cellulose produced after 14 days, it is shown that sucrose offers significant improvement in reducing human health and environmental toxicity compared to all other sugars for 2% w/v cultures, especially xylitol. This reduction may be in part to the sourcing of sucrose from sugar cane at a sugar refinery, thus eliminating large contributions of carcinogens (50.9%) and ecotoxicity (19.9%) observed when extracting xylitol via biomass hydrolysis. However, hormonally active agents were found to be the leading category for sucrose, accounting for 41.9% of total impact. Although sucrose resulted in more than ten times reduction in total impacts compared to xylitol (0.77×10⁻⁵ vs 1.41×10⁻⁴ mPts), both carbon sources, at 2%, produced comparable yields of microbial cellulose throughout 14 days of culture. Thus, xylitol's low cellulose production efficiency accompanied with the highest human health and environmental toxicity suggests that it is not suitable for green biofabrication of microbial biotextiles with improved circularity and scalability. Interestingly, although mannitol and glucose yield the highest cellulose in 14 days, glucose offers significant improvement to reduce total impacts by 71% (1.5×10⁻⁵ vs 5.2×10⁻⁵ mPts), while mannitol enhances biomaterial mechanical properties. Thus, the ability to not only tune yield, but also performance properties by biofabricating with carbon sources that have varying human health and environmental impacts, incentivizes the transition to a circular materials economy, offering a versatile platform for textile-based applications.

Various carbon sources and concentrations for G. xylinus culture medium were identified to biofabricate mCellulose. Although glucose, fructose, sucrose, mannitol, and xylitol supported microbial growth, differences observed in subsequent cellulose production, surface porosity, crystal structure, and mechanical behavior confirmed the modification of material properties during biosynthesis. Taken together, these results indicate that mCellulose performance properties can be tailored during culture, thus supporting the development of a versatile platform for non-medical and medical textile-based applications.

It was desirable to explore green bioprocessing methodology for stabilizing mCellulose material properties. Currently, the cattle industry is the leading driver of deforestation, in which chrome tanning of leather produces large scale carcinogenic, mutagenic, and teratogenic pollution. Thus, biofabrication strategies that develop multifunctional biotextiles with a circular life cycle that meet key design criteria such as, low toxicity, rapid renewability, scalability, and degradability would be helpful. Although microbial cellulose offers a biodegradable, natural approach, the biopolymer is limited to translation to textile applications due to its hygroscopicity resulting in instable mechanics. Accordingly, it is desirable to investigate a supplementary green chemistry approach inspired by traditional tanning that utilizes lecithin as a bio-phosphorylation treatment. This approach may not only improve mechanical reproducibility, but also enhance tensile strength and ductility, as a result of chemically modulating cellulose crosslinking. Ultimately, this invention indicates that this mCellulose approach coupled with green bioprocessing demonstrates significant potential within a circular materials economy.

The linear economy that has been the dominant production model in the textile industry since the Industrial Revolution not only threatens ecological and human health, but also drives climate instability. Specifically, the textile industry's reliance upon industrial agriculture for cellulosic fibers, as well as, nonrenewable petrochemicals to produce synthetic fibers, dyes, tanning and finishing agents, makes it ecologically one of the most polluting industries, and biggest global contributors to climate change. These chemically and energy intensive processes account for 1.2 billion tons of CO₂ emissions per year, 20% of global waste water, and 35% of primary microplastics in marine environments. Of note, the cattle industry that produces dairy, beef, and leather goods is the leading source of deforestation, while processing leather treatment via chrome tanning produces large scale carcinogenic, mutagenic, and teratogenic pollution. Specifically, tannery effluents used to convert raw hide into leather are ranked as the highest pollutants among all industrial wastes, due to the large volume of highly chemically intensive colored chemicals that contain various organic and inorganic compounds (sodium chlorides and sulphates), toxic metallic compounds, and putrefying suspended matter. In an attempt to mitigate use of animals for textiles and their environmental effects, leather alternatives have been fabricated from non-biodegradable petrochemical-based raw materials, finishing and coating agents. However, these synthetic alternatives contain endocrine-disrupting chemicals that absorb, accumulate, and distribute persistent organic pollutants through ecosystems, in which breakdown into nano- and micro-plastics have been recently discovered in marine and human tissues. Thus, scalable, biodegradable materials that support a circular economy, reducing toxicity (water and carbon footprints) throughout a product's life cycle are desirable.

Microbial cellulose is a highly crystalline and chemically pure extracellular polysaccharide secreted by gram-negative bacteria, such as Acetobacter xylinum. Under specific aerobic culturing conditions these bacteria biofabricate unaligned cellulose nanofibrils (10-100 nm diameter) that coagulate into a three-dimensional layered hydrogel (>98% water), known as a pellicle, at the air-culture media interface. Compared to plant cellulose, microbial cellulose has gained significant interest due to its inherent physiochemical properties, including high purity (free of hemicellulose and lignin), high tensile strength, high degree of polymerization, crystallinity (80-90%), high hygroscopicity and swelling capacity, moldability, biodegradability in composting environments, and biocompatibility. Owing to these unique properties, mCellulose presents a distinctive opportunity to achieve minimal-waste production through biosynthesis of cellulose that self-assemble into the shape of the growth vessel without impacting human health and ecotoxicity. Its use as a rapidly renewable raw material can eliminate the land use, water-intensive footprint and chemically and energy intensive processes observed during the production and purification of agricultural and wood pulp cellulose. In fact, the amount of nanocellulose processed from eucalyptus for 7 years grown on a 10,000 m² area of land is biofabricated at a higher purity by microbial fermentation in 22 days within a 500 m³ bioreactor without extensive chemical processing steps. mCellulose is distinctly characterized by high degrees of polymerization and crystallinity, and the possibility to further tailor structural and chemical properties during biosynthesis. However, textile application of this biodegradable, natural approach is limited by the significant hygroscopicity of as-fabricated mCellulose, resulting in unstable mechanics. Conventional strategies to promote textile mechanical stability include heavy metal and synthetic plasticizer treatments that introduce environmental toxicity, reduce biodegradability and biocompatibility, and enhance flammability. Thus, innovative green chemistry approaches were implemented to support a circular material economy, not only reducing toxicity throughout a biomaterial's life cycle, but also improving mechanical stability and functional performance.

Traditional tanning techniques, that include brain and organ tanning followed by smoke tanning, have been utilized as post processing strategies to produce resilient, water-repellent leather from animal hides. These non-toxic processes are hypothesized to be aldehyde-based tanning that covalently crosslink collagen fibrils within hides into leather. Briefly, aldehydes produced by smoke tanning react with collagen amine groups, resulting in imine functional groups. Further reaction with neighboring collagen amines promotes crosslinking between fibrils. Additional brain tanning introduces lecithin molecules, in which associated phosphate and choline functional groups stabilize the collagen crosslinking.

Thus, the effects of paleo-inspired green bioprocessing-based phosphatidylcholine and smoke tanning treatments were explored to stabilize microbial cellulose's mechanical properties without altering chemical composition and nanofibrous morphology. This would be achieved by separately determining the influence of a soybean-based lecithin phosphatidylcholine emulsions and aldehyde smoking on mCellulose, followed by a combined treatment. Given that these tanning processes successfully alter collagen structure, it is believed that they would similarly modify cellulose nanofibrils via crosslinking.

Ultimately, it is desirable to establish a green bioprocessing method for crosslinking mCellulose textiles. To this end, before and after tanning treatments, mCellulose will be evaluated to determine modifications in surface morphology, tensile properties, crystal structures, as well as in the local chemical bonding environments.

The linear economy that has been the dominant production model since the Industrial Revolution not only threatens ecological and human health, but also drives climate instability. Specifically, the textile industry's reliance upon industrial agriculture for cellulosic fibers, as well as, nonrenewable petrochemicals to produce synthetic fibers, dyes, tanning and finishing agents, makes it ecologically one of the most polluting industries, and biggest global contributors to climate change. Thus, scalable, biodegradable materials and post processing strategies that support a circular economy, reducing toxicity (water and carbon footprints) throughout a product's life cycle would be desirable.

Here, microbial cellulose was explored as a biomaterial approach to achieve minimal-waste production. Characteristic to many naturally occurring biopolymers, the hygroscopicity of as-fabricated microbial cellulose results in unstable mechanical properties that prevent translation to the textile industry. More specifically, this study focused upon establishing a green bioprocessing method for stabilizing microbial cellulose mechanics without introducing toxicity and negative climate impacts. Inspired by traditional brain (phosphatidylcholine) and smoke (aldehyde) tanning known to crosslink collagen fibrils into leather, soybean-based lecithin phosphatidylcholine was utilized to chemically crosslink the mCellulose nanofibril network. The effects of lecithin and aldehyde tanning, separately and sequentially, on as-fabricated mCellulose were evaluated, in which the resulting biomaterials' fiber morphology, hydrophilicity, tensile and thermal properties, as well as, chemical, molecular, and elemental compositions were analyzed. To this end, it was anticipated that phosphatidylcholine treatment enhanced and promoted reproducibility of microbial cellulose mechanics without introducing heavy coatings.

First, cellulose was biofabricated following a bottom-up approach of microbial biosynthesis. A symbiotic colony of bacteria and yeast metabolized sucrose into cellulose nanofibrils at the air-media interface under static conditions until a pellicle with a thickness of 2 cm was produced. Hydrated as-fabricated mCellulose was immersed into a lecithin emulsion at room temperature for 24-48 hours. Additionally, dried samples were exposed to hydrocarbon-rich smoke for 1 hour with the temperature ranging between 160-210 °F. Once treated, all samples were imaged by SEM to evaluate the surface and fiber morphology. It was observed that the nanofiber diameter and structure was unaffected by lecithin and aldehyde tanning, suggesting that the emulsion does not coat the fibers, but is rather removed with rinsing. However, these results were not supported by energy dispersive x-ray analysis, which was used to determine differences in surface elemental composition. Specifically, phosphorus concentration was significantly four times higher for LT in comparison to as-fabricated samples (2.02% vs. 0.57%, \hat{ }p<0.05), indicating interaction between lecithin's phosphatidylcholine groups and cellulose fibrils. A similar trend was observed after determining the degree of swelling, such that lecithin significantly increased mCellulose swelling, suggesting an enhanced hydrophilic network and hydrogen bonding environment that may improve mechanics.

Following hydrophilicity analysis, tensile properties of mCellulose, before and after treatments, were evaluated. As expected, as-fabricated mechanical properties, including Young's Modulus, and tensile strength, varied between two stress strain profiles, likely associated to the biopolymer's hygroscopicity. As a result, pristine mCellulose either exhibited high tensile strength with brittleness (MC 1: 17.4±4.6 MPa) or low tensile strength with greater ductility (MC 2: 12.0±5.8 MPa). Variability within mechanical properties was maintained in aldehyde tanned mCellulose. On the other hand, lecithin tanning stabilized and significantly increased elastic modulus, toughness, and tensile strength, which is likely attributed to potential crosslinking between phosphatidylcholine groups and cellulose fibrils. Notably, the improved mechanical properties were not represented by enhanced bulk crystallinity. This unique structure-function relationship, entailing an increase in tensile strength and ductility without differences in microstructure, has been similarly observed for phosphorylated cellulose with low phosphorus concentrations.

In addition to mechanical properties, flame-retardance is a notable design criterion for performance textiles, but industrial flame-retardant chemicals are considered hazardous substances linked to autoimmune diseases, neurological and reproductive problems, birth defects, and cancer. Specifically, phosphorous compounds have been reported to reduce polymer flammability, thus, phosphorylation of microbial cellulose with LT, as a green treatment, was determined. When exposed to a direct 2054 oC flame, both MC and LT deflected the flame, without propagation, however, structural morphology was altered with char formation. Images of the postburn samples not only reveal that mCellulose retains microscale layered assembly, but also nanofibers preserve their shape and structural integrity, with evidence of intumescent behavior. Flame testing of as-fabricated MC samples of varying thicknesses revealed that the mass loss due to combustion decreased with increasing mCellulose thickness until 0.9 mm, at which point mass loss was minimal. The degree of mCellulose thickness, as a consequence of the nanoscale self-organization of fibers into a layered structure, has the ability to limit the availability of the requisite oxygen and heat needed to ignite the material and sustain a flame. Moreover, intumescent bubbles formed on top of the fibers, due to gas expansion of the intumescent carbon layer, act as a double effect of slowing down the release of combustible volatiles and reducing the heat transmission. As expected, flame testing of LT samples resulted in less mass loss due to combustion in comparison to MC samples of the same thicknesses. Specifically, the addition of phosphorus compounds, with lecithin tanning, has been reported to enhance the dehydration and carbonization reactions that form thermally stable charred structures, thus, acting as a barrier, reducing the heat transmission from the flame to the bulk, and the amount of cellulose degradation products that feed the flame. Collectively, this suggested that the inherent nano- and microscale layered assembly of mCellulose, production of intumescent bubble morphology, as well as, chemical modification with lecithin promoted non-toxic flame retardancy and stability. Furthermore, investigation of mCellulose thermal-oxidative decomposition through TGA supported flame testing analysis. Lecithin treated samples maintained a higher hydration state due to significantly greater amounts of moisture-based weight (8.5±0.3%) lost by 200 °C, which supported the observation of higher swelling ability (n=5, \hat{ }p<0.05). Additionally, LT showed increased polymer decomposition by 240 °C, and char deposition by 680 °C compared to as-fabricated mCellulose. Thus, this data further corroborated that lecithin treatment lowers the decomposition temperature of MC and redirects the combustion pathway from formation of levoglucosan towards formation of foaming char as an insulating oxygen barrier, resulting in the outstanding flame resistance.

Reviewing the IR spectra, differences between as-fabricated and LT mCellulose were observed. While the appearance of P═O (1207.4, 1230 and 1252 cm⁻¹) and —P—OH (898 cm⁻¹) resonances in LT may be attributed to the deposition of phosphatidylcholine molecules, the additional sharpening of hydroxyl at (3450-3000 cm⁻¹), methyl, and methylene vibrational bands confirmed a modified bond environment through crosslinking. Collectively, the data suggests that the lecithin-based phosphatidylcholine treatment modified mCellulose crosslinking through phosphate, exocyclic CH₂, and carboxyl groups at well-defined hydroxyl binding sites (FIG. 46b inset). Methylene groups may react with cellulose hydroxyl groups at the 6^{th} carbon position (FIG. 46b inset), which are reported to react ten times faster than binding with the other —OH site. Carboxyl groups detected in LT with XPS has been shown to form ester bonds with hydroxyl groups, establishing stronger hydrogen bonding compared to as-fabricated mCellulose. Further, the determination of lateral order index and total crystallinity index with FTIR showed a higher degree of overall order with greater crystallinity in LT, supporting the development of chemical crosslinking through methyl, phosphate, and carboxyl groups. In this context, greater crosslinking achieved enhanced elastic moduli between LT vs. MC (196.4 vs. 134.6 MPa). The stabilization and improvement of mechanical properties with LT may therefore be attributed to disruption of intrinsic hydrogen bonds between cellulose chains at reactive hydroxyl, methyl, and phosphate group sites, leading to an increased interaction between cellulose chains that effectively transfers and distributes applied stress. Additionally, the superior flame retardance promoted by incorporation of phosphates may also be enhanced by the addition of methyl groups with lecithin tanning, which can decrease the rate of combustion during pyrolysis.

Thus, these results determine that lecithin enzyme tanning changes the bonding environment of microbial cellulose biofilms, such that the addition of —OH and C—H chemical bonding increases elastic modulus, hydration states, and thermal properties. Furthermore, harnessing biofabrication with green bioprocessing inspired by ancient practices for the development of microbial nanocellulose biotextile demonstrated a platform embracing a circular materials economy.

Microbial cellulose biofabrication coupled with green bioprocessing inspired by traditional tanning practices was developed to engineer a high-performance biomaterial, that supports a circular materials economy. This approach investigated the effects of lecithin phosphatidylcholine and aldehyde tanning as post-fabrication treatments for as-fabricated mCellulose, known to be characterized by its instable mechanical properties. The resultant mCellulose biomaterial modified via bio-phosphorylation with lecithin yielded enhanced tensile strength and ductility, as a result of crosslinking through hydroxyl, phosphate, and methylene groups, increasing the interaction between cellulose chains. Additionally, phosphorylation redirected the combustion pathway from levoglucosan production towards the formation of char as an insulating oxygen barrier, promoting flame retardance. Ultimately, this demonstrates that lecithin-based green chemistry can stabilize mCellulose mechanics, further elucidating the potential of this biofabrication platform to support a circular materials economy.

Effect of Carbon Source on G. xylinus Growth

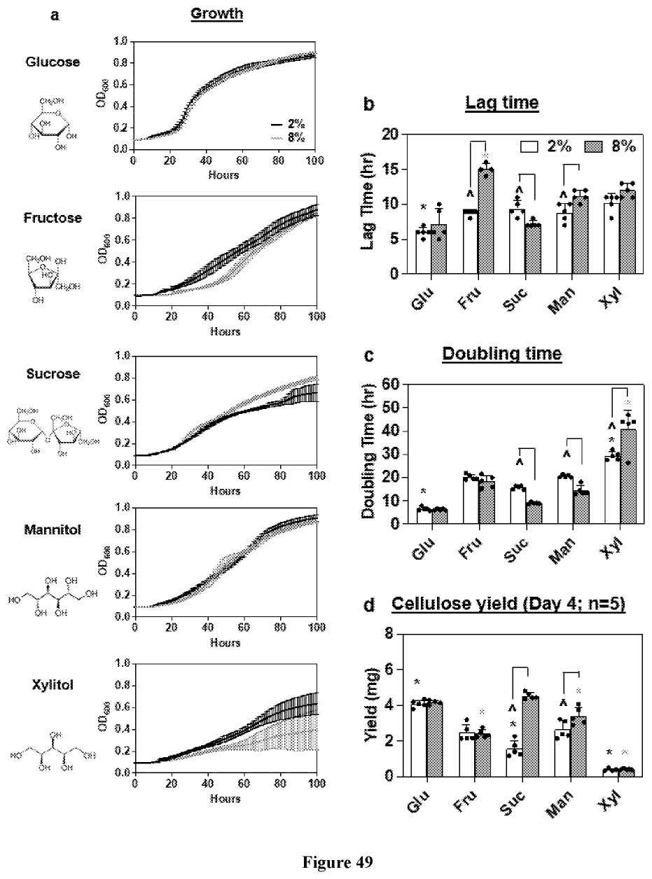

G. xylinus' growth throughout its lag, log, stationary, and death phases are directly affected by the available carbon source in the fermentation media. To investigate the effects of carbon source and concentration on microbial growth over time, the turbidity of culture medium incorporating either glucose, fructose, sucrose, mannitol, or xylitol at 2% and 8% w/v was measured (FIG. 49a). While viability was maintained with all carbon sources for over 100 hours, significantly lower lag times were measured at 6.00±0.70 and 7.20±2.16 hours in 2% and 8% w/v glucose-based cultures, respectively, when compared to other sugars (n=5, FIG. 49b). Surprisingly, differences in lag time between concentrations were only observed in fructose-based cultures (n=5, \hat{ }p<0.05). Additionally, microbial population doubling reduced to 6 hours during the exponential phase when supplemented with glucose, indicative of greater proliferation (n=5, FIG. 49c). However, no significant differences were observed between low and high glucose concentrations. Utilization of sucrose and mannitol increased population doubling times, in which higher concentrations for each not only mitigated this effect, but also significantly increased mCellulose production by day 4 (FIG. 49d). Notably, xylitol-based cultures had the largest population doublings ranging from 29 to 44 hours in 2% and 8% w/v, respectively, with significantly reduced mCellulose production after four days.

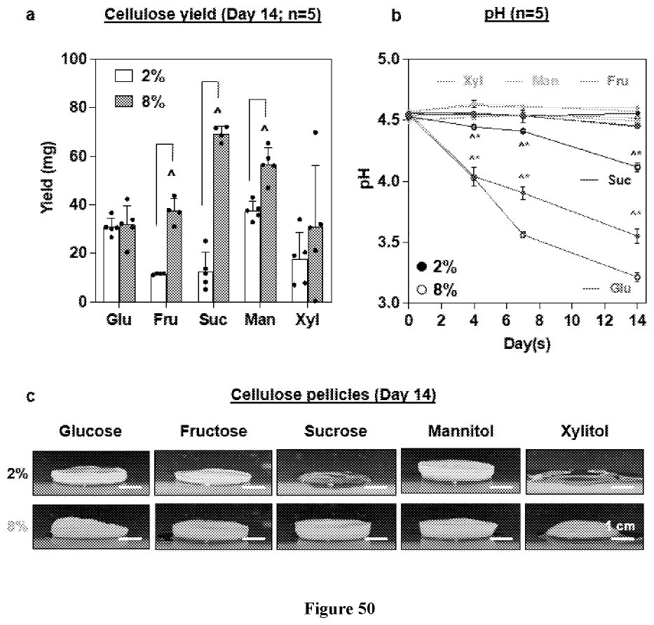

Furthermore, acidity measurements of the media revealed that pH decreased over time in glucose-based cultures, with a significantly greater acidic environment at higher concentrations by day 7 (n=5, FIG. 50b). The pH of 8% w/v sucrose-based medium similarly decreased by day 4 but to a lesser degree, likely due to the metabolism of the disaccharide into glucose monomers, however, differences were not observed at 2% w/v (n=5, \hat{ }p<0.05). The pH did not differ between all other groups (fructose, mannitol, and xylitol) and was maintained at the initial pH value measurement of 4.5. While microbial cell response indicates that both 2% and 8% w/v concentrations of glucose not only lowers microbial lag and doubling time compared to other sugars, it also significantly decreases medium pH throughout the first 100 hours of culture. Glucose offers significant advantages to promote early cell proliferation, possibly as a result of the direct metabolic pathway into glycolysis and increased acidic environment from gluconic acid production. However, although G. xylinus is an acidophilic bacterium that can survive in pH ranges between 3.5 to 6.3, it has been shown that a low pH environment (<pH 4) has a significant effect on metabolic enzyme activity, reducing cell viability and growth during culture. Alternatively, differences in doubling time between glucose and mannitol were minimized without affecting pH by increasing the latter's concentrations to promote cell growth, suggesting that microbial cell response can be modulated by carbon source and concentration.

The Effect of Carbon Source on mCellulose Bioproduction

Depending on the available carbon source in the culture medium, both G. xylinus' growth and mCellulose production can also be altered. The synthesized nanocellulose forms an envelope that surrounds microbes known to protect against UV irradiation, high temperatures, desiccation, and harsh environmental conditions, as well as promote bacterial adhesion, and nutrient transport for survival. The synthesis of mCellulose by G. xylinus is a part of its carbon metabolism, in which different initial pathways are utilized to synthesize glucose as the central metabolic product. Briefly, all carbon sources were metabolized throughout a four-phase enzymatic reaction into glucose-6-phosphate, glucose-1-phosphate, UDP-glucose, and polymerized into 1,4-β-glucan chains. Carbon substrates, such as arabinose, arabitol, fructose, galactose, lactose, malic acid, maltose, mannitol, mannose, and xylose, have been reported to be transformed into glucose through different biochemical and enzyme pathways, in which differences in their bioavailability and chemical structure will alter cellulose production rates and structural characteristics. Based on our previous research, human toxicity is the largest ecological impact for producing mCellulose due to the refinery processes for manufacturing sugars utilized in the fermentation media. Thus, the selection of the carbon source utilized during cultivation inform significant mCellulose production with low environmental impact processes.

To determine the effects of carbon source and concentration on cellulose production, the yield and material properties of mCellulose biofabricated using 2% and 8% w/v concentrations of glucose, fructose, sucrose, mannitol, and xylitol as media nutrients were measured. All sugars supported the biofabrication of hydrated mCellulose biofilms, at the air-media interface over 14 days, with cylindrical geometries and a light-brown color, as a result of the shape of the culturing vessel and a Maillard reaction during culture, respectively (FIG. 50c). Purification and lyophilization of mCellulose resulted in dehydrated, white biofilms. While synthesis was observed with all carbon sources, cellulose production significantly enhanced at a higher concentration (8% w/v) by day 14 when utilizing fructose, sucrose, and mannitol (n=5), with no differences seen in glucose and xylitol groups. In 2% w/v groups, mCellulose yield by day 14 was 30.98±3.61, 11.61±0.33, 12.78±7.62, 37.58±3.75, and 17.68±11.01 mg for glucose, fructose, sucrose, mannitol, and xylitol, respectively, resulting in significantly enhanced production with mannitol (n=5). Moreover, cellulose production via 2% w/v mannitol-based media significantly increased overtime (n=5). An increase in concentration to 8% w/v yielded 32.00±7.80, 37.47 5.17, 69.40±3.14, 56.82±6.64, and 30.84±25.23 mg in glucose, fructose, sucrose, mannitol, and xylitol groups, respectively, resulting in sucrose supporting greater production between groups (n=5), and over time by day 7 (n=5).

Here, in terms of mCellulose yield, it was observed that mannitol at low concentrations significantly increased cellulose production throughout 14 days of culture. Cellulose production by Komagataeibacter has been shown to be enhanced in mannitol as a result of an up-regulation of the fructose-mannose metabolic pathway, a non-conventional chemical production pathway, caused by continuous cultivation in mannitol over 200 days. However, in previous work by Wang et al. investigating bacterial cellulose synthesis by Komagataeibacter from varying carbon sources, 2% fructose and mannitol were observed to support the highest yields in 14 days, rather than glucose and sucrose, albeit pre-cultures (24 hours) utilized glucose. Thus, in this study, given that mannitol was utilized in pre-culture, this suggests that G. xylinus adaptation mechanism optimizes specific mannitol-utilization pathways to maximize cellulose yield, but not bacterial growth. Additionally, when higher concentrations at 8% w/v were utilized, cellulose production only significantly increased in fructose, sucrose, and mannitol cultures. This suggests that the effect of carbon source and concentration on cellulose synthesis is acetic acid bacterial species dependent, in which G. xylinus has a specific affinity to upregulate guanosine 5′-triphosphate 3′-diphosphate (pppGpp) genes, also known as a stringent response, responsible for cellular reprogramming to optimize resource allocation, when subjected to environmental shifts. Notably, it is likely that with additional environmental stresses such as a significant decrease in pH (<pH 4) in glucose-based media, mCellulose yield was hindered at high concentrations due to a lack of adaptation mechanisms to tune metabolic pathways. Collectively, a multitude of carbon sources simultaneously support G. xylinus growth and cellulose production at different rates, indicating it is notable to further characterize the nanofibril mCellulose matrix.

### Microbial Cellulose Nanofiber Characterization

Throughout the biofabrication of mCellulose, sugars are initially metabolized into cellulose nanofibrils, and subsequently assembled into a coagulated 3D layered hydrogel, offering unique advantages to tune and meet key design criteria by strategically selecting the carbon source during fermentation. The structural and chemical properties of mCellulose biofabricated from glucose, fructose, sucrose, mannitol, and xylitol-based medium for 14 days of culture were assessed to determine the effects of carbon source and concentration on nanofibril formation.

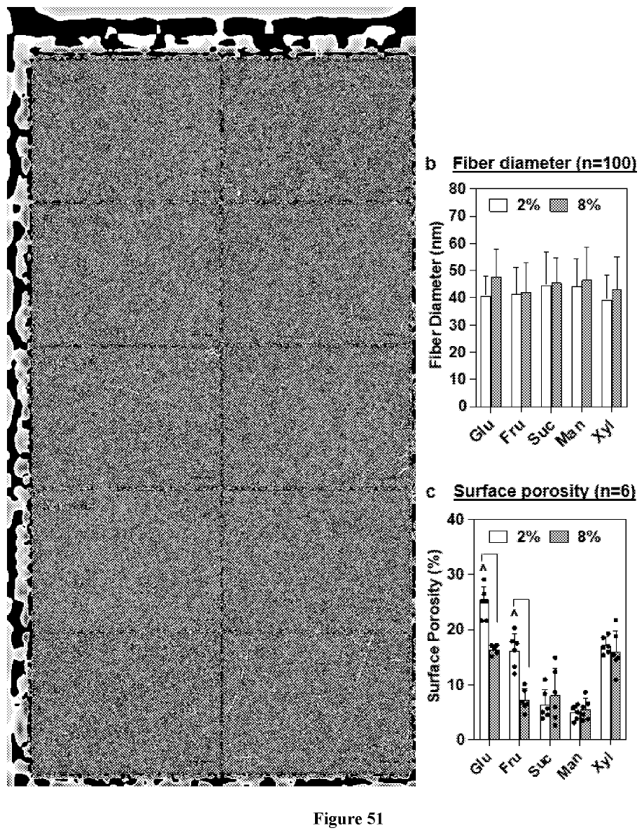

All carbon sources supported biofabrication of mCellulose biofilms with unaligned nanofibrous morphologies evident via scanning electron microscopy (FIG. 51a). The conventional 2% w/v glucose-based HS media was used to yield nanofibers with a diameter measuring 40.82±7.17 nm, with no significant difference in either fiber morphology or diameter when alternative monosaccharides, disaccharides, and poly-alcohols at 2% or 8% w/v were utilized (FIG. 51b). Of note, the surface porosity between individual nanofibers was significantly reduced in fructose (8% w/v), sucrose (2% and 8% w/v), and mannitol (2% and 8% w/v) groups, indicating mCellulose structure and fiber production was affected by carbon source (n=6, FIG. 51c).

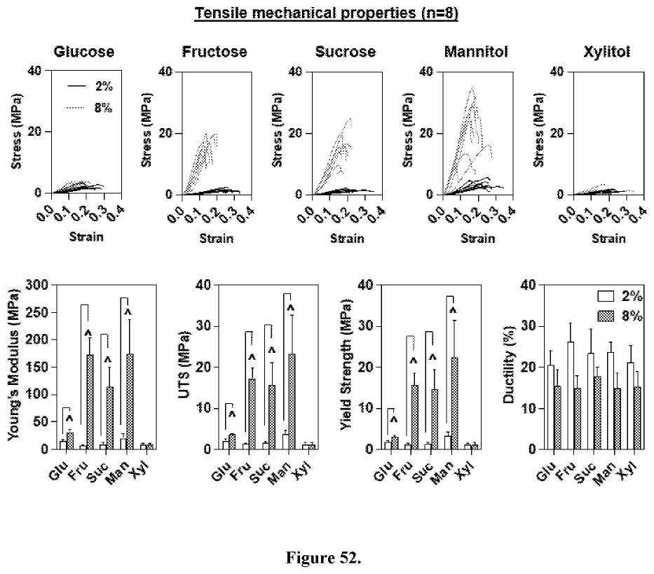

After morphological characterization, the mechanical properties were evaluated for dried mCellulose biofilms biofabricated from various carbon sources and concentrations (FIG. 52). While the ductility remained similar between carbon source and concentration (2% and 8% w/v), the ultimate tensile strength (Glucose: 2.08±0.40 vs. 3.61±0.26 MPa; Fructose: 1.33±0.21 vs. 17.26±2.49 MPa; Sucrose: 1.52±0.36 vs. 15.73±5.37 MPa; Mannitol: 3.63±1.07 vs. 23.34±9.40 MPa), Young's Modulus (Glucose: 14.52±2.75 vs. 31.21±4.53 MPa; Fructose: 7.10±1.04 vs. 173.69±29.53 MPa; Sucrose: 8.95±3.27 vs. 114.60±35.47 MPa; Mannitol: 20.32±7.66 vs. 174.67±62.68 MPa), and yield strength (Glucose: 1.76±0.39 vs. 3.10±0.32 MPa; Fructose: 1.15±0.25 vs. 15.79±2.77 MPa; Sucrose: 1.35±0.36 vs. 14.57±4.79 MPa; Mannitol: 3.28±0.89 vs. 22.39±9.01 MPa) significantly increased at 8% w/v (n=8), with the exception of xylitol.

Not surprisingly, differences observed in cellulose production are reflected in the tensile mechanical properties and surface porosity of mCellulose. Microbial cellulose has been reported to have outstanding young's moduli of 66 GPa elastic moduli, however, the relationship between associated mechanical properties to nanofibril production and arrangement, as a result of carbon source and concentration, has not been explored. Collectively, significantly greater tensile mechanical properties in 2% w/v groups with mannitol was supported by its greater cellulose production by day 14, as well as, its low surface porosity (5%), indicating that mannitol synthesized denser biofilms. However, differences in material properties and surface porosity between fructose, sucrose, and mannitol groups were minimized at higher sugar concentrations, although sucrose resulted in an 85% and 22% increase in cellulose production compared to fructose and mannitol, respectively. Additionally, differences in material properties were not found in xylitol groups between concentrations, supporting that reduced yields with high porosity can be associated with inferior mechanical behavior. This is similar to the results observed by Ishihara et al., who found that bacterial cellulose production efficiency was significantly reduced in seventeen strains of acetic acid bacteria when cultured with D-xylose.

Native cellulose I is a composite of two crystalline configurations comprised of cellulose I+(triclinic) and Iβ(monoclinic), however, allomorph ratio within the nanofibril has been shown to be species-dependent, and essential for structural stability. Here, molecular level insights on chemical structure and its relation to varied mechanical properties as a result of carbon source and concentration were determined by FTIR-ATR and X-ray diffraction.

The chemical composition of mCellulose biofabricated from various carbon sources was assessed using FTIR-ATR (FIG. 53a). Chemical fingerprints of cellulose I were confirmed via ATR (n=5), in which characteristic peaks for hydroxyl O—H stretching (3300 cm⁻¹), methyl C—H stretching (2900 cm⁻¹ 1, 1434 cm⁻¹), antisymmetric bridge C—O—H stretching (1161 cm⁻¹), C—O bending (1106 cm⁻¹, 1057 cm⁻¹), as well as, β—glucosidic linkages between monomer units (895 cm⁻¹) were observed, with larger differences in peak intensities observed at higher concentrations.

Surprisingly, differences in cellulose crystal structure were observed with X-ray diffraction analysis. While all fibers synthesized with varying carbon sources and concentrations exhibited cellulose I Bragg peaks at 2θ=14.6°, 16.9°, and 22.8°, corresponding to the (1-10), (110), and (200) planes (FIG. 53b), only the inferior mechanical properties of xylitol-based groups are reflected in bulk microstructure crystallinity measurements. A peak deconvolution method demonstrated comparable crystallinity indexes between glucose, fructose, sucrose, and mannitol groups (85%), along with a significant decrease to 70.97±7.87 and 74.01±7.19% in 2% and 8% w/v xylitol, respectively (n=5, FIG. 53c).

Further, crystallinity index values do not exhibit a correlation with corresponding crystallite sizes in glucose, fructose, and sucrose-based groups at either concentration (FIG. 53d). Although greater crystal sizes were observed in mCellulose synthesized from glucose and sucrose with an increase to 8% w/v, no significant differences were found. On the other hand, crystallite size of cellulose produced from mannitol was found to significantly increase with increasing concentration (5.20±0.25 vs. 5.85±0.03 nm, n=5), while xylitol was found to significantly decrease (5.71±0.33 vs. 5.23±0.24 nm, n=5).

Discriminant analysis of two d-spacings (d₁ and d₂), also known as Z-value, was measured to determine differences in mCellulose allomorph structure based upon biofabrication with various carbon sources (FIG. 53e). Cellulose synthesized from glucose, fructose, and sucrose were observed to be mainly rich in I+ (Z>0; bacterial-algal), thus supporting the findings of comparable crystallite sizes. It was found that the mass fraction of I+ decreased with increasing concentration of the prior sugars within the culture medium, corresponding to promoted crystallization of the Iβ allomorph. This unique allomorph response to concentration may be attributed to greater amounts of excreted water-soluble polysaccharides adhering to fibril subunits that are extruded into medium during biosynthesis, which has been shown to interfere with the aggregation of adjacent microfibrils into cellulose ribbons. Specifically, the production of acetan and levan has been reported to be dependent upon the carbon substrate, resulting in the interruption of cellulose crystallization that not only reduce hydrogen bonding between microfibrils, but also favor smaller crystal and enhanced Iβ formation. Interestingly, the 2% w/v mannitol and 8% w/v xylitol groups, with the lowest crystal sizes, showed Z-values less than zero which indicate that the mCellulose produced belonged to the Iβ-dominant type (cotton-ramie). However, a respective increase and decrease in concentration of the prior groups resulted in I+-rich structures with greater crystal sizes. Thus, the production of particular cellulose allomorphs is correlated with crystal size, such that cellulose Iβ is dominantly synthesized as smaller crystallite sized nanofibrils, while I+ are larger crystallite sized fibers. To this end, the weaker interchain hydrogen bonding network between adjacent cellulose sheets characterized in 2% w/v mannitol, containing the predominate Iβ allomorph, did not support its superior mechanical behavior.

### Evaluation of Polyol as Bioplasticizers

Bioplasticizers have been reported to alter polymer chain spacing, characteristically increasing ductility and decreasing strength of biomaterial systems compared to as-fabricated states. Plasticizer type and concentration can be used to modulate mechanical performance, in which greater concentrations promote anti-plasticization behavior. Here, mCellulose was successfully biofabricated (after 14 days of G. xylinus culture in 2% w/v mannitol at 30 °C) and processed with various concentrations ofpolyols, including glycerol and sorbitol, respectively. A suite of characterization techniques were used to evaluate the bioplasticizing effect of the polyol treatment on the resulting biopolymer morphology, structure, chemistry, and mechanical performance.

Sorbitol-Treated mCellulose

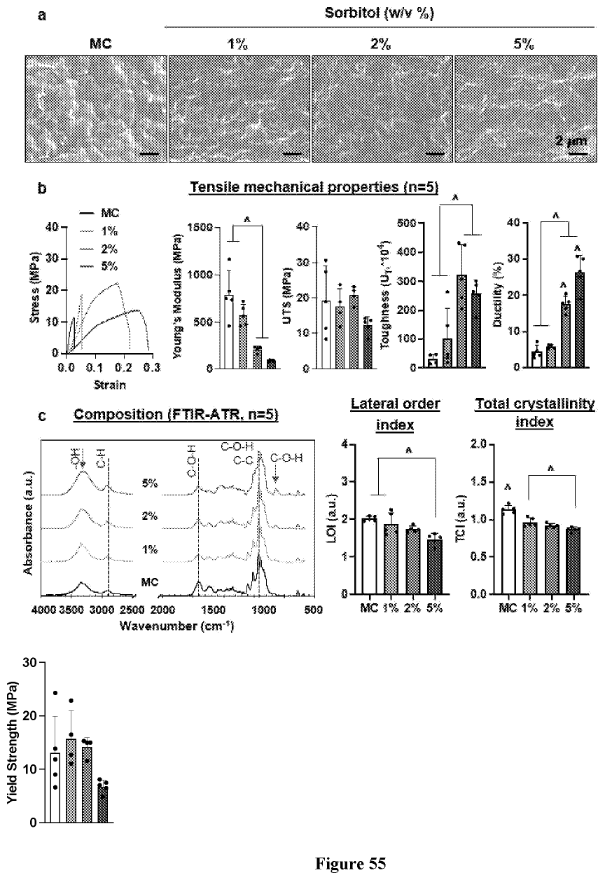

Microbial cellulose surface morphology was evaluated by SEM, in which as-fabricated groups had a densely packed fibrous surface with low porosity (FIG. 55a). Fibrous structure was continually observed without any presence of plasticizer coverage as sorbitol incorporation into the mCellulose network increased.

Dried mechanical properties of as-fabricated and sorbitol-treated mCellulose were evaluated under uniaxial tension (FIG. 55b). It was observed that as-fabricated microbial cellulose had a high young's moduli measuring at 787.98±253.55 MPa, with an accompanying low ductility of 4.31±1.76%. Comparatively, sorbitol-treated samples exhibited lower young's moduli, however, elongation at break was accordingly enhanced. As sorbitol incorporation increased from 1 to 2% w/v, Young's Modulus significantly decreased from 573.48±111.67 to 248.12±85.20 MPa, while ductility significantly enhanced from 6.77±2.20% to 17.48±2.18%. mCellulose plasticized with 5% w/v sorbitol significantly increased ductility, measuring at 26.26±4.75%, compared to all other groups, though its Young's Modulus was statistically significantly lower than as-fabricated and 1% w/v samples. Correspondingly, the toughness was also calculated for as-fabricated, 1, 2, and 5% w/v sorbitol, measuring 31.11±17.17, 101.96±103.25, 322.71±103.76, and 256.29±48.87 (*10⁴) UT, respectively, in which significant increases were measured at 2 and 5% w/v incorporation. The ultimate tensile stress was observed to be comparable between all groups, regardless of the incorporation of sorbitol.

Lastly, the chemical properties of mCellulose before and after sorbitol treatment was assessed by FTIR-ATR (FIG. 55c). Characteristic peaks of mCellulose were identified at 3345 cm⁻¹ (O—H hydroxyl stretching), 2898 cm⁻¹ (C—H methyl stretching), 1633 cm⁻¹ (—OH symmetrical stretching), and 1058 cm⁻¹ C—O—H/C—C stretching). For the spectra of sorbitol-plasticized mCellulose, the absorptions between 3600-3000 cm⁻¹ and 902-845 cm⁻¹, attributed to O—H hydroxyl stretching, increased in peak intensity as plasticizer concentration increased, likely due to the large amounts of —OH groups observed in sorbitol. Additionally, a small increase in peak intensity and shift in peak location from 2898 cm⁻¹ to 2916 cm⁻¹ was observed, correlating with the C—H vibrational stretching in sorbitol molecules. Further FTIR analysis utilizing peak ratios to measure the crystallinity index confirmed vibrational changes throughout the bulk of mCellulose due to increasing sorbitol incorporation. It was observed that both lateral order index and total crystallinity index decreased with increasing sorbitol concentration treatment, with significant differences initially found by 5% w/v for LOI and 1% w/v for TCI compared to as-fabricated.

Glycerol-Treated mCellulose

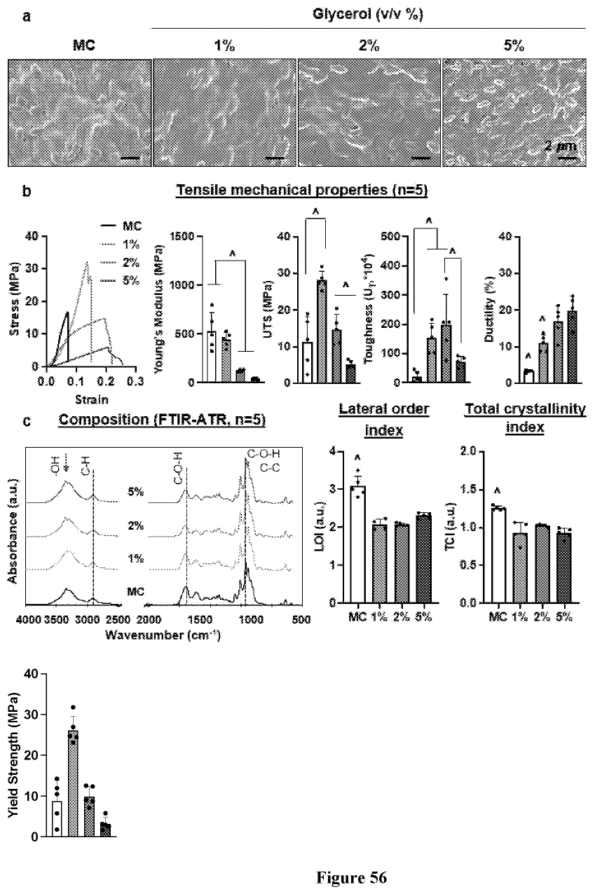

Surface architecture and fiber morphology of as-fabricated and glycerol-treated microbial cellulose were imaged by scanning electron microscopy (FIG. 56a). as-fabricated exhibited a dense randomly organized fibrous structure morphologically, however, the inherent fibrous structure became less visible with increasing glycerol treatment from 1 to 5% v/v, as a result of plasticizer's homogeneous coverage on mCellulose. Interestingly, while fiber morphology was still present at 1% v/v, 2 and 5% v/v groups displayed a smooth layer, subjected to the formation of micro-cracks and pores when exposed to the accelerating voltage of SEM.

Dry mechanical properties were measured for both as-fabricated and glycerol-treated (1, 2, and 5% v/v) mCellulose (FIG. 56b). As-fabricated groups showed a characteristic tradeoff between young's moduli (527.76±190.26 MPa) and ductility (3.23±0.43%). With increasing glycerol concentration from 0 to 1% v/v, young's moduli was maintained around 485 MPa, however, ductility was significantly enhanced to 10.89±2.20%. Greater glycerol treatments of 2 and 5% v/v significantly decreased young's moduli to 125.42±13.86 MPa and 39.83±6.06 MPa, respectively, in which subsequent ductility exhibited a positive correlation to concentration up to 19.74±3.99%. As a result, mCellulose toughness was observed to significantly increase up to 2% v/v (198.58±103.30*10⁴ UT) in comparison to as-fabricated (20.88±18.67*104 UT), but lower moduli reduced toughness values at 5% v/v. Similarly, ultimate tensile strength significantly increased at 1% v/v, measuring 28.10±2.46 MPa, followed by statistically lower values, measured in higher glycerol concentrations.

The chemical composition of mCellulose treated with and without glycerol was assessed using FTIR-ATR (FIG. 56c). Similar to as-fabricated, characteristic mCellulose peaks were identified, including 3345 cm⁻¹ (O—H hydroxyl stretching), 2898 cm⁻¹ (C—H methyl stretching), 1633 cm⁻¹ (—OH symmetrical stretching), and 1058 cm⁻¹ C—O—H/C—C stretching), supporting that no chemical modifications to as-fabricated samples from varying biofabricated cultures occurred due to purification procedures. Incorporation of glycerol to mCellulose increased peak intensity of the absorptions between 3600-3000 cm⁻¹, indicating an increase in the presence of hydroxyl groups. Furthermore, LOI and TCI measurements significantly decreased after bioplasticizing mCellulose (as-fabricated) with glycerol (1, 2, and 5% v/v), revealing the formation of less ordered structures with lower degrees of crystallinity.

Collectively, these data demonstrate that bioplasticizing mCellulose with sorbitol and glycerol yields the development of biomaterials with tunable mechanical performance that can be applied for various applications. Notably, optimal bioplasticizer concentrations of sorbitol and glycerol was determined to be 2% w/v and 1% v/v, respectively, due to the ability to enhance toughness and ductility, without compromising toughness, tensile strength, and nanofibril structure.

Evaluating mCellulose Crosslinking via Tannic Acid-Iron Complexes

Expanding on the potential of green bioprocessing to modulate mCellulose performance properties and inspired by naturally derived biomolecules, the effect of tannic acid as a crosslinking agent was evaluated. Tannic acid is a naturally occurring polyphenol extracted from various plants that is often utilized as a tannin agent in non-medical (leather) and medical textile applications. Addition of tannic acid as a crosslinker for chitosan, collagen, and gelatin has been reported to enhance tensile strength when pH conditions are suitable for hydrogen bonding between catechol and biopolymer hydroxyl groups, indicating it is an optimal biomolecule for stabilizing mCellulose mechanical behavior.

Functionally, tannic acid initially complexes with biopolymers at multiple binding sites through hydrogen bonding to form dynamic moieties, in which the combination of multiple hydroxyl-based biomolecules as plasticizers into polymer networks have been found to improve biofilm stability compared to their individual counterparts, due to the formation of plasticizer—plasticizer interactions. Based upon tannic acid's ability to facilitate the development of supramolecular materials by coordinating complexation with metal ions and biopolymers, as well as its naturally derived sourcing, tannic acid was continuously investigated as a green bioprocessing strategy in this study. Complexation of metals, such as iron, magnesium, and zinc, with tannic acid and polymers has been reported to be dependent upon balancing both hydrogen and coordinate bond interactions, in which tuning pH allows for modular material performance.

Here, a third group of microbial cellulose was biofabricated for 14 days in 2% w/v mannitol at 30 °C, and was successfully crosslinked at various pH (1.99, 5.73, and 9.14) after consecutive immersions in 0.5% w/v tannic acid and 0.5% w/v iron chloride baths for 1 day and 2 hours, respectively. Following crosslinking, TA-Fe-mCellulose was characterized to evaluate the mechanical performance of tannin-metal-cellulose complexation as a green bioprocessing technique, in which all results were compared to control groups (as-fabricated and MCTA (0.5% w/v TA).

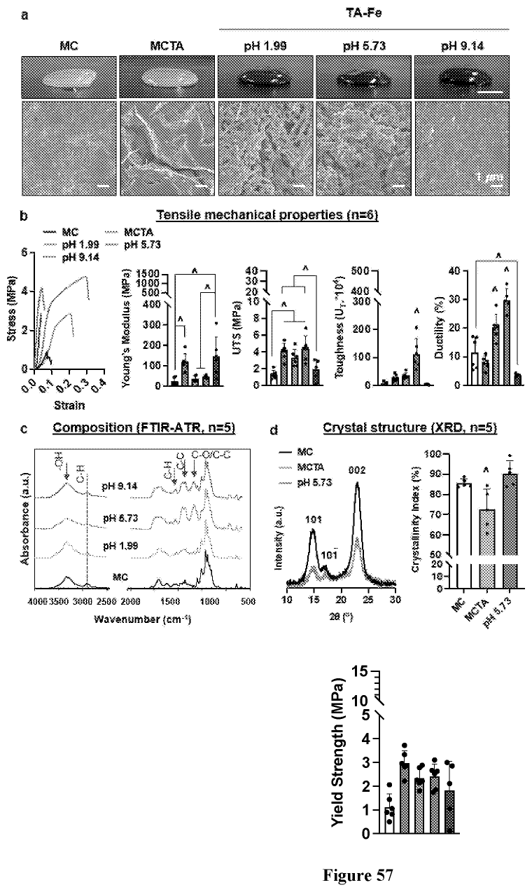

Microbial cellulose bulk macroscale (wet) and fibrous nanofiber (dry) morphology was assessed by representative photographs and SEM, respectively (FIG. 57a). As-fabricated mCellulose purified after biofabrication revealed pellicles with semi-transparent white color. The addition of tannic acid-treated microbial cellulose into FeCl₃ solutions instantaneously changed the color of hydrated biofilms from brown to black in all pH environments, indicating the simultaneous formation of polymer-catechol-metal bonds. Although the color of polymer-TA-metal systems has been reported to be an indicator of the formation of iron-based mono-, bi-, tri-complexes as pH is altered, there were no observable differences in mCellulose color at any pH, thus, samples were further morphologically inspected using SEM. As-fabricated microbial cellulose exhibited a dense randomly organized nanofibrous structure. In contrast, the nanofibers observed in MCTA groups were non-homogeneously covered with tannin aggregates. Interestingly, the immersion of TA-treated mCellulose into FeCl₃ at pH 1.99 and 5.73 exhibited the formation of TA-Fe nanoparticles adhered to nanofibers, while further pH modification to 9.14 displayed randomly oriented nanofibers similar to as-fabricated mCellulose. The loss of TA coverage and nanoparticle features suggest that alkaline conditions formed unstable interactions within the polymer-catechol-metal network, thus removing hydrogen (polymer-TA) and coordinate (TA-Fe) bonding.

Following, dried mechanical properties of mCellulose were evaluated utilizing uniaxial tensile testing (FIG. 57b). The immense hygroscopicity of mCellulose that hinders mechanical stability and performance is mitigated by tannin-iron complexation at pH 5.73, which resulted in a 1282% and 195% increase in toughness (112.76±55.10 UT (*10⁴)) and ductility (29.85±4.03%), respectively, without compromising ultimate tensile strength or yield strength. Further complexation conducted at pH 9.14 resulted in an increase in young's modulus accompanied by a reduction in ultimate tensile strength, toughness, and ductility comparable to the mechanical behavior of mCellulose treated with and without 0.5% w/v tannic acid, indicating unstable metal coordination. This unique pH-responsive modulation may be attributed to the balance between biopolymer, tannic acid, and metal interactions, in which acidic conditions not only influence catechol groups to be protonated as a hydrogen donor for cellulose hydroxyl groups, but also promote the formation of mono-complexes with iron. Conversely, although alkaline conditions support the formation of stable tri-complexes, weaker mechanical were achieved due to the oxidation of catechol into unstable quinone groups that prevent functionalization with cellulose's hydroxyl groups. Thus, these results demonstrate that mechanical toughness, ductility, and strength, as well as elastic and shear moduli, can be tuned by a pH-responsive system that influences the interactions within the polymer-catechol-metal network.

The chemical composition of mCellulose treated with and without tannic acid was assessed using FTIR-ATR (FIG. 57c). Characteristic as-fabricated mCellulose peaks were identified, including 3345 cm⁻¹ (O—H hydroxyl stretching), 2898 cm⁻¹ (C—H methyl stretching of CH₂ and CH₃), 1633 cm⁻¹ (—OH symmetrical stretching), and 1058 cm⁻¹ C—O—H/C—C/C—O—C pyranose ring stretching). The chemical structure of pure tannic acid was also confirmed by ATR, where characteristic peaks for phenolic hydroxyl (—OH) groups, carboxylic esters (C═O), aromatic C═C groups, aromatic C—C group, and benzene ring vibrations were observed at broad bands between 3600-3000 cm⁻¹, 1692 cm⁻¹, 1605/1529/1444 cm⁻¹, 1312 cm⁻¹, and 1174/1033/1012 cm⁻¹ respectively. Microbial cellulose treated with 0.5% w/v TA maintained the biopolymer's characteristic spectra, while introducing small increases in peak intensities at 3345 cm⁻¹ (OH groups), 1312 cm⁻¹ (benzene ring C—C), and 1205 cm⁻¹ (C—O, C—C), thus, indicating incorporation of the tannin. Complexation with iron at pH 1.99 further increased peak intensity around 3345 cm−1, representing greater hydroxyl groups. However, an increase in pH to 5.73 and 9.14 irreversibly decreased peak intensity of the hydroxyl broad band between 3600-3000 cm⁻¹, increased the relative absorbance of methyl stretching (1428 cm⁻¹), aromatic C—C groups (1312 cm−1), and C—O/C—C groups (1205 cm⁻¹), while maintaining characteristic peaks assigned for mCellulose.

Microbial cellulose crystal structure was assessed by X-ray diffraction (FIG. 57d). While all groups exhibited cellulose I Bragg peaks at 2θ=14.6°, 16.9°, and 22.8°, corresponding to the (1-10), (110), and (200) planes, a peak deconvolution method indicated a minor increase in crystallinity index in mCellulose complexed with tannic acid and iron at pH 5.73 (as-fabricated: 85.61±2.15% vs. pH 5.73: 90.34±6.25%). Collectively, the tensile properties and crystallinity index of as-fabricated and pH 5.73 suggests that complexation with iron alters mCellulose bulk structure and mechanical behavior, as a result of chemical crosslinking.

Collectively, the data suggests that tannic acid-metal complexation as a bioprocessing treatment modifies mCellulose crosslinking through binding biopolymer hydroxyl groups with phenol compounds, in which further coordination bonding with Fe(III) can be altered by pH. It is proposed that simple immersion of hydrated mCellulose into both aqueous tannic acid and iron solutions not only covers the surface, but also penetrates the fibril network, increasing the space between biopolymer molecules that supports greater mobility within cellulose chains. Under acidic conditions, phenol groups within catechol compounds become protonated, promoting its application as a hydrogen donor to bind to hydrogen-accepting polymers. The increase in hydroxyl and carboxyl groups detected with FTIR, along with a non-homogenous layer of tannin deposition observed with SEM, for tannic acid-treated samples only demonstrated enhanced young's moduli, tensile strength, and yield strength, indicating the formation of cross-linking interactions. Of note, carboxyl groups can form ester bonds with available hydroxyl groups, forming stronger linkages than the hydrogen bonds between as-fabricated cellulose chains, further supporting greater mechanical performance. Complexation with Fe(III) at pH 1.99 suggests the formation of mono-coordinate bonds that support greater stability between cellulose chains, in which increased ductility with maintained strength. At pH 5.73, increased crystallinity observed with XRD, as well as, increased relative FTIR absorbance of methyl stretching, indicated the formation of stable and mobile coordinate and methyl bridges, supporting optimal toughness (MC 3: 7.25±4.88 (* 10⁴) UT vs. pH 5.73: 112.76±55.09 (* 10⁴) UT) and ductility (MC 3: 11.28±5.57% vs. pH 5.73: 29.85±4.03%) with retained strength. The decrease in tensile and viscoelastic properties under alkaline conditions at pH 9.14 reinforce the claim that chemical crosslinks through hydroxyl, methyl, and coordinate bonding can be altered based upon pH. Thus, the enhancement and stabilization of mechanical toughness and strength with cellulose-tannin-iron complexes may be accredited to the prevention of hydrogen bonding between cellulose chains by increasing chemical interaction with hydroxyl, methyl, and coordinate bonds that distributes applied stress.

### Antimicrobial Activity

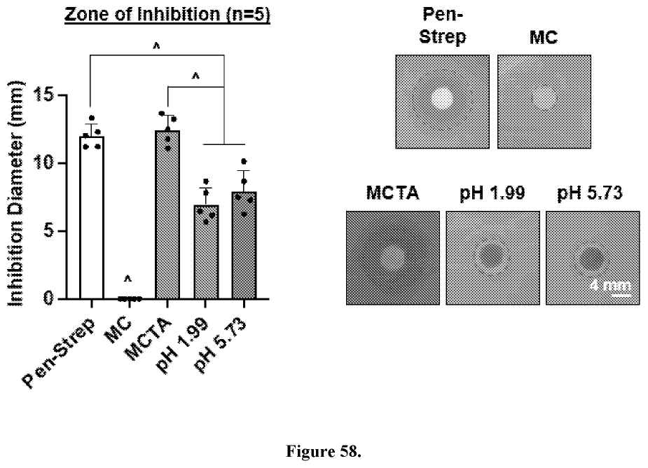

Tannic acid exhibits antimicrobial and anti-biofilm properties against various pathogenic bacterial strains, such as E. coli, S. aureus, and P. aeruginosa. Mechanistically, tannic acid has been reported to not only complex with enzymes within bacterial membranes, inhibiting quorum sensing, but also complex with metals in the environment, preventing bacteria from absorbing essential metal ions for survival. Testing the antimicrobial potential of supramolecular tannic acid bioprocessing, microbial cellulose complexed with tannic acid and iron were utilized in a Kirby-Bauer disk diffusion assay to determine its effect on gram-negative E. coli (FIG. 58). As-fabricated mCellulose exhibited no inhibition zones after 24 hours of culture, suggesting that pristine groups do not inherently have antimicrobial properties. However, mCellulose films only treated with 0.5% w/v tannic acid significantly increased inhibition zones to 12.44±1.05 mm, which was comparable to control disks containing 2% penicillin-streptomycin. Further complexation between mCellulose, tannic acid, and Fe(III) at pH 1.99 and 5.73 significantly suppressed the diameter of the inhibition zone to 6.95±1.23 and 7.95±1.49 mm, respectively. This suggests that iron ions crosslink the polymer network, limiting the polyphenol's bioavailability and subsequent leaching.

### Environmental Impact Assessment

It is desirable to assess the environmental impacts of any technology, systematically to quantify environmental performance and ensure progress towards climate and sustainable development goals. A cradle-to-gate life cycle impact assessment was implemented to compare the human health and environmental manufacturing impacts between various carbon sources, as well as post-production treatments utilized for the biofabrication of mCellulose biotextiles. Both normalization and weighting factors were applied to convert the manufacturing impacts of 10 categories (Human Health: fossil fuel depletion, carcinogenics, hormonally active agents, respiratory effects, and smog; Environmental: acidification, ecotoxicity, eutrophication, global warming, ozone depletion) into milliPoints.

### Environmental Performance of Carbon Sources

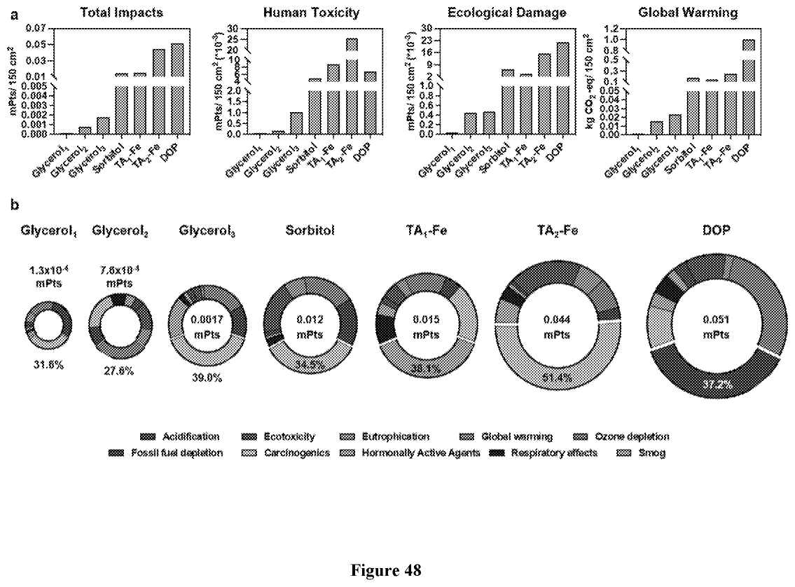

The cradle-to-gate LCA was applied to compare the environmental performance of glucose, fructose, sucrose, mannitol, and xylitol used as carbon sources for mCellulose biofabrication (FIG. 48). Here, manufacturing impacts were converted into milliPoints per yielded amount of cellulose after 14 days. Notably, impacts from each sugar have low magnitudes due to benchtop biofabrication, however, these data provide insights for green biofabrication at scale. Upon normalizing to the respective amount of cellulose produced after 14 days, it is shown that sucrose offers significant improvement in reducing human health and environmental toxicity compared to all other sugars for 2% w/v cultures, especially xylitol. This reduction may be in part to the sourcing of sucrose from sugar cane at a sugar refinery, thus eliminating large contributions of carcinogens (50.9%) and ecotoxicity (19.9%) observed when extracting xylitol via biomass hydrolysis. However, hormonally active agents were found to be the leading category for sucrose, accounting for 41.9% of total impact. Although sucrose resulted in more than ten times reduction in total impacts compared to xylitol (0.77×10⁻⁵ vs 1.41×10⁻⁴ mPts), both carbon sources, at 2%, produced comparable yields of microbial cellulose throughout 14 days of culture. Thus, xylitol's low cellulose production efficiency accompanied with the highest human health and environmental toxicity suggests that it is not suitable for green biofabrication of microbial biotextiles with improved circularity and scalability. Interestingly, although mannitol and glucose yield the highest cellulose in 14 days, glucose offers significant improvement to reduce total impacts by 71% (1.5×10⁻⁵ vs 5.2×10⁻⁵ mPts), while mannitol enhances biomaterial mechanical properties. Thus, the ability to not only tune yield, but also performance properties by biofabricating with carbon sources that have varying human health and environmental impacts, incentivizes the transition to a circular materials economy, offering a versatile platform for textile-based applications.

### Environmental Performance of Post-processing via Bioplasticizer and Cross-linking Agents

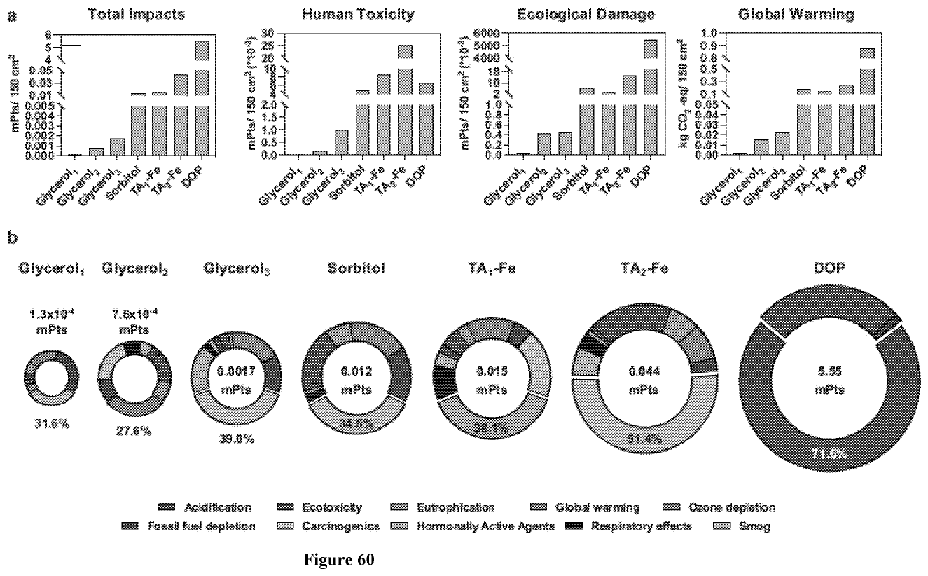

Similarly, the cradle-to-gate LCA was applied to compare the environmental performance of glycerol, sorbitol, and tannic acid-Fe(III), to the traditional plasticizer di-2ethylhexyl phthalate (FIG. 60). Here, manufacturing impacts were converted into milliPoints per 150 cm² of mCellulose.

Di-2ethylhexyl phthalate, a plasticizer used in the production of most polyvinyl chloride plastic, was observed to have the largest total impact (5.55 mPts) in comparison to glycerol, sorbitol, and tannic acid-Fe(III) (FIG. 60a). Of note, ecological damage (5.47 mPts) caused by acidification (71.6%) was identified as the impact hotspot contributing to the dramatically poor environmental performance of di-2ethylhexyl phthalate.

In terms of the impacts between green bioprocessing strategies, tannic acid—iron (III)—based methods were observed to have the largest total impacts when modelled against sorbitol (2% w/v) and glycerol (1% v/v). However, depending on the source of tannic acid, resulting human toxicity, ecological damage, and global warming impacts were varied. Here, tannic acid was demonstrated to be extracted from either polyphenol waste biomass (TA1-Fe) or hot water (TA₂-Fe), in which sourcing from the latter resulted in three times greater total impacts (0.044>0.015 mPts). Interestingly, it was found that tannic acid contributes significantly more to these impacts than its counterpart iron chloride, however, differences were observed in terms of each sources major influence to human toxicity. While carcinogenics accounted for 51.4% of the total impacts of tannic acid sourced from hot water, hormonally active agents accounted for 38.1% of tannic acid extracted from polyphenol waste biomass. Further LCA analysis indicated that the TA-Fe and sorbitol post-processing treatments have comparable carbon footprint (0.144-0.249 kg CO₂-eq per 150 cm²), while one hundred times greater than the glycerol process.

Sorbitol manufactured by enzymatic hydrolysis slightly reduced total impacts to 0.012 mPts, while ecological damage was measured to be the process' largest impact category (0.0064 mPts). The use of glycerol as a plasticizer post-production treatment offers a significant improvement in human health and ecological performance, and reduction in greenhouse gas emissions (0.0017 kg CO2-eq per 150 cm²) compared to sorbitol and tannic acid-iron ion procedures. Notably, the source of glycerol also altered the resulting impacts, albeit values remained between ten to one-hundred times lower than sorbitol and TA-Fe. While the production of glycerol manufactured by epichlorohydrin obtained in waste oil (glycerol3) resulted in the highest total impacts, measuring at 0.0017 mPts, glycerol from vegetable oil at an esterification plant (glycerol1) decreased total impacts to 1.3×10⁻⁴ mPts. This pronounced reduction in impacts with glycerol can be accounted for the elimination of human toxicity comprised by carcinogenics, which were observed to be 34.5% and 51.4% of the bulk total impacts within sorbitol and TA₂-Fe, respectively. However, carcinogenics was identified as the largest impact (hotspot) for both glycerol produced from vegetable oil (glycerol1, 31.6%), and epichlorohydrin waste oil (glycerol₃, 39.0%).

Overall, bioprocessing with sorbitol, glycerol, and TA-Fe offered significant improvements in environmental performance compared to traditional DOP-based plasticizers, which has the largest total impacts of the group. Although tannic acid—iron (III)—based processing methods were observed to have the largest total impact (0.015-0.044 mPts) when compared against sorbitol and glycerol, crosslinking processes may limit the polyphenol's subsequent leaching into the environment, circumventing the characteristic migration of plasticizers. This is supported by observations from previous reports by Martinez et al., who investigated the effects of crosslinking strategies on modulation of drug release profiles from polymers, in which stronger crosslinking reduced drug release. Of note, LCA analysis was performed based upon laboratory technical grade processing procedures, in which further examination of utilizing renewable sources of plasticizers and crosslinkers may reduce human toxicity, ecological damage, and carbon footprint impacts. Thus, the ability to control the source of bioprocessing chemicals supports the implementation of green chemistry strategies for tuning microbial cellulose performance within a circular materials economy.

Harnessing microbial biofabrication coupled with green bioprocessing techniques, the development of mCellulose with enhanced and stabilized mechanical behavior was achieved without the significant human health and environmental impacts observed with conventional plasticizers. Due to the complexity of acetic acid bacteria's carbon metabolism, this biofabrication platform allows for utilizing a vast catalogue of carbon sources with different eco- and human toxicity to synthesize the same biopolymer, albeit at varying efficiencies and physiochemical properties. In particular, the use of mannitol significantly increased cellulose production throughout 14 days of culture with enhanced tensile mechanical performance. Furthermore, two post-processing approaches utilizing glycerol and sorbitol as bioplasticizers offers enhancement of toughness in a concentration dependent manner, eliminating the use of endocrine-disrupting phthalates for improving performance properties. An alternative crosslinking procedure incorporating tannic acid and iron ion complexation modified mCellulose with apparent antimicrobial properties, as well as, enhanced toughness and strength, which was strongly dependent upon pH conditions. Life cycle assessment revealed the application of glycerol reduced manufacturing impacts over one magnitude lower than tannic acid and iron complexation treatment, albeit the source of glycerol production (vegetable oil vs. epichlorohydrin obtained in waste oil) can further mitigate impacts.

Future prospects aim to determine the viability of agro-industrial waste streams as low impact waste-to-resource strategies for biofabricating high performance microbial cellulose biotextiles within a circular economy. Additionally, controlled crosslinking of microbial cellulose with tannic acid-based metal complexation for high performance alternatives to cotton, synthetic films (low density polyethylene), and biomedical textiles will be explored. Further implementation of naturally occurring polyphenol-cellulose chelation to different metal ions shows immense potential to modulate mechanical performance of biotextiles with antibacterial properties, while modulating bioactive molecule leaching and migration out of the bulk material. Thus, this biofabrication and post-processing platform supports the implementation of green chemistry strategies for tuning mCellulose within a circular materials economy, meeting key design criteria such as low toxicity, rapid renewability, and high performance.

Microbial cellulose Complex

Microbial cellulose is a highly crystalline and chemically pure extracellular polysaccharide, free of hemicellulose and lignin, secreted by gram-negative bacteria, such as Acetobacter xylinum (Huang et al., 2014). Under specific aerobic culturing conditions these bacteria biofabricate unaligned cellulose nanofibrils (10-100 nm diameter) that coagulate into a three-dimensional layered hydrogel (>98% water) (Mohite et al., 2018; Gelin et al., 2007), known as a pellicle, at the air-culture media interface. Compared to plant cellulose, microbial cellulose has gained significant interest due to its inherent physiochemical properties, including high tensile strength, high degree of polymerization (Dahman, 2009), crystallinity (80-90%), high hygroscopicity and swelling capacity, moldability, biodegradability in composting environments, and biocompatibility (Choi, Shin, 2020). Owing to these unique properties, mCellulose is an advanced biomimetic material that is attractive for biofabrication applications, such as sustainable material alternatives to cotton and synthetic textiles, mitigating the large environmental impact of the textile industry, and as a tissue engineered scaffold for regeneration of musculoskeletal tissues.

However, the large quantity of hydroxyl groups that support the immense hygroscopicity of mCellulose results in unstable mechanical performance, hindering further translation to textile applications (Kaminski et al., 2020; Schiros et al., 2022). Common to unprocessed naturally-derived biopolymers, as-fabricated mCellulose exhibits varied stress-strain behaviors in both hydrated and dry states, thus demonstrating its inherent trade-off between tensile strength and ductility (Kaminski et al., 2020; Schiros et al., 2022).

Strategies to improve the mechanical stability of polymers have included the subsequent implementation of post-processing methods incorporating synthetic plasticizers, coatings, and heavy metals that not only compromise bulk material biodegradability, but also promote environmental contamination and human toxicity (Howell, Lazar, 2019; Howell, Sun, 2018). In particular, traditional plasticization with phthalate esters, such as di(2-ethylhexyl)phthalate (DEHP) and di(isononyl)phthalate (DINP), is often employed for polymeric materials to be processed with decreased inherent brittleness (Howell, Lazar, 2019; Rahman, Brazel, 2004). However, the inherent behavior of phthalates to leach, migrate, and evaporate out of bulk materials have facilitated the wide distribution of non-biodegradable nano- and micro-plastics into the ecosystem and food chain. Furthermore, the consistent accumulation and absorption into the environment has led to the discovery of micro-plastics in human blood, intestines, placenta, as well as a rise of endocrine system disruption-based human diseases associated to phthalates (Ragusa et al., 2021; Deng et al., 2017; Cho et al., 2010; Franco et al., 2007; Wittassek et al., 2011). Thus, development of green bioprocessing strategies in conjunction to biofabrication would be desirable to minimize human health and environmental impacts.

The development of bioplasticizers derived from renewable bio-sources with low migration potential from a polymeric matrix are of particular interest due to their nontoxic, manufacturing inputs (Bocqu6 et al., 2016). Common bioplasticizers include polyols, monosaccharides, oligosaccharides, lipids, and hyperbranched poly(esters) (Howell, Lazar, 2019; Lee et al., 2018), in which coatings have the ability to increase mobility between polymer chains, thus reducing intermolecular forces between molecules and material brittleness (Cobos et al., 2017; Sanyang et al., 2015). However, the source and concentration of plasticizer has a significant influence on the resulting biomaterial physical, mechanical, and thermal properties, as well as the environmental impact of manufacturing. Among these, glycerol and sorbitol have been demonstrated to be suitable polyol-based bioplasticizing agents for incorporation into starch (Sanyang et al., 2015; Mikkonen et al., 2009; Laohakunjit, Noomhorm, 2004), chitosan (Suyatma et al., 2005), whey protein (McHugh, Krochta, 1994), and cellulose (Cielecka et al., 2019; Sun et al., 2018) systems with modifications of their mechanical performance. Functionally, glycerol and sorbitol contain hydroxyl end groups that form hydrogen bonds between biopolymer molecules, preventing the bulk material from creating too many hydrogen bonds with itself (FIG. 1). Thus, an increase in the space between biopolymer molecules supports greater mobility of polymeric chains for improved biomaterial ductility, however, at the expense of reduced mechanical resistance. Additionally, glycerol is further hindered by its migration out of bulk biomaterial systems (Mikkonen et al., 2009).

Despite the great abundance of hydroxyl groups within microbial cellulose that are available for hydrogen bonding with polyol-based bioplasticizers, these post-processing approaches are hindered by a multitude of challenges including: anti-plasticization behavior at variable concentrations, and progressive leaching that does not support strategic elimination of human health and ecological impacts. To mitigate these challenges, it would be desirable investigate a green, low impact crosslinking strategy as an alternative bioprocessing method for enhancing and stabilizing microbial cellulose mechanical performance.

Naturally produced polyphenols from plants, such as tannic acid, have diverse bonding abilities to crosslink with biopolymers (chitosan (Li et al., 2020; Rivero et al., 2010; Rubentheren et al., 2015), collagen (Heijmen et al., 1997), and gelatin (Cao et al., 2007; Zhang, Do, et al., 2010), given that pH conditions are suitable for facilitating interactions (Chen et al., 2022; Guo et al., 2014; Guo et al., 2016; Phan et al., 2016). Functionally, tannic acid is capable of complexing macromolecules at multiple binding sites with catechol groups, via hydrogen bonding or ionic interactions, to form dynamic moieties (Erel-Unal, Sukhishvili, 2008; Shutava et al., 2005). Depending on the chemical structure of the macromolecule, concentration of polymer and tannic acid, and pH, tannic acid's facilitated interactions have been reported to not only enhance structural stability, but also introduce inherent antibacterial and antiviral properties from the polyphenol (Akiyama et al., 2001; Chung et al., 1998; Colak et al., 2010; Lee et al., 2013). Of note, tannic acid's chelation ability to coordinate metal ions, such as iron (Heijmen et al., 1997; Fan et al., 2015; Fan et al., 2017; Zhang, Yang et al., 2020; Ejima et al., 2013), magnesium (Zhang, Sun, et al., 2020), and zinc (Ninan et al., 2016), with polymers has demonstrated the platform's capacity to form supramolecular networks with pH-tunable interactions that modulate the polyphenol's bioavailability and subsequent leaching. However, resulting material performance is controlled by the balance between biopolymer, tannic acid, and metal interactions, such that although catechol groups become unstable in neutral and basic environments due to its oxidation into a quinone form, metal coordination is enhanced by transitioning from mono-complexes (acidic) to tri-complexes (basic) (Fan et al., 2017; Ejima et al., 2013). Despite research success, utilization of tannic acid-metal complexation as a pH-sensitive green bioprocessing procedure for modulating multilayered cellulose ductility and strength has not been exhibited. Thus, strategic development of tannic acid crosslinking may provide enhanced control of microbial cellulose mechanical performance.

Ultimately, this application aims to develop various bioprocessing methods for enhancing and stabilizing microbial cellulose mechanical properties, while minimizing manufacturing impacts on human health and on the environment. In the following sections, the optimization of bioplasticizing microbial cellulose with sorbitol or glycerol, as well as crosslinking with tannic acid and iron ion complexation would be investigated (FIG. 1). To this end, post-processed mCellulose films would be evaluated for their resulting morphology, tensile mechanical behavior, and chemical and crystal structure, in which these green processing procedures would be further assessed for their manufacturing impacts across human health, ecological damage, and global warming categories.

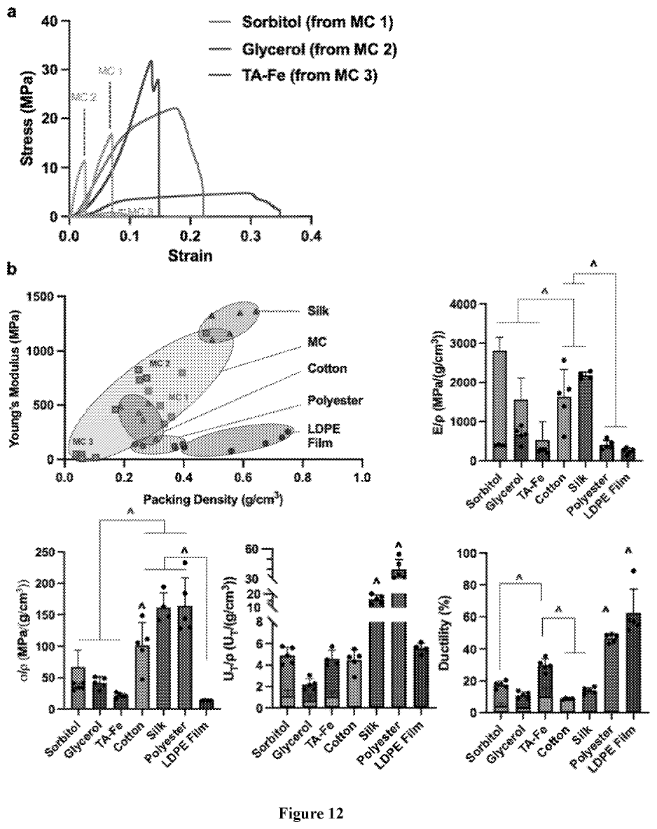

Microbial cellulose is a highly crystalline extracellular polysaccharide, secreted by gram-negative bacteria, such as Acetobacter xylinum (Huang et al., 2014). Under specific aerobic culturing conditions these bacteria biofabricate unaligned cellulose nanofibrils (10-100 nm diameter) that coagulate into a three-dimensional layered hydrogel (>98% water) (Mohite et al., 2018; Gelin et al., 2007), known as a pellicle, at the air-culture media interface. Although mCellulose offers immense advantages (high tensile strength, high degree of polymerization (Dahman, 2009), and biocompatibility) to meet key design criteria for high performance, circular economy textiles (Schiros et al., 2022), its hygroscopicity results in unstable mechanical performance, hindering further translation to textile applications (Kaminski et al., 2020; Schiros et al., 2022). Despite maintaining bacterial culture conditions at 30 °C for 14 days in 2% w/v mannitol, three biofabricated (MC 1, MC 2, and MC 3) mCellulose batches displayed varying stress-strain profiles in their dried state, suggesting the typical tradeoff between tensile strength and ductility (FIG. 12a). While mCellulose young's moduli, ranging from 24.85±16.95 to 787.98±253.55 MPa, exhibited a positive correlation to its inherent packing density, the resulting specific tensile strength, specific toughness, and ductility was observed to be comparable between batches (FIG. 12b; grey bars). However, as-fabricated groups exhibited significantly lower specific toughness than conventional textiles such as cotton, silk, polyester, and low density polyethene films. Surprisingly, similar ductility was observed in as-fabricated mCellulose, cotton, and silk up to strain values of 11.28±5.57% (MC 3), 8.52±0.45%, and 13.77±1.95%, respectively, albeit polyester and LDPE films presented superior elongation at break. Collectively, these data demonstrate microbial biofabrication yields cellulose with unstable and inferior mechanics required to become high performance materials that contend against traditional textiles. Strategies to improve the mechanical stability of polymers have included the subsequent implementation of post-processing methods incorporating synthetic plasticizers that not only compromise bulk material biodegradability, but also promote environmental contamination and human toxicity (Howell, Lazar, 2019; Howell, Sun, 2018), therefore making the development of green bioprocessing an immense importance. Thus, the effects of bio-plasticizing (sorbitol and glycerol) and -crosslinking (tannin-metal complexation) strategies on the structure and mechanical performance of microbial cellulose were compared.

Bioplasticizers have been reported to alter polymer chain spacing, characteristically increasing ductility and decreasing strength of biomaterial systems compared to as-fabricated states (Cobos et al., 2017; Sanyang et al., 2015). Plasticizer type and concentration can be used to modulate mechanical performance, in which greater concentrations promote anti-plasticization behavior (Sanyang et al., 2015). This is not only supported by the positive correlation between concentration and ductility observed in both 5% sorbitol and glycerol groups, reaching up to 26.26±4.75% and 19.74±3.98%, respectively, but also a decrease in toughness and tensile strength at 5% w/v sorbitol (256.29±48.87 (* 10⁴) U_{T}; 12.24±2.37 MPa) and 5% v/v glycerol (71.01±18.53 (*10⁴) U_{T}; 5.20±0.95 MPa) (FIG. 3&4).

Comparative morphological analysis of microbial cellulose after bioplasticization indicated obvious differences in surface microstructure. While nanofibers were observed without any coverage in all sorbitol groups, glycerol homogenously covered the fibers in a concentration dependent manner, specifying that biomolecule incorporation via immersion technique is affected by plasticizer type and concentration (FIG. 3&4). Of note, the mass of plasticized mCellulose significantly increased with 2% sorbitol (w/v) or glycerol (v/v) treatments, measuring double the weight of as-fabricated groups, while thickness was only significantly greater by 5%. These data suggest that although there is great compatibility between sorbitol or glycerol and mCellulose to similarly alter mechanical performance, the type of plasticizer incorporates with the biopolymer surface and penetrates into the nanofibril layered network differently. In addition, increased concentrations likely introduce too much space between cellulose molecules that reduce polymer chain strength, resulting in bulk mass originating from the plasticizer.

Maintenance of cellulose chemical structure after post-processing was verified using FTIR-ATR, where characteristic peaks of mCellulose were identified at 3345 cm⁻¹ (O—H hydroxyl stretching), 2898 cm⁻¹ (C—H methyl stretching), 1633 cm⁻¹ (—OH symmetrical stretching), and 1058 cm⁻¹ C—O—H/C—C stretching) (Schiros et al., 2022; Atykyan et al., 2020). As a result of immersing microbial cellulose with hydroxyl-based plasticizers, minor chemical composition modifications were observed in spectra between 3600-3000 cm⁻¹, indicating an increase in the presence of hydroxyl groups and subsequent hydrogen bonding (FIG. 3&4) (Cielecka et al., 2019; Sun, Zheng et al., 2018; Sun, Liang et al., 2018; Chen et al., 2008; AlOmar et al., 2016). Surprisingly, although characteristic cellulose peaks remained present, indicating that chemically the biomaterial's bulk structure is not altered, FTIR peak ratios, expressed as Total Crystallinity Index (TCI), showed a lower degree of order and crystallinity for treated groups compared to as-fabricated mCellulose.

Collectively, these data demonstrate that bioplasticizing mCellulose with sorbitol and glycerol yields the development of biomaterials with tunable mechanical performance that can be applied for various applications. Notably, optimal bioplasticizer concentrations of sorbitol and glycerol was determined to be 2% w/v and 1% v/v, respectively, due to the ability to enhance toughness and ductility, without compromising toughness, tensile strength, and nanofibril structure.

Expanding on the potential of green bioprocessing to modulate mCellulose performance properties and inspired by naturally derived biomolecules, this study additionally evaluated the effect of tannic acid as a crosslinking agent. Tannic acid is a naturally occurring polyphenol extracted from various plants that is often utilized as a tannin agent in non-medical (leather) and medical textile applications (Chen et al., 2022; Guo et al., 2014; Guo et al., 2016; Phan et al., 2016). Addition of tannic acid as a crosslinker for chitosan (Li et al., 2020; Rivero et al., 2010; Rubentheren et al., 2015), collagen (Heijmen et al., 1997), and gelatin (Cao et al., 2007; Zhang et al., 2010) has been reported to enhance tensile strength when pH conditions are suitable for hydrogen bonding between catechol and biopolymer hydroxyl groups (Chen et al., 2022; Guo et al., 2014; Guo et al., 2016; Phan et al., 2016), indicating it is an optimal biomolecule for stabilizing mCellulose mechanical behavior. Functionally, tannic acid initially complexes with biopolymers at multiple binding sites through hydrogen bonding to form dynamic moieties (Erel-Unal, Sukhishvili, 2008; Shutava et al., 2005), in which the combination of multiple hydroxyl-based biomolecules as plasticizers into polymer networks have been found to improve biofilm stability compared to their individual counterparts, due to the formation of plasticizer—plasticizer interactions (Adhikari et al., 2010; Fu et al., 2011; Krogars et al., 2003). Applying this strategy to microbial cellulose's nanofibrous network, glycerol (2% v/v) and tannic acid (1 and 2% w/v) immersions were utilized as consecutive treatments (24 hours each) to characterize the mechanical behavior of mCellulose with polyol—catechol interactions (FIG. 5). As a result, tensile properties revealed that young's modulus (72.59±38.83 MPa), tensile strength (8.57±4.37 MPa), toughness (119.63±58.75 (*10⁴) U_{T}), and ductility (20.89±0.36%) were comparable before and after 1% w/v tannic acid treatment on glycerol (2% v/v) plasticized samples, in which greater TA concentrations at 2% w/v also had no effect. Additionally, mass and thickness measurements also remained comparable after tannic acid treatment(s), further confirming a lack of interaction between catechol, polyol, and cellulose's hydroxyl groups.

Based upon tannic acid's ability to facilitate the development of supramolecular materials by coordinating complexation with metal ions and biopolymers, as well as its naturally derived sourcing, tannic acid was continuously investigated as a green bioprocessing strategy. Complexation of metals, such as iron (Heijmen et al., 1997; Fan et al., 2015; Fan et al., 2017; Zhang, Yang et al., 2020; Ejima et al., 2013), magnesium (Zhang, Sun et al., 2020), and zinc (Ninan et al., 2016), with tannic acid and polymers has been reported to be dependent upon balancing both hydrogen and coordinate bond interactions, in which tuning pH allows for modular material performance. Here, tannic acid-treated microbial cellulose was immersed into 0.5% w/v FeCl₃ aqueous baths to facilitate complexation between metal ions and the biopolymer, in which pH values of iron solutions were either maintained at 1.99 or adjusted to 5.73 or 9.14. The addition of tannic acid-treated microbial cellulose into FeCl₃ solutions instantaneously changed the color of hydrated biofilms from brown to black in all pH environments, indicating the simultaneous formation of polymer-catechol-metal bonds (FIG. 6a. Although the color of polymer-TA-metal systems has been reported to be an indicator of the formation of iron-based mono-, bi-, tri-complexes as pH is altered (Fan et al., 2017; Ejima et al., 2013), there were no observable differences in mCellulose color at any pH, thus, samples were further morphologically inspected using SEM. As-fabricated microbial cellulose exhibited a dense randomly organized nanofibrous structure. In contrast, the nanofibers observed in MCTA groups were non-homogeneously covered with tannin aggregates. Interestingly, the immersion of TA-treated mCellulose into FeCl₃ at pH 1.99 and 5.73 exhibited the formation of TA-Fe nanoparticles adhered to nanofibers, while further pH modification to 9.14 displayed randomly oriented nanofibers similar to as-fabricated MC 3. The loss of TA coverage and nanoparticle features suggest that alkaline conditions formed unstable interactions within the polymer-catechol-metal network, thus removing hydrogen (polymer-TA) and coordinate (TA-Fe) bonding. Following, the complexation between cellulose, catechol, and iron ions were verified by mechanical properties (tensile and viscoelastic), structural and chemical composition, and anti-bacterial properties.

The immense hygroscopicity of mCellulose that hinders mechanical stability and performance is mitigated by tannin-iron complexation at pH 5.73, which resulted in a 1282% and 195% increase in toughness and ductility, respectively, without compromising ultimate tensile strength or yield strength (FIG. 6b). Further complexation conducted at pH 9.14 resulted in an increase in young's modulus accompanied by a reduction in ultimate tensile strength, toughness, and ductility comparable to the mechanical behavior of mCellulose treated with and without 0.5% w/v tannic acid, indicating unstable metal coordination. In addition, the effect of pH on polymer-catechol-iron interaction was also determined by examining the viscoelastic properties of hydrated microbial cellulose via shear-strain controlled rheometry (FIG. 8). Of note, the elastic and shear moduli increased when tannic acid-treated microbial cellulose was initially complexed with Fe(III) at pH 1.99, however, significant decreases in G' and |G*| were observed with increased pH to 9.14. This unique pH-responsive modulation may be attributed to the balance between biopolymer, tannic acid, and metal interactions, in which acidic conditions not only influence catechol groups to be protonated as a hydrogen donor for cellulose hydroxyl groups, but also promote the formation of mono-complexes with iron. Conversely, although alkaline conditions support the formation of stable tri-complexes, weaker mechanical and viscoelastic performance were achieved due to the oxidation of catechol into unstable quinone groups (Fan et al., 2017) that prevent functionalization with cellulose's hydroxyl groups. Thus, these results demonstrate that mechanical toughness, ductility, and strength, as well as elastic and shear moduli, can be tuned by a pH-responsive system that influences the interactions within the polymer-catechol-metal network.

Not surprisingly, differences in crystal structure are subtly reflected in the tensile mechanical properties of tannin-iron treated mCellulose measured by X-ray diffraction (FIG. 7a). While all groups exhibited cellulose I Bragg peaks at 2θ=14.6°, 16.9°, and 22.8°, corresponding to the (1-10), (110), and (200) planes (Samuel, Adefusika, 2019), a peak deconvolution method indicated a minor increase in crystallinity index in mCellulose complexed with tannic acid and iron at pH 5.73 (MC 3: 85.61±2.15% vs. pH 5.73: 90.34±6.25%; FIG. 7b), albeit not significant. Collectively, the tensile properties and crystallinity index of as-fabricated (MC 3) and pH 5.73 suggests that complexation with tannic acid-iron alters mCellulose bulk structure and mechanical behavior, as a result of pH-influenced chemical crosslinking.

Chemical and elemental fingerprints observed with FTIR-ATR and EDXA, respectively, provided mechanistic insights on microbial cellulose crosslinked with tannic acid and Fe(III) complexation. As mCellulose treated with 0.5% w/v TA maintained the biopolymer's characteristic spectra, small increases in peak intensities were introduced at 3345 cm⁻¹ (O—H groups), 1312 cm⁻¹ (benzene ring C—C), and 1205 cm⁻¹ (C—O, C—C), indicating incorporation of the tannin (Zhang, Sun, et al., 2020; Ninan et al., 2016; Lee et al., 2018; Muhoza et al., 2019; Iglesias et al., 2001). Further, complexation with iron at pH 1.99 increased peak intensity around 3345 cm⁻¹, representing greater hydroxyl groups (FIG. 9b). However, an increase in pH to 5.73 and 9.14 irreversibly decreased peak intensity of the hydroxyl broad band between 3600-3000 cm⁻¹, increased the relative absorbance of methyl stretching (1428 cm⁻¹), aromatic C—C groups (1312 cm⁻¹), and C—O/C—C groups (1205 cm⁻¹), while maintaining characteristic peaks assigned for mCellulose. FTIR peak ratios for total crystallinity index measurements revealed an increased degree of crystallinity when tannin-iron complexation occurred at pH 5.73, thus supporting the observed enhancement in tensile mechanical behavior and crystallinity observed with X-ray diffraction. Iron deposition was well distributed within microbial cellulose fibril network at pH 1.99, in which EDXA intensity profiles indicated significant decrease in Fe presence with an increase in pH to 9.14, suggesting less complexation in alkaline conditions (FIG. 9 d-f).

Collectively, the data suggests that tannic acid-metal complexation as a bioprocessing treatment modifies mCellulose crosslinking through binding biopolymer hydroxyl groups with phenol compounds, in which further coordination bonding with Fe(III) can be altered by pH. It is proposed that simple immersion of hydrated mCellulose into both aqueous tannic acid and iron solutions not only covers the surface, but also penetrates the fibril network, increasing the space between biopolymer molecules that supports greater mobility within cellulose chains. Under acidic conditions, phenol groups within catechol compounds become protonated, promoting its application as a hydrogen donor to bind to hydrogen-accepting polymers. The increase in hydroxyl and carboxyl groups detected with FTIR, along with a non-homogenous layer of tannin deposition observed with SEM, for tannic acid-treated samples only demonstrated enhanced young's moduli, tensile strength, and yield strength, indicating the formation of cross-linking interactions. Of note, carboxyl groups can form ester bonds with available hydroxyl groups, forming stronger linkages than the hydrogen bonds between as-fabricated cellulose chains, further supporting greater mechanical performance. Complexation with Fe(III) at pH 1.99 suggests the formation of mono-coordinate bonds that support greater stability between cellulose chains, in which increased ductility with maintained strength was associated with significantly higher crosslinking elastic moduli (MC 3: 11.93±2.29 kPa vs. pH 1.99: 19.53±2.31 kPa). At pH 5.73, lower iron deposition probed by EDXA, increased crystallinity observed with XRD, as well as, increased relative FTIR absorbance of methyl stretching, indicated the formation of stable and mobile coordinate and methyl bridges, supporting optimal toughness (MC 3: 7.25±4.88 (* 10⁴) U_{T} vs. pH 5.73: 112.76±55.09 (* 10⁴) U_{T}) and ductility (MC 3: 11.28±5.57% vs. pH 5.73: 29.85±4.03%) with retained strength. The decrease in tensile and viscoelastic properties under alkaline conditions reinforce the claim that chemical crosslinks through hydroxyl, methyl, and coordinate bonding can be altered based upon pH. Thus, the enhancement and stabilization of mechanical toughness and strength with cellulose-tannin-iron complexes may be accredited to the prevention of hydrogen bonding between cellulose chains by increasing chemical interaction with hydroxyl, methyl, and coordinate bonds that distributes applied stress.

Tannic acid exhibits antimicrobial and anti-biofilm properties against various pathogenic bacterial strains, such as E. coli, S. aureus, and P. aeruginosa (Akiyama et al., 2001; Chung et al., 1998; Colak et al., 2010; Lee et al., 2013; Zhang, Sun et al., 2020). Mechanistically, tannic acid has been reported to not only complex with enzymes within bacterial membranes, inhibiting quorum sensing (Lee et al., 2013), but also complex with metals in the environment, preventing bacteria from absorbing essential metal ions for survival (Akiyama et al., 2001; Chung et al., 1998). Testing the antimicrobial potential of supramolecular tannic acid bioprocessing, microbial cellulose complexed with tannic acid and iron were utilized in a Kirby-Bauer disk diffusion test against gram-negative E. coli (FIG. 10). As-fabricated mCellulose exhibited no inhibition zones after 24 hours of culture, indicating that pristine groups do not inherently have antimicrobial properties. Although dried mCellulose films only treated with 0.5% w/v tannic acid significantly increased inhibition zones to 12.44±1.05 mm, samples complexed with Fe(III) at pH 3 and 5-6 significantly suppressed the diameter of the inhibition zone, further indicating that iron ions crosslink the polymer network, limiting the polyphenol's bioavailability and subsequent leaching.

Despite the instability of as-fabricated microbial cellulose's mechanical properties, optimal bioprocessing strategies based upon bioplasticizing or crosslinking were determined to be either 2% w/v sorbitol, 1% v/v glycerol, or pH 5.73 TA-Fe complexation. Optimal bioprocessing groups (blue bars) exhibited increased specific toughness (TA-Fe-pH 5.73: 5.33±1.79 U_{T}/g/cm³), comparable to cotton and LDPE films, albeit silk (16.02±3.21 U_{T}/g/cm³) and polyester (39.67±10.06 UT/g/cm³) remained significantly greater (FIG. 12b). Surprisingly, plasticizing and crosslinking increased ductility higher than cotton and silk samples, however, polyester and LDPE films presented superior elongation at break. Here, bioprocessing mCellulose demonstrates the development of biofabricated textiles with enhanced mechanical performance that are within range to contend against traditional textiles.

To evaluate the human toxicity and ecological damage impacts of traditional plasticizers compared to optimal bioprocessing strategies (2% w/v sorbitol, 1% v/v glycerol, and pH 5.73 TA-Fe), a life cycle impact assessment was performed using databases within EcoInvent (Wernet et al., 2016). In the chemical industry, traditional plasticization with phthalate esters is often implemented for polyvinyl chloride plastic to be processed with decreased inherent brittleness Howell, Lazar, 2019; Rahman, Brazel, 2004). The manufacturing impact of phthalates was demonstrated in LCA by modelling the immense ecological and human damage, and global warming produced by di-2ethylhexyl phthalate. Bioprocessing with sorbitol, glycerol, and TA-Fe offered significant improvements in environmental performance compared to traditional DOP-based plasticizers, which has the largest total impacts of the group. Although tannic acid—iron (III)—based processing methods were observed to have the largest total impact (0.015-0.044 mPts) when compared against sorbitol and glycerol, crosslinking processes may limit the polyphenol's subsequent leaching into the environment, circumventing the characteristic migration of plasticizers. This is supported by observations from previous reports by Martinez et al., who investigated the effects of crosslinking strategies on modulation of drug release profiles from polymers, in which stronger crosslinking reduced drug release (Martinez et al., 2014). Of note, LCA analysis was performed based upon laboratory technical grade processing procedures, in which further examination of utilizing renewable sources of plasticizers and crosslinkers may reduce human toxicity, ecological damage, and carbon footprint impacts (Schiros et al., 2022). Thus, the ability to control the source of bioprocessing chemicals supports the implementation of green chemistry strategies for tuning microbial cellulose performance within a circular materials economy.

Harnessing green bioprocessing techniques coupled with microbial biofabrication, the development of mCellulose with enhanced and stabilized mechanical behavior was achieved without the significant human health and environmental impacts observed with conventional plasticizers. Two post-processing approaches utilizing glycerol and sorbitol as bioplasticizers offers enhancement of toughness in a concentration dependent manner, eliminating the use of endocrine-disrupting phthalates for improving performance properties. An alternative crosslinking procedure incorporating tannic acid and iron ion complexation modified mCellulose with apparent antimicrobial properties, as well as, enhanced toughness and strength, which was strongly dependent upon pH conditions. Life cycle assessment revealed the application of glycerol reduced manufacturing impacts over one magnitude lower than tannic acid and iron complexation treatment, albeit the source of glycerol production can further mitigate impacts. In particular, glycerol sourced from vegetable oil reduced both human toxicity and ecological damage impacts by a magnitude lower than glycerol manufactured by epichlorohydrin obtained in waste oil.

Further, this invention explored microbial cellulose crosslinking with tannic acid-based metal complexation for high performance alternatives to cotton, synthetic films (low density polyethylene), and biomedical textiles. Further implementation of naturally occurring polyphenol-cellulose chelation to different metal ions shows immense potential to modulate mechanical performance of biotextiles with antibacterial properties, while reducing bioactive molecule leaching and migration out of the bulk material. Thus, the post-processing platform developed here supports the implementation of green chemistry strategies for tuning mCellulose within a circular materials economy, meeting key design criteria such as low toxicity, rapid renewability, and high performance.

### Purification

Biofabrication has emerged as a manufacturing platform using bioinspired raw materials, such as cells, biomolecules, extracellular matrices, and biomaterials, to develop living and non-living biological products, through an abundance of bioassembly-based technologies (Mironov et al., 2009). In particular, tissue engineering has adopted biofabrication approaches for the development of biodegradable electrospun scaffolds as grafts to promote cell guided regeneration of tissues damaged through injury or disease (Langer, 1993). Such approaches have been implemented to electrospin synthetic (PGA (Lin et al., 1999), PCL (Gurlek et al., 2017), PLLA (Lu et al., 2005), PLGA (James et al., 2011)), and natural (silk (Chen et al., 2003; Altman et al., 2002), collagen (Dunn et al., 1995; Bellincampi et al., 1998), hyaluronic acid (Cristino et al., 2005), alginate (Majima et al., 2005), chitosan (Hansson et al., 2012)) polymers to address significant clinical challenges, such as anterior cruciate ligament (ACL) rupture, which is hampered by 250,000 annual reconstructions accounting for more than $2.6 billion in healthcare costs (Nicholls et al., 2018). However, traditional electrospinning procedures solubilize polymers with industrial solvents, such as dimethylformamide (DMF), dichloromethane (DCM), or trifluoroethanol (TFE), which all have associated ecological damage and health impacts, restricting them from use in pharmaceuticals by the U.S. Food and Drug Administration (FDA) (Harmonisation ICf. Impurities, 2018). Thus, green manufacturing for tissue engineering has become an emerging approach to mimic the biological properties of the natural tissue, and promote regeneration, while mitigating negative environmental impacts (Smith, Grande, 2015; Mosher et al., 2021).

Inspired by the synthesis and modular bottom-up assembly of nanocellulose by microbes that meet key design criteria for sustainable high-performance nanofibers (Schiros et al., 2022), microbial cellulose has emerged as a green manufactured biomaterial strategy for tissue engineering (Svensson et al., 2005; Favi et al., 2013; Klemm et al., 2001; Zaborowska et al., 2010; Vielreicher et al., 2018; Kim et al., 2011; Loh et al., 2018; Torres et al., 2012). Microbial cellulose is a highly crystalline and chemically pure extracellular polysaccharide, free of hemicellulose and lignin, secreted by gram-negative bacteria, such as Acetobacter xylinum (Huang et al., 2014). Under specific aerobic culturing conditions these bacteria biofabricate unaligned cellulose nanofibrils (10-100 nm diameter) that coagulate into a three-dimensional layered hydrogel (>98% water) (Mohite et al., 2018; Gelin et al., 2007), known as a pellicle, at the air-culture media interface. Compared to plant cellulose, microbial cellulose is an advanced biomimetic material due to its inherent physiochemical properties, including high tensile strength, high degree of polymerization (Dahman, 2009), crystallinity (80-90%), high hygroscopicity and swelling capacity, moldability, biodegradability in composting environments, and biocompatibility (Choi, Shin, 2020). In particular, microbial cellulose has gained significant interest for biomedical applications, owing to the fabrication of a hydrated nanofibrous network morphologically similar to collagen fibrils within the eukaryotic extracellular matrix (ECM).

Moreover, its ability to support cell viability and cellular biosynthesis as a biomimetic extracellular matrix scaffold has been demonstrated for in-vitro tissue engineering applications such as, skin (Sulaeva et al., 2015), cartilage (Svensson et al., 2005; Favi et al., 2013), blood vessels (Klemm et al., 2001), menisci (Bodin et al., 2007), bone (Zaborowska et al., 2010; Vielreicher et al., 2018), and neuronal (P6rtile et al., 2012) constructs (Huang et al., 2014; Gama et al., 2016). Research success within in-vivo systems has further shown microbial cellulose's biocompatibility and capability for tissue integration without inducing inflammation or the formation of fibrotic capsules around the implanted scaffold (Klemm et al., 2001; Helenius et al., 2006).

Despite the concern of eliciting an immunogenic response from a gram-negative bacterial based biomaterial, purified microbial cellulose-based scaffolds for wound dressings, such as Dermafill® (Portal et al., 2009), and XCell® (Frankel et al., 2004), have been successfully translated to clinic, achieving the United States Food and Drug Administration's (FDA) requirements (<0.5 endotoxin units (EU)/mL or <20 EU/device) for endotoxin concentration in medical devices (FDA U. Guidance for industry, 2012). However, the purification of microbial cellulose is an immense challenge in laboratory and scaled-up industrial settings. The post-processing sterilization of microbial cellulose consists of not only removing gram-negative bacterial content, including membrane-based lipopolysaccharides (LPS), also known as endotoxins, but also eliminating traces of chemical compounds utilized during purification. Thus, a hydrated biofilm composed of only cellulose nanofibers and water may be prepared. Endotoxins are pyrogenic phospholipids, comprised of O-antigen glycan, core oligosaccharide, and hydrophobic toxic lipid A, known to trigger an immune system response (Gorbet, Sefton, 2005; Heine et al., 2001). Briefly, the presence of LPS in the blood stream initiates an immune response cascade, in which endotoxins are bound by transmembrane toll-like receptor 4 (TLR-4) on host innate immune cells, resulting in the secretion of pro-inflammatory cytokines (TNF-α, IL-6, IL-1β, and IFN-γ) during inflammation that potentially leads to anaphylactic shock (Gorbet, Sefton, 2005; Heine et al., 2001; Ronni et al., 2003; Saitoh, Miyake, 2006).

As endotoxin compounds are inherently heat-resistant, traditional purification techniques, such as high-pressure autoclave sterilization, are not appropriate as the only approach for microbial cellulose purification. Thus, purification strategies for microbial cellulose has led to facile approaches for the deactivation and removal of endotoxins using sodium hydroxide at low concentrations via immersion techniques (Svensson et al., 2005; Favi et al., 2013; Klemm et al., 2001; Zaborowska et al., 2010; Vielreicher et al., 2018; Kim et al., 2011; Loh et al., 2018; Torres et al., 2012). However, very few studies evaluate their purification approaches to efficiently eliminate endotoxins, as well as remove chemical compounds during sterilization from thin microbial cellulose networks, prior to use as biomimetic extracellular matrix scaffold. Despite this, there is additional concern for the utilization of similar purification processes for microbial cellulose scaffolds of varying thicknesses and porosities (Martinez et al., 2014; Pigaleva et al., 2019). Thus, there is a need to evaluate the efficacy of conventional purifying approaches for preparing endotoxin-ridden microbial cellulose scaffolds that do not elicit an immune system response.

Ultimately, this application aims to evaluate a purification procedure to support the preparation of microbial cellulose into a biocompatible, biomimetic nanofibrous scaffold that is suitable for in-vitro cell culture. A sequential chemical and high-pressure steam sterilization approach, based upon traditional purification procedures, would be assessed for the reduction of bacterial presence and endotoxin content. To this end, the biocompatibility of mCellulose would be determined by the inflammatory response of naïve THP-1 macrophages, and through anterior cruciate ligament fibroblast culture.

The emergence of biofabrication for tissue engineering has led to the development of biomaterial-based scaffolds that promote cell guided regeneration of tissues damaged through injury or disease (Langer, 1993). In particular, the design of nanofiber-based materials as a biomimetic approach is widely accepted for the regeneration of connective tissues, such as ligaments and tendons (Moffat et al., 2009; Lu et al., 2005; Kumbar et al., 2008), bone (Yoshimoto et al., 2003), and cartilage (Garrigues et al., 2014; Li et al., 2003), due to its ability to structurally mimic natural collagenous extracellular matrix of these tissues. The inherent high surface-to-volume ratio of nanofibers, as well as the ability to tune fiber diameter and alignment provides a platform demonstrated to direct and enhance cell adhesion (Moffat et al., 2009), thus supporting subsequent cell proliferation and matrix deposition (Folkman, Moscona, 1978). In addition, the surface chemistry of nanofibrous scaffolds can be tuned by fabricating with functionalizable synthetic (PGA (Lin et al., 1999), PCL (Gurlek et al., 2017), PLLA (Lu et al., 2005), PLGA (James et al., 2011)), and natural (silk (Chen et al., 2003; Altman et al., 2002), collagen (Dunn et al., 1995; Bellincampi et al., 1998), hyaluronic acid (Cristino et al., 2005), alginate (Majima et al., 2005), chitosan (Hansson et al., 2012)) polymers, promoting cell responses.

Following biomaterial implantation, cells of the host's innate immune system, such as monocytes and macrophages, are the first to respond, driving the motivation for investigating fibrous scaffold nano- and micro-environmental cues that dictate host immune response for improved functional tissue integration. Thus, the development and fabrication of fibrous matrices for regeneration of connective tissues met key design criteria, including a biomimetic structure replicating the extracellular matrix that does not elicit intensified inflammatory immune responses. In an attempt to understand the effect of fibrous scaffolds on the immune system, Bowlin et al. evaluated the in-vitro innate and acquired immune responses to electrospun poly(p-dioxanone) unaligned fibers blended with varying ratios of collagen type I or elastin (Garg et al., 2009; Smith et al., 2010; Cao et al., 2010). While significant immunosuppressive responses were observed for acquired immunity, it was found that these biomaterials had very few effects on innate immunity with comparable growth factor (VEGF and aFGF) secretion profiles between human peripheral blood monocyte-derived macrophages seeded upon fibers and tissue culture plastic polystyrene. To this end, although the presence of these fibrous biomaterials was observed to have minimal influence on innate macrophage response, further investigation of tailored micro-environmental cues is relevant to modulate immune cell response (Sridharan et al., 2015).

Topographical cues, such as fiber diameter and alignment, play a notable role on macrophage behavior (cell adherence, morphology, and secretion of cytokine molecules) (Cao et al., 2010; Garg et al., 2013; Saino et al., 2011). For example, Cao et al. investigated the in-vitro and in-vivo response to unaligned and aligned poly(ε-caprolactone) (PCL) nanofiber scaffolds (Cao et al., 2010). While in-vitro results demonstrated minimal human primary monocyte attachment to aligned matrices compared to unaligned scaffolds, in-vivo studies revealed that alignment enhanced cell penetration into the scaffold, and reduced thin fibrous capsule formation, eluding to minimal pro-inflammatory host response (Cao et al., 2010). In contrast, Saino et al. assessed the attachment of RAW 264.7 macrophages onto unaligned and aligned poly(L-lactic) (PLLA) scaffolds, in which it was found that greater cell adherence occurs on aligned fiber matrices compared to unaligned counterparts during 24 hours of in-vitro culture (Saino et al., 2011). Of note, it was reported that secretion of pro-inflammatory cytokines was strictly dependent upon fiber diameter rather than fiber alignment, such that nanofiber scaffolds minimized inflammatory responses compared to microfibers (Saino et al., 2011). However, these results are not supported by Garg et al., whom indicated that increasing the diameter of poly(p-dioxanone) supported mouse bone marrow-derived macrophages to express greater tissue regenerative phenotypes and reduced pro-inflammatory expression (Garg et al., 2013). Thus, the varying effects that scaffold topography cues, such as fiber diameter and alignment, have on macrophage attachment and activation indicates that other micro-environmental stimuli (surface chemistry, dynamic loading) may provide greater modulation for cell response.

Implantation of biomaterial scaffolds that aim to promote cell guided regeneration of load-bearing tissues, such as ligaments and tendons, are subjected to mechanical forces that subsequently stimulate infiltrating cells. Thus, in addition to topographical stimuli, mechanical loading of fibrous based scaffolds is investigated as an additional micro-environmental cue to activate and modulate macrophage phenotype (Ballotta et al., 2014; Schoenenberger et al., 2020; Xu et al., 2022). Previous work evaluating human THP-1 naïve M0 macrophage response to mechanical stimulation on unaligned PCL scaffolds revealed dynamic strains as low as 3% at 1 Hz elicited an pro-inflammatory response within 24 hours (8 hours of loading) of culture, in which higher strains at 7% enhanced M1-like activation (Schoenenberger et al., 2020). In addition, Ballotta et al. reported that human peripheral blood mononuclear macrophages (hPBMCs) seeded upon unaligned poly(ε-caprolactone) bisurea scaffolds subjected to 7% cyclic stretch at 0.8 Hz upregulated pro- and anti-inflammatory cytokine markers, as well as, deposited increased α-SMA after 24 hours, supporting the development of a reparative response (Ballotta et al., 2014). Taken together, these data demonstrate that various microenvironmental cues can polarize macrophages towards pro- and anti-inflammatory phenotypes, in which identification of successful design criteria can modulate host immune response for improved tissue integration.

The e effect of sterilization techniques to purify microbial-based cellulose was investigated, in which further biocompatibility was assessed through cell culture with human THP-1 derived macrophages and bovine anterior cruciate ligament fibroblasts. It is shown that sequential immersion of microbial cellulose into cellulase, 70% ethanol, and 0.1 M sodium hydroxide, followed by autoclaving, throughout three days significantly reduced both bacterial cell viability and culture medium residues, as indicated by cell membrane rupture of bacteria through the bulk of biofilm, the removal of brown melanoidin depositions produced from a Maillard reaction between amino acids and reducing sugars during biofabrication (Ellis, 1959; Martins et al., 2000), and depletion of sodium (1.88±0.36% to 0.064±0.060%) and phosphorus (3.44±0.602% to 0.00±0.00%). As the drop of sodium content can be explained by the removal of sodium-based compounds from Hestrin-Schramm culture medium, reduction of phosphorus suggests a decrease in bacterial presence, as bacterial membranes are comprised of phospholipids.

Both 70% ethanol and 0.1 M NaOH were specifically utilized to lyse bacteria, and release their inherent membrane-based lipopolysaccharides, thus hydrolyzing the toxic lipid A and reducing pyogenic components from microbial cellulose (Sandle, 2011). Although sodium hydroxide treatment at a vast range of concentrations (0.1-3 M) is widely accepted for traditional purification of microbial cellulose (Gama et al., 2016; Rajwade et al., 2015; Zhang et al., 2015; Favi et al., 2013; Vielreicher et al., 2018; P6rtile et al., 2012; Martinez et al., 2012; Zhijiang et al., 2011), it has been shown that large molarities (>2.5 M) can chemically and structurally alter Cellulose I to Cellulose II within crystalline domains (Oh et al., 2005; Dinand et al., 2002; Zugenmaier, 2001). Here, while cellulose chemical structure was observed to be maintained with alkaline treatment, endotoxin content, measured by a Limulus Amebocyte Lysate assay, was found to significantly increase from 5.23±0.46 (*10⁴) to 6.83±0.14 (*10⁴) EU after 24-hour immersion in 0.1 M NaOH prior to washing. Following, extensive washing of microbial cellulose with deionized water to remove NaOH in conjunction with autoclaving significantly reduced endotoxin content from 6.83±0.14 (*10⁴) to 0.49±0.46 (*10⁴) EU, albeit not significant to as-fabricated groups displaying intact bacterial membranes. Collectively, this purification process was not able to reduce concentrations lower than FDA's requirement (20 EU/device) for medical devices (FDA U. Guidance for industry, 2012). Alternatively, in previous work by Martinez et al. using perfusion with 0.5 M NaOH to purify microbial cellulose, the opposite effect was observed, with a decrease in endotoxin content after alkaline treatment for 28 days, suggesting hydrolyzing is more efficient at higher concentrations with longer treatments (Martinez et al., 2014). Interestingly, the use of extensive washing with deionized water to remove endotoxins observed in this invention is supported by Martinez et al. results demonstrating that endotoxin content can be further reduced by continuously washing biofilms in endotoxin-free water for 14 days as a secondary depyrogenation process (Martinez et al., 2014). Of note, the purification procedure utilized in this invention was performed with conventional sterilization techniques on thin microbial cellulose biofilms (7 mm), in which the demand for thicker purified films may suggest alternate strategies to improve solvent diffusion and removal from the biomaterial. Recent work has shown that purifying dense (>20 mm) microbial cellulose biofilms with sodium hydroxide within a perfusion system (Martinez et al., 2014), as well as, under high pressure supercritical carbon dioxide (Pigaleva et al., 2019), provides immense potential as effective sterilization approaches for large scale manufacturing.

With respect to the biocompatibility of microbial cellulose, results from this application show that no significant pro- or anti-inflammatory responses were observed when culturing THP-1 macrophages on purified cellulose scaffolds throughout four days. Synthetic PLGA:PCL microfiber scaffolds were utilized as control due to its well-established biocompatibility, in which there were no observed differences in immune response with purified microbial cellulose. Although less macrophages adhered to purified microbial cellulose than PLGA:PCL, cells on both scaffolds released comparable TNF-α, IL-1β, IL-6, IL-IRA, and IL-10 per cell, and maintained a round morphology resembling naïve M0 phenotypes (Lee et al., 2013), suggesting an equivalent immune cell response. This is similar to results observed by Daneshmandi et al., who investigated the response of murine peritoneal macrophages to microbial cellulose (Daneshmandi et al., 2011). In-vitro studies demonstrated that microbial cellulose did not elicit a significant cytotoxic nor stimulatory effect from peritoneal macrophages throughout 48-hour culture. Additional investigation of the in-vivo response to microbial cellulose by Martinez et al. and Jing et al., revealed that microbial cellulose is non-cytotoxic, although it induces a slight foreign body response with minimal infiltration of macrophages, as well as, increases inflammatory cytokine expression, respectively (Martinez et al., 2014; Jing et al., 2020). Interestingly, the findings of this application also indicated as-fabricated microbial cellulose as well as 70% ethanol treated microbial cellulose did not elicit an immune response from THP-1 macrophages. Although it was observed that there was interaction between G. xylinus and naïve macrophages, total amounts of TNF-α, IL-1β, IL-6, IL-1RA, and IL-10 were comparable to secretion from macrophages cultured on PLGA:PCL and purified microbial cellulose. Therefore, it is possible that the effects of G. xylinus on macrophage activation can be investigated by other pro-inflammatory pathways such as reactive oxygen species production (Junka et al., 2017).

Additionally, purified microbial cellulose biocompatibility was evaluated using bovine anterior cruciate ligament fibroblasts. It is shown that throughout 14 days of culture, cells remained viable and proliferated, with significant increases in cell number at each time point. In comparison to unaligned PLGA:PCL microfiber scaffolds, purified microbial cellulose had significantly less cells adhered during culture, despite initially seeding both scaffolds with similar cell densities. After 1 day in culture, fibroblasts seeded on purified microbial cellulose displayed distinct larger round morphologies without pronounced elongated spindles, in comparison to the characteristic spindled morphology observed on PLGA:PCL scaffolds. However, by day 14 fibroblasts exhibited an elongated morphology. Differences observed in cell attachment and morphology by day 1 in culture may suggest that differences in surface stiffness altered cell phenotypes. Previous work has shown that fibroblasts exhibit flatter morphologies on stiffer substrates than on softer matrices as a result of greater traction force effecting their migration on surfaces (Lo et al., 2000; Bray, 2000). Here, fibroblasts were observed to maintain a round morphology by day 1 on soft hydrated microbial cellulose scaffolds with Young's Moduli of 2 MPa, unlike the spread and elongated morphology demonstrated on PLGA:PCL scaffolds with elastic moduli of 20 MPa. These observations corroborate previous reports of altered mesenchymal stem cell or dermal fibroblast morphology when cultured on substrates such as bacterial cellulose and tissue culture polystyrene (Favi et al., 2013; Loh et al., 2018). It is possible that fibroblasts cultured upon microbial cellulose have retracted filopodium and lamellipodium for cell spreading, supporting their round morphology, further limiting migration and proliferative capacity.

To this end, this invention indicates that mCellulose can be purified by conventional sterilization techniques to remove bacterial content and culture medium residue, supporting the fabrication of a biocompatible substrate that not only prevents macrophage activation, but is also favorable for cells to attach, proliferate, and remain viable. This purification system demonstrates the ability to reduce bacterial cell viability within the biomaterial using simple immersion practices, without altering cellulose chemical structure. Overall pro- and anti-inflammatory immune response and cell attachment remained low on mCellulose scaffolds, suggesting that this substrate may minimize inflammatory response by infiltrating immune cells upon implantation. Clinically, it is proposed that this microbial cellulose approach may use G. xylinus to biofabricate a graft to a designed macro- and micro-morphology, in which after subsequent purification and post-processing, the scaffold may be implanted. It is desirable to investigate optimal purification strategies to further reduce endotoxin content, as well as, control mCellulose fiber alignment and porosity to achieve improved cell-biomaterial interaction.

This application investigated a sterilization procedure, based upon conventional chemical and high-pressure steam approaches, for purifying microbial cellulose biofilms. It was demonstrated that sequential immersion of biofilms into cellulase, 70% ethanol, and 0.1 M sodium hydroxide, followed by autoclaving, reduced both bacterial cell viability and endotoxin concentration, without modifying cellulose chemical structure. To this end, purified microbial cellulose did not stimulate a pro-inflammatory response from naïve THP-1 macrophages, and supported fibroblast viability and proliferation for two weeks. Further, these results demonstrate the potential of microbial cellulose as a biocompatible scaffold.

### Substrate Alignment and Mechanical Stimulation

The role of chemical induction, microfiber matrix alignment, and mechanical stimulation on guiding macrophage polarization was evalauated by determining the relative contribution of each cue as well as their combined influence. Here, THP-1 macrophages were successfully seeded, and polarized onto unaligned and aligned PLGA:PCL microfiber matrices, in which it was demonstrated that mechanical loading can facilitate immunomodulation of macrophage response. It is shown that guidance of macrophage phenotype with mechanical stimulation is only possible when induced pro-inflammatory M1 cells are cultured on microfibers, however, matrix orientation dictates modulation towards greater pro- or anti-inflammatory response. On unaligned meshes, loading M1-induced macrophages reduced the secretion of M1 cytokines TNF-α, IL-1β, and IL-6, as well as enhanced M2-based IL-10, IL-1RA, and TARC production, indicating that attached cells are transitioned toward an anti-inflammatory phenotype. In contrast, loading aligned fibers seeded with M1-induced macrophages elicited a greater pro-inflammatory response, with reduced anti-inflammatory cytokine secretion, signifying a transition toward a pro-inflammatory phenotype. Although results from this application suggest that microfiber matrix and chemical induction as stimulatory cues can separately promote macrophage activation, it is the combined stimulation between polarization and mechanical loading that plays a relevant role in immunomodulation of macrophage response, in which further tuning of fiber alignment can alter cell phenotype.

In evaluating initial cell attachment, significantly greater naïve cells attached to unaligned PLGA:PCL than aligned matrices after monocyte differentiation. Macrophage attachment onto substrates with varying topographies has been reported to influence immune response activation towards pro- or anti-inflammatory phenotypes (Saino et al., 2011; Schoenenberger et al., 2020; Shaw et al., 1990). Thus, these findings suggest that greater attachment on unaligned PLGA:PCL corresponds to enhanced macrophage activation compared to aligned meshes. Cytokines are known to bias macrophage fate within a spectrum of pro- or anti-inflammatory phenotypes, influencing the initiation and intensity of host immune response (Saino et al., 2011; Arango, Descoteaux, 2014). Here, further investigation of cytokine secretion after initial cell attachment revealed that cells adhered to aligned microfibers significantly released less M1: IL-1β, IL-6, and M2: IL-10, IL-IRA, and TARC per cell, proposing that aligned PLGA:PCL topographies may minimize pro- or anti-inflammatory responses in-vivo compared to unaligned counterparts. These observations corroborate previous reports of minimal human primary monocyte attachment onto aligned PCL scaffolds compared to unaligned matrices in-vitro (Cao et al., 2010; Schoenenberger et al., 2020). Additional in-vivo studies showed that aligned PCL scaffolds have greater potential as a tissue engineered scaffold than unaligned meshes due to enhanced cell penetration and thin fibrous capsule formation, eluding to minimal host response (Cao et al., 2010). Thus, given these cell-biomaterial interactions in an early inflammation stage (24 hours), macrophages may have reduced activation on aligned topographies that resemble healthy tissue, however, activation is enhanced when microenvironments mimic disorganized collagen matrix (injured tissue) (Schoenenberger et al., 2020). Conversely, greater macrophage-like RAW 264.7 cell adherence was observed on aligned micro- and nano-fiber poly(L-lactic) (PLLA) matrices compared to unaligned counterparts during 24 hours of in-vitro culture (Saino et al., 2011). Thus, the role of scaffold topography in promoting macrophage attachment and activation may be also dependent upon polymer surface chemistry.

In terms of cell density, naïve macrophages proliferated faster with greater population doublings when cultured on loaded aligned matrices, resulting in a significantly greater cell number by day 4 of culture. Although characterized by poor cell attachment compared to unaligned matrices at day 2, these findings suggest that matrix alignment coupled with mechanical stimulation are the primary cues for naïve macrophage proliferation. These observations validate previous reports of enhanced macrophage proliferation with mechanical stimulation (Xu et al., 2022; Sager et al., 2016), such as in studies by Xu et al. who applied tensile stimulation to bone-marrow-derived macrophages, revealing that loading induces proliferation through upregulation of mechanosensory ion channels, such as Piezol (Xu et al., 2022). Despite differences in loading regimen, a significantly greater M0 macrophage cell number on aligned PLGA:PCL microfibers in response to loading may be attributed to upregulation of mechanosensory channels that enhance proliferation during mechanical stimuli. Additionally, in previous work by Subramony et al., who investigated the effects of matrix alignment and mechanical stimulation on mesenchymal stem cell differentiation, it was suggested that mechanical loading may enhance cell growth compared to unloaded cultures as a means of improved nutrient diffusion (Subramony et al., 2014). However, the results of this study reveal mechanical loading on unaligned meshes had no observed differences on naive M0 macrophage cell number to unloaded groups, indicating that greater cell density is characteristic of the interactive effect between matrix aligned topography and mechanical loading. Further, although polarized (M1 and M2) macrophages remained viable on both types of meshes by day 4, there were no observed differences in cell number after loading scaffolds seeded with M1-induced macrophages. Thus, this suggests that macrophages lose proliferative potential on loaded aligned microfibers once polarized to a pro-inflammatory phenotype via down regulation of mechanosensory ion channels.

In terms of matrix production, collagen and GAG deposition were observed for näive M0, and polarized (M1 and M2) cells on aligned and unaligned matrices that were stimulated with or without mechanical loading, albeit at much lower magnitude than previously observed for human mesenchymal stem cells (Subramony et al., 2014) and bovine articular chondrocytes (Mosher et al., 2021). This is similar to observations from previous reports by Ballotta et al., who investigated the effects of strain-dependent modulation on human peripheral blood mononuclear macrophages seeded upon unaligned poly(ε-caprolactone) bisurea scaffolds (Ballotta et al., 2014). In-vitro results indicated that hPBMCs synthesized GAG and Collagen III throughout a week of culture, contributing to early stage ECM synthesis, though loading at varying strains (0-12%) did not affect deposition (Ballotta et al., 2014). In contrast to these findings, the results of this study indicate that mechanical stimulation of aligned fibers seeded with M1-induced macrophages significantly increased per-cell collagen deposition, suggesting that co-stimulation interaction between polarization, matrix alignment, and mechanical loading may be required for promoted early stage ECM synthesis.

With respect to the roles of chemical induction and matrix alignment on macrophage activation, it was observed that M1-induced macrophages seeded on aligned scaffolds significantly increased per-cell secretion of anti-inflammatory cytokines (IL-10, IL-1RA, and TARC) compared to unaligned groups. Although a decrease in pro-inflammatory markers was not measured between unaligned and aligned groups, these findings suggest that M1 macrophages may identify aligned PLGA:PCL matrices as healthy microenvironments, supporting increased cell activation towards repair. Similar to Cao et al., who observed aligned PCL nanofibers minimized host pro-inflammatory response in-vivo, this suggests that nanofiber organization can play a primary role within inflammatory environments to drive anti-inflammatory response (Cao et al., 2010). Of note, with the exception of greater IL-10 per cell production measured in unaligned groups, there was minimal production and no observed differences in pro- or anti-inflammatory cytokine production of naïve M0 macrophages between culturing on aligned and unaligned matrices for 4 days. As this may elude to naïve cells polarizing toward a pro-inflammatory phenotype in response to microenvironments mimicking disorganized collagen matrix (injured tissue), the effect of mechanical loading with physiological strains was investigated as a definite cue for activating M0 macrophages.

Further, with respect to the roles of mechanical stimulation and matrix alignment on macrophage activation, there were no observed differences in pro- or anti-inflammatory cytokine per-cell secretion from naïve M0 cells before and after loading unaligned and aligned meshes with 1% strain. Previous work assessing human THP-1 naïve M0 macrophage response to mechanical stimulation revealed strains as low as 3% at 1 Hz elicited an pro-inflammatory response within 24 hours (8 hours of loading) of culture, in which higher strains at 7% enhanced M1-like activation (Schoenenberger et al., 2020). Ultimately, these results indicate that co-stimulation between PLGA:PCL matrix alignment and mechanical loading at low (1%) physiologic-relevant strains cannot modulate macrophage activation, thus further exogenous cues, such as higher mechanical strains or chemical induction, may be required.

Lastly, the findings of this study provide indication of an interactive effect between chemical induction, microfiber matrix alignment, and mechanical stimulation. Here, co-stimulation with pro-inflammatory polarization and mechanical loading may be deployed for modulating macrophage phenotype, however matrix alignment is the key determinant for guiding phenotypes towards greater pro- or anti-inflammatory states. On unaligned meshes, loading M1-induced macrophages reduced the secretion of M1 cytokines TNF-α, IL-1β, and IL-6, as well as enhanced M2-based IL-10, IL-1RA, and TARC production, indicating that attached cells are transitioned toward an anti-inflammatory phenotype. In contrast, loading aligned fibers seeded with M1-induced macrophages elicited a greater pro-inflammatory response, with reduced anti-inflammatory cytokine secretion, signifying a transition toward a pro-inflammatory state. Interestingly, this enhanced secretion of TNF-α per cell with M1 macrophages on loaded aligned fibers was also associated with significantly greater matrix deposition of collagen per cell. Although the use of mechanical loading, as an individual cue, has been shown to promote greater collagen production for MSCs on aligned and unaligned nanofibers (Subramony et al., 2013), outcomes utilizing comparable combination of exogenous cues on pro-inflammatory macrophages has not been elucidated.

Macrophages are known to not only degrade the extracellular matrix with metalloproteases, but also play an role in tissue regeneration and repair, in which different phenotypes are a result of specific environmental cues (Novak, Koh, 2013). Recent work shows that THP-1-derived macrophages have the ability to express upwards of 37 human collagen mRNAs (Schnoor et al., 2008; Weitkamp et al., 1999), suggesting the potential for contributing to structural integrity of matrices during tissue repair in pro- and anti-inflammatory states. As the correlation between TNF-α stimulation and enhanced collagen production has been reported in myofibroblast culture due to reduced matrix metalloprotease-2 expression and collagen degradation (Theiss et al., 2005), there is much more to be discovered for macrophage ECM signaling pathways during modulation of cell-matrix interactions.

To this end, the findings of this application indicate the varying roles of chemical induction, microfiber matrix alignment, and mechanical stimulation in macrophage activation, thus providing a modulatory platform for guiding inflammatory responses and early matrix deposition. Understanding the response as well as the resulting biochemical mechanisms of macrophages when influenced by exogenous cues and mechanotransduction is notable for connective tissue injury and repair. Here, it is shown that guidance of macrophage phenotype with an ACL physiologic relevant mechanical stimulation is only possible when induced pro-inflammatory M1 cells are cultured on microfibers, however, matrix orientation dictates modulation towards either pro-(aligned) or anti-(unaligned) inflammatory response. It would be desirable to investigate the incorporation of unaligned and aligned microbial cellulose scaffolds as a matrix platform for modulation of macrophage phenotype. Since previous work has established that nanofibers minimize inflammatory responses from macrophages compared to microfibers (Saino et al., 2011), the inherent green biofabrication of nanofibril cellulose in conjunction with mechanical loading offers great potential to guide immune cell activation.

This application investigated the coupled effects of chemical induction, microfiber matrix alignment, and mechanical stimulation on macrophage polarization. It was determined that low-strain mechanical loading of naïve M0 macrophages on aligned and unaligned PLGA:PCL microfibers did not guide cells toward either pro- or anti-inflammatory phenotypes. Although differences in M0 cell attachment and cell density on loaded or unloaded microfibers (aligned and unaligned) were observed, this suggests that ACL physiological-relevant mechanical loading alone does not guide naïve macrophage activation. Rather, it was demonstrated that loading M1-induced macrophages on unaligned matrices reduced the secretion of pro-inflammatory cytokines, while enhancing anti-inflammatory protein production, compared to the unloaded response of M1 macrophages. In contrast, loading aligned fibers seeded with M1-induced macrophages elicited a greater pro-inflammatory response, with reduced anti-inflammatory cytokine secretion. Ultimately, application of mechanical loading while tuning matrix alignment has a greater combined effect as stimulatory cues on pro-inflammatory cell response, than on naïve macrophages.

### REFERENCES

1. Huang Y, Zhu C, Yang J, Nie Y, Chen C and Sun D. Recent advances in bacterial cellulose. Cellulose. 2014; 21(1): 1-30.
2. Mohite B, Koli S and Patil S. Bacterial cellulose-based hydrogels: Synthesis, Properties, and Applications. Cellulose-based superabsorbent hydrogels: Springer Nature. 2018; 1-22.
3. Gelin K, Bodin A, Gatenholm P, Mihranyan A, Edwards K and Stromme M. Characterization of water in bacterial cellulose using dielectric spectroscopy and electron microscopy. Polymer. 2007; 48(26): 7623-7631.
4. Dahman Y. Nanostructured biomaterials and biocomposites from bacterial cellulose nanofibers. Journal of Nanoscience and Nanotechnology. 2009; 9(9): 5105-5122.
5. Choi S M and Shin E J. The Nanofication and Functionalization of Bacterial Cellulose and Its Applications. Nanomaterials. 2020; 10(3): 406.
6. Change NC. The price of fast fashion. https://www.nature. com/articles/s41558-017-0058-9 Acesso em. 2018; 7(06): 2018.
7. Chemiefaser I. Global chemical fiber production from 2000 to 2018, by fiber type (in 1,000 metric tons). 2019;
8. Weltbank. The Bangladesh Responsible Sourcing Initiative: A New Model for Green Growth?2014.
9. Kirchain R, Olivetti E, Miller TR and Greene S. Sustainable apparel materials. Materials Systems Laboratory, Massachusetts Institute of Technology, Cambridge. 2015;
10. De Falco F, Di Pace E, Cocca M and Avella M. The contribution of washing processes of synthetic clothes to microplastic pollution. Scientific reports. 2019; 9(1): 1-11.
11. Costa A, Rocha MAV and Sarubbo L. Bacterial cellulose: an ecofriendly biotextile. International Journal of Textile and Fashion Technology. 2017; 7(11-26.
12. Chan CK, Shin J and Jiang SXK. Development of tailor-shaped bacterial cellulose textile cultivation techniques for zero-waste design. Clothing and Textiles Research Journal. 2018; 36(1): 33-44.
13. Revin V, Liyaskina E, Nazarkina M, Bogatyreva A and Shchankin M. Cost-effective production of bacterial cellulose using acidic food industry by-products. brazilian journal of microbiology. 2018; 49(151-159.
14. Gama M, Gatenholm P and Klemm D. Bacterial nanocellulose: a sophisticated multifunctional material. CRC press. 2016.
15. Yilgor C, Yilgor Huri P and Huri G. Tissue engineering strategies in ligament regeneration. Stem cells international. 2012; 2012(
16. Nicholls M, Aspelund T, Ingvarsson T and Briem K. Nationwide study highlights a second peak in ACL tears for women in their early forties. Knee Surgery, Sports Traumatology, Arthroscopy. 2018; 26(2): 648-654.
17. COOPER R R, Misol S and Stimmel P. Tendon and ligament insertion: a light and electron microscopic study. JBJS. 1970; 52(1): 1-170.
18. Kannus P and Jarvinen M. Conservatively treated tears of the anterior cruciate ligament. Long-term results. JBJS. 1987; 69(7): 1007-1012.
19. Vacanti JP and Langer R. Tissue engineering: the design and fabrication of living replacement devices for surgical reconstruction and transplantation. The lancet. 1999; 354(S32-S34.
20. Erisken C, Zhang X, Moffat K L, Levine W N and Lu H H. Scaffold fiber diameter regulates human tendon fibroblast growth and differentiation. Tissue Engineering Part A. 2013; 19(3-4): 519-528.
21. Moffat K L, Kwei AS-P, Spalazzi J P, Doty S B, Levine W N and Lu H H. Novel nanofiber-based scaffold for rotator cuff repair and augmentation. Tissue Engineering Part A. 2009; 15(1): 115-126.
22. Harmonisation ICf Impurities: Guideline for Residual Solvents Q3C (R7). International Council for Harmonisation Geneva, Switzerland. 2018.
23. Smith B D and Grande D A. The current state of scaffolds for musculoskeletal regenerative applications. Nature Reviews Rheumatology. 2015; 11(4): 213-222.
24. Mosher C Z, Brudnicki P A, Gong Z, Childs H R, Lee S W, Antrobus R M, Fang E C, Schiros T N and Lu H H. Green electrospinning for biomaterials and biofabrication. Biofabrication. 2021; 13(3): 035049.
25. Chen J, Altman G H, Karageorgiou V, Horan R, Collette A, Volloch V, Colabro T and Kaplan D L. Human bone marrow stromal cell and ligament fibroblast responses on RGD-modified silk fibers. Journal of Biomedical Materials Research Part A: An Official Journal of The Society for Biomaterials, The Japanese Society for Biomaterials, and The Australian Society for Biomaterials and the Korean Society for Biomaterials. 2003; 67(2): 559-570.
26. Altman G H, Horan R L, Lu H H, Moreau J, Martin I, Richmond J C and Kaplan D L. Silk matrix for tissue engineered anterior cruciate ligaments. Biomaterials. 2002; 23(20): 4131-4141.
27. Dunn M G, Liesch J B, Tiku M L and Zawadsky J P. Development of fibroblast-seeded ligament analogs for ACL reconstruction. Journal of biomedical materials research. 1995; 29(11): 1363-1371.
28. Bellincampi L D, Closkey R F, Prasad R, Zawadsky J P and Dunn M G. Viability of fibroblast-seeded ligament analogs after autogenous implantation. Journal of Orthopaedic Research. 1998; 16(4): 414-420.
29. Cristino S, Grassi F, Toneguzzi S, Piacentini A, Grigolo B, Santi S, Riccio M, Tognana E, Facchini A and Lisignoli G. Analysis of mesenchymal stem cells grown on a three-dimensional HYAFF 11®-based prototype ligament scaffold. Journal of Biomedical Materials Research Part A: An Official Journal of The Society for Biomaterials, The Japanese Society for Biomaterials, and The Australian Society for Biomaterials and the Korean Society for Biomaterials. 2005; 73(3): 275-283.
30. Majima T, Funakosi T, Iwasaki N, Yamane S-T, Harada K, Nonaka S, Minami A and Nishimura S-I. Alginate and chitosan polyion complex hybrid fibers for scaffolds in ligament and tendon tissue engineering. Journal of Orthopaedic Science. 2005; 10(3): 302-307.
31. Hansson A, Hashom N, Falson F, Rousselle P, Jordan O and Borchard G. In vitro evaluation of an RGD-functionalized chitosan derivative for enhanced cell adhesion. Carbohydrate polymers. 2012; 90(4): 1494-1500.
32. Silva M, Ferreira F N, Alves N M and Paiva M C. Biodegradable polymer nanocomposites for ligament/tendon tissue engineering. Journal ofNanobiotechnology. 2020; 18(1): 23.
33. Leong N L, Petrigliano F A and McAllister D R. Current tissue engineering strategies in anterior cruciate ligament reconstruction. Journal of biomedical materials research Part A. 2014; 102(5): 1614-1624.
34. Moniri M, Boroumand Moghaddam A, Azizi S, Abdul Rahim R, Bin Ariff A, Zuhainis Saad W, Navaderi M and Mohamad R. Production and status of bacterial cellulose in biomedical engineering. Nanomaterials. 2017; 7(9): 257.
35. Wang S-S, Han Y-H, Chen J-L, Zhang D-C, Shi X-X, Ye Y-X, Chen D-L and Li M. Insights into bacterial cellulose biosynthesis from different carbon sources and the associated biochemical transformation pathways in Komagataeibacter sp. W1. Polymers. 2018; 10(9): 963.
36. Singhsa P, Narain R and Manuspiya H. Physical structure variations of bacterial cellulose produced by different Komagataeibacter xylinus strains and carbon sources in static and agitated conditions. Cellulose. 2018; 25(3): 1571-1581.
37. Ruka D R, Simon G P and Dean K M. Altering the growth conditions of Gluconacetobacter xylinus to maximize the yield of bacterial cellulose. Carbohydrate polymers. 2012; 89(2): 613-622.
38. Shah N, Ul-Islam M, Khattak W A and Park J K. Overview of bacterial cellulose composites: a multipurpose advanced material. Carbohydrate polymers. 2013; 98(2): 1585-1598.
39. O'brien F J. Biomaterials & scaffolds for tissue engineering. Materials today. 2011; 14(3): 88-95.
40. Sridharan R, Cameron A R, Kelly D J, Kearney C J and O'Brien F J. Biomaterial based modulation of macrophage polarization: a review and suggested design principles. Materials Today. 2015; 18(6): 313-325.
41. Ogle M E, Segar C E, Sridhar S and Botchwey E A. Monocytes and macrophages in tissue repair: implications for immunoregenerative biomaterial design. Experimental Biology and Medicine. 2016; 241(10): 1084-1097.
42. Gardner A B, Lee S K, Woods E C and Acharya A P. Biomaterials-based modulation of the immune system. BioMed research international. 2013; 2013(
43. Franz S, Rammelt S, Scharnweber D and Simon J C. Immune responses to implants—a review of the implications for the design of immunomodulatory biomaterials. Biomaterials. 2011; 32(28): 6692-6709.
44. Pelling A E and Hickey R J. Cellulose biomaterials for tissue engineering. Frontiers in Bioengineering and Biotechnology. 2019; 7(45.
45. Galloway M T, Lalley A L and Shearn J T. The role of mechanical loading in tendon development, maintenance, injury, and repair. The Journal of bone and joint surgery. American volume. 2013; 95(17): 1620.
46. Subramony S D, Su A, Yeager K and Lu H H. Combined effects of chemical priming and mechanical stimulation on mesenchymal stem cell differentiation on nanofiber scaffolds. Journal of biomechanics. 2014; 47(9): 2189-2196.
47. Subramony S D, Dargis B R, Castillo M, Azeloglu E U, Tracey M S, Su A and Lu H H. The guidance of stem cell differentiation by substrate alignment and mechanical stimulation. Biomaterials. 2013; 34(8): 1942-1953.
48. Mironov V, Trusk T, Kasyanov V, Little S, Swaja R and Markwald R. Biofabrication: a 21st century manufacturing paradigm. Biofabrication. 2009; 1(2): 022001.
49. USDoCaIT A. The Medical Technology Industry in the United States. 2020.
50. Moroni L, Boland T, Burdick J A, De Maria C, Derby B, Forgacs G, Groll J, Li Q, Malda J and Mironov VA. Biofabrication: a guide to technology and terminology. Trends in biotechnology. 2018; 36(4): 384-402.
51. Langer R. VacantiJP. Tissue engineering. Science. 1993; 260(5110): 920-926.
52. Saxena I M and Brown Jr R M. Cellulose biosynthesis: current views and evolving concepts. Annals of botany. 2005; 96(1): 9-21.
53. Williams W S and Cannon R E. Alternative environmental roles for cellulose produced by Acetobacter xylinum. Appl. Environ. Microbiol. 1989; 55(10): 2448-2452.
54. Suresh S. Biosynthesis and assemblage of extracellular cellulose by bacteria. Handbook of Environmental Materials Management. Springer, Chain. 2018; 1-43.
55. Bielecki S, Krystynowicz A, Turkiewicz M and Kalinowska H. Bacterial cellulose. Biopolymers Online: Biology•Chemistry•Biotechnology• Applications. 2005; 5(
56. Hestrin S and Schramm M. Synthesis of cellulose by Acetobacter xylinum. 2. Preparation of freeze-dried cells capable of polymerizing glucose to cellulose. Biochemical Journal. 1954; 58(2): 345.
57. Rajwade J, Paknikar K and Kumbhar J. Applications of bacterial cellulose and its composites in biomedicine. Applied microbiology and biotechnology. 2015; 99(6): 2491-2511.
58. Ha S-J, Galazka J M, Kim S R, Choi J-H, Yang X, Seo J-H, Glass N L, Cate J H and Jin Y—S. Engineered Saccharomyces cerevisiae capable of simultaneous cellobiose and xylose fermentation. Proceedings of the National Academy of Sciences. 2011; 108(2): 504-509.
59. Auta R, Adamus G, Kwiecien M, Radecka I and Hooley P. Production and characterization of bacterial cellulose before and after enzymatic hydrolysis. African Journal of Biotechnology. 2017; 16(10): 470-482.
60. Wang S, Jiang F, Xu X, Kuang Y, Fu K, Hitz E and Hu L. Super-Strong, Super-Stiff Macrofibers with Aligned, Long Bacterial Cellulose Nanofibers. Advanced Materials. 2017; 29(35): 1702498.
61. Wang X, Wang Q and Xu C. Nanocellulose-Based Inks for 3D Bioprinting: Key Aspects in Research Development and Challenging Perspectives in Applications-A Mini Review. Bioengineering. 2020; 7(2): 40.
62. Greca LG, Lehtonen J, Tardy BL, Guo J and Rojas OJ. Biofabrication of multifunctional nanocellulosic 3D structures: a facile and customizable route. Materials Horizons. 2018; 5(3): 408-415.
63. Wang G, Chen X F, Shi X D, Yu L J, Liu B F and Yang G. Bio-fabrication of patterned cellulose nano-fibers. Trans Tech Publ. 2008.
64. Muthu S S. Roadmap to sustainable textiles and clothing: Eco-friendly raw materials, technologies, and processing methods. Springer. 2014.
65. Hasanbeigi A and Price L. A technical review of emerging technologies for energy and water efficiency and pollution reduction in the textile industry. Journal of Cleaner Production. 2015; 95(30-44.
66. Sajn N. Environmental impact of the textile and clothing industry. What consumers need to know, European Parliamentary Research Service. 2019;
67. Sandin G and Peters G M. Environmental impact of textile reuse and recycling-A review. Journal of Cleaner Production. 2018; 184(353-365.
68. Bruce N, Hartline N, Karba S, Ruff B, Sonar S and Holden P. Microfiber pollution and the apparel industry. University of California Santa Barbara, Bren School of Environmental Science & Management (accessed 19 Aug. 2016) http://brenmicroplastics. weebly. com/uploads/5/1/7/0/51702815/bren-patagonia_final_report. pdf 2016;
69. Agenda G F. The Boston Consulting Group.(2017). Pulse of the fashion industry. 2018; 139(
70. Hanif M A, Nadeem R, Rashid U and Zafar M N. Assessing pollution levels in effluents of industries in city zone of Faisalabad, Pakistan. Journal of Applied Science. 2005; 5(10): 1713-1717.
71. Akan J, Moses E, Ogugbuaja V and Abah J. Assessment of tannery industrial effluents from Kano metropolis, Kano State, Nigeria. Journal of Applied Sciences. 2007; 7(19): 2788-2793.
72. Altaf M M, Masood F and Malik A. Impact of long-term application of treated tannery effluents on the emergence of resistance traits in Rhizobium sp. isolated from Trifolium alexandrinum. Turkish Journal of Biology. 2008; 32(1): 1-8.
73. Khan A G. Relationships between chromium biomagnification ratio, accumulation factor, and mycorrhizae in plants growing on tannery effluent-polluted soil. Environment International. 2001; 26(5-6): 417-423.
74. Bhuiyan MAH, Suruvi N I, Dampare S B, Islam M, Quraishi S B, Ganyaglo S and Suzuki S. Investigation of the possible sources of heavy metal contamination in lagoon and canal water in the tannery industrial area in Dhaka, Bangladesh. Environmental monitoring and assessment. 2011; 175(1-4): 633-649.
75. Browne M A, Crump P, Niven S J, Teuten E, Tonkin A, Galloway T and Thompson R. Accumulation of microplastic on shorelines woldwide: sources and sinks. Environmental science & technology. 2011; 45(21): 9175-9179.
76. Boucher J and Friot D. Primary microplastics in the oceans: a global evaluation of sources. IUCN Gland, Switzerland. 2017.
77. Gam H J and Banning J. Addressing sustainable apparel design challenges with problem-based learning. Clothing and Textiles Research Journal. 2011; 29(3): 202-215.
78. Bhatia S. Pollution control in textile industry. CRC Press. 2017.
79. Collet C. Biotextiles: Evolving Textile Design Practices for the Bioeconomy and the Emerging Organism Industry. 2018;
80. Jang W D, Hwang J H, Kim H U, Ryu J Y and Lee S Y. Bacterial cellulose as an example product for sustainable production and consumption. Microbial biotechnology. 2017; 10(5): 1181-1185.
81. Kongruang S. Bacterial cellulose production by Acetobacter xylinum strains from agricultural waste products. Springer. 2007.
82. Carreira P, Mendes J A, Trovatti E, Serafim L S, Freire C S, Silvestre A J and Neto C P. Utilization of residues from agro-forest industries in the production of high value bacterial cellulose. Bioresource technology. 2011; 102(15): 7354-7360.
83. Zhang J, Chang P, Zhang C, Xiong G, Luo H, Zhu Y, Ren K, Yao F and Wan Y. Immobilization of lecithin on bacterial cellulose nanofibers for improved biological functions. Reactive and Functional Polymers. 2015; 91(100-107.
84. Frone A N, Panaitescu D M, Chiulan I, Nicolae C A, Casarica A, Gabor A R, Trusca R, Damian C M, Purcar V and Alexandrescu E. Surface treatment of bacterial cellulose in mild, eco-friendly conditions. Coatings. 2018; 8(6): 221.
85. Lin V S, Lee M C, O'Neal S, McKean J and Sung K P. Ligament tissue engineering using synthetic biodegradable fiber scaffolds. Tissue engineering. 1999; 5(5): 443-451.
86. Gurlek A C, Sevinc B, Bayrak E and Erisken C. Synthesis and characterization of polycaprolactone for anterior cruciate ligament regeneration. Materials Science and Engineering: C. 2017; 71(820-826.
87. Lu HH, Cooper Jr JA, Manuel S, Freeman J W, Attawia M A, Ko F K and Laurencin CT. Anterior cruciate ligament regeneration using braided biodegradable scaffolds: in vitro optimization studies. Biomaterials. 2005; 26(23): 4805-4816.
88. James R, Toti U S, Laurencin C T and Kumbar S G. Electrospun nanofibrous scaffolds for engineering soft connective tissues. Springer. 2011.
89. Panas-Perez E, Gatt C J and Dunn M G. Development of a silk and collagen fiber scaffold for anterior cruciate ligament reconstruction. Journal of Materials Science: Materials in Medicine. 2013; 24(1): 257-265.
90. Chung A S, Hwang H S, Das D, Zuk P, McAllister D R and Wu B M. Lamellar stack formation and degradative behaviors of hydrolytically degraded poly (ε-caprolactone) and poly (glycolide-ε-caprolactone) blended fibers. Journal of Biomedical Materials Research Part B: Applied Biomaterials. 2012; 100(1): 274-284.
91. Wong V W, Gurtner G C and Longaker M T. Wound healing: a paradigm for regeneration. Elsevier. 2013.
92. Lin P—H, Sermersheim M, Li H, Lee PH, Steinberg SM and Ma J. Zinc in wound healing modulation. Nutrients. 2018; 10(1): 16.
93. Li J, Chen J and Kirsner R. Pathophysiology of acute wound healing. Clinics in dermatology. 2007; 25(1): 9-18.
94. Visse R and Nagase H. Matrix metalloproteinases and tissue inhibitors of metalloproteinases: structure, function, and biochemistry. Circulation research. 2003; 92(8): 827-839.
95. Abercrombie M, Flint M and James D. Wound contraction in relation to collagen formation in scorbutic guinea-pigs. Development. 1956; 4(2): 167-175.
96. Barrientos S, Stojadinovic O, Golinko MS, Brem H and Tomic-Canic M. Growth factors and cytokines in wound healing. Wound repair and regeneration. 2008; 16(5): 585-601.
97. West R V and Hamer C D. Graft selection in anterior cruciate ligament reconstruction. JAAOS-Journal of the American Academy of Orthopaedic Surgeons. 2005; 13(3): 197-207.
98. Sadtler K, Allen B W, Estrellas K, Housseau F, Pardoll D M and Elisseeff J H. The scaffold immune microenvironment: biomaterial-mediated immune polarization in traumatic and nontraumatic applications. Tissue Engineering Part A. 2017; 23(19-20): 1044-1053.
99. Hays P L, Kawamura S, Deng X—H, Dagher E, Mithoefer K, Ying L and Rodeo S A. The role of macrophages in early healing of a tendon graft in a bone tunnel. JBJS. 2008; 90(3): 565-579.
100. Petersen W and Tillmann B. Structure and vascularization of the cruciate ligaments of the human knee joint. Anatomy and embryology. 1999; 200(3): 325-334.
101. Mosser D M and Edwards J P. Exploring the full spectrum of macrophage activation. Nature reviews immunology. 2008; 8(12): 958-969.
102. Kishore V, Bullock W, Sun X, Van Dyke Wδ and Akkus O. Tenogenic differentiation of human MSCs induced by the topography of electrochemically aligned collagen threads. Biomaterials. 2012; 33(7): 2137-2144.
103. David L, Grood E S, Noyes F R and Zernicke R E. Biomechanics of ligaments and tendons. Exercise and sport sciences reviews. 1978; 6(1): 125-182.
104. Lee C H, Shin H J, Cho I H, Kang Y-M, Kim I A, Park K-D and Shin J-W. Nanofiber alignment and direction of mechanical strain affect the ECM production of human ACL fibroblast. Biomaterials. 2005; 26(11): 1261-1270.
105. Vallés G, Bensiamar F, Crespo L, Arruebo M, Vilaboa N and Saldana L. Topographical cues regulate the crosstalk between MSCs and macrophages. Biomaterials. 2015; 37(124-133.
106. McWhorter F Y, Wang T, Nguyen P, Chung T and Liu W F. Modulation of macrophage phenotype by cell shape. Proceedings of the National Academy of Sciences. 2013; 110(43): 17253-17258.
107. Luu T U, Gott S C, Woo B W, Rao M P and Liu W F. Micro- and nanopatterned topographical cues for regulating macrophage cell shape and phenotype. ACS applied materials & interfaces. 2015; 7(51): 28665-28672.
108. Sulaeva I, Henniges U, Rosenau T and Potthast A. Bacterial cellulose as a material for wound treatment: Properties and modifications. A review. Biotechnology advances. 2015; 33(8): 1547-1571.
109. Svensson A, Nicklasson E, Harrah T, Panilaitis B, Kaplan D, Brittberg M and Gatenholm P. Bacterial cellulose as a potential scaffold for tissue engineering of cartilage. Biomaterials. 2005; 26(4): 419-431.
110. Favi P M, Benson R S, Neilsen N R, Hammonds R L, Bates C C, Stephens C P and Dhar M S. Cell proliferation, viability, and in vitro differentiation of equine mesenchymal stem cells seeded on bacterial cellulose hydrogel scaffolds. Materials Science and Engineering: C. 2013; 33(4): 1935-1944.
111. Klemm D, Schumann D, Udhardt U and Marsch S. Bacterial synthesized cellulose-artificial blood vessels for microsurgery. Progress in polymer science. 2001; 26(9): 1561-1603.
112. Bodin A, Concaro S, Brittberg M and Gatenholm P. Bacterial cellulose as a potential meniscus implant. Journal of tissue engineering and regenerative medicine. 2007; 1(5): 406-408.
113. Zaborowska M, Bodin A, Backdahl H, Popp J, Goldstein A and Gatenholm P. Microporous bacterial cellulose as a potential scaffold for bone regeneration. Acta biomaterialia. 2010; 6(7): 2540-2547.
114. Vielreicher M, Kralisch D, Volkl S, Sternal F, Arkudas A and Friedrich O. Bacterial nanocellulose stimulates mesenchymal stem cell expansion and formation of stable collagen-I networks as a novel biomaterial in tissue engineering. Scientific reports. 2018; 8(1): 1-14.
115. P6rtile R, Moreira S, Andrade F, Domingues L and Gama M. Bacterial cellulose modified using recombinant proteins to improve neuronal and mesenchymal cell adhesion. Biotechnology progress. 2012; 28(2): 526-532.
116. Martinez H, Brackmann C, Enejder A and Gatenholm P. Mechanical stimulation of fibroblasts in micro-channeled bacterial cellulose scaffolds enhances production of oriented collagen fibers. Journal of Biomedical Materials Research Part A. 2012; 100(4): 948-957.
117. Singh M, Ray A R and Vasudevan P. Biodegradation studies on periodate oxidized cellulose. Biomaterials. 1982; 3(1): 16-20.
118. Mou K, Li J, Wang Y, Cha R and Jiang X. 2, 3-Dialdehyde nanofibrillated cellulose as a potential material for the treatment of MRSA infection. Journal of Materials Chemistry B. 2017; 5(38): 7876-7884.
119. Lagin N T. Development of biodegradable antibacterial cellulose based hydrogel membranes for wound healing. International journal of biological macromolecules. 2014; 67(22-27.
120. Wang B, Lv X, Chen S, Li Z, Sun X, Feng C, Wang H and Xu Y. In vitro biodegradability of bacterial cellulose by cellulase in simulated body fluid and compatibility in vivo. Cellulose. 2016; 23(5): 3187-3198.
121. Hu Y and Catchmark J M. In vitro biodegradability and mechanical properties of bioabsorbable bacterial cellulose incorporating cellulases. Acta biomaterialia. 2011; 7(7): 2835-2845.
122. Lee K Y, Buldum G, Mantalaris A and Bismarck A. More than meets the eye in bacterial cellulose: biosynthesis, bioprocessing, and applications in advanced fiber composites. Macromolecular bioscience. 2014; 14(1): 10-32.
123. Kurosumi A, Sasaki C, Yamashita Y and Nakamura Y. Utilization of various fruit juices as carbon source for production of bacterial cellulose by Acetobacter xylinum NBRC 13693. Carbohydrate Polymers. 2009; 76(2): 333-335.
124. Keshk S and Sameshima K. Evaluation of different carbon sources for bacterial cellulose production. African Journal of Biotechnology. 2005; 4(6): 478-482.
125. Ishihara M, Matsunaga M, Hayashi N and Tisler V. Utilization of D-xylose as carbon source for production of bacterial cellulose. Enzyme and Microbial Technology. 2002; 31(7): 986-991.
126. Angela C, Young J, Kordayanti S and Devanthi PVP. Isolation and screening of microbial isolates from kombucha culture for bacterial cellulose production in sugarcane molasses medium. KnE Life Sciences. 2020;
127. Yamamoto H, Horii F and Hirai A. In situ crystallization of bacterial cellulose II. Influences of different polymeric additives on the formation of celluloses Iα and ID at the early stage of incubation. Cellulose. 1996; 3(1): 229-242.
128. Szymanska-Chargot M, Cybulska J and Zdunek A. Sensing the structural differences in cellulose from apple and bacterial cell wall materials by Raman and FT-IR spectroscopy. Sensors. 2011; 11(6): 5543-5560.
129. Rondeau-Mouro C, Bouchet B, Pontoire B, Robert P, Mazoyer J and Buleon A. Structural features and potential texturising properties of lemon and maize cellulose microfibrils. Carbohydrate Polymers. 2003; 53(3): 241-252.
130. Kataoka Y and Kondo T. Quantitative analysis for the cellulose Iα crystalline phase in developing wood cell walls. International Journal of Biological Macromolecules. 1999; 24(1): 37-41.
131. Åkerholm M, Hinterstoisser B and Salmén L. Characterization of the crystalline structure of cellulose using static and dynamic FT-IR spectroscopy. Carbohydrate research. 2004; 339(3): 569-578.
132. Park S, Baker J O, Himmel M E, Parilla P A and Johnson D K. Cellulose crystallinity index: measurement techniques and their impact on interpreting cellulase performance. Biotechnology for biofuels. 2010; 3(1): 1-10.
133. Wada M, Okano T and Sugiyama J. Allomorphs of native crystalline cellulose I evaluated by two equatorial d-spacings. Journal of Wood Science. 2001; 47(2): 124-128.
134. Krishna I M, Manickam V, Shah A and Davergave N. Environmental management: science and engineering for industry. Butterworth-Heinemann. 2017.
135. Wernet G, Bauer C, Steubing B, Reinhard J, Moreno-Ruiz E and Weidema B. The ecoinvent database version 3 (part I): overview and methodology. The International Journal of Life Cycle Assessment. 2016; 21(1218-1230.
136. Ryberg M, Vieira M D, Zgola M, Bare J and Rosenbaum R K. Updated US and Canadian normalization factors for TRACI 2.1. Clean Technologies and Environmental Policy. 2014; 16(329-339.
137. Kaminski K, Jarosz M, Grudzien J, Pawlik J, Zastawnik F, Pandyra P and Kolodziejczyk A M. Hydrogel bacterial cellulose: A path to improved materials for new eco-friendly textiles. Cellulose. 2020; 27(9): 5353-5365.
138. Molina-Ramirez C, Castro M, Osorio M, Torres-Taborda M, Gómez B, Zuluaga R, Gómez C, Gañán P, Rojas OJ and Castro C. Effect of different carbon sources on bacterial nanocellulose production and structure using the low pH resistant strain Komagataeibacter medellinensis. Materials. 2017; 10(6): 639.
139. Samuel O S and Adefusika A M. Influence of size classifications on the structural and solid-state characterization of cellulose materials. IntechOpen. 2019.
140. Gloria T P, Lippiatt B C and Cooper J. Life cycle impact assessment weights to support environmentally preferable purchasing in the United States. Environmental science & technology. 2007; 41(21): 7551-7557.
141. Gullo M, Sola A, Zanichelli G, Montorsi M, Messori M and Giudici P. Increased production of bacterial cellulose as starting point for scaled-up applications. Applied Microbiology and Biotechnology. 2017; 101(22): 8115-8127.
142. Coban E P and Biyik H. Evaluation of different pH and temperatures for bacterial cellulose production in HS (Hestrin-Scharmm) medium and beet molasses medium. African Journal of microbiology research. 2011; 5(9): 1037-1045.
143. Zahan K A, Pa'e N and Muhamad II. Monitoring the effect of pH on bacterial cellulose production and Acetobacter xylinum 0416 growth in a rotary discs reactor. Arabian Journal for Science and Engineering. 2015; 40(1881-1885.
144. Watanabe K and Yamanaka S. Effects of oxygen tension in the gaseous phase on production and physical properties of bacterial cellulose formed under static culture conditions. Bioscience, biotechnology, and biochemistry. 1995; 59(1): 65-68.
145. Anguluri K, La China S, Brugnoli M, Cassanelli S and Gullo M. Better under stress: Improving bacterial cellulose production by Komagataeibacter xylinus K2G30 (UMCC 2756) using adaptive laboratory evolution. Frontiers in Microbiology. 2022; 13(1-13.
146. Steinchen W, Zegarra V and Bange G. (p) ppGpp: magic modulators of bacterial physiology and metabolism. Frontiers in Microbiology. 2020; 11(2072.
147. Embuscado M E, Marks J S and Bemiller J N. Bacterial cellulose. I. Factors affecting the production of cellulose by Acetobacter xylinum. Food Hydrocolloids. 1994; 8(5): 407-418.
148. Nishiyama Y, Langan P and Chanzy H. Crystal structure and hydrogen-bonding system in cellulose Iβ from synchrotron X-ray and neutron fiber diffraction. Journal of the American Chemical Society. 2002; 124(31): 9074-9082.
149. Focher B, Palma M, Canetti M, Torri G, Cosentino C and Gastaldi G. Structural differences between non-wood plant celluloses: evidence from solid state NMR, vibrational spectroscopy and X-ray diffractometry. Industrial Crops and Products. 2001; 13(3): 193-208.
150. Ishida T, Mitarai M, Sugano Y and Shoda M. Role of water-soluble polysaccharides in bacterial cellulose production. Biotechnology and bioengineering. 2003; 83(4): 474-478.
151. Bae S and Shoda M. Production of bacterial cellulose by Acetobacter xylinum BPR2001 using molasses medium in ajar fermentor. Applied microbiology and biotechnology. 2005; 67(1): 45-51.
152. McKenna B A, Mikkelsen D, Wehr J B, Gidley M J and Menzies N W. Mechanical and structural properties of native and alkali-treated bacterial cellulose produced by Gluconacetobacter xylinus strain ATCC 53524. Cellulose. 2009; 16(6): 1047-1055.
153. Goldman E D, Weisse M, Harris N and Schneider M. Estimating the role of seven commodities in agriculture-linked deforestation: Oil palm, soy, cattle, wood fiber, cocoa, coffee, and rubber. Technical Note, World Resources Institute. 2020;
154. Tadesse G L, Guya T K and Walabu M. Impacts of tannery effluent on environments and human health: a review article. Advances in Life Science and Technology. 2017; 54(10):
155. Altieri MA. Ecological impacts of industrial agriculture and the possibilities for truly sustainable farming. Monthly Review. 1998; 50(3): 60.
156. Muthu SS. Assessing the environmental impact oftextiles and the clothing supply chain. Woodhead publishing. 2020.
157. Zhongming Z, Linong L, Xiaona Y, Wangqiang Z and Wei L. 35% of microplastics released into the world's oceans are from synthetic textiles. 2018;
158. Ragusa A, Svelato A, Santacroce C, Catalano P, Notarstefano V, Carnevali O, Papa F, Rongioletti MCA, Baiocco F and Draghi S. Plasticenta: First evidence of microplastics in human placenta. Environment International. 2021; 146(106274.
159. Deng Y, Zhang Y, Lemos B and Ren H. Tissue accumulation of microplastics in mice and biomarker responses suggest widespread health risks of exposure. Scientific reports. 2017; 7(1): 1-10.
160. Chen G, Wu G, Chen L, Wang W, Hong F F and Jönsson L J. Comparison of productivity and quality of bacterial nanocellulose synthesized using culture media based on seven sugars from biomass. Microbial Biotechnology. 2019; 12(4): 677-687.
161. Li Q, McGinnis S, Wong A and Renneckar S. Nanocellulose life cycle assessment. ACS Sustainable Chemistry & Engineering. 2013; 1(8): 919-928.
162. Donini ÍA, De Salvi D T, Fukumoto F K, Lustri W R, Barud H S, Marchetto R, Messaddeq Y and Ribeiro S J. Biosynthesis and recent advances in production of bacterial cellulose. Eclética Quimica. 2010; 35(165-178.
163. Howell B A and Lazar S T. Biobased plasticizers from glycerol/adipic acid hyperbranched poly (ester) s. Industrial & Engineering Chemistry Research. 2019; 58(37): 17227-17234.
164. Puntener A G and Moss S. Ötzi, the Iceman and his leather clothes. CHIMIA International Journal for Chemistry. 2010; 64(5): 315-320.
165. Heth C L. The skin they were in: leather and tanning in antiquity. ACS Publications. 2015.
166. Poletto M, Ornaghi H L and Zattera A J. Native cellulose: structure, characterization and thermal properties. Materials. 2014; 7(9): 6105-6119.
167. Fan M, Dai D and Huang B. Fourier transform infrared spectroscopy for natural fibres. Fourier transform-materials analysis. 2012; 3(45-68.
168. Oh S Y, Yoo D I, Shin Y, Kim H C, Kim H Y, Chung Y S, Park W H and Youk J H. Crystalline structure analysis of cellulose treated with sodium hydroxide and carbon dioxide by means of X-ray diffraction and FTIR spectroscopy. Carbohydrate research. 2005; 340(15): 2376-2391.
169. O'Connor R T, DuPré E F and Mitcham D. Applications of infrared absorption spectroscopy to investigations of cotton and modified cottons: part I: physical and crystalline modifications and oxidation. Textile Research Journal. 1958; 28(5): 382-392.
170. Carrillo F, Colom X, Sunol J and Saurina J. Structural FTIR analysis and thermal characterisation of lyocell and viscose-type fibres. European Polymer Journal. 2004; 40(9): 2229-2234.
171. Montaudo G, Scamporrino E and Vitalini D. The effect of ammonium polyphosphate on the mechanism of thermal degradation of polyureas. Journal of Polymer Science: Polymer Chemistry Edition. 1983; 21(11): 3321-3331.
172. Wang W, Kan Y, Pan H, Pan Y, Li B, Liew K and Hu Y. Phosphorylated cellulose applied for the exfoliation of LDH: An advanced reinforcement for polyvinyl alcohol. Composites Part A: Applied Science and Manufacturing. 2017; 94(170-177.
173. Ablouh E-H, Brouillette F, Taourirte M, Sehaqui H, El Achaby M and Belfkira A. A highly efficient chemical approach to producing green phosphorylated cellulosic macromolecules. RSC advances. 2021; 11(39): 24206-24216.
174. Suflet D M, Chitanu G C and Popa V I. Phosphorylation of polysaccharides: new results on synthesis and characterisation of phosphorylated cellulose. Reactive and Functional Polymers. 2006; 66(11): 1240-1249.
175. Ghanadpour M, Wicklein B, Carosio F and Wigberg L. All-natural and highly flame-resistant freeze-cast foams based on phosphorylated cellulose nanofibrils. Nanoscale. 2018; 10(8): 4085-4095.
176. Zhu N, Cui F, Hu K and Zhu L. Biomedical modification of poly (1-lactide) by blending with lecithin. Journal of Biomedical Materials Research Part A: An Official Journal of The Society for Biomaterials, The Japanese Society for Biomaterials, and The Australian Society for Biomaterials and the Korean Society for Biomaterials. 2007; 82(2): 455-461.
177. Zhang S, Li S—N, Wu Q, Li Q, Huang J, Li W, Zhang W and Wang S. Phosphorus containing group and lignin toward intrinsically flame retardant cellulose nanofibril-based film with enhanced mechanical properties. Composites Part B: Engineering. 2021; 212(108699.
178. Butt C M, Congleton J, Hoffman K, Fang M and Stapleton H M. Metabolites of organophosphate flame retardants and 2-ethylhexyl tetrabromobenzoate in urine from paired mothers and toddlers. Environmental science & technology. 2014; 48(17): 10432-10438.
179. Laufer G, Kirkland C, Morgan A B and Grunlan J C. Intumescent multilayer nanocoating, made with renewable polyelectrolytes, for flame-retardant cotton. Biomacromolecules. 2012; 13(9): 2843-2848.
180. Fang F, Chen X, Zhang X, Cheng C, Xiao D, Meng Y, Ding X, Zhang H and Tian X. Environmentally friendly assembly multilayer coating for flame retardant and antimicrobial cotton fabric. Progress in Organic Coatings. 2016; 90(258-266.
181. Carosio F, Alongi J, Paravidino C and Frache A. Improving the flame retardant efficiency of layer by layer coatings containing deoxyribonucleic acid by post-diffusion of hydrotalcite nanoparticles. Materials. 2017; 10(7): 709.
182. Hebeish A and Guthrie T. The chemistry and technology of cellulosic copolymers. Springer Science & Business Media. 2012.
183. Schiros T N, Antrobus R, Farias D, Chiu Y-T, Joseph C T, Esdaille S, Sanchirico G K, Miquelon G, An D and Russell S T. Microbial nanocellulose biotextiles for a circular materials economy. Environmental Science: Advances. 2022; 1(3): 276-284.
184. Howell B A and Sun W. Biobased plasticizers from tartaric acid, an abundantly available, renewable material. Industrial & Engineering Chemistry Research. 2018; 57(45): 15234-15242.
185. Rahman M and Brazel C S. The plasticizer market: an assessment of traditional plasticizers and research trends to meet new challenges. Progress in polymer science. 2004; 29(12): 1223-1248.
186. Cho S—C, Bhang S—Y, Hong Y—C, Shin M-S, Kim B—N, Kim J-W, Yoo H-J, Cho I H and Kim H—W. Relationship between environmental phthalate exposure and the intelligence of school-age children. Environmental health perspectives. 2010; 118(7): 1027-1032.
187. Franco A, Prevedouros K, Alli R and Cousins I T. Comparison and analysis of different approaches for estimating the human exposure to phthalate esters. Environment international. 2007; 33(3): 283-291.
188. Wittassek M, Koch H M, Angerer J and Bruning T. Assessing exposure to phthalates—the human biomonitoring approach. Molecular nutrition & food research. 2011; 55(1): 7-31.
189. Bocqué M, Voirin C, Lapinte V, Caillol S and Robin J J. Petro-based and bio-based plasticizers: chemical structures to plasticizing properties. Journal of Polymer Science Part A: Polymer Chemistry. 2016; 54(1): 11-33.
190. Lee K W, Chung J W and Kwak S—Y. Highly branched polycaprolactone/glycidol copolymeric green plasticizer by one-pot solvent-free polymerization. ACS Sustainable Chemistry & Engineering. 2018; 6(7): 9006-9017.
191. Cobos M, Gonzilez B, Fernández M J and Fernindez M D. Chitosan-graphene oxide nanocomposites: Effect of graphene oxide nanosheets and glycerol plasticizer on thermal and mechanical properties. Journal of Applied Polymer Science. 2017; 134(30): 45092.
192. Sanyang M L, Sapuan S M, Jawaid M, Ishak M R and Sahari J. Effect of plasticizer type and concentration on tensile, thermal and barrier properties of biodegradable films based on sugar palm (Arenga pinnata) starch. Polymers. 2015; 7(6): 1106-1124.
193. Mikkonen K S, Heikkinen S, Soovre A, Peura M, Serimaa R, Talja R A, Helén H, Hyvönen L and Tenkanen M. Films from oat spelt arabinoxylan plasticized with glycerol and sorbitol. Journal of Applied Polymer Science. 2009; 114(1): 457-466.
194. Laohakunjit N and Noomhorm A. Effect of plasticizers on mechanical and barrier properties of rice starch film. Starch-Stärke. 2004; 56(8): 348-356.
195. Suyatma N E, Tighzert L, Copinet A and Coma V. Effects ofhydrophilic plasticizers on mechanical, thermal, and surface properties of chitosan films. Journal of Agricultural and Food Chemistry. 2005; 53(10): 3950-3957.
196. McHugh T H and Krochta J M. Sorbitol-vs glycerol-plasticized whey protein edible films: integrated oxygen permeability and tensile property evaluation. Journal of agricultural and food chemistry. 1994; 42(4): 841-845.
197. Cielecka I, Szustak M, Kalinowska H, Gendaszewska-Darmach E, Ryngajilo M, Maniukiewicz W and Bielecki S. Glycerol-plasticized bacterial nanocellulose-based composites with enhanced flexibility and liquid sorption capacity. Cellulose. 2019; 26(5409-5426.
198. Sun Y, Meng C, Zheng Y, Xie Y, He W, Wang Y, Qiao K and Yue L. The effects of two biocompatible plasticizers on the performance of dry bacterial cellulose membrane: a comparative study. Cellulose. 2018; 25(5893-5908.
199. Li T, Hu X, Zhang Q, Zhao Y, Wang P, Wang X, Qin B and Lu W. Poly (acrylic acid)-chitosan@tannic acid double-network self-healing hydrogel based on ionic coordination. Polymers for Advanced Technologies. 2020; 31(7): 1648-1660.
200. Rivero S, Garcia M A and Pinotti A. Crosslinking capacity of tannic acid in plasticized chitosan films. Carbohydrate Polymers. 2010; 82(2): 270-276.
201. Rubentheren V, Ward T A, Chee C Y and Tang C K. Processing and analysis of chitosan nanocomposites reinforced with chitin whiskers and tannic acid as a crosslinker. Carbohydrate polymers. 2015; 115(379-387.
202. Heijmen F, Du Pont J, Middelkoop E, Kreis R and Hoekstra M. Cross-linking of dermal sheep collagen with tannic acid. Biomaterials. 1997; 18(10): 749-754.
203. Cao N, Fu Y and He J. Mechanical properties of gelatin films cross-linked, respectively, by ferulic acid and tannin acid. Food Hydrocolloids. 2007; 21(4): 575-584.
204. Zhang X, Do M D, Casey P, Sulistio A, Qiao G G, Lundin L, Lillford P and Kosaraju S. Chemical modification of gelatin by a natural phenolic cross-linker, tannic acid. Journal of agricultural and food chemistry. 2010; 58(11): 6809-6815.
205. Chen C, Yang H, Yang X and Ma Q. Tannic acid: A crosslinker leading to versatile functional polymeric networks: A review. RSC advances. 2022; 12(13): 7689-7711.
206. Guo J, Ping Y, Ejima H, Alt K, Meissner M, Richardson J J, Yan Y, Peter K, Von Elverfeldt D and Hagemeyer C E. Engineering multifunctional capsules through the assembly of metal-phenolic networks. Angewandte Chemie International Edition. 2014; 53(22): 5546-5551.
207. Guo J, Tardy B L, Christofferson A J, Dai Y, Richardson J J, Zhu W, Hu M, Ju Y, Cui J and Dagastine R R. Modular assembly of superstructures from polyphenol-functionalized building blocks. Nature nanotechnology. 2016; 11(12): 1105-1111.
208. Phan ADT, D'Arcy B R and Gidley M J. Polyphenol-cellulose interactions: effects of pH, temperature and salt. Internationaljournal of food science & technology. 2016; 51(1): 203-211.
209. Erel-Unal I and Sukhishvili S A. Hydrogen-bonded multilayers of a neutral polymer and a polyphenol. Macromolecules. 2008; 41(11): 3962-3970.
210. Shutava T, Prouty M, Kommireddy D and Lvov Y. pH responsive decomposable layer-by-layer nanofilms and capsules on the basis of tannic acid. Macromolecules. 2005; 38(7): 2850-2858.
211. Akiyama H, Fujii K, Yamasaki O, Oono T and Iwatsuki K. Antibacterial action of several tannins against Staphylococcus aureus. Journal of antimicrobial chemotherapy. 2001; 48(4): 487-491.
212. Chung K-T, Lu Z and Chou M. Mechanism of inhibition of tannic acid and related compounds on the growth of intestinal bacteria. Food and Chemical Toxicology. 1998; 36(12): 1053-1060.
213. Colak S M, Yapici B M and Yapici A N. Determination of antimicrobial activity of tannic acid in pickling process. Romanian Biotechnological Letters. 2010; 15(3): 5325-5330.
214. Lee J-H, Park J-H, Cho H S, Joo S W, Cho M H and Lee J. Anti-biofilm activities of quercetin and tannic acid against Staphylococcus aureus. Biofouling. 2013; 29(5): 491-499.
215. Fan L, Ma Y, Su Y, Zhang R, Liu Y, Zhang Q and Jiang Z. Green coating by coordination of tannic acid and iron ions for antioxidant nanofiltration membranes. RSC advances. 2015; 5(130): 107777-107784.
216. Fan H, Wang L, Feng X, Bu Y, Wu D and Jin Z. Supramolecular hydrogel formation based on tannic acid. Macromolecules. 2017; 50(2): 666-676.
217. Zhang W, Yang Z—Y, Tang R—C, Guan J-P and Qiao Y—F. Application of tannic acid and ferrous ion complex as eco-friendly flame retardant and antibacterial agents for silk. Journal of Cleaner Production. 2020; 250(119545.
218. Ejima H, Richardson J J, Liang K, Best J P, van Koeverden M P, Such G K, Cui J and Caruso F. One-step assembly of coordination complexes for versatile film and particle engineering. Science. 2013; 341(6142): 154-157.
219. Zhang Z—Y, Sun Y, Zheng Y-D, He W, Yang Y—Y, Xie Y-J, Feng Z—X and Qiao K. A biocompatible bacterial cellulose/tannic acid composite with antibacterial and anti-biofilm activities for biomedical applications. Materials Science and Engineering: C. 2020; 106(110249.
220. Ninan N, Forget A, Shastri V P, Voelcker N H and Blencowe A. Antibacterial and anti-inflammatory pH-responsive tannic acid-carboxylated agarose composite hydrogels for wound healing. ACS applied materials & interfaces. 2016; 8(42): 28511-28521.
221. Atykyan N, Revin V and Shutova V. Raman and F T-I R Spectroscopy investigation the cellulose structural differences from bacteria Gluconacetobacter sucrofermentans during the different regimes of cultivation on a molasses media. AMB Express. 2020; 10(1): 1-11.
222. Wang Y, Gao J, Yin Q and Hao H. Isolation and characterization of a new polymorph of D-sorbitol. Crystal Research and Technology. 2012; 47(4): 409-414.
223. Mohsin M, Hossin A and Haik Y. Thermomechanical properties of poly (vinyl alcohol) plasticized with varying ratios of sorbitol. Materials Science and Engineering: A. 2011; 528(3): 925-930.
224. Somashekarappa H, Prakash Y, Hemalatha K, Demappa T and Somashekar R. Preparation and characterization of HPMC/PVP blend films plasticized with sorbitol. Indian Journal of Materials Science. 2013; 2013(
225. Sun G, Liang T, Tan W and Wang L. Rheological behaviors and physical properties of plasticized hydrogel films developed from x-carrageenan incorporating hydroxypropyl methylcellulose. Food Hydrocolloids. 2018; 85(61-68.
226. Chen P, Tian H, Zhang L and Chang P R. Structure and properties of soy protein plastics with E-caprolactone/glycerol as binary plasticizers. Industrial & engineering chemistry research. 2008; 47(23): 9389-9395.
227. AlOmar M K, Hayyan M, Alsaadi M A, Akib S, Hayyan A and Hashim M A. Glycerol-based deep eutectic solvents: Physical properties. Journal of Molecular Liquids. 2016; 215(98-103.
228. Lee H—Y, Hwang C—H, Kim H-E and Jeong S—H. Enhancement of bio-stability and mechanical properties of hyaluronic acid hydrogels by tannic acid treatment. Carbohydrate polymers. 2018; 186(290-298.
229. Muhoza B, Xia S and Zhang X. Gelatin and high methyl pectin coacervates crosslinked with tannic acid: The characterization, rheological properties, and application for peppermint oil microencapsulation. Food Hydrocolloids. 2019; 97(105174.
230. Iglesias J, Garcia de Saldaña E and Jaén J. On the tannic acid interaction with metallic iron. Hyperfine Interactions. 2001; 134(109-114.
231. Yuan J and Cheng B. A strategy for nonmigrating highly plasticized PVC. Scientific reports. 2017; 7(1): 9277.
232. Adhikari B, Chaudhary D S and Clerfeuille E. Effect of plasticizers on the moisture migration behavior of low-amylose starch films during drying. Drying Technology. 2010; 28(4): 468-480.
233. Fu Z-q, Wang L-j, Li D, Wei Q and Adhikari B. Effects of high-pressure homogenization on the properties of starch-plasticizer dispersions and their films. Carbohydrate Polymers. 2011; 86(1): 202-207.
234. Krogars K, Heinämäki J, Karjalainen M, Niskanen A, Leskelä M and Yliruusi J. Enhanced stability of rubbery amylose-rich maize starch films plasticized with a combination of sorbitol and glycerol. Internationaljournal of pharmaceutics. 2003; 251(1-2): 205-208.
235. Martinez A W, Caves J M, Ravi S, Li W and Chaikof E L. Effects of crosslinking on the mechanical properties, drug release and cytocompatibility of protein polymers. Acta biomaterialia. 2014; 10(1): 26-33.
236. Kim J, Cai Z, Lee H S, Choi G S, Lee D H and Jo C. Preparation and characterization of a bacterial cellulose/chitosan composite for potential biomedical application. Journal of Polymer Research. 2011; 18(4): 739-744.
237. Loh EYX, Mohamad N, Fauzi M B, Ng M H, Ng S F and Mohd Amin MCI. Development of a bacterial cellulose-based hydrogel cell carrier containing keratinocytes and fibroblasts for full-thickness wound healing. Scientific reports. 2018; 8(1): 2875.
238. Torres F G, Commeaux S and Troncoso O P. Biocompatibility of bacterial cellulose based biomaterials. Journal of functional biomaterials. 2012; 3(4): 864-878.
239. Helenius G, Bäckdahl H, Bodin A, Nannmark U, Gatenholm P and Risberg B. In vivo biocompatibility of bacterial cellulose. Journal of Biomedical Materials Research Part A: An Official Journal of The Society for Biomaterials, The Japanese Society for Biomaterials, and The Australian Society for Biomaterials and the Korean Society for Biomaterials. 2006; 76(2): 431-438.
240. Portal O, Clark W A and Levinson D J. Microbial cellulose wound dressing in the treatment of nonhealing lower extremity ulcers. Wounds: a compendium of clinical research and practice. 2009; 21(1): 1-3.
241. Frankel V H, Serafica G C and Damien C J. Development and testing of a novel biosynthesized XCell for treating chronic wounds. Surgical technology international. 2004; 12(27-33.
242. FDA U. Guidance for industry: Pyrogen and endotoxins testing: questions and answers. U S FDA: Montgomery, M D, USA. 2012;
243. Gorbet M B and Sefton M V. Endotoxin: the uninvited guest. Biomaterials. 2005; 26(34): 6811-6817.
244. Heine H, Rietschel E T and Ulmer A. The biology of endotoxin. Molecular biotechnology. 2001; 19(279-296.
245. Ronni T, Agarwal V, Haykinson M, Haberland M E, Cheng G and Smale S T. Common interaction surfaces of the toll-like receptor 4 cytoplasmic domain stimulate multiple nuclear targets. Molecular and Cellular Biology. 2003; 23(7): 2543-2555.
246. Saitoh S I and Miyake K. Mechanism regulating cell surface expression and activation of Toll-like receptor 4. The Chemical Record. 2006; 6(6): 311-319.
247. Martinez Åvila H, Schwarz S, Feldmann E-M, Mantas A, von Bomhard A, Gatenholm P and Rotter N. Biocompatibility evaluation of densified bacterial nanocellulose hydrogel as an implant material for auricular cartilage regeneration. Applied microbiology and biotechnology. 2014; 98(7423-7435.
248. Pigaleva M A, Bulat M V, Gromovykh T I, Gavryushina I A, Lutsenko S V, Gallyamov M O, Novikov I V, Buyanovskaya A G and Kiselyova O I. A new approach to purification of bacterial cellulose membranes: What happens to bacteria in supercritical media?The Journal of Supercritical Fluids. 2019; 147(59-69.
249. Reneker D H and Chun I. Nanometre diameter fibres of polymer, produced by electrospinning. Nanotechnology. 1996; 7(3): 216.
250. Daigneault M, Preston J A, Marriott H M, Whyte M K and Dockrell D H. The identification of markers of macrophage differentiation in PMA-stimulated THP-1 cells and monocyte-derived macrophages. PloS one. 2010; 5(1): e8668.
251. Park E, Jung H, Yang H, Yoo M, Kim C and Kim K. Optimized THP-1 differentiation is required for the detection of responses to weak stimuli. Inflammation research. 2007; 56(45-50.
252. Chanput W, Mes J J, Savelkoul H F and Wichers H J. Characterization of polarized THP-1 macrophages and polarizing ability of LPS and food compounds. Food & function. 2013; 4(2): 266-276.
253. Chanput W, Mes J J and Wichers H J. THP-1 cell line: an in vitro cell model for immune modulation approach. International immunopharmacology. 2014; 23(1): 37-45.
254. Kim Y-J, Sah R L, Doong J-Y H and Grodzinsky A J. Fluorometric assay of DNA in cartilage explants using Hoechst 33258. Analytical biochemistry. 1988; 174(1): 168-176.
255. Ellis G. The maillard reaction. Elsevier. 1959.
256. Martins S I, Jongen W M and Van Boekel M A. A review of Maillard reaction in food and implications to kinetic modelling. Trends in food science & technology. 2000; 11(9-10): 364-373.
257. Sandle T. A practical approach to depyrogenation studies using bacterial endotoxin. Journal of GXP Compliance. 2011; 15(4): 90.
258. Zhijiang C, Chengwei H and Guang Y. Retracted: Preparation and characterization of a bacterial cellulose/chitosan composite for potential biomedical application. Journal of applied polymer science. 2011; 121(3): 1488-1494.
259. Dinand E, Vignon M, Chanzy H and Heux L. Mercerization of primary wall cellulose and its implication for the conversion of cellulose I→cellulose I I. Cellulose. 2002; 9(7-18.
260. Zugenmaier P. Conformation and packing of various crystalline cellulose fibers. Progress in polymer science. 2001; 26(9): 1341-1417.
261. Lee H S, Stachelek S J, Tomczyk N, Finley M J, Composto R J and Eckmann D M. Correlating macrophage morphology and cytokine production resulting from biomaterial contact. Journal of biomedical materials research Part A. 2013; 101(1): 203-212.
262. Daneshmandi S, Hajimoradi M, Soleimani N and Sattari M. Modulatory effect of Acetobacter xylinum cellulose on peritoneal macrophages. Immunopharmacology and immunotoxicology. 2011; 33(1): 164-168.
263. Jing Y, Ma X, Xu C, Tian H-1 and Chen S-w. Repair of dural defects with electrospun bacterial cellulose membranes in a rabbit experimental model. Materials Science and Engineering: C. 2020; 117(111246.
264. Junka A, Fijalkowski K, Z4bek A, Mikolajewicz K, Chodaczek G, Szymczyk P, Smutnicka D, Zywicka A, Sedghizadeh P P and Dziadas M. Correlation between type of alkali rinsing, cytotoxicity of bio-nanocellulose and presence of metabolites within cellulose membranes. Carbohydrate polymers. 2017; 157(371-379.
265. Lo C-M, Wang H—B, Dembo M and Wang Y-l. Cell movement is guided by the rigidity of the substrate. Biophysical journal. 2000; 79(1): 144-152.
266. Bray D. Cell movements: from molecules to motility. Garland Science. 2000.
267. Kumbar S, James R, Nukavarapu S and Laurencin C. Electrospun nanofiber scaffolds: engineering soft tissues. Biomedical materials. 2008; 3(3): 034002.
268. Yoshimoto H, Shin Y, Terai H and Vacanti J. A biodegradable nanofiber scaffold by electrospinning and its potential for bone tissue engineering. Biomaterials. 2003; 24(12): 2077-2082.
269. Garrigues N W, Little D, Sanchez-Adams J, Ruch D S and Guilak F. Electrospun cartilage-derived matrix scaffolds for cartilage tissue engineering. Journal of biomedical materials research Part A. 2014; 102(11): 3998-4008.
270. Li W J, Danielson K G, Alexander P G and Tuan R S. Biological response of chondrocytes cultured in three-dimensional nanofibrous poly (ϵ-caprolactone) scaffolds. Journal of Biomedical Materials Research Part A: An Official Journal of The Society for Biomaterials, The Japanese Society for Biomaterials, and The Australian Society for Biomaterials and the Korean Society for Biomaterials. 2003; 67(4): 1105-1114.
271. Folkman J and Moscona A. Role of cell shape in growth control. Nature. 1978; 273(5661): 345-349.
272. Garg K, Sell S, Madurantakam P and Bowlin G. Angiogenic potential of human macrophages on electrospun bioresorbable vascular grafts. Biomedical Materials. 2009; 4(3): 031001.
273. Smith M J, Smith D C, Bowlin G L and White K L. Modulation of murine innate and acquired immune responses following in vitro exposure to electrospun blends of collagen and polydioxanone. Journal of Biomedical Materials Research Part A: An Official Journal of The Society for Biomaterials, The Japanese Society for Biomaterials, and The Australian Society for Biomaterials and the Korean Society for Biomaterials. 2010; 93(2): 793-806.
274. Smith M J, White Jr K L, Smith D C and Bowlin G L. In vitro evaluations of innate and acquired immune responses to electrospun polydioxanone-elastin blends. Biomaterials. 2009; 30(2): 149-159.
275. Cao H, Mchugh K, Chew S Y and Anderson J M. The topographical effect of electrospun nanofibrous scaffolds on the in vivo and in vitro foreign body reaction. Journal of Biomedical Materials Research Part A: An Official Journal of The Society for Biomaterials, The Japanese Society for Biomaterials, and The Australian Society for Biomaterials and the Korean Society for Biomaterials. 2010; 93(3): 1151-1159.
276. Garg K, Pullen N A, Oskeritzian C A, Ryan J J and Bowlin G L. Macrophage functional polarization (M1/M2) in response to varying fiber and pore dimensions of electrospun scaffolds. Biomaterials. 2013; 34(18): 4439-4451.
277. Saino E, Focarete M L, Gualandi C, Emanuele E, Cornaglia A I, Imbriani M and Visai L. Effect of electrospun fiber diameter and alignment on macrophage activation and secretion of proinflammatory cytokines and chemokines. Biomacromolecules. 2011; 12(5): 1900-1911.
278. Ballotta V, Driessen-Mol A, Bouten C V and Baaijens F P. Strain-dependent modulation of macrophage polarization within scaffolds. Biomaterials. 2014; 35(18): 4919-4928.
279. Schoenenberger A D, Tempfer H, Lehner C, Egloff J, Mauracher M, Bird A, Widmer J, Maniura-Weber K, Fucentese S F and Traweger A. Macromechanics and polycaprolactone fiber organization drive macrophage polarization and regulate inflammatory activation of tendon in vitro and in vivo. Biomaterials. 2020; 249(120034.
280. Xu H, Guan J, Jin Z, Yin C, Wu S, Sun W, Zhang H and Yan B. Mechanical force modulates macrophage proliferation via Piezol-AKT-Cyclin D1 axis. The FASEB Journal. 2022; 36(8): e22423.
281. De Zee M, Bojsen-Møller F and Voigt M. Dynamic viscoelastic behavior of lower extremity tendons during simulated running. Journal of Applied Physiology. 2000; 89(4): 1352-1359.
282. Nirmalanandhan V S, Shearn J T, Juncosa-Melvin N, Rao M, Gooch C, Jain A, Bradica G and Butler D L. Improving linear stiffness of the cell-seeded collagen sponge constructs by varying the components of the mechanical stimulus. Tissue Engineering Part A. 2008; 14(11): 1883-1891.
283. Riboh J, Chong A K, Pham H, Longaker M, Jacobs C and Chang J. Optimization of flexor tendon tissue engineering with a cyclic strain bioreactor. The Journal of hand surgery. 2008; 33(8): 1388-1396.
284. Hanson A D, Marvel S W, Bernacki S H, Banes A J, van Aalst J and Loboa E G. Osteogenic effects of rest inserted and continuous cyclic tensile strain on hASC lines with disparate osteodifferentiation capabilities. Annals of biomedical engineering. 2009; 37(5): 955-965.
285. Reddy G K and Enwemeka C S. A simplified method for the analysis of hydroxyproline in biological tissues. Clinical biochemistry. 1996; 29(3): 225-229.
286. Enobakhare B O, Bader D L and Lee D A. Quantification of sulfated glycosaminoglycans in chondrocyte/alginate cultures, by use of 1, 9-dimethylmethylene blue. Analytical biochemistry. 1996; 243(1): 189-191.
287. Farndale R W, Sayers C A and Barrett A J. A direct spectrophotometric microassay for sulfated glycosaminoglycans in cartilage cultures. Connective tissue research. 1982; 9(4): 247-248.
288. Shaw L M, Messier J M and Mercurio A M. The activation dependent adhesion of macrophages to laminin involves cytoskeletal anchoring and phosphorylation of the alpha 6 beta 1 integrin. The Journal of cell biology. 1990; 110(6): 2167-2174.
289. Arango Duque G and Descoteaux A. Macrophage cytokines: involvement in immunity and infectious diseases. Frontiers in immunology. 2014; 5(491.
290. Sager H B, Hulsmans M, Lavine K J, Moreira M B, Heidt T, Courties G, Sun Y, Iwamoto Y, Tricot B and Khan O F. Proliferation and recruitment contribute to myocardial macrophage expansion in chronic heart failure. Circulation research. 2016; 119(7): 853-864.
291. Novak M L and Koh T J. Macrophage phenotypes during tissue repair. Journal of leukocyte biology. 2013; 93(6): 875-881.
292. Schnoor M, Cullen P, Lorkowski J, Stolle K, Robenek H, Troyer D, Rauterberg J and Lorkowski S. Production of type V I collagen by human macrophages: a new dimension in macrophage functional heterogeneity. The Journal of Immunology. 2008; 180(8): 5707-5719.
293. Weitkamp B, Cullen P, Plenz G, Robenek H and Rauterberg J. Human macrophages synthesize type VIII collagen in vitro and in the atherosclerotic plaque. The FASEB journal. 1999; 13(11): 1445-1457.
294. Theiss A L, Simmons J G, Jobin C and Lund P K. Tumor necrosis factor (TNF) α increases collagen accumulation and proliferation in intestinal myofibroblasts via TNF receptor 2. Journal of Biological Chemistry. 2005; 280(43): 36099-36109.
295. Kadier A, Ilyas R, Huzaifah M, Harihastuti N, Sapuan S, Harussani M, Azlin M, Yuliasni R, Ibrahim R and Atikah M. Use of industrial wastes as sustainable nutrient sources for bacterial cellulose (B C) production: Mechanism, advances, and future perspectives. Polymers. 2021; 13(19): 3365.
296. Tsouko E, Kourmentza C, Ladakis D, Kopsahelis N, Mandala I, Papanikolaou S, Paloukis F, Alves V and Koutinas A. Bacterial cellulose production from industrial waste and by-product streams. International journal of molecular sciences. 2015; 16(7): 14832-14849.
297. Chang K-L, Hung T-C, Hsieh B—S, Chen Y—H, Chen T-F and Cheng H-L. Zinc at pharmacologic concentrations affects cytokine expression and induces apoptosis of human peripheral blood mononuclear cells. Nutrition. 2006; 22(5): 465-474.
298. Gao H, Dai W, Zhao L, Min J and Wang F. The role of zinc and zinc homeostasis in macrophage function. Journal of immunology research. 2018; 2018(
299. Grangreiff K, Reinhold D and Wedemeyer H. The role of zinc in liver cirrhosis. Annals of hepatology. 2016; 15(1): 7-16.

1. A microbial cellulose material comprising:
an acid;
a metal ion; and
microbial cellulose, wherein
the acid is a polyphenol,
the metal ion is selected from a group consisting of iron ion (Fe²⁺), iron ion (Fe³⁺), copper ion (Cu²⁺), aluminum ion (Al³⁺), magnesium ion (Mg²⁺), zinc ion (Zn²⁺), nickel ion (Ni²⁺), germanium ion (Ge⁴⁺), titanium (Ti⁴⁺), molybdenum (Mo⁶⁺) and tungsten ion (W⁶⁺), and
the microbial cellulose is a bioplasticized microbial cellulose.

2. The microbial cellulose material of claim 1, wherein
the polyphenol is phenolic acid,
the metal ion is one or more selected from a group consisting of Fe²⁺, Fe³⁺, Al³⁺, Cu²⁺, Mg²⁺ and Zn²⁺, and
the microbial cellulose is one or more selected from a group consisting of bioplasticized by polyol, monosaccharide, oligosaccharide, lipid and hyperbranched polyester.

3. The microbial cellulose material of claim 2, wherein
the phenolic acid is selected from a group consisting of hydrolysable tannin, condensed tannin, phlorotannin, tannic acid, caffeic acid, ferulic acid, protocatechuic acid, p-hydroxybenzoic acid, vanillic acid, p-coumaric acid, gallic acid, syringic acid, and sinapinic acid,
the metal ion is one or more selected from a group consisting of Fe²⁺, Fe³⁺, Cu²⁺, Mg²⁺ and Zn²⁺, and
the microbial cellulose is bioplasticized by polyol;
preferably,
the phenolic acid is selected from a group consisting of hydrolysable tannin, condensed tannin, phlorotannin, and tannic acid,
the metal ion is one or more selected from a group consisting of Fe2+, Fe3+, Mg2+ and Zn2+, and
the polyol is selected from a group consisting of glycerol, sorbitol, erythritol, polyester polyols, xylitol, isomalt, lactitol, maltitol, polycaprolactone polyol, mannitol, and polypropylene glycol;
more preferably,
the phenolic acid is tannic acid,
the metal ion is Fe3+, and
the polyol is glycerol or sorbitol.

4. (canceled)

5. (canceled)

6. The microbial cellulose material of claim 1, wherein
a Young's modulus of the microbial cellulose material is from 20 MPa to 300 MPa,
an ultimate tensile strength of the microbial cellulose material is from 0.1 Mpa to 10 Mpa,
a toughness of the microbial cellulose material is from 0.1*10⁴ joule per cubic metre (J·m⁻³) to 180*10⁴ J·m⁻³, and
a ductility of the microbial cellulose material is from 1% to 40%,
preferably, wherein when the pH of the microbial cellulose material is from 1 to 3,
the Young's modulus of the microbial cellulose material is from 20 MPa to 60 MPa,
the ultimate tensile strength of the microbial cellulose material is from 2 Mpa to 5 Mpa,
the toughness of the microbial cellulose material is from 10*104 J·m⁻³ to 50*104 J·m⁻³, and
the ductility of the microbial cellulose material is from 15% to 25%: or
wherein when the pH of the microbial cellulose material is from 4 to 6.5,
the Young's modulus of the microbial cellulose material is from 30 MPa to 60 MPa,
the ultimate tensile strength of the microbial cellulose material is from 2 Mpa to 7 Mpa,
the toughness of the microbial cellulose material is from 50*104 J·m⁻³ to 180*104 J·m⁻³, and
the ductility of the microbial cellulose material is from 25% to 35%; or
wherein when the pH of the complex is from 8-10,
the Young's modulus of the microbial cellulose material is from 60 MPa to 250 MPa,
the ultimate tensile strength of the microbial cellulose material is from 0.1 Mpa to 3.5 Mpa,
the toughness of the microbial cellulose material is from 0.1*104 J·m⁻³ to 5*104 J·m⁻³, and
the ductility of the microbial cellulose material is from 2% to 5%.

7. (canceled)

8. (canceled)

9. (canceled)

10. The microbial cellulose material of claim 1, wherein (a) the microbial cellulose material has an anti-bacteria activity; (b) the microbial cellulose material has a 4-8 mm zone of inhibition; preferably, the microbial cellulose material has a 6-10 mm zone of inhibition.

11. (canceled)

12. (canceled)

13. A process of preparing the microbial cellulose material of claim 1, wherein the process comprises the steps of:
(a) preparing a bioplasticized microbial cellulose;
(b) bathing the bioplasticized microbial cellulose from step (a) in a polyphenol solution;
(c) bathing the bioplasticized microbial cellulose from step (b) in a metal ion solution; and
(d) optionally adding a base to the metal ion solution in step (c).

14. The process of claim 13, wherein
the bioplasticized microbial cellulose from step (a) is first bathed in water and then dried before step (b),
in step (a), the bioplasticized microbial cellulose is bathed in the polyphenol solution for a period of 12-72 hours; preferably, for a period of 12-36 hours; more preferably, for a period of 12-24 hours; more preferably, for 24 hours,
the bioplasticized microbial cellulose from step (b) is first bathed in water and then dried before step (c), and
in step (b), the bioplasticized microbial cellulose is bathed in the metal ion solution for a period of 0.5-12 hours; preferably, for a period of 0.5-6 hours; more preferably, for a period of 1-3 hours; more preferably, for 2 hours.

15. The process of claim 14, wherein the bioplasticized microbial cellulose is prepared by bathing hydrated microbial cellulose in a polyol solution.

16. The process of claim 15, wherein (a) the hydrated microbial cellulose is bathed in the polyol solution for a period of 12-72 hours; preferably, for a period of 12-36 hours; more preferably, for a period of 12-24 hours; more preferably, for 24 hours; (b) the polyol is selected from a group consisting of glycerol, sorbitol, erythritol, polyester polyols, xylitol, isomalt, lactitol, maltitol, polycaprolactone polyol, mannitol, and polypropylene glycol; preferably, the polyol is glycerol or sorbitol; (c) the polyphenol is a phenolic acid; preferably, the phenolic acid is selected from a group consisting of hydrolysable tannin, condensed tannin phlorotannin, tannic acid, caffeic acid, ferulic acid, protocatechuic acid, p-hydroxybenzoic acid, vanillic acid, p-coumaric acid, gallic acid, syringic acid, and sinapinic acid; more preferably, the phenolic acid is selected from a group consisting of hydrolysable tannin, condensed tannin phlorotannin, and tannic acid; more preferably, the phenolic acid is tannic acid.

17. (canceled)

18. (canceled)

19. The process of claim 15, wherein a concentration of the glycerol or sorbitol is 0.1%-20% weight per volume (w/v): preferably, 0.5%-10% weight per volume (w/v); more preferably, l %-5% weight per volume (w/v); or the tannic acid has a concentration of 0.1-10% w/v;
preferably, 0.1-5% w/v, more preferably, 0.1-2 w/v.

20. (canceled)

21. (canceled)

22. (canceled)

23. (canceled)

24. (canceled)

25. (canceled)

26. (canceled)

27. The process of claim 13, wherein the metal ion is Fe²⁺, Fe³⁺, Cu²⁺, Al³⁺, Mg²⁺, Zn²⁺, Ni²⁺, Ge⁴⁺, Ti⁴⁺, Mo⁶⁺, or W⁶⁺; preferably, the metal ion is Fe2+, Fe3+, Al3+, Cu2+, Mg2+, or Zn2+; more preferably, the metal ion is Fe2+, Fe3+, Mg2+, or Zn2+, more preferably, the metal ion is Fe3+.

28. (canceled)

29. (canceled)

30. (canceled)

31. The process of claim 27, wherein
(a) the metal ion solution is iron chloride (FeCl₃), iron oxide (Fe(OH)₃), iron fluoride (FeF₃), iron thiocyanates (Fe(SCN)₃), or iron salicylate (C₂₁H₁₅FeO₉); preferably, the metal ion solution is FeCl₃, Fe(OH)₃, or FeF₃; more preferably, the metal ion solution is FeCl₃, or FeF₃; more preferably, the metal ion solution is FeCl₃: or (b) the base in step (d) is an inorganic base, preferably, the inorganic base is sodium hydroxide (NaOH), potassium hydroxide (KOH), magnesium hydroxide (Mg(OH)2), calcium hydroxide (Ca(OH)2), or ammonia (NH3); more preferably, the inorganic base is NaOH, or KOH; more preferably, the inorganic base is NaOH.

32. (canceled)

33. The process of claim 13, further comprising rinsing and drying the bioplasticized microbial cellulose from step (c).

34. A process of purifying a microbial cellulose film comprising:
(a) bathing the microbial cellulose film in a cellulase solution;
(b) bathing the microbial cellulose film from step (a) in an alcohol solution;
(c) bathing the microbial cellulose film from step (b) in a basic solution; and
(d) applying a high-pressure steam to the microbial cellulose film in step (c).

35. The process of claim 34, wherein
the microbial cellulose film in step (a) is hydrated,
the microbial cellulose film in step (a) is washed with water before step (b),
the microbial cellulose film in step (a) is bathed in the cellulase solution for a period of 0.5-10 hours; preferably, for a period of 0.5-5 hours; more preferably, for a period of 0.5-2 hours; more preferably, for 1 hour,
the microbial cellulose film in step (c) is washed with water before step (d); preferably, the microbial cellulose film in step (c) is washed with water for a period of 12-72 hours; more preferably, for a period of 12-36 hours; more preferably, for a period of 12-24 hours; more preferably, for 24 hours,
the microbial cellulose film is bathed in the basic solution for a period of 12-72 hours; more preferably, for a period of 12-36 hours; more preferably, for a period of 12-24 hours; more preferably, for 24 hours, and
the high-pressure steam is applied to the microbial cellulose in step (c) for a period of 5-120 minutes; preferably, for a period of 10-60 minutes; more preferably, for a period of 20-40 minutes; more preferably, for a period of 30 minutes.

36. The process of claim 35, wherein (a) a concentration of the cellulase solution is 0.1-10% volume/volume (v/v); preferably, the concentration of the cellulase solution is 0.1-5% v/v; more preferably, the concentration of the cellulase solution is 0.5-2% v/v; more preferably, the concentration of the cellulase solution is 1-2% v/v; more preferably, the concentration of the cellulase solution is 1% v/v; (b) the alcohol solution in step (b) is one of ethanol, methanol, isopropanol, ethylene glycol and glycerol; preferably, the alcohol solution in step (b) is one of ethanol, methanol and isopropanol; more preferably, the alcohol solution in step (b) is ethanol; or (c) the purified microbial cellulose film supports fibroblast viability; and does not stimulate a pro-inflammatory response from macrophages; preferably, the macrophage is naïve THP-1 macrophages.

37. (canceled)

38. The process of claim 33, wherein (a) the concentration of the ethanol is 50-99%; preferably, the concentration of the ethanol is 60-90%; more preferably, the concentration of the ethanol is 60-80%; more preferably, the concentration of the ethanol is 80%; (b) the basic solution is NaOH, KOH, Mg(OH)2 Ca(OH)2, or NH3; more preferably, the basic solution is NaOH, or KOH; more preferable, the basic solution is NaOH.

39. (canceled)

40. The process of claim 38, wherein a concentration of NaOH is 0.01-5M; preferably, the concentration of NaOH is 0.01-2M; more preferably, the concentration of NaOH is 0.05M-1M; more preferably, the concentration of NaOH is 0.1 M.

41. The process of claim 34,
wherein in step (d), the high-pressure steam is applied at a temperature of 100-400 °C; more preferably, the high-pressure steam is applied at a temperature of 150-350 °C;
more preferably, the high-pressure steam is applied at a temperature of 200-300 °C; more preferably, the high-pressure steam is applied at a temperature of 200 °C, and wherein in step (d), the high-pressure steam has a pressure over 15 psi, 50 psi, 100 psi, 200 psi, 250 psi, 500 psi, or 800 psi.

42. The process of claim 34, wherein
a Young's modulus of the purified microbial cellulose film is from 1 MPa to 10 MPa; preferably, from 1.2 MPa to 5 MPa; more preferably, from 1.2 MPa to 3 MPa; more preferable from 1.5 MPa to 2 MPa,
an ultimate tensile strength of the purified microbial cellulose film is from 0.1 Mpa to 5 Mpa; preferably, from 0.2 MPa to 3 MPa; more preferably, from 0.2 MPa to 2 MPa; more preferably, from 0.2 MPa to 1 MPa; more preferably, from 0.2 MPa to 0.5 MPa; more preferably, from 0.2 MPa to 0.4 MPa,
a yield strength of the purified microbial cellulose film is from 0.1 MPa to 5 MPa; preferably, from 0.1 MPa to 3 MPa; more preferably, from 0.1 MPa to 2 MPa; more preferably, from 0.2 MPa to 1 MPa; more preferably, from 0.2 MPa to 0.5 MPa; more preferably, from 0.2 MPa to 0.4 MPa, and
a ductility of the purified microbial cellulose film is about 10%-50% of the ductility of the microbial cellulose film before purification.

43. (canceled)
